(19) United States
(12) Patent Application Publication
(10) Pub. No.: US 2019/0006609 A1
(43) Pub. Date: Jan. 3, 2019

(54) ORGANIC FUNCTIONAL COMPOUND FOR PREPARING ORGANIC ELECTRONIC DEVICE AND APPLICATION THEREOF

(71) Applicant: GUANGZHOU CHINARAY OPTOELECTRONIC MATERIALS LTD., Guangzhou (CN)

(72) Inventors: Junyou PAN, Guangzhou (CN); Xi Yang, Guangzhou (CN)

(21) Appl. No.: 16/028,051

(22) Filed: Jul. 5, 2018

Related U.S. Application Data
(63) Continuation of application No. PCT/CN2016/103660, filed on Oct. 28, 2016.

(30) Foreign Application Priority Data
Jan. 7, 2016 (CN) 201610013162.8

(51) Int. Cl. H01L 51/50; H01L 51/00; C07D 209/86; C07D 403/04; C07D 403/14; C07D 487/04

(52) U.S. Cl. CPC H01L 51/5016; H01L 51/5072; H01L 51/5096; H01L 51/0005; C07D 209/86; C07D 403/04; C07D 403/14; C07D 487/04; H01L 51/0072; H01L 51/006; H01L 51/0058; H01L 51/0054; H01L 51/0067; H01L 51/0085; H01L 51/5056; H01L 51/5092

(57) ABSTRACT

The present invention discloses an organic functional compound for preparing an organic electronic device and an application thereof. The organic functional compound has a general formula (I). The organic functional compound comprises an organic functional group and a solubilizing group, thereby imparting a good solubility and film-forming ability. The organic functional compound also excels in maintaining the performance of the functional group in a device. The organic functional compound and a composition or mixture comprising the organic functional compound have a good printability and film-forming ability, facilitating solution-processing, particularly in printing techniques, and obtaining a high-performance small-molecule organic electronic device, particularly an organic electroluminescent device.

FSG]ₖ (I)

### CROSS-REFERENCE TO THE RELATED APPLICATIONS

This application is a continuation application, under 35 U.S.C. § 111(a), of international application No. PCT/CN2016/103660, filed Oct. 28, 2016, wherein the entirety of said application is incorporated herein by reference. International application No. PCT/CN2016/103660 claims priority to Chinese Patent Application No. CN 201610013162.8, filed Jan. 7, 2016.

### TECHNICAL FIELD

The present disclosure relates to the field of organic electronic device, and in particular to an organic functional compound for preparing organic electronic devices and applications thereof.

### BACKGROUND

Due to the diversity in the synthesis of organic semiconductor material, relatively low preparing cost, and excellent optoelectronic properties, the organic light-emitting diode (OLED) shows great potential in the application of optoelectronic device such as flat panel display and lighting.

In order to improve the light-emitting efficiency of the organic light-emitting diode, a multilayer device structure is generally adopted to realize the separation of functions such as charge injection, transporting, and light emission, thereby improving the light-emitting efficiency and lifetime of the device. At present, the method for realizing a multilayer photoelectric device mainly focuses on vacuum evaporation layer-by-layer. However, the vacuum evaporation process has a high cost and high requirement on processing process, such as an extremely precise shadow mask, thereby limiting the application of organic light-emitting diode as an large-area, low-cost display and lighting device. In contrast, solution process such as inkjet printing and roll-to-roll becomes a very promising technology for preparing an optoelectronic device, especially organic light-emitting diode display, due to the outstanding advantages: no requirement for precision shadow mask, room temperature process, and high material utilization. In order to achieve the printing process, suitable printing inks and materials are the critical.

π-conjugated polymer with good solubility and good film-forming properties, becoming a research hotspot in solution processed organic photoelectric devices in recent years. However, the molecular weights, molecular weight distributions, molecular configurations, and purities are always different from batch to batch, resulting in poor repeatability of materials and corresponding device. Meanwhile, the organic light-emitting diodes based on polymer still have lower performance than evaporated small molecule organic light-emitting diodes.

Compared with polymer, small molecule has a more definite molecular structure, a more mature purification process and more excellent device performance, so that they are more promising to realize the wide application of the organic light-emitting diode in the field of display and lighting. However, the lower molecular weight and rigid aromatic molecular structure make the solubility and film-forming property of the small-molecule material be poor, particularly, make it difficult to form a non-hollow amorphous film with a regular appearance. Although the solubility of certain compound can be improved by modifying the molecular structure, the resulting electronic devices are not as good as those obtained by vacuum evaporation. Currently, small molecule organic light-emitting diodes with high performance are still prepared by vacuum evaporation. There is still no solution on materials for solution processing small molecule organic light-emitting diodes. Therefore, designing and synthesizing organic small molecule functional compounds with good solubility and film-forming properties and corresponding printing inks are particularly important for realizing printed organic light-emitting diodes with high performance.

### SUMMARY

In view of the above, it is necessary to provide an organic functional compound for preparing an organic electronic device having good solubility and film-forming property, and application thereof.

The technical solution of the present disclosure is as follows.

An organic functional compound for preparing an electronic device, wherein the compound has a general structural formula of

FSG]ₖ,

wherein, F is an organic functional structural unit, SG is a solubilizing structural unit, k is an integer of 1-10; SGs are the same or different when k is greater than 1;

the solubilizing structural unit SG has a general structural formula of

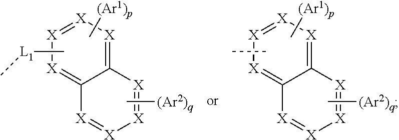

wherein, L₁, Ar¹, and Ar² are each independently selected from aryl or heteroaryl group; p is an integer of 0-3, q is an integer of 0-4, and p+q≥2; in one embodiment, p is 1, q is 1 or 2; the dashed line represents a bond for bonding with the organic functional structural unit F;

X is selected from N or CR¹, adjacent Xs are not simultaneously N, and X is C at the position where Ar¹ and Ar2 are connected; R¹ is selected from at least one of the following groups: H; D; linear alkyl containing 1-20 carbon atoms, linear alkoxy containing 1-20 carbon atoms or linear thioalkoxy containing 1-20 carbon atoms; branched or cyclic alkyl containing 3-20 carbon atoms, branched or cyclic alkoxy or containing 3-20 carbon atoms or branched or cyclic thioalkoxy containing 3-20 carbon atoms; silyl; substituted ketone group containing 1-20 carbon atoms; alkoxycarbonyl containing 2-20 carbon atoms; aryloxycarbonyl containing 7-20 carbon atoms; cyano; carbamoyl; haloformyl; formyl; isocyano; isocyanate; thiocyanate; isothiocyanate group; hydroxy; nitro; CF₃; Cl; Br; F; crosslinkable group; substituted or unsubstituted aromatic or heteroaromatic ring systems containing 5-40 ring atoms; and aryloxy group containing 5-40 ring atoms or heteroaryloxy group containing 5-40 ring atoms; and any combination thereof; wherein one or more of the groups each may combine with the ring bonded thereto to form a monocyclic or polycyclic aliphatic or aromatic ring system.

In one of the embodiments, a molecular weight of the organic functional compound is at least 600 g/mol. In another embodiment, the molecular weight of the organic functional compound is at least 800 g/mol. In a further embodiment, the molecular weight of the organic functional compound is at least 1000 g/mol.

In one of the embodiments, the organic functional structural unit F is selected from groups formed by the following materials: hole injection materials, hole transport materials, hole blocking materials, electron injection materials, electron transport materials, electron blocking materials, organic matrix materials, singlet emitters, triplet emitters, thermally activated delayed fluorescent materials and organic dyes.

In one of the embodiments, the solubilizing structural unit SG is selected from one of the groups shown by the following structural formulas:

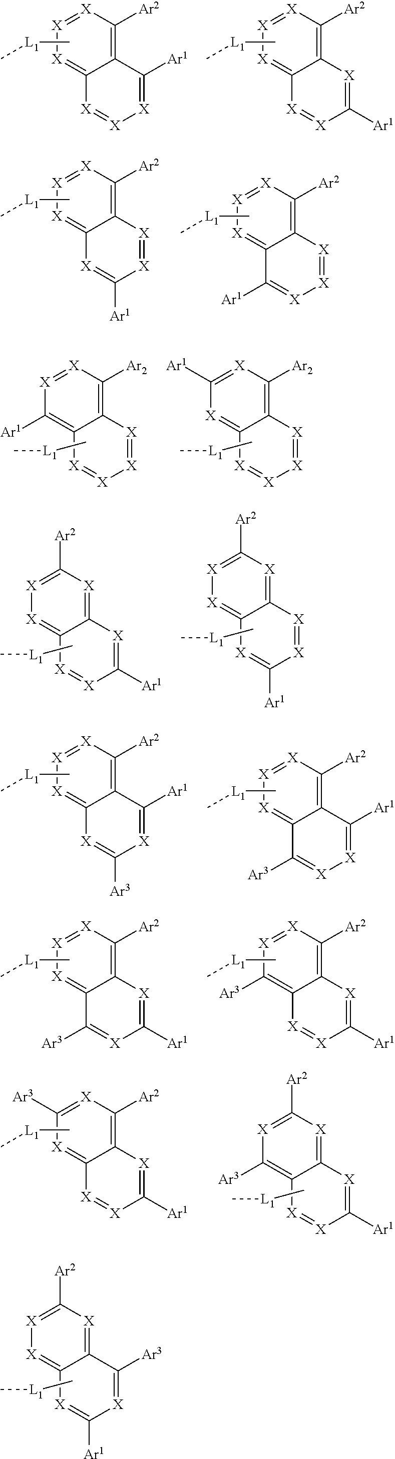

Ar³ is selected from aryl or heteroaryl groups.

In one of the embodiments, the solubilizing structural unit SG is selected from one of the groups shown by the following structural formulas:

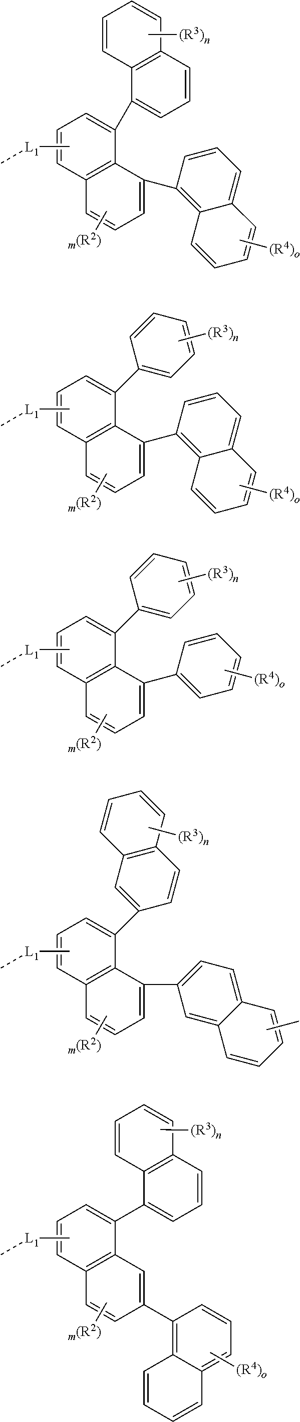

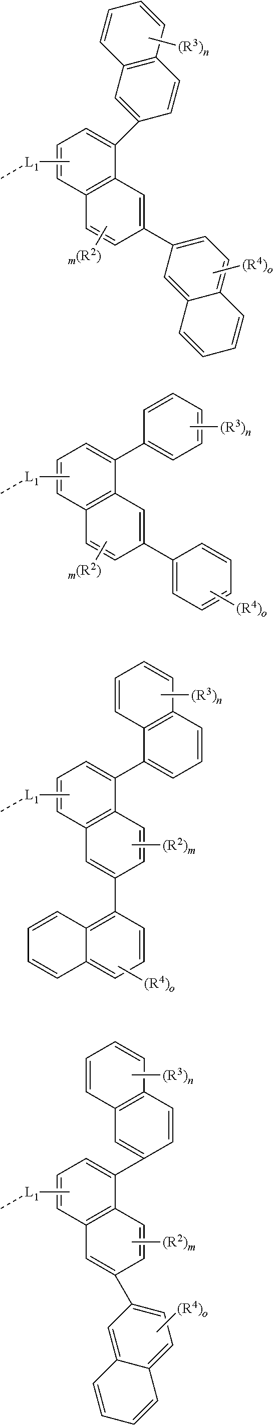

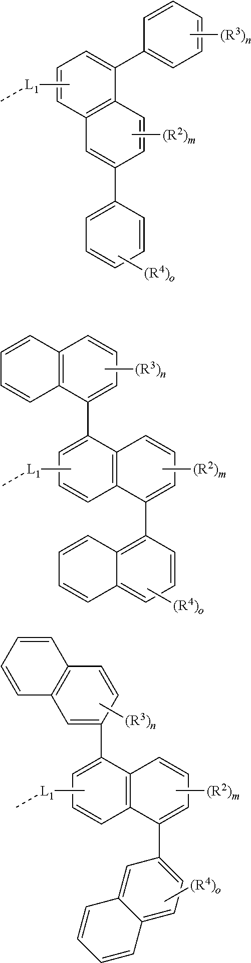

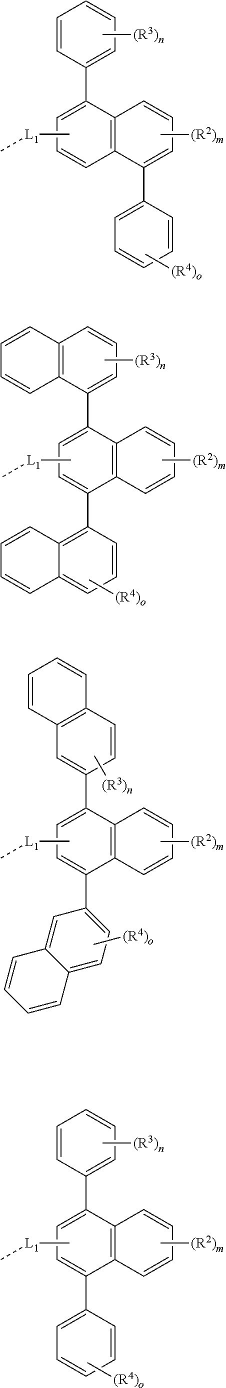

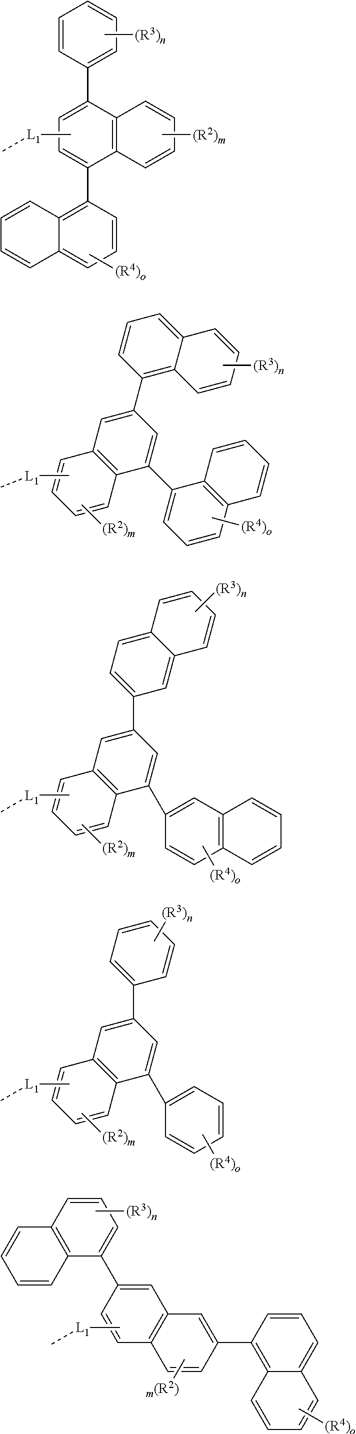

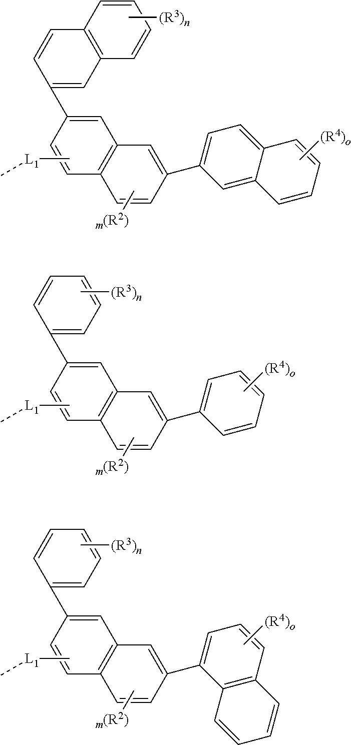

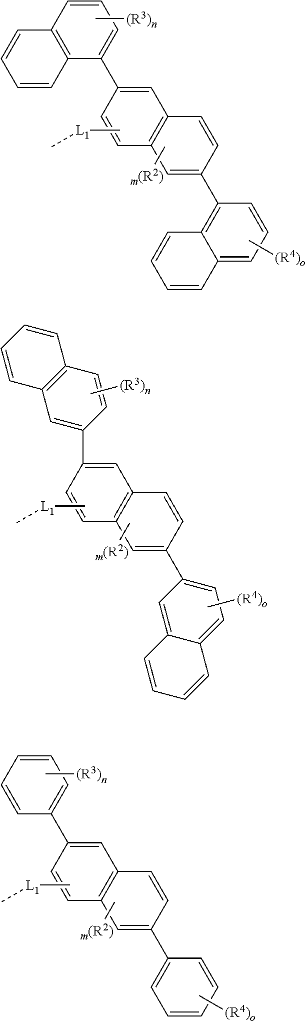

wherein, R², R³ and R⁴ are each independently selected from at least one of the following groups: H; D; linear alkyl containing 1-20 carbon atoms, linear alkoxy containing 1-20 carbon atoms or linear thioalkoxy containing 1-20 carbon atoms; branched or cyclic alkyl containing 3-20 carbon atoms, branched or cyclic alkoxy containing 3-20 carbon atoms or branched or cyclic thioalkoxy containing 3-20 carbon atoms; silyl; substituted keto groups containing 1-20 carbon atoms; alkoxycarbonyl containing 2-20 carbon atoms; aryloxycarbonyl containing 7-20 carbon atoms; cyano; carbamoyl; haloformyl; formyl; isocyano; isocyanate group; thiocyanate group; isothiocyanate group; hydroxy; nitro; CF₃; Cl; Br; F; crosslinkable group; substituted or unsubstituted aromatic or heteroaromatic ring system containing 5-40 ring atoms; and aryloxy group containing 5-40 ring atoms or heteroaryloxy group containing 5-40 ring atoms; and any combination thereof; wherein one or more of the groups each may combine with the ring bonded thereto may to form a monocyclic or polycyclic aliphatic or aromatic ring system.

m is selected from 0, 1, 2, 3, 4 or 5; n and o are each independently selected from 0, 1, 2, 3, 4, 5, 6 or 7.

In one of the embodiments, the total amount of SP³ hybridized groups in the organic functional compound is not more than 30% of the total molecular weight. In another embodiment, the total of SP³ hybridized groups in the organic functional compound is not more than 20% of the total molecular weight. In a further embodiment, the total SP³ hybridized groups in the organic functional compound is not more than 10% of the total molecular weight.

In one of the embodiments, the glass transition temperature of the organic functional compound is not less than 100° C. In another embodiment, the glass transition temperature of the organic functional compound is not less than 120° C. In a further embodiment, the glass transition temperature of the organic functional compound is not less than 140° C. In still a further embodiment, the glass transition temperature of the organic functional compound is not less than 160° C.

In one of the embodiments, the weight ratio of the organic functional structural unit F and the solubilizing structural unit SG is (2:1)-(1:20).

A formulation for preparing an organic electronic device comprises one organic solvent and one organic functional compound as described in any of the above embodiments.

In one of the embodiments, the organic functional compound is a host material.

In one of the embodiments, the formulation further comprises a luminescent material.

In one of the embodiments, the organic solvent is selected from at least one of group consisting of aromatic or heteroaromatic, ester, aromatic ketone or aromatic ether, aliphatic ketone or aliphatic ether, alicyclic or olefinic compound, and inorganic ester compound.

In one of the embodiments, a viscosity of the formulation is in the range of 1 cPs to 100 cPs at 25° C.; and/or a surface tension of the formulation is in the range of 19 dyne/cm to 50 dyne/cm at 25° C.

A mixture for preparing an organic electronic device comprises the organic functional compound as described in any one of the above embodiments and another organic functional material which is selected from the following group : hole injection materials (HIM), hole transport materials (HTM), hole blocking materials (HBM), electron injection materials (EIM), electron transport materials (ETM), electron blocking materials (EBM), organic matrix materials (also known as host material), singlet light emitters (i.e. fluorescent light emitter), triplet light emitters (i.e. phosphorescent light emitter) and organic dyes.

Use of the organic functional compound according to any of the above embodiments, the formulation or the mixture according to any of the above embodiments, in the preparation of an organic electronic device is also provided.

An organic electronic device including the organic functional compound, the formulation, or the mixture according to any of the above embodiments is also provided.

In one of the embodiments, the organic electronic device is an organic light-emitting diode (OLED), an organic photovoltaic cell (OPV), an organic light-emitting electrochemical cell (OLEEC), an organic field effect transistor (OFET), an organic light-emitting field effect transistor, an organic laser, an organic spintronic device, an organic sensor, or an organic plasmon emitting diode.

A method for preparing an organic electronic device is also provided, wherein the method includes applying the organic functional compound according to any of the above embodiments, the formulation, or the mixture according to any of the above embodiments on a substrate by printing or coating process to form a functional layer.

In one of the embodiments, the printing or coating process is inkjet printing, nozzle printing, letterpress printing, screen printing, dip coating, spin coating, blade coating, roller printing, torsion roller printing, lithography, flexographic printing, rotary printing, spray coating, brush coating, pad printing, or slot die coating.

In one of the embodiments, the functional layer has a thickness of 5 nm to 1000 nm.

The organic functional compound for preparing an organic electronic device includes an organic functional structural unit and a solubilizing structural unit, and has good solubility and film-forming property, meanwhile, the organic functional compound well maintains performance of the organic functional structural unit in the device. The organic functional compound, and the formulation, mixture and the like including the organic functional compound, have good printability and film-forming property, and facilitate the realization of high-performance small-molecule organic electronic device, especially organic electroluminescent device, by solution processing, especially printing process, thereby providing a technical solution with a low-cost and a high-efficiency for manufacture.

### BRIEF DESCRIPTION OF THE DRAWINGS

FIG. 1 is a schematic structural diagram of a light-emitting device according to an embodiment; wherein, 101 is a substrate, 102 is an anode, 103 is a hole injection layer and/or a hole transport layer, 104 is a light-emitting layer, 105 is an electron injection layer and/or an electron transport layer, and 106 is a cathode.

### DETAILED DESCRIPTION OF EMBODIMENTS

In order to facilitate understanding of the present disclosure, the disclosure will be described more fully hereinafter. The present disclosure may be embodied in many different forms and should not be limited to the embodiments set forth herein. Rather, these embodiments are provided so that this disclosure will be thorough and complete.

Unless otherwise defined, all terms (including technical and scientific terms) used herein have the same meaning as commonly understood by skilled person in the art to which this disclosure belongs. The terms used herein is for the purpose of describing embodiments only and is not intended to limit the present disclosure. As used herein, the term “and/or” includes any and all combinations of one or more of the associated listed items.

An organic functional compound for preparing an electronic device according to an embodiment has a structural formula of

FSG]ₖ.

Wherein, F is an organic functional structural unit; SG is a solubilizing structural unit; k is an integer of 1-10; in one embodiment, k is an integer greater than or equal to 2; in another embodiment, k is an integer greater than or equal to 3; multiple SG are the same or different when k is greater than 1. In one embodiment, the organic functional compound includes two, three or more solubilizing structural units SG.

An organic functional compound containing a plurality of solubilizing structural units has a relatively high molecular weight, and a higher molecular weight can exhibit more excellent solubility. Therefore, in the present embodiment, the organic functional compound has a molecular weight of at least 600 g/mol. In another embodiment, the organic functional compound has a molecular weight of at least 800 g/mol. In a further embodiment, the organic functional compound has a molecular weight of at least 900 g/mol. In still a further embodiment, the organic functional compound has a molecular weight of at least 1000 g/mol.

The structural formula of the solubilizing structural unit SG is

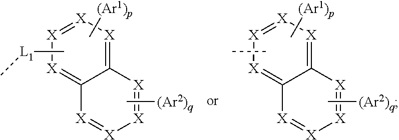

Wherein Ar¹ and Ar² are each independently selected from aryl or heteroaryl groups; Ar¹ and Ar² can be substituted with one or more substituents.

p is an integer of 0-3, q is an integer of 0-4, and p+q≥2; in one embodiment, p is 1 and q is 1 or 2.

L₁ is selected from aryl or heteroaryl groups; wherein the dashed line represents a bond for bonding to the organic functional unit F.

X is selected from N or CR¹, adjacent Xs are not simultaneously N, and X is C at the position where Ar¹ and Ar² are connected.

R¹ is selected from at least one of the following groups: H; D; linear alkyl containing 1-20 carbon atoms, linear alkoxy containing 1-20 carbon atoms or linear thioalkoxy containing 1-20 carbon atoms; branched or cyclic alkyl containing 3-20 carbon atoms, branched or cyclic alkoxy containing 3-20 carbon atoms or branched or cyclic thioalkoxy containing 3-20 carbon atoms; silyl; substituted ketone group containing 1-20 carbon atoms; alkoxycarbonyl containing 2-20 carbon atoms; aryloxycarbonyl containing 7-20 carbon atoms; cyano; carbamoyl; haloformyl; formyl; isocyano; isocyanate group; thiocyanate group; isothiocyanate group; hydroxy; nitro; CF₃; Cl; Br; F; crosslinkable group; substituted or unsubstituted aromatic or heteroaromatic ring systems containing 5-40 ring atoms; aryloxy group containing 5-40 ring atoms or heteroaryloxy group containing 5-40 ring atoms; and any combination thereof; wherein one or more of the groups each may combine with the ring bonded thereto to form a monocyclic or polycyclic aliphatic or aromatic ring system.

The organic functional compound of the present embodiment has a relatively high glass transition temperature that is not less than 100° C. In an embodiment, the glass transition temperature is not less than 120° C. In another embodiment, the glass transition temperature is not less than 140° C. In a further embodiment, the glass transition temperature is not less than 160° C.

The organic functional structural unit F contained in the organic functional compound of the present embodiment is not subject to any limitation, may be any known or newly developed functional compound for an organic electronic device, and is adapted to convert a known functional compound for an organic electronic device into a soluble compound. Therefore, it is not necessary to adjust the electronic property of the organic functional structural unit F; by introducing a solubilizing structural unit SG, the solubility of the functional compound can be achieved, and at the same time the optoelectronic property of the functional unit thereof can be maintained.

In the present embodiment, the organic functional structural unit F is selected from groups formed by one of the following materials: hole injection materials, hole transport materials, hole blocking materials, electron injection materials, electron transport materials, electron blocking materials, organic matrix materials, singlet emitters, triplet emitters, thermally activated delayed fluorescent materials, and organic dyes; particularly, the organic functional structural unit F is a light-emitting metal organic complex.

In the present embodiment, “host material” and “matrix material” have the same meaning and are interchangeable.

In the present embodiment, “metal organic complex” and “organometallic complex” have the same meaning and are interchangeable.

Organic functional material is described in further detail hereinafter. The organic functional material described below may be selected as the organic functional structural unit F, and may also be another functional material which can form a mixture with the organic functional compound.

1. Hole Injection Layer Material (HIM), Hole Transport Layer Material (HTM), and Electron Blocking Layer Material (EBM)

Suitable organic HIM/HTM materials may be selected from compounds having the following structural units: phthalocyanine, porphyrin, amine, aromatic amine, biphenyl triarylamine, thiophene, fused thiophene such as dithiophenethiophene and thiophthene, pyrrole, aniline, carbazole, indolocarbazole, and derivatives thereof. In addition, suitable HIM also includes self-assembling monomers such as compounds containing phosphonic acid and sliane derivatives, metal complexes and cross-linking compounds.

The electron-blocking layer (EBL) used is typically used to block electrons from adjacent functional layers, particularly light emitting layers. In contrast to a light-emitting device without a blocking layer, the presence of EBL usually results in an increase in luminous efficiency. The electron-blocking material (EBM) of the electron-blocking layer (EBL) requires a higher LUMO than the adjacent functional layer, such as the light emitting layer. In another embodiment, the EBM has a greater level of excited energy than the adjacent light-emitting layer, such as a singlet or triplet level, depending on the light emitter. In still another embodiment, the EBM has a hole-transport function. HIM/HTM materials, which typically have high LUMO levels, can be used as EBM.

Examples of cyclic aromatic amine-derived compounds that can be used as HIM, HTM, or EBM may include, but are not limited to, the general structure as follows:

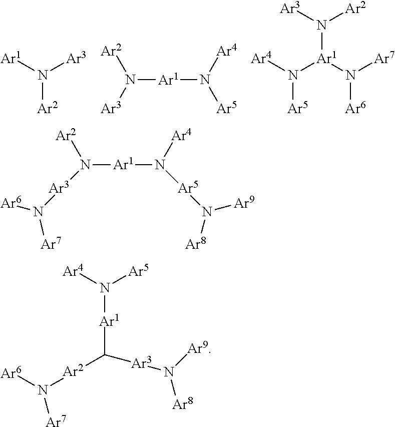

Ar¹-Ar⁹ may be independently selected from: cyclic aromatic compound such as benzene, biphenyl, triphenyl, benzo, naphthalene, anthracene, phenalene, phenanthrene, fluorene, pyrene, chrysene, perylene, azulene; aromatic heterocycle compound such as dibenzothiophene, dibenzofuran, furan, thiophene, benzofuran, benzothiophene, carbazole, pyrazole, imidazole, triazole, isoxazole, thiazole, oxadiazole, oxytriazole, dioxazole, thiadiazole, pyridine, pyridazine, pyrimidine, pyrazine, triazine, oxazine, oxathiazine, oxadiazine, indole, benzimidazole, indazole, indolizine, benzoxazole, benzisoxazole, benzothiazole, quinoline, isoquinoline, cinnoline, quinazoline, quinoxaline, naphthalene, phthalein, pteridine, xanthene, acridine, phenazine, phenothiazine, phenoxazine, dibenzoselenophene, benzoselenophene, benzofuropyridine, indolocarbazole, pyridylindole, pyrrolodipytine, furodipyridine, benzothieopyridine, thienopyridine, benzoselenophenepyridine and selenophenodipyridine; groups each containing 2-10 ring structures that may be the same or different types of cyclic aromatic or aromatic heterocyclic groups and linked one another directly or through at least one of the following groups: e.g. an oxygen atom, a nitrogen atom, a sulfur atom, a silicon atom, a phosphorus atom, a boron atom, a chain structure unit, and an aliphatic ring group. Wherein, Ar¹-Ar⁹ may be further substituted with a substituent which may be selected from at least one of hydrogen, alkyl, alkoxy, amino, alkene, alkyne, aralkyl, heteroalkyl, aryl and heteroaryl.

In one embodiment, Ar¹-Ar⁹ may be each independently selected from the groups comprising the following structures:

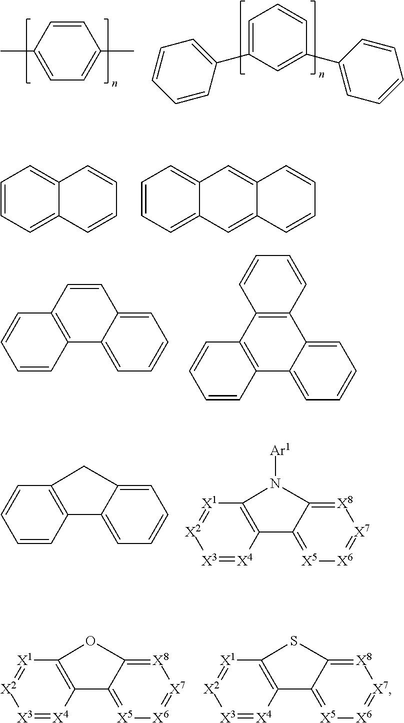

wherein, n is an integer of 1-20; X¹-X⁸ are each independently selected from CH or N; Ar¹ is as defined above.

Examples of metal complex that can be used as HTM or HIM include but are not limited to the following general structure:

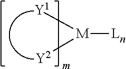

wherein, M is a metal having an atomic weight of more than 40, in one embodiment, M is Ir, Pt, Os and Zn; (Y¹-Y²) is a bidentate ligand, Y¹ and Y² are independently selected from C, N, O, P and S; L is ancillary ligand; m is an integer whose value is from 1 to the maximum coordination number of the metal M; m+n is the maximum coordination number of the metal M.

In an embodiment, (Y¹-Y²) may be a 2-phenylpyridine derivative. In another embodiment, (Y¹-Y²) may be a carbene ligand.

The metal complex has a HOMO greater than −5.5 eV (relative to the vacuum level).

In one embodiment, suitable examples that can be used as HIM/HTM compounds are as follows.

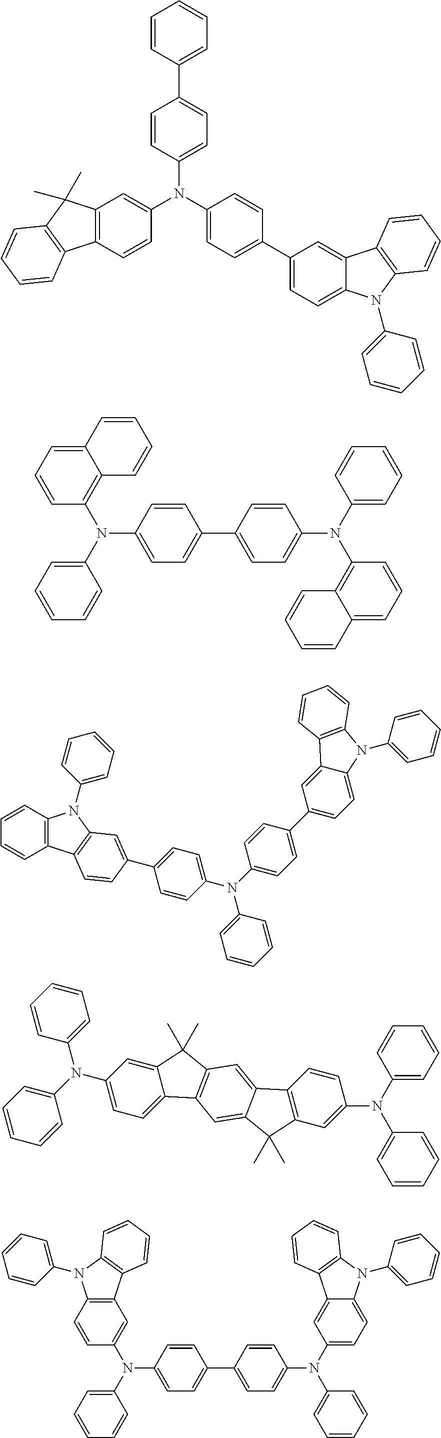

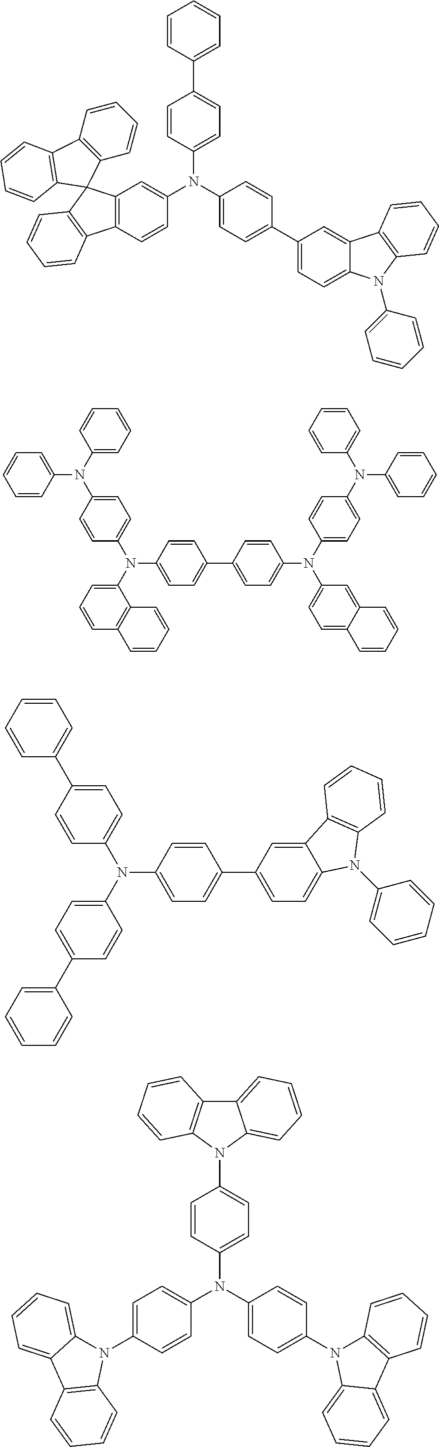 [SMILES: c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1 ; c1ccc(N(c2ccccc2)c2ccc(N(c3ccc(-c4ccc(N(c5ccc(N(c6ccccc6)c6ccccc6)cc5)c5cccc6ccccc56)cc4)cc3)c3ccc4ccccc4c3)cc2)cc1 ; c1ccc(-c2ccc(N(c3ccc(-c4ccccc4)cc3)c3ccc(-c4ccc5c(c4)c4ccccc4n5-c4ccccc4)cc3)cc2)cc1 ; c1ccc2c(c1)c1ccccc1n2-c1ccc(N(c2ccc(-n3c4ccccc4c4ccccc43)cc2)c2ccc(-n3c4ccccc4c4ccccc43)cc2)cc1]

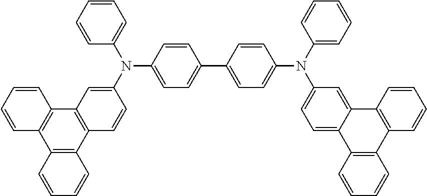 [SMILES: c1ccc(N(c2ccc(-c3ccc(N(c4ccccc4)c4ccc5c6ccccc6c6ccccc6c5c4)cc3)cc2)c2ccc3c4ccccc4c4ccccc4c3c2)cc1]

2. Triplet Host Material

Examples of triplet host material are not particularly limited. Any metal complex or organic compound may be used as a host material as long as its triplet energy is higher than that of a light emitter, particularly a triplet light emitter or phosphorescent light emitter. Examples of metal complex that can be used as a triplet host include, but are not limited to, the following general structure:

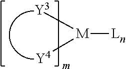

wherein, M is a metal; (Y³-Y⁴) is a bidentate ligand, Y³ and Y⁴ are independently selected from C, N, O, P or S; L is an ancillary ligand; m is an integer whose value is from 1 to the maximum coordination number of the metal; m+n is the maximum coordination number of this metal.

In a further embodiment, the metal complex that can be used as a triplet host has the following form:

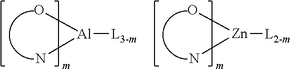

(O—N) is a bidentate ligand in which the metal coordinates with O and N atom.

In other embodiments, M may also be selected from Ir and Pt.

Examples of organic compounds that can be used as a triplet host material are selected from: cyclic aromatic compounds, such as benzene, biphenyl, triphenyl, benzo, fluorene; aromatic heterocyclic compounds, such as dibenzothiophene, dibenzofuran, dibenzoselenophen, furan, thiophene, benzofuran, benzothiophene, benzoselenophen, carbazole, indolocarbazole, pyridine indole, pyrrole dipyridine, pyrazole, imidazole, triazole, oxazole, thiazole, oxadiazole, oxatriazole, dioxazole, thiadiazole, pyridine, pyridazine, pyrimidine, pyrazine, triazine, oxazine, oxathiazine, oxadiazine, indole, benzimidazole, indazole, oxazole, dibenzoxazole, benzisoxazole, benzothiazole, quinoline, isoquinoline, cinnoline, quinazoline, quinoxaline, naphthalene, phthalein, pteridine, xanthene, acridine, phenazine, phenothiazine, phenoxazine, benzofuropyridine, furopyridine, benzothiophene pyridine, thiophene pyridine, benzoselenophenepyridine and selenophenodipyridine; groups having a structure of 2-10 ring atoms, which may be the same or different types of cyclic aromatic or aromatic heterocyclic groups and linked to each other directly or through at least one of the following groups: an oxygen atom, a nitrogen atom, a sulfur atom, a silicon atom, a phosphorus atom, a boron atom, a chain structure unit, and an aliphatic ring group. Wherein, each ring atom may be further substituted with a substituent which may be selected from the group consisting of hydrogen, alkyl, alkoxy, amino, alkene, alkyne, aralkyl, heteroalkyl, aryl and heteroaryl.

In a further embodiment, the triplet host material may be selected from compounds containing at least one of the following groups:

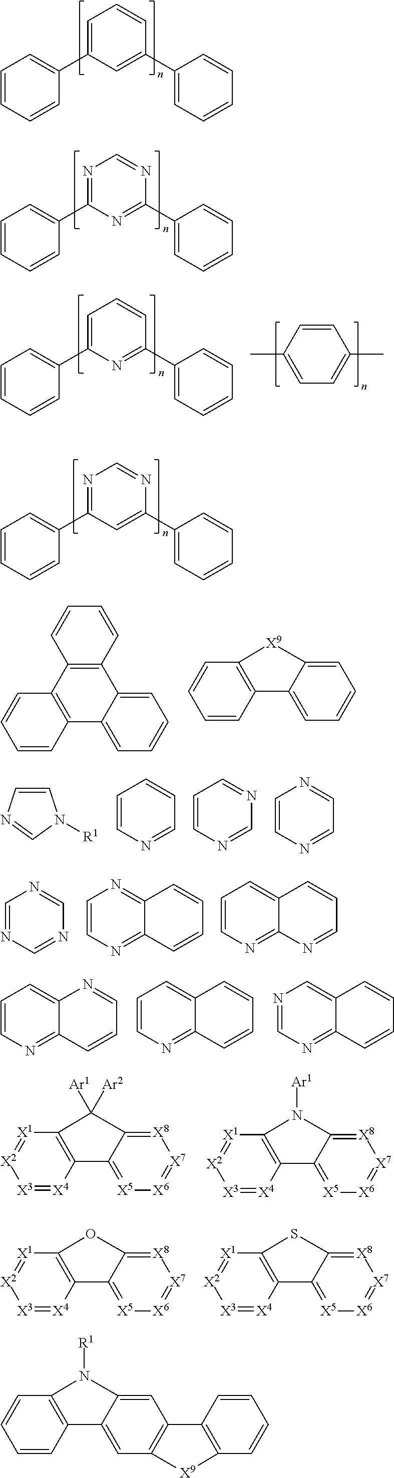

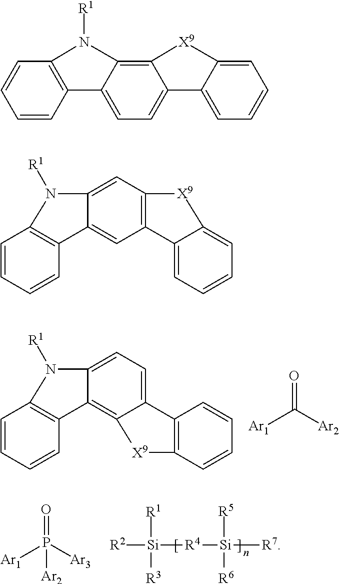

R¹-R⁷ may be independently selected from the following groups: hydrogen, alkyl, alkoxy, amino, alkene, alkyne, aralkyl, heteroalkyl, aryl and heteroaryl, and have the same meaning as Ar¹, Ar² and Ar³ described above when they are aryl or heteroaryl; n is an integer of 0-20; X¹-X⁸ are selected from CH or N; and X⁹ is selected from CR¹R² or NR¹.

Examples of triplet host materials are as follows.

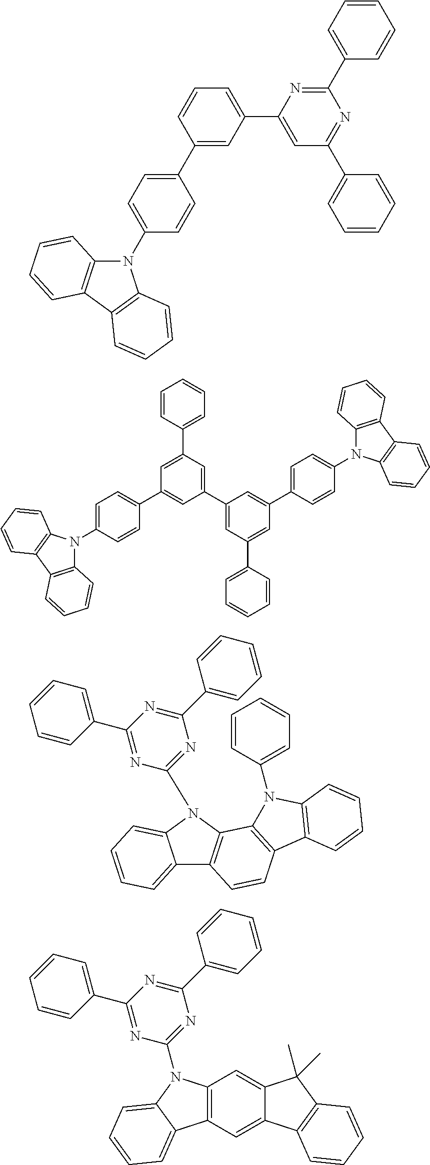

3. Singlet Matrix Material (Singlet Host):

Examples of the singlet host materials are not particularly limited. Any organic compound can be used as the host as long as its singlet energy is higher than that of the light emitter, particularly the singlet light emitter or the fluorescent light emitter.

Examples of organic compounds that can be used as singlet host materials may be selected from: cyclic aromatic compounds such as benzene, biphenyl, triphenyl, benzo, naphthalene, anthracene, phenalene, phenanthrene, fluorene, pyrene, chrysene, perylene, azulene; aromatic heterocycles compounds such as dibenzothiophene, dibenzofuran, dibenzoselenophene, furan, thiophene, benzofuran, benzothiophene, benzoselenophene, carbazole, indolocarbazole, pyridine indole, pyrrolodipytine, pyrazole, imidazole, triazole, isoxazole, thiazole, oxadiazole, oxytriazole, dioxazole, thiadiazole, pyridine, pyridazine, pyrimidine, pyrazine, triazine, oxazine, oxathiazine, oxadiazine, indole, benzimidazole, indazole, indolizine, benzoxazole, benzoxazole, benzothiazole, quinoline, isoquinoline, cinnoline, quinazoline, quinoxaline, naphthalene, phthalein, pteridine, xanthene, acridine, phenazine, phenothiazine, phenoxazine, benzofuropyridine, furan dipyridine, benzothiophene pyridine, thiophenyldipyridine, benzoselenophenepyridine and selenophenodipyridine; groups having a structure of 2-10 ring atoms, which may be the same or different types of cyclic aromatic or aromatic heterocyclic groups and linked to each other directly or through at least one of the following groups: an oxygen atom, a nitrogen atom, a sulfur atom, a silicon atom, a phosphorus atom, a boron atom, a chain structure unit, and an aliphatic ring group.

In a further embodiment, the singlet host material may be selected from compounds containing at least one of the following groups:

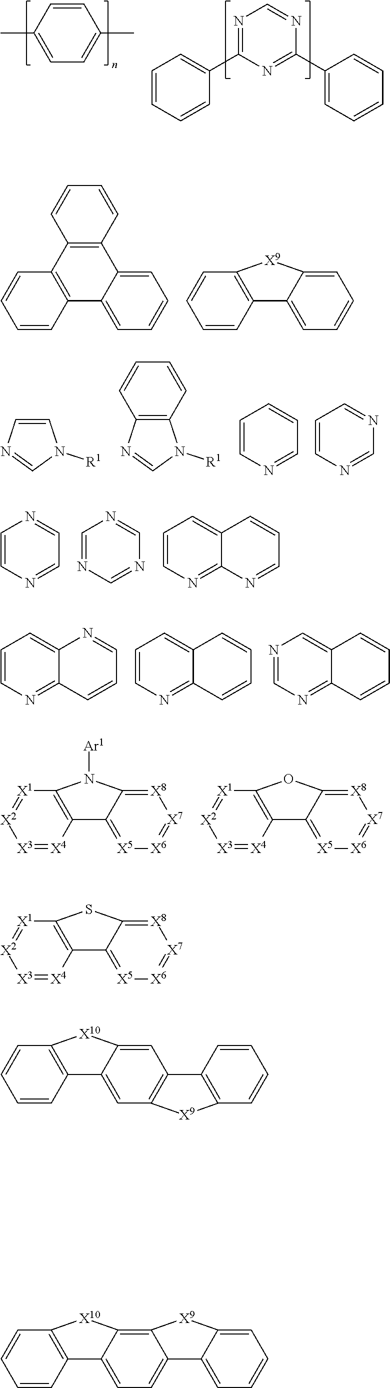

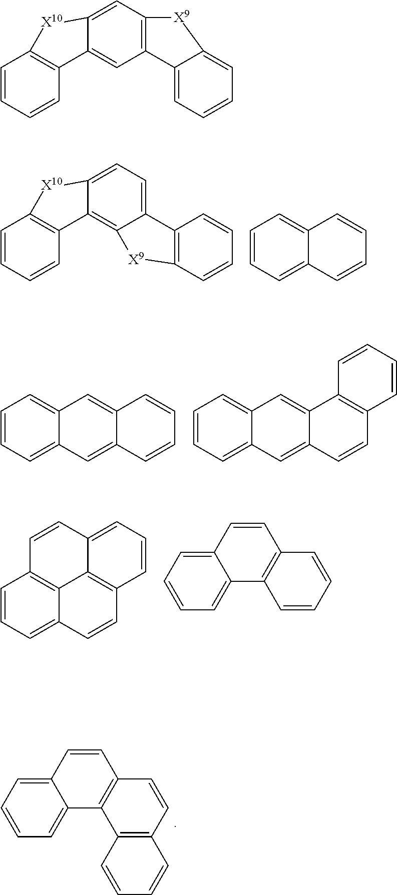

Wherein, R¹ may be selected from the following groups: hydrogen, alkyl, alkoxy, amino, alkene, alkynyl, aralkyl, heteroalkyl, aryl and heteroaryl; Ar¹ is aryl or heteroaryl, and has the same meaning as Ar¹ defined in the above HTM; n is an integer of 0-20; X¹ to X⁸ are each independently selected from CH or N; X⁹ and X¹⁰ are each independently selected from CR¹R² or NR¹.

In one embodiment, examples of the anthryl singlet host material are as follows.

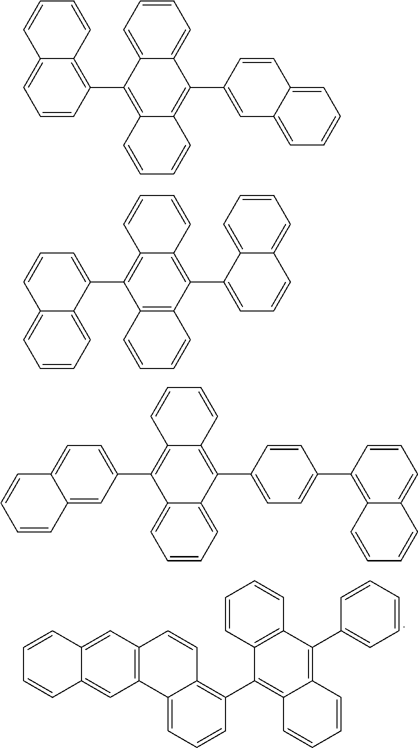

4. Singlet Emitter

Singlet emitter usually has a relatively long conjugated π electron system.

In a further embodiment, the singlet emitter may be selected from monobasic styrylamine, binary styrylamine, ternary styrylamine, quaternary styrylamine, styrene phosphine, styrene ether, or aryl amine.

Mono-styrylamine is a compound which includes an unsubstituted or substituted styryl group and at least one amine, such as an aromatic amine. Di-styrylamine is a compound which includes two unsubstituted or substituted styryl groups and at least one amine, such as an aromatic amine. Tri-styrylamine is a compound which includes three unsubstituted or substituted styryl groups and at least one amine, such as an aromatic amine. Tera-styrylamine is a compound which includes four unsubstituted or substituted styryl groups and at least one amine, such as an aromatic amine. An exemplary styrene is stilbene, which may be further substituted. The definitions of the corresponding phosphines and ethers are similar to those of amines. Arylamine or aromatic amine is a compound which includes three unsubstituted or substituted aromatic or heterocyclic ring systems directly bonded to nitrogen. In one embodiment at least one of the aromatic or heterocyclic ring systems is a fused ring system, such as a fused ring system containing at least 14 aromatic ring atoms. Wherein, exemplary examples are aromatic anthracenamine, aromatic anthryl diamine, aromatic pyrenamine, aromatic pyrenediamine, aromatic chryseneamine and aromatic chrysenediamine. An aromatic anthraceneamine is a compound in which a binary arylamine group is directly coupled to an anthracene, preferably e.g. at the position 9. An aromatic anthryl diamine is a compound in which two binary arylamine groups are directly coupled to an anthracene, e.g. at the position 9, 10. Aromatic pyrenamine, aromatic pyrenyl diamine, aromatic chrysenamine and aromatic chrysenyl diamine are analogously defined, wherein the binary arylamine group is, for example, coupled to the position 1 or 1, 6 of the pyrene.

In one embodiment, singlet emitters are compounds based on vinylamine and aromatic amine.

In a further embodiment, singlet emitter may be selected from indenofluorene-amine and indenofluorene-diamine, benzoindenofluorene-amine and benzoindenofluorene-diamine, dibenzoindenofluorene-amine or dibenzoindenofluorenone-diamine.

Other materials that can be used as singlet emitters include polycyclic aromatic hydrocarbon compounds, in particular, the derivatives of the following compounds: anthracene such as 9,10-Di(2-naphthyl anthracene), naphthalene, tetraphenyl, xanthene, phenanthrene, pyrene such as 2,5,8,11-tetra-t-butylpyrene, indenopyrene, phenylene such as 4,4′-bis(9-ethyl-3-carbazole vinyl)-1,1′-biphenyl, periflanthene, decacyclene, hexabenzobenzene, fluorene, spirobifluorene, arylpyrene (as disclosed in US20060222886), arylene ethylene ((as disclosed in U.S. Pat. No. 5,121,029, U.S. Pat. No. 5,130,603), cyclopentadiene such as tetraphenyl cyclopentadiene, rubrene, coumarin, rhodamine, quinacridone, pyran such as 4-(dicyanomethylene)-6-(4-p-dimethylaminostyryl-2-methyl)-4H-pyran (DCM), thiopyran, bis(azinyl)imine boron compound (US 2007/0092753 A1), bis(azinyl)methylene compound, carbostyryl compound, oxazinone, benzoxazole, benzothiazole, benzimidazole and diketopyrrolopyrrole.

Some suitable examples of singlet light emitters are listed below:

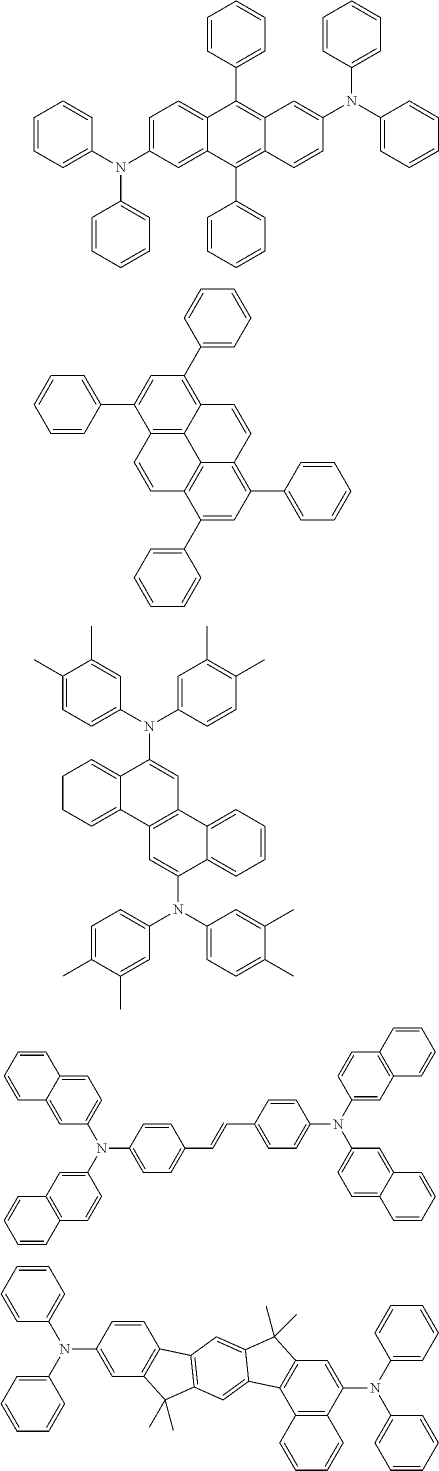

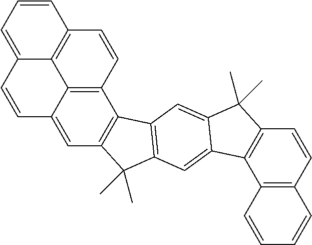 [SMILES: CC1(C)c2cc3c(cc2-c2c1ccc1ccccc21)C(C)(C)c1cc2ccc4cccc5ccc(c1-3)c2c45]

5. Triplet Emitter (Phosphorescent Light Emitter)

A triplet emitter is also known as a phosphorescent light emitter. In a further embodiment, the triplet emitter is a metal complex having the general formula M(L)n. Wherein M is a metal atom; L may be the same or different each time it appears, and L is an organic ligand that is bonded or coordinated to the metal atom M through one or more positions; n is an integer greater than or equal to 1, such as 1, 2, 3, 4, 5 or 6. In one embodiment, these metal complexes are coupled to polymer through one or more positions, for example, through organic ligands.

In a further embodiment, the metal atom M is selected from transition metal elements or lanthanide elements or actinide elements, such as Ir, Pt, Pd, Au, Rh, Ru, Os, Sm, Eu, Gd, Tb, Dy, Re, Cu or Ag. In still a further embodiment, the metal atom M is Os, Ir, Ru, Rh, Re, Pd or Pt.

In one embodiment, the triplet light emitter includes a chelating ligand, i.e. ligand, which coordinates with the metal via at least two binding sites. In another embodiment, the triplet light emitter includes two or three same or different bidentate or multidentate ligands. Chelating ligands help to increase the stability of metal complex.

Examples of the organic ligand may be selected from: phenylpyridine derivatives, 7,8-benzoquinoline derivatives, 2-(2-thienyl)pyridine derivatives, 2-(1-naphthyl)pyridine derivatives, or 2-phenylquinoline derivatives. All of these organic ligands may be substituted, for example, substituted with fluoromethyl or trifluoromethyl. In one embodiment, the ancillary ligand may be selected from acetate acetone or picric acid.

In a further embodiment, the metal complex that can be used as a triplet tight emitter has the following form:

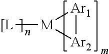

wherein, M is a metal, e.g. a transition metal element or a lanthanide element or an actinide element; Ar₁ may be the same or different each time it appears and Ar₁ is a cyclic group and includes at least one donor atom, i.e., an atom with a lone pair of electrons such as nitrogen or phosphorus, through which lone pair of electrons the cyclic group is coordinately coupled with metal; Ar₂ may be the same or different each time it appears and Ar₂ is a cyclic group and includes at least one carbon atom through which the cyclic group is coupled with metal. Ar₁ and Ar₂ are coupled together by a covalent bond, and each may carry one or more substituent groups, and they may also be coupled together by a substituent group; L may be the same or different each time it appears, and L is an ancillary ligand, e.g. a bidentate chelating ligand, such as a monoanionic bidentate chelating ligand; m is 1, 2 or 3, e.g. 2 or 3, such as 3; n is 0, 1, or 2, e.g. 0 or 1, such as 0;

Some suitable examples of triplet light emitters are listed below.

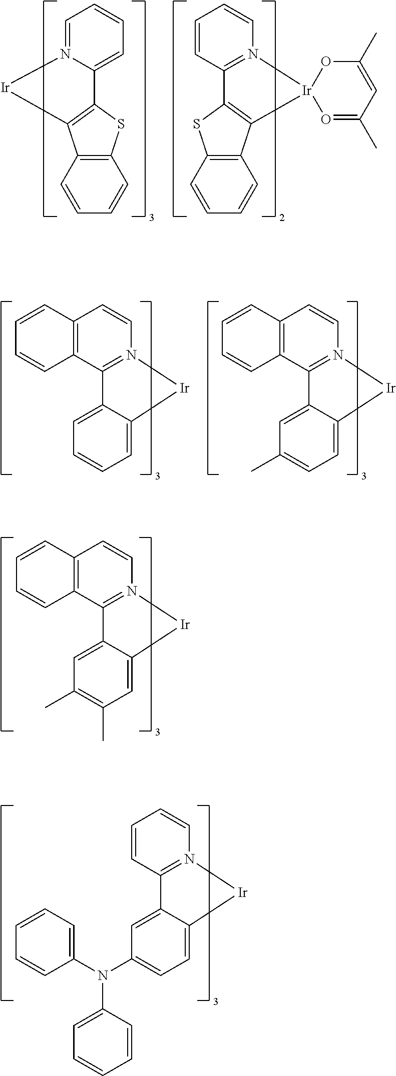

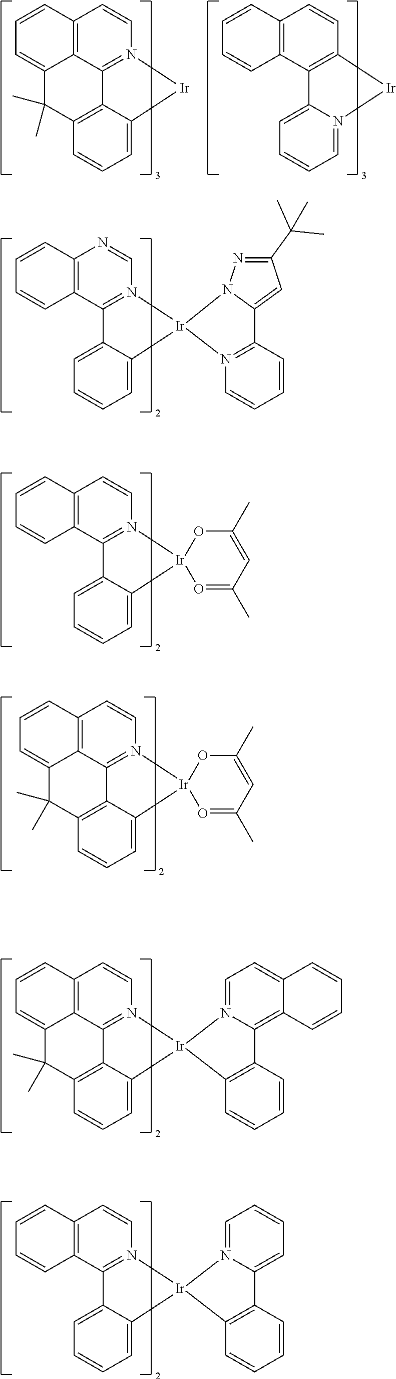

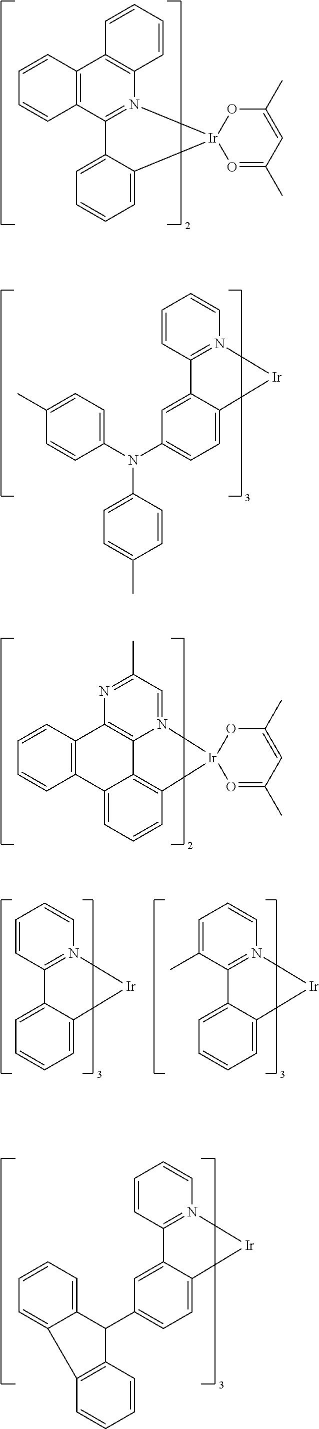

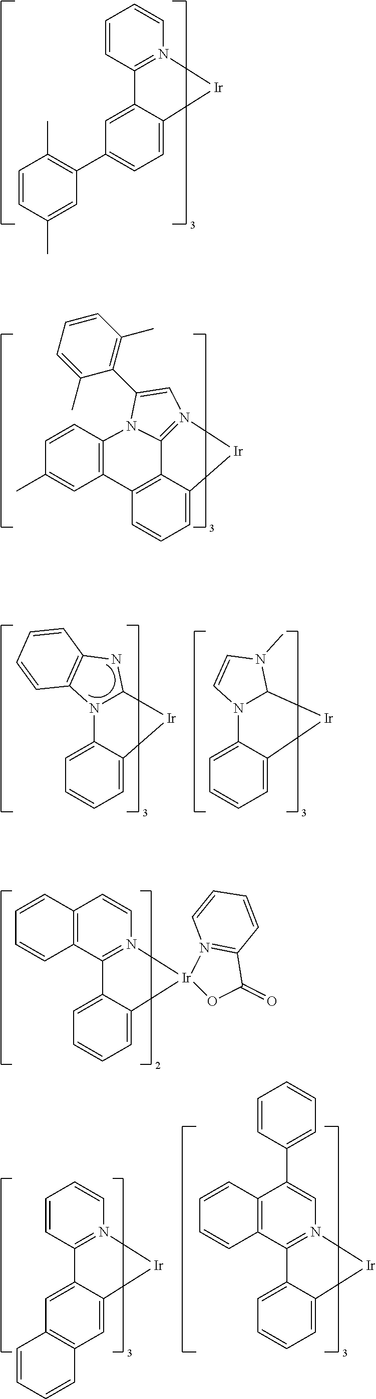

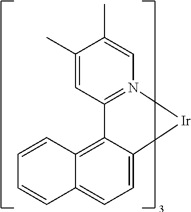

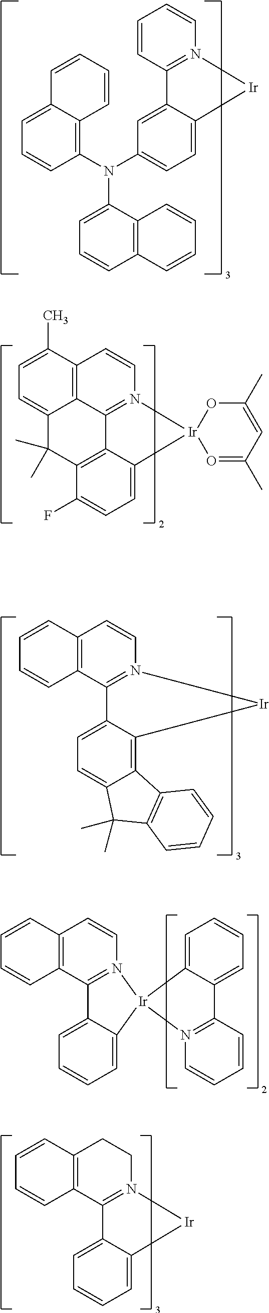

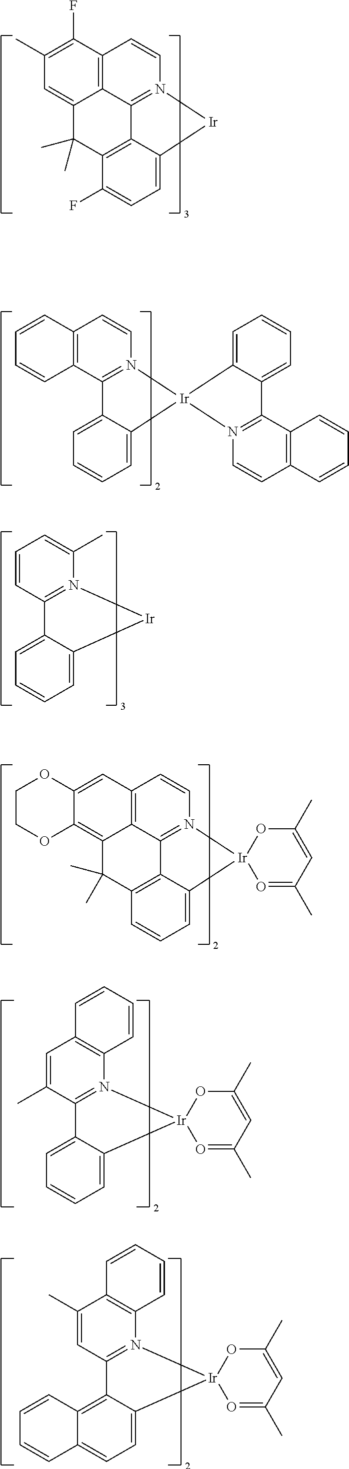

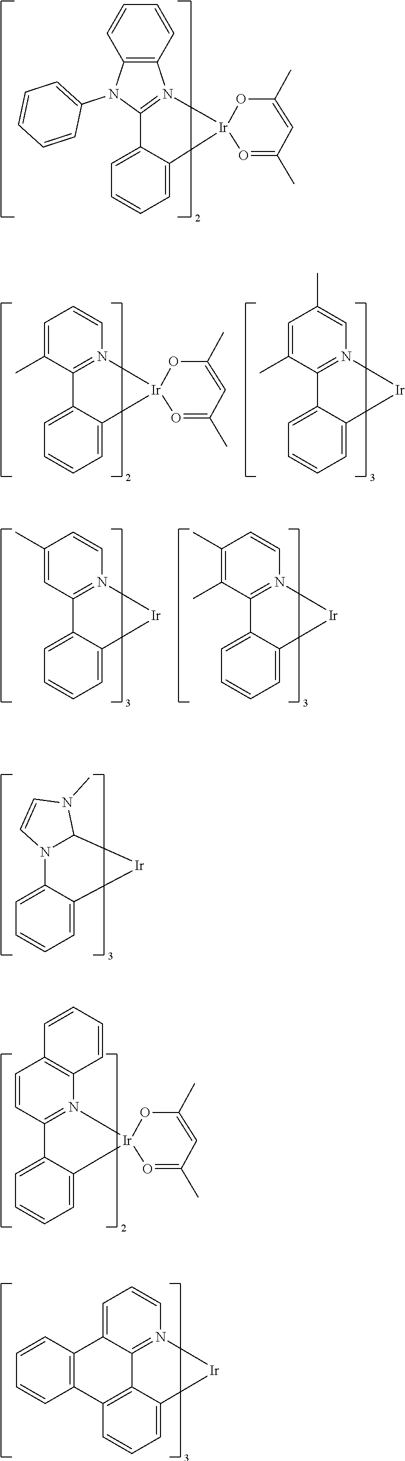

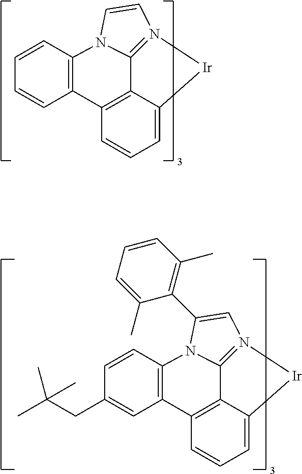

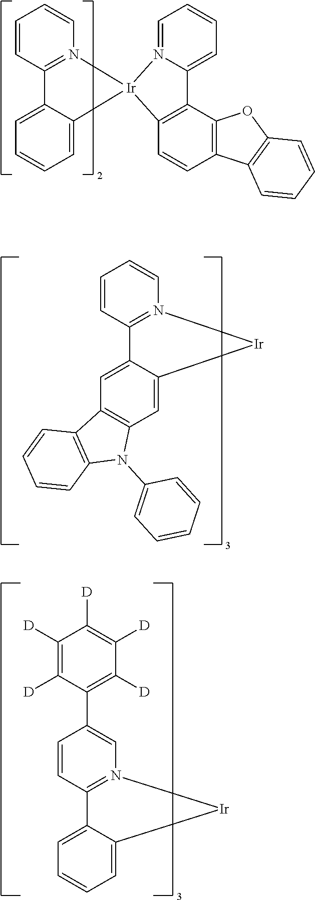

6. Thermally Activated Delayed Fluorescent (TADF) Materials

Traditional organic fluorescent materials can only emit light using 25% singlet exciton formed by electrical excitation, and the device has low internal quantum efficiency (the highest efficiency is 25%). The phosphorescent material enhances the intersystem crossing due to the strong spin-orbit coupling of the heavy atom center, and may emit light effectively using the singlet exciton and the triplet exciton formed by the electric excitation, so that internal quantum efficiency of the device can reach 100%. However, the problems, e.g. the phosphor materials are expensive and poor in material stability, the device efficiency roll-off is serious, etc., limit its application in OLED. Thermally activated delayed fluorescent material is the third generation of organic light-emitting material developed after organic fluorescent material and organic phosphorescent material. Such materials generally have a small singlet-triplet energy level difference (ΔEst), and triplet excitons can be converted to singlet excitons by reverse intersystem crossing. Thus, singlet excitons and triplet excitons formed under electric excitation can be fully utilized. The internal quantum efficiency of the device can reach 100%. At the same time, due to the controllable material structure, the stable properties, the low price, and no need of using precious metals, thus the application prospect in the OLED field is promising.

TADF material needs to have a smaller singlet-triplet energy level difference, e.g. ΔEst<0.3 eV; in one embodiment, ΔEst<0.2 eV; in another embodiment, ΔEst<0.1 eV. In a further embodiment, TADF material has a relatively small ΔEst, and in another preferred embodiment, TADF has better fluorescence quantum efficiency.

Some suitable examples of TADF luminescent materials are listed below.

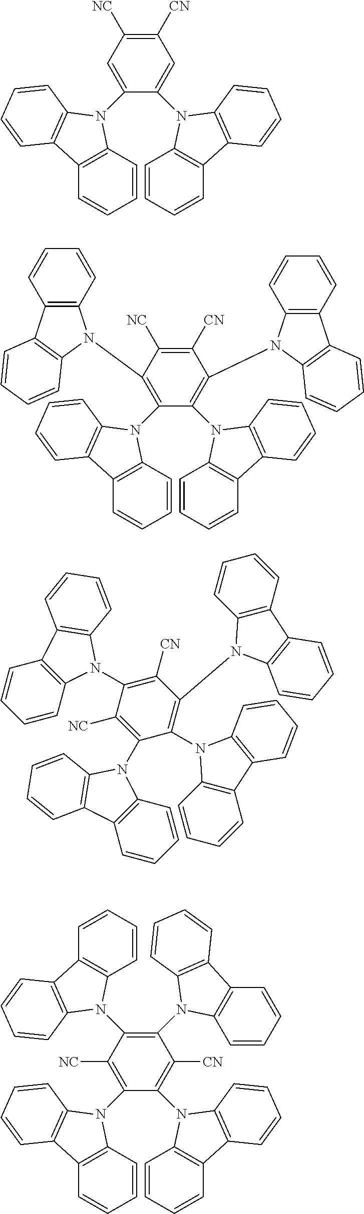

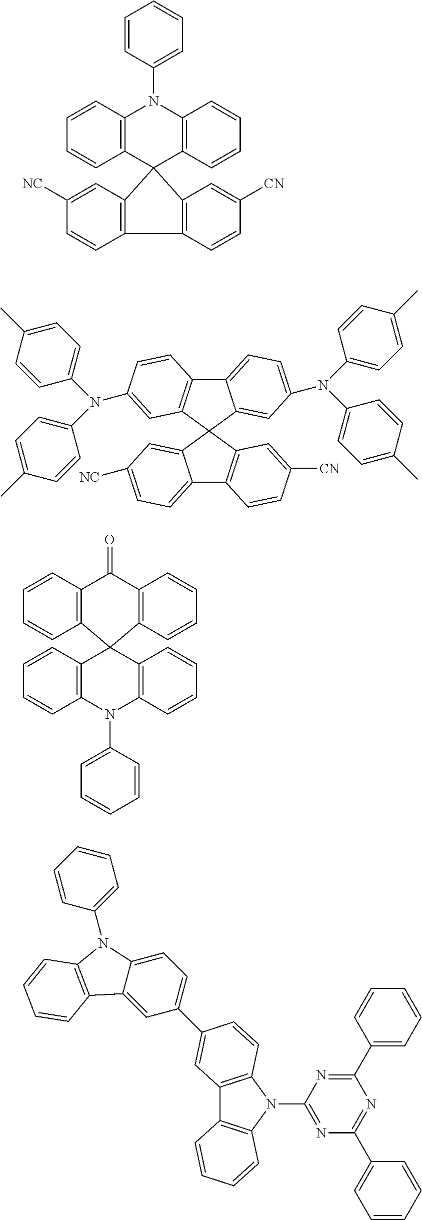

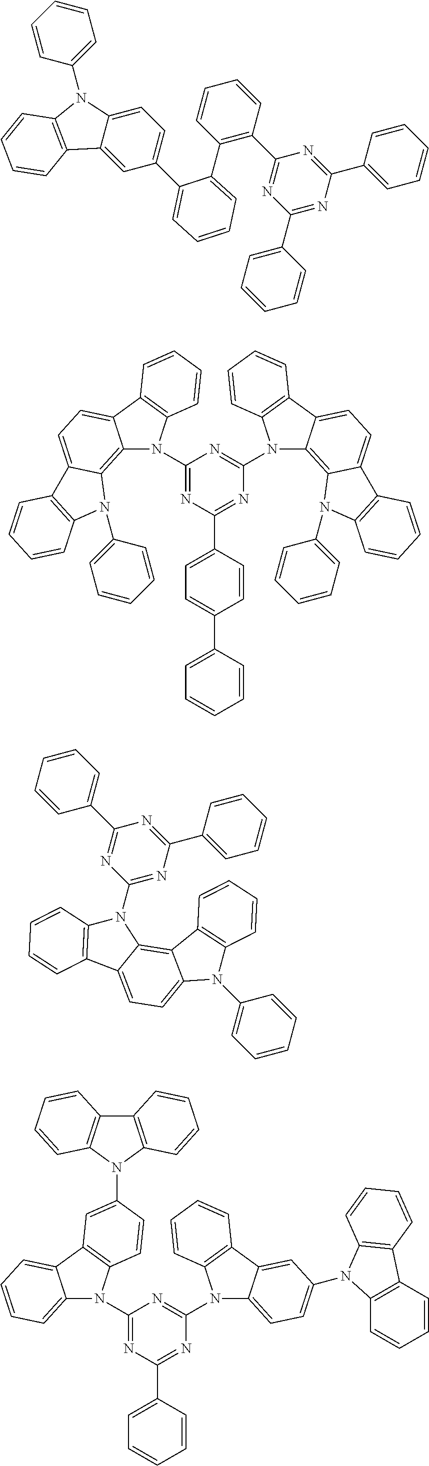

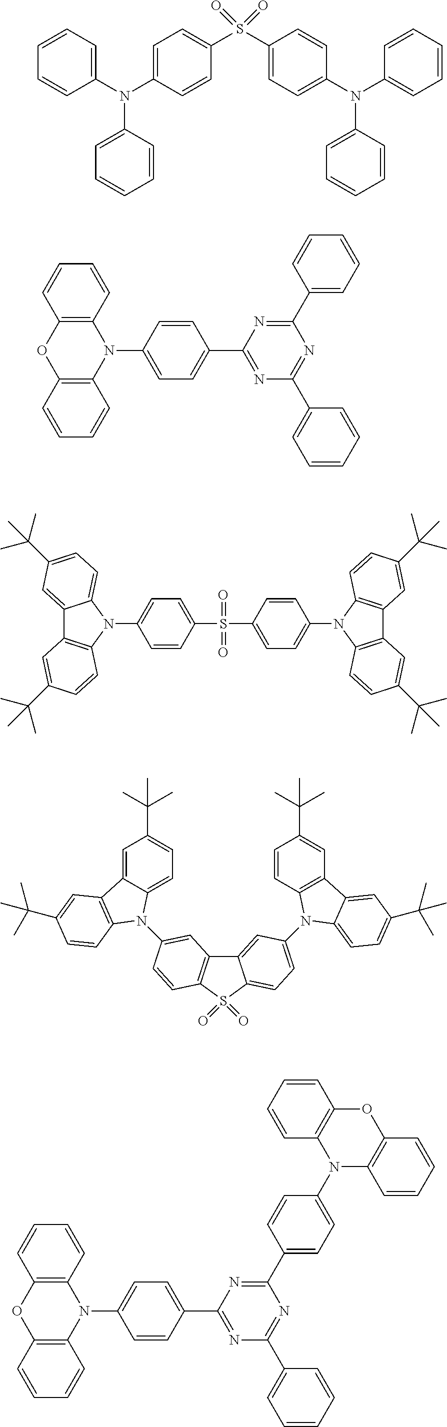

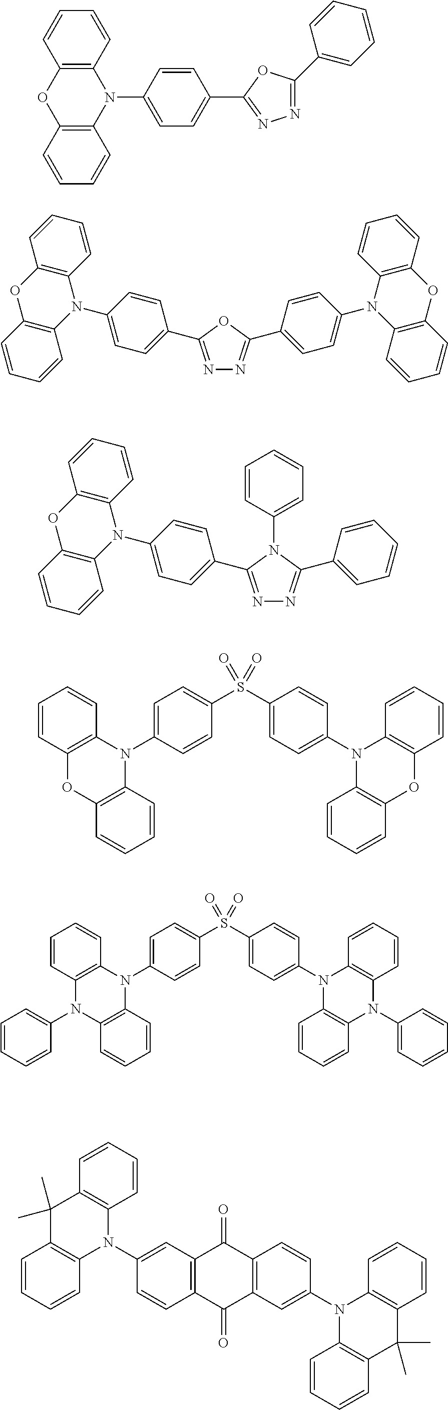

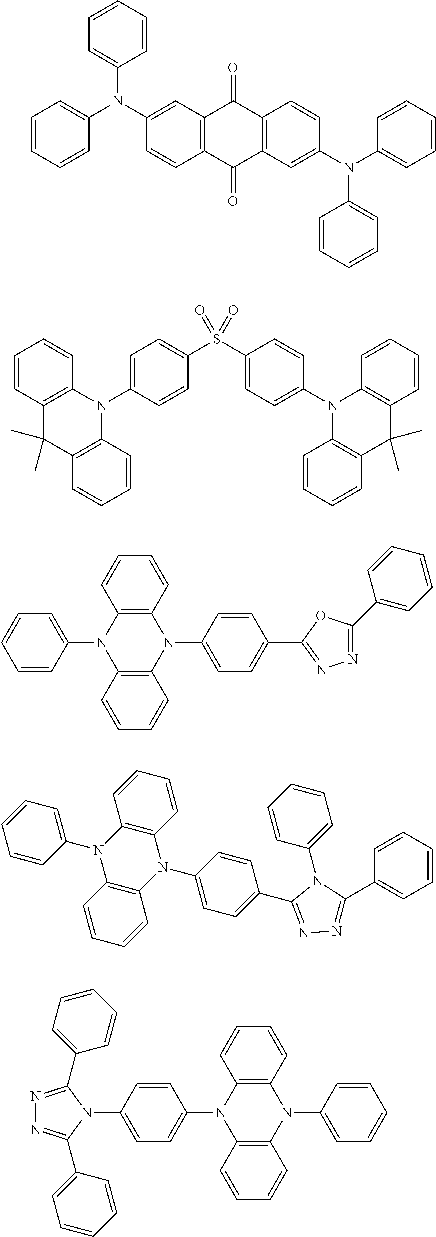

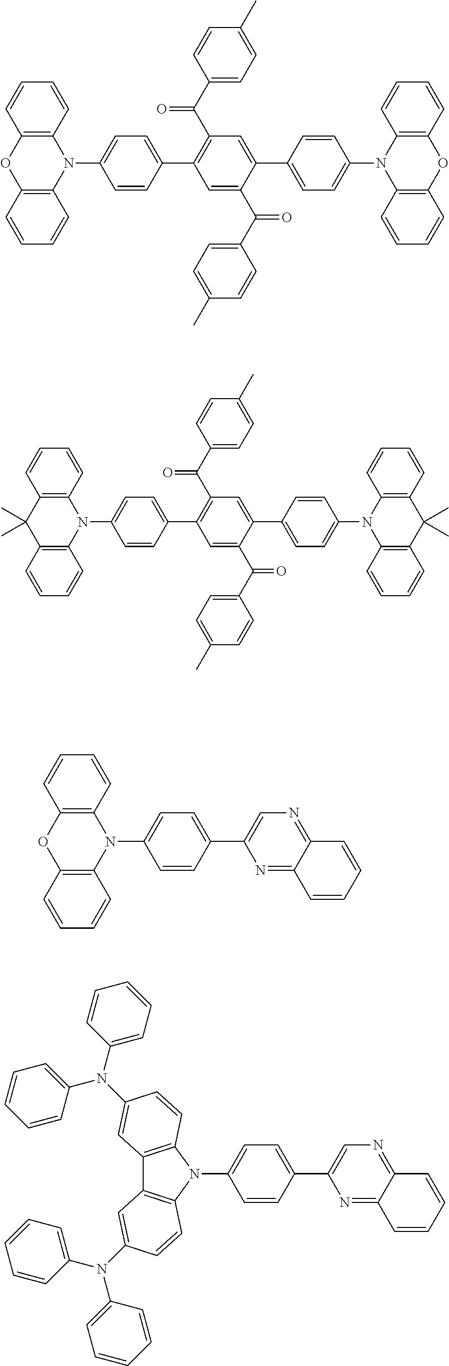

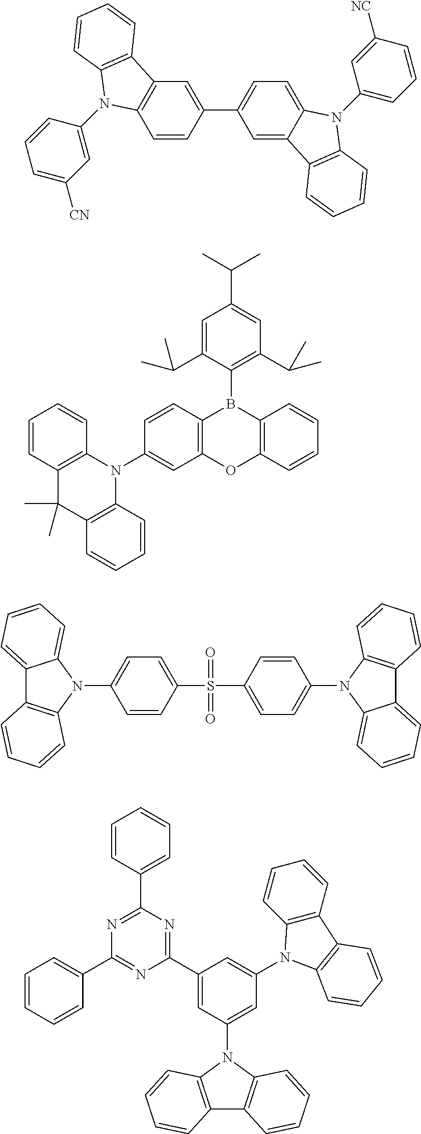 [SMILES: N#Cc1cccc(-n2c3ccccc3c3cc(-c4ccc5c(c4)c4ccccc4n5-c4cccc(C#N)c4)ccc32)c1 ; CC(C)c1cc(C(C)C)c(B2c3ccccc3Oc3cc(N4c5ccccc5C(C)(C)c5ccccc54)ccc32)c(C(C)C)c1 ; O=S(=O)(c1ccc(-n2c3ccccc3c3ccccc32)cc1)c1ccc(-n2c3ccccc3c3ccccc32)cc1 ; c1ccc(-c2nc(-c3ccccc3)nc(-c3cc(-n4c5ccccc5c5ccccc54)cc(-n4c5ccccc5c5ccccc54)c3)n2)cc1]

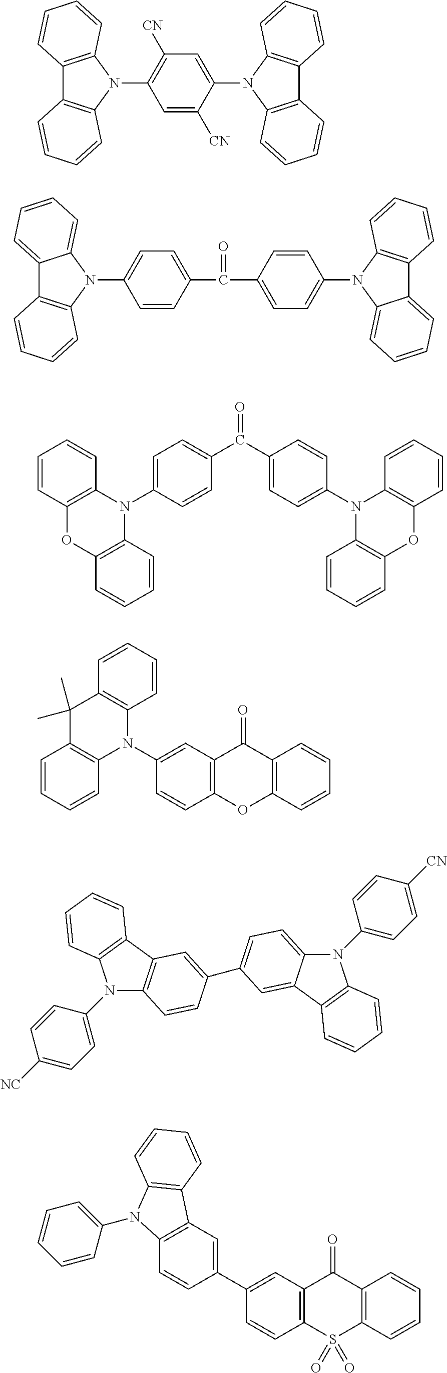

In some exemplary embodiments, in the general formula of the organic functional compound, p=1, q=1, that is, the general formula of the solubilizing structural unit SG is

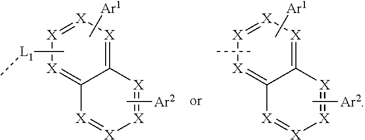

Some exemplary general formulas of solubilizing structural unit SG are listed below.

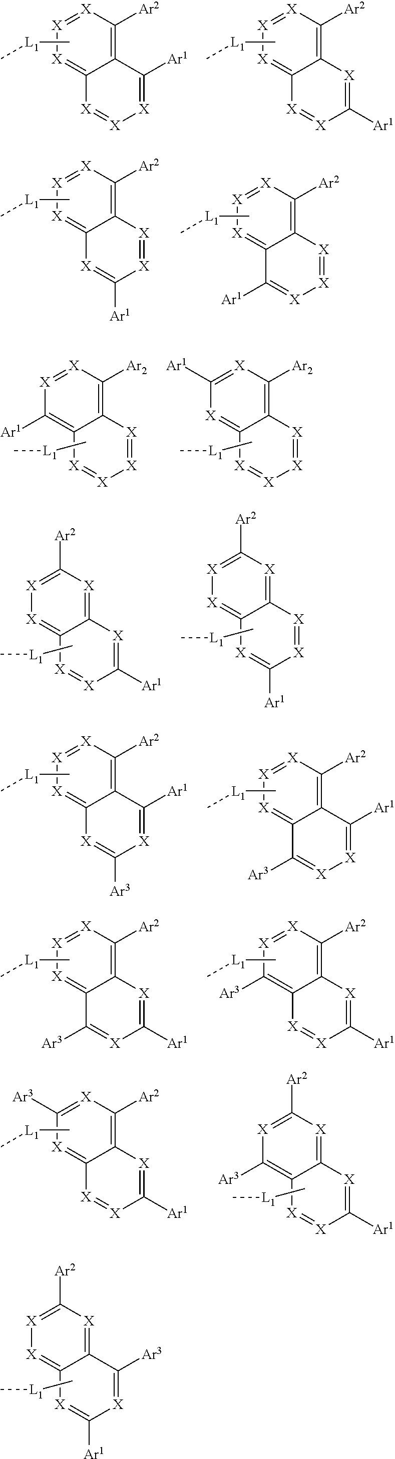

Ar³ is selected from aryl or heteroaryl groups.

In one embodiment, in the solubilizing structural unit SG described above, L1, Ar¹, Ar², and Ar³ are the same or different and are selected from unsubstituted or substituted aryl or heteroaryl group containing 2-20 carbon atoms. In one embodiment, the aryl group contains 5-15 carbon atoms in the ring system, such as 5-10 carbon atoms, the heteroaryl group contains 2-15 carbon atoms in the ring system, such as 2-10 carbon atoms, and at least one heteroatom, provided that the total number of carbon atoms and heteroatoms is at least 4. In one embodiment, the heteroatom is selected from Si, N, P, O, S and/or Ge. In another embodiment, the heteroatom is selected from Si, N, P, O and/or S.

The aromatic or aryl groups described herein refer to hydrocarbyl comprising at least one aromatic ring, including monocyclic groups and polycyclic ring systems. A heteroaromatic or heteroaryl group refers to a hydrocarbyl (containing a heteroatom) having at least one heteroaromatic ring, including monocyclic groups and polycyclic ring systems. These polycyclic rings may have two or more rings, wherein two carbon atoms are shared by two adjacent rings, i.e., fused rings. At least one of these polycyclic rings is aromatic or heteroaromatic. For the present embodiment, the aromatic or heteroaromatic groups include not only aromatic or heteroaromatic systems, but also the systems in which a plurality of aryls or heteroaryls may be interrupted by short non-aromatic units (<10% non-H atoms, e.g. <5% non-H atoms, such as C, N, or O atoms), thus, the groups of the system such as 9,9′-spirobifluorene, 9,9-diarylfluorene, triarylamine, diaryl ether and the like also belong to the aromatic groups of the present embodiment.

Specifically, examples of the aromatic group include: benzene, naphthalene, anthracene, phenanthrene, perylene, tetracene, pyrene, benzopyrene, triphenylene, acenaphthene, fluorene, and derivatives thereof. Aromatic group is the group formed by aromatic, and the heteroaromatic group below and non-aromatic ring group are defined similarly.

Examples of the heteroaromatic group include: furan, benzofuran, thiophene, benzothiophene, pyrrole, pyrazole, triazole, imidazole, oxazole, oxadiazole, thiazole, tetrazole, indole, carbazole, pyrrolozimidazole, pyrrolopyrrole, thienopyrrole, thienothiophene, furopyrrole, furofuran, thienofuran, benzisoxazole, benzisothiazole, benzimidazole, pyridine, pyrazine, pyridazine, pyrimidine, triazine, quinoline, isoquinoline, cinnoline, quinoxaline, phenanthridine, perimidine, quinazoline, quinazolinone, and derivatives thereof.

Exemplary aryl or heteroaryl groups are selected from benzene, naphthalene, phenanthrene, pyridine, pyrene or thiophene.

In the solubilizing structural unit SG of the present embodiment, L₁, Ar¹, Ar², or Ar³ may be selected from the following groups:

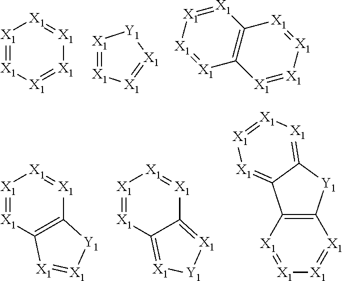

Wherein X₁ is selected from CR⁵ or N; Y₁ is selected from CR⁶R⁷, SiR⁸R⁹, NR¹⁰, C(═O), S or O;

R⁵, R⁶, R⁷, R⁸, R⁹ and R¹⁰ are each independently selected from the following groups: H; D; linear alkyl, containing 1-20 carbon atoms, linear alkoxy containing 1-20 carbon atoms or linear or thioalkoxy containing 1-20 carbon atoms; branched or cyclic alkyl containing 3-20 carbon atoms, branched or cyclic alkoxy containing 3-20 carbon atoms or branched or cyclic thioalkoxy group containing 3-20 carbon atoms; silyl; substituted ketone group containing 1-20 carbon atoms; alkoxycarbonyl containing 2-20 carbon atoms; aryloxycarbonyl containing 7-20 carbon atoms; cyano (—CN); carbamoyl (—C(═O)NH2); haloformyl (—C(═O)—X, wherein X represents a halogen atom); formyl (—C(═O)—H); isocyano; isocyanate group; thiocyanate group; isothiocyanate group; hydroxy; nitro; CF₃; Cl; Br; F; crosslinkable groups; substituted or unsubstituted aromatic containing 5-40 ring atoms or substituted or unsubstituted heteroaromatic group containing 5-40 ring atoms; and any combination thereof; wherein one or more of the groups R⁵, R⁶, R⁷, R⁸, R⁹, R¹⁰ each may combine with another one or more of the groups or combine with the ring bonded thereto to form a monocyclic or polycyclic aliphatic or aromatic ring system.

In one embodiment, L₁, Ar¹, Ar², and Ar³ are each independently selected from one of the following groups:

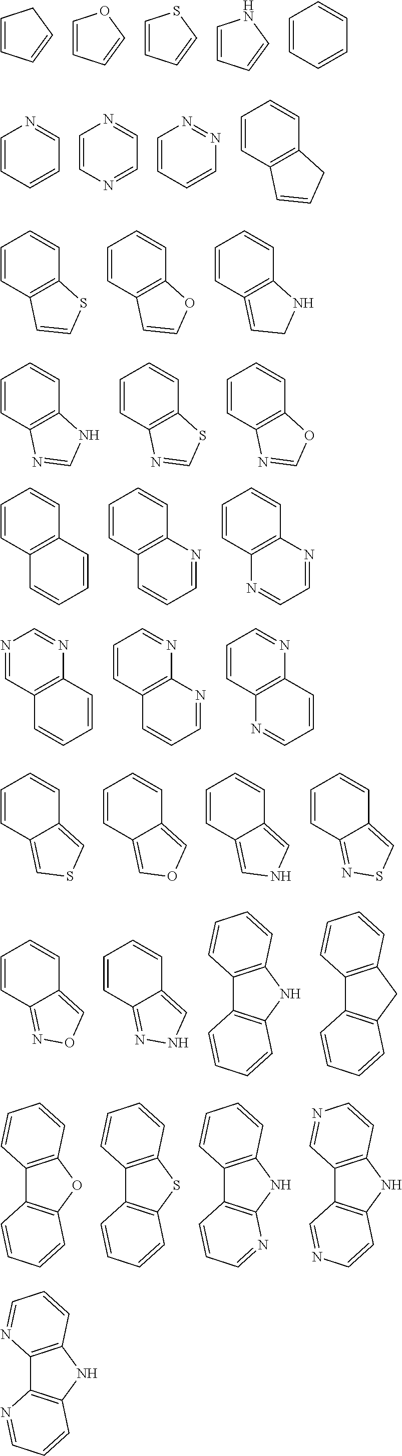

In a further embodiment, the general formula of the solubilizing structural unit SG is

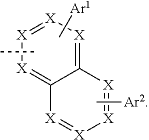

In another exemplary embodiment, Ar¹, Ar², and Ar³ can be the same or different and are selected from phenyl or naphthyl.

In some other embodiments, the solubilizing structural unit SG as described above are selected from the following structural formulas:

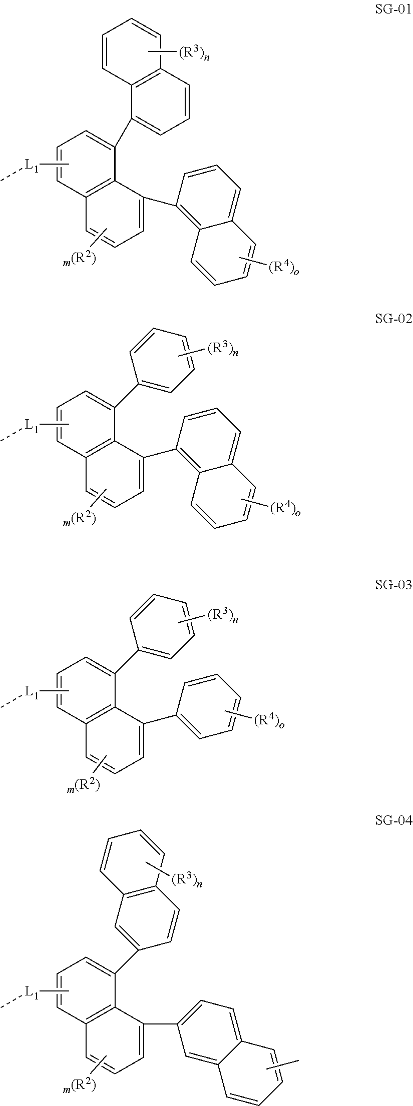

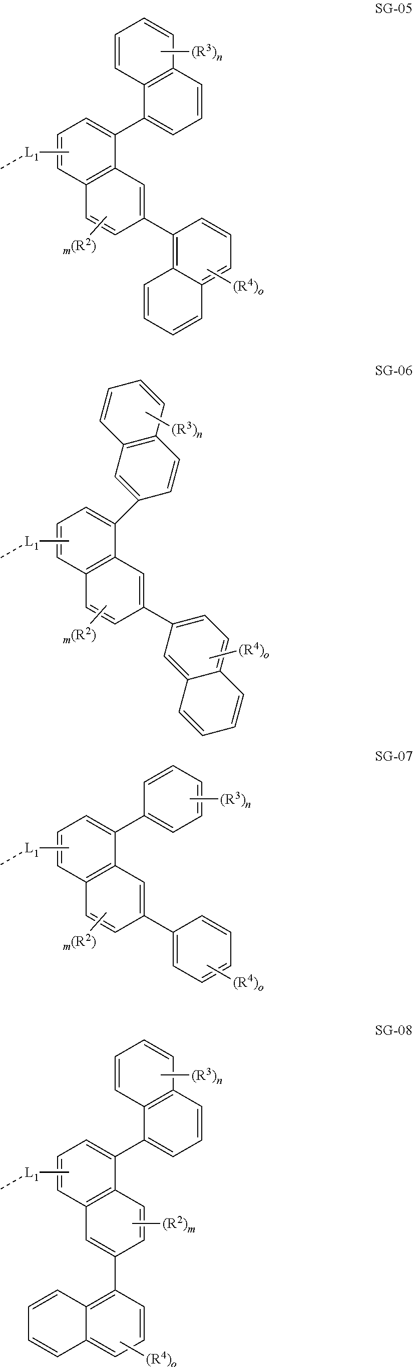

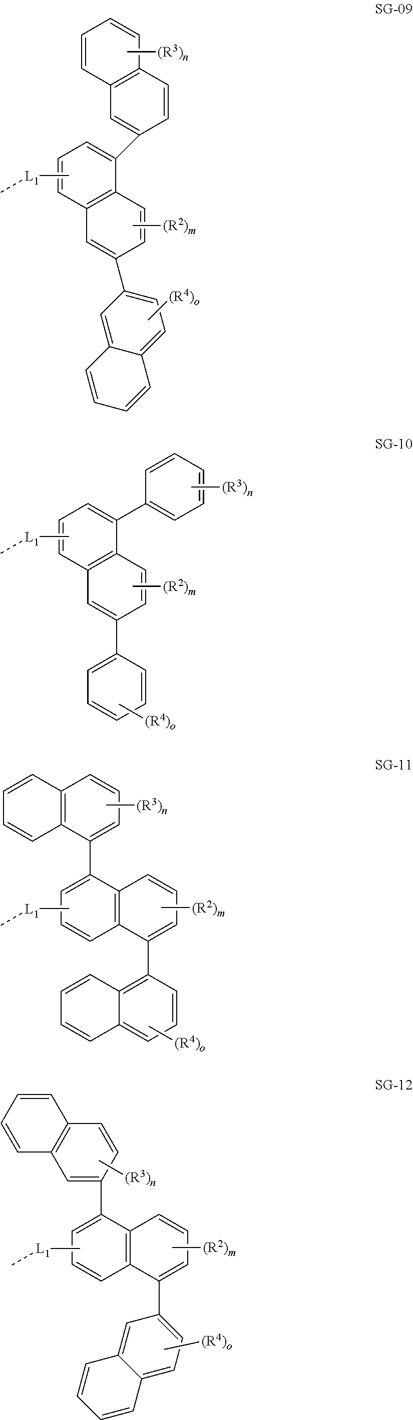

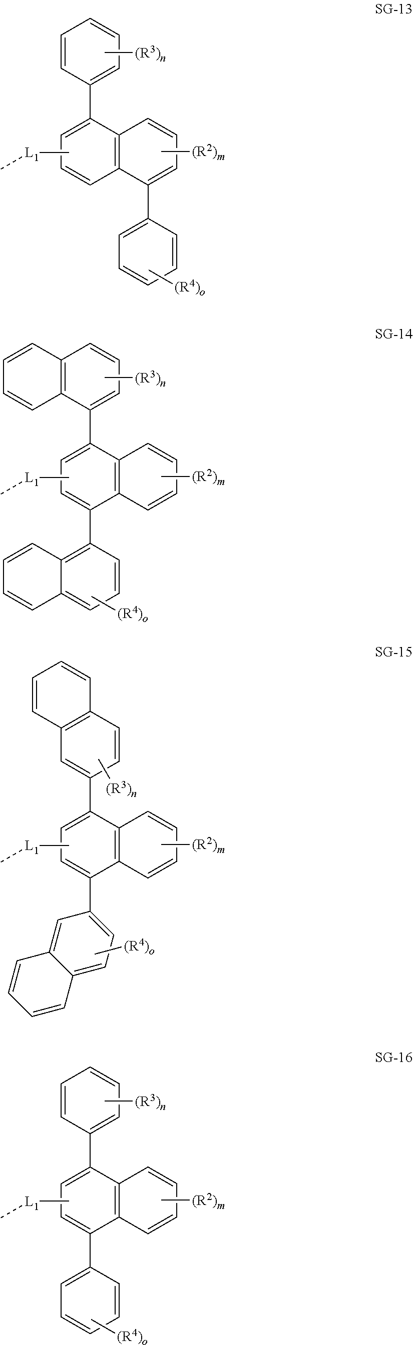

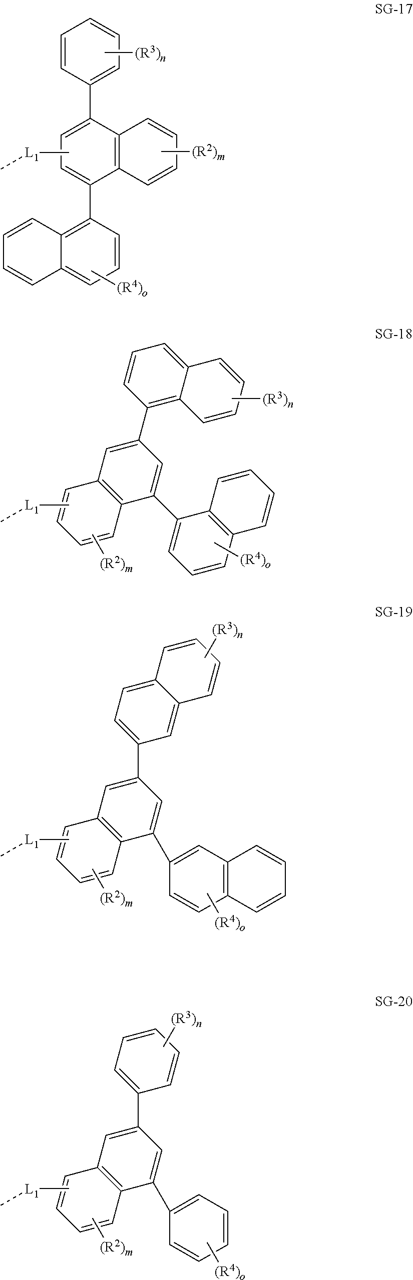

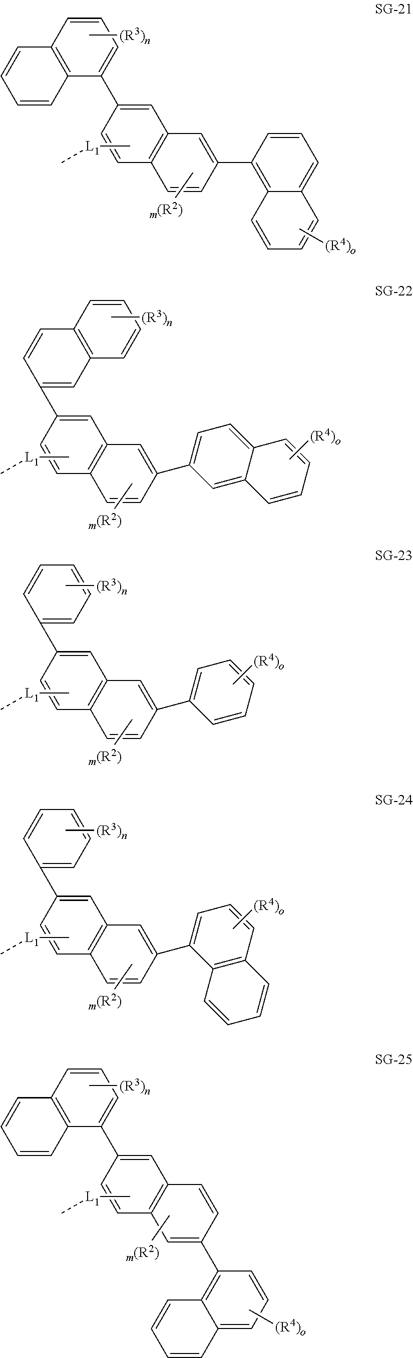

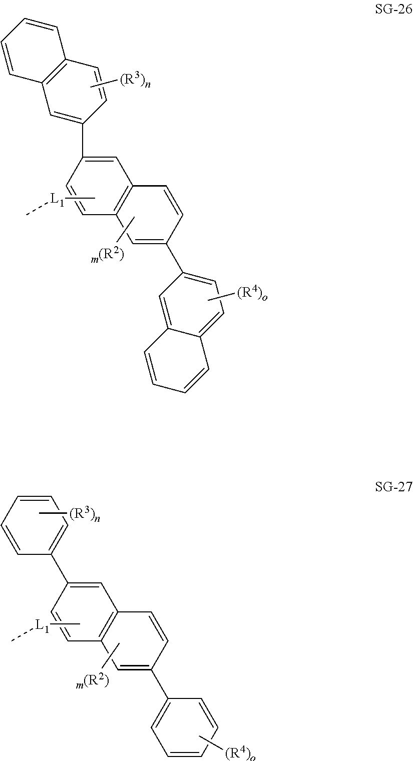

wherein, R², R³ and R⁴ are each independently selected from at least one of the following groups: H; D; linear alkyl containing 1-20 carbon atoms, linear alkoxy containing 1-20 carbon atoms or linear thioalkoxy containing 1-20 carbon atoms; branched or cyclic alkyl containing 3-20 carbon atoms, branched or cyclic alkoxy containing 3-20 carbon atoms or branched or cyclic thioalkoxy containing 3-20 carbon atoms; silyl; substituted ketone group containing 1-20 carbon atoms; alkoxycarbonyl containing 2-20 carbon atoms; aryloxycarbonyl containing 7-20 carbon atoms; cyano; carbamoyl; haloformyl; formyl; isocyano; isocyanate group; thiocyanate group; isothiocyanate group; hydroxy; nitro; CF₃; Cl; Br; F; crosslinkable group; substituted or unsubstituted aromatic containing 5-40 ring atoms; substituted or unsubstituted heteroaromatic ring system containing 5-40 ring atoms; aryloxy containing 5-40 ring atoms or heteroaryloxy group containing 5-40 ring atoms;

m is selected from 0, 1, 2, 3, 4 or 5; n and o are each independently selected from 0, 1, 2, 3, 4, 5, 6 or 7.

In further embodiment, the groups R², R³, R⁴ represent hydrogen (m, n and o=0), linear alkyl containing 1-20 carbon atoms or linear alkoxy containing 1-20 carbon atoms, or branched alkyl containing 3-30 carbon atoms or branched alkoxy containing 3-20 carbon atoms.

In some further embodiments, the solubilizing structural unit SG described above is selected from, but not limited to, the following structures:

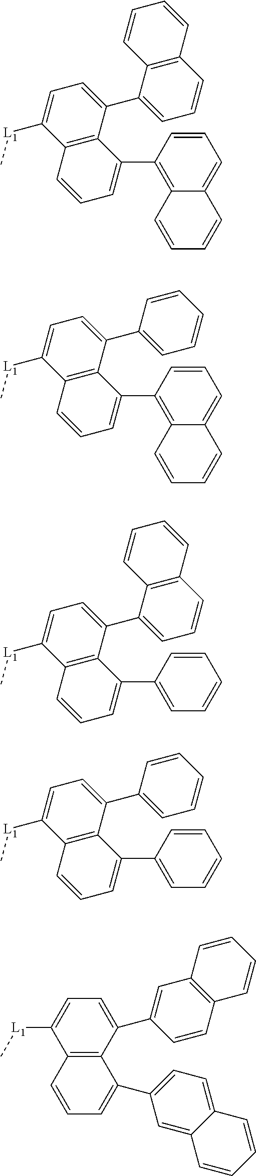

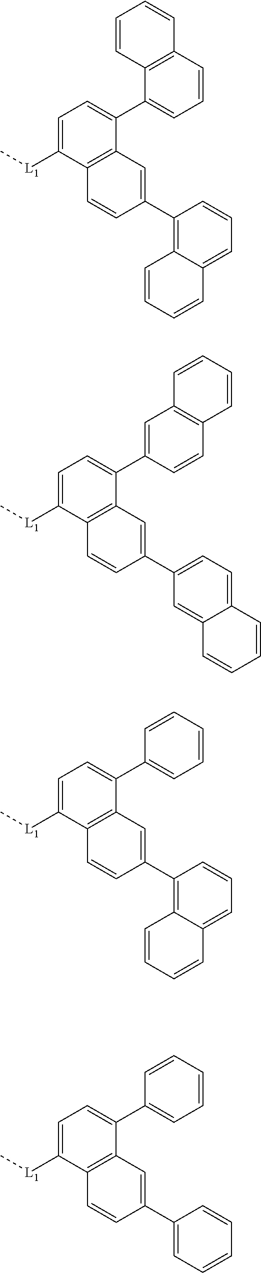

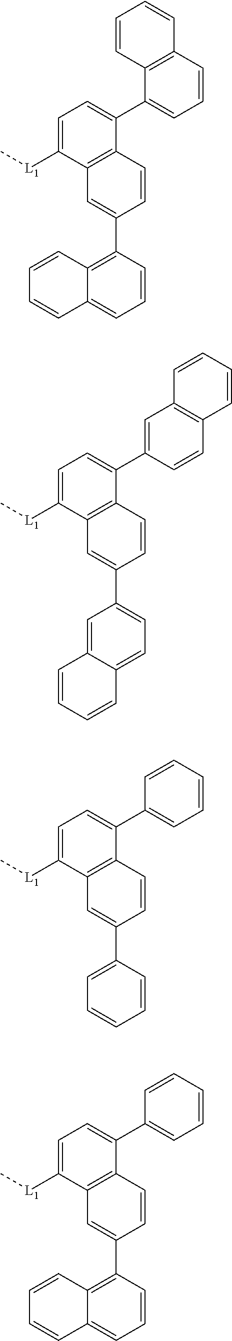

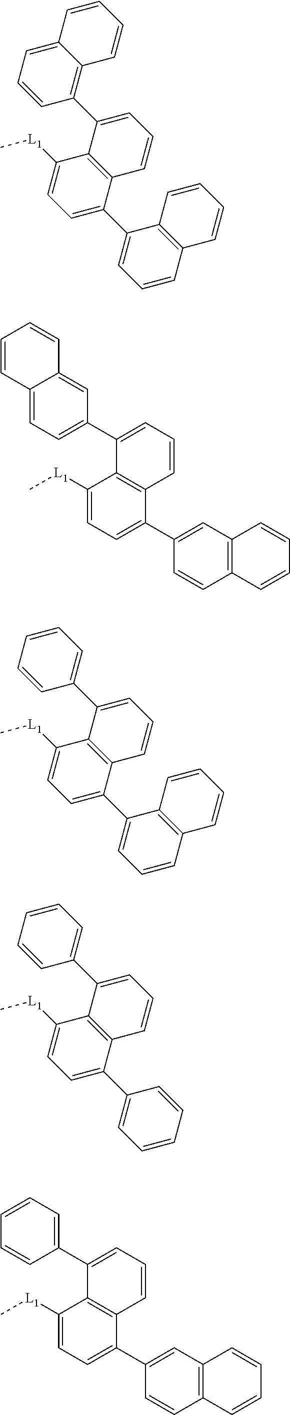

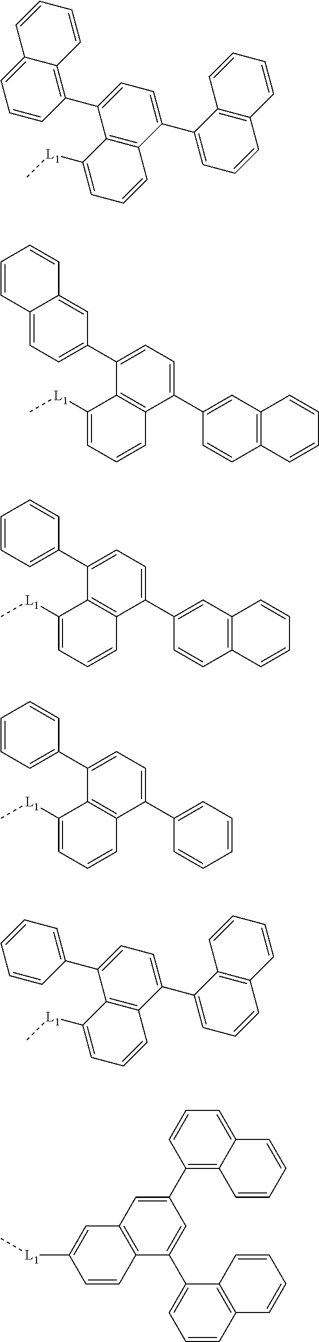

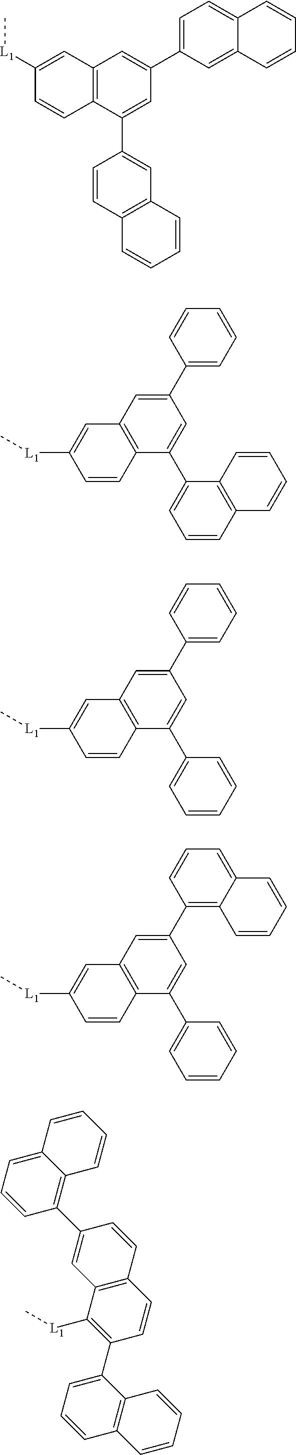

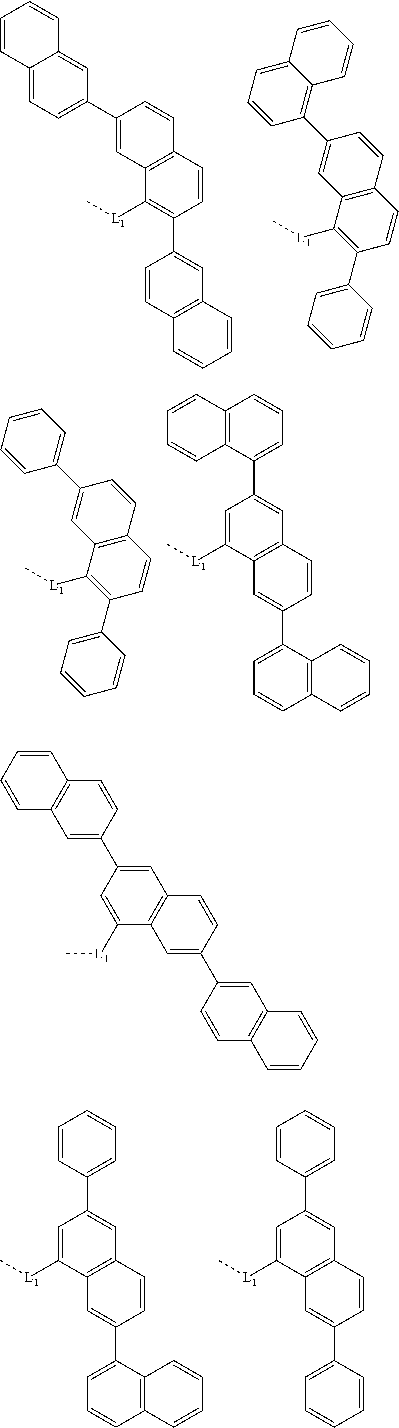

In further embodiment, L₁ is selected from the following structures:

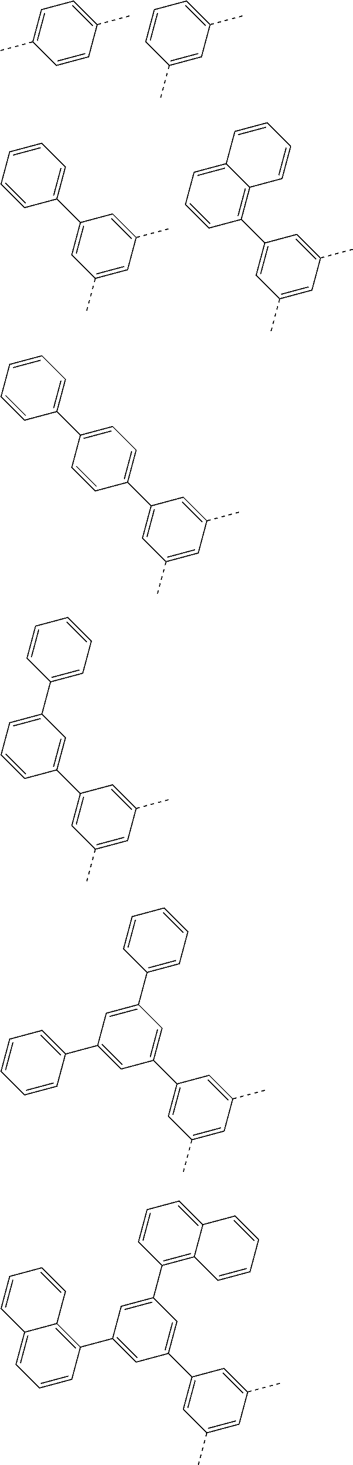

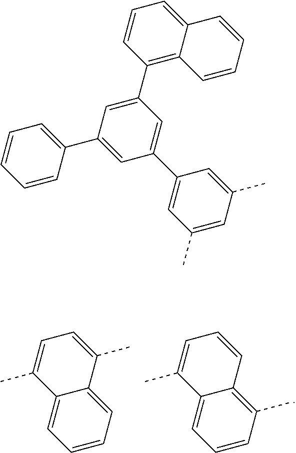

In the structures of SG-01 to SG-27, for example, R¹, R², R³, R⁴ are each independently selected from: F; Cl; Br; I; N(Ar)₂; CN; NO_{2;} Si(R¹)₃; B(OR′)₂; C(═O)Ar; C(═O)R′; P(═O)(Ar)₂; P(═O)(R′)₂; S(═O)Ar; S(═O)R′; S(═O)₂Ar; S(═O)₂R′; —CR′═CR′Ar; OSO₂R′; linear alkyl containing 1-40 carbon atoms, especially containing 1-20 carbon atoms, linear alkoxy containing 1-40 carbon atoms, especially containing 1-20 carbon atoms or linear thioalkoxy containing 1-40 carbon atoms, e.g. especially containing 1-20 carbon atoms; or branched or cyclic alkyl containing 3-40 carbon atoms, especially containing 3-20 carbon atoms, branched or cyclic alkoxy containing 3-40 carbon atoms, especially containing 3-20 carbon atoms or branched or cyclic thioalkoxy containing 3-40 carbon atoms, e.g. especially containing 3-20 carbon atoms. Each of these groups may be substituted with one or more groups R′; wherein, one or more non-adjacent CH₂ groups may be replaced by R′C═CR′, C═C, Si(R′)₂, Ge(R′)₂, Sn(R′)₂, C═O, C═S, C═Se, C═NR′, P(═O)(R′), SO, SO₂, NR′, O, S, or CONR′, and wherein, one or more H atoms may be replaced by F, Cl, Br, I, CN, or NO_{2;} a crosslinkable group, or an aromatic or heteroaromatic ring system containing 5-60 ring atoms may be substituted with one or more groups R′ in each case, or an aryloxy or heteroaryloxy containing 5-60 ring atoms may be substituted with one or more group R′ or any combination thereof, wherein, two or more substituents R may also form mono- or polycyclic aliphatic or aromatic ring systems with one another. R′ is independently selected from H, or an aliphatic or aromatic hydrocarbyl group containing 1-20 carbon atoms in each case, and Ar is an aryl or heteroaryl group containing 2-30 carbon atoms.

In addition, in the present embodiment, the alkyl containing 1-40 carbon atoms, wherein the individual H atom or CH₂ group may be substituted with the above group or group R, is for example a group selected from: methyl, ethyl, n-propyl, isopropyl, cyclopropyl, n-butyl, isobutyl, sec-butyl, tert-butyl, cyclobutyl, methylbutyl, n-pentyl, sec-pentyl, cyclopentyl, n-hexyl, cyclohexyl, n-heptyl, cycloheptyl, n-octyl, cyclooctyl, ethylhexyl, trifluoromethyl, pentafluoroethyl, trifluoroethyl, vinyl, propenyl, butenyl, pentenyl, cyclopentenyl, hexenyl, cyclohexenyl, heptenyl, cycloheptenyl, octenyl, cyclooctenyl, ethynyl, propynyl, butynyl, pentynyl, hexynyl and octynyl. The alkoxy containing 1-40 carbon atoms is methoxy, trifluoromethoxy, ethoxy, n-propoxy, isopropoxy, n-butoxy, isobutoxy, sec-butoxy tert-butoxy or methyl butoxy.

The total amount of SP³ hybridized groups in the organic functional compound of the present embodiment is not more than 30% of the molecular weight, e.g. in one embodiment, the total amount of SP³ hybridized groups in the organic functional compound of the present embodiment is not more than 20% of the molecular weight, such as in still one embodiment, the total amount of SP³ hybridized groups in the organic functional compound of the present embodiment is not more than 10% of the total molecular weight. The presence of fewer SP³ hybridized groups can effectively ensure the thermal stability of the compound and ensure the stability of the device.

The weight ratio of the structural unit F to the structural unit SG in the organic functional compound of the present embodiment ranges from 2:1 to 1:20, e.g. in one embodiment, the weight ratio of the structural unit F to the structural unit SG in the organic functional compound of the present embodiment ranges from 1:1 to 1:5, such as in one embodiment, the weight ratio of the structural unit F to the structural unit SG in the organic functional compound of the present embodiment ranges from 1:1 to 1:3.

Some examples of the organic functional compound described in the present embodiment are listed below

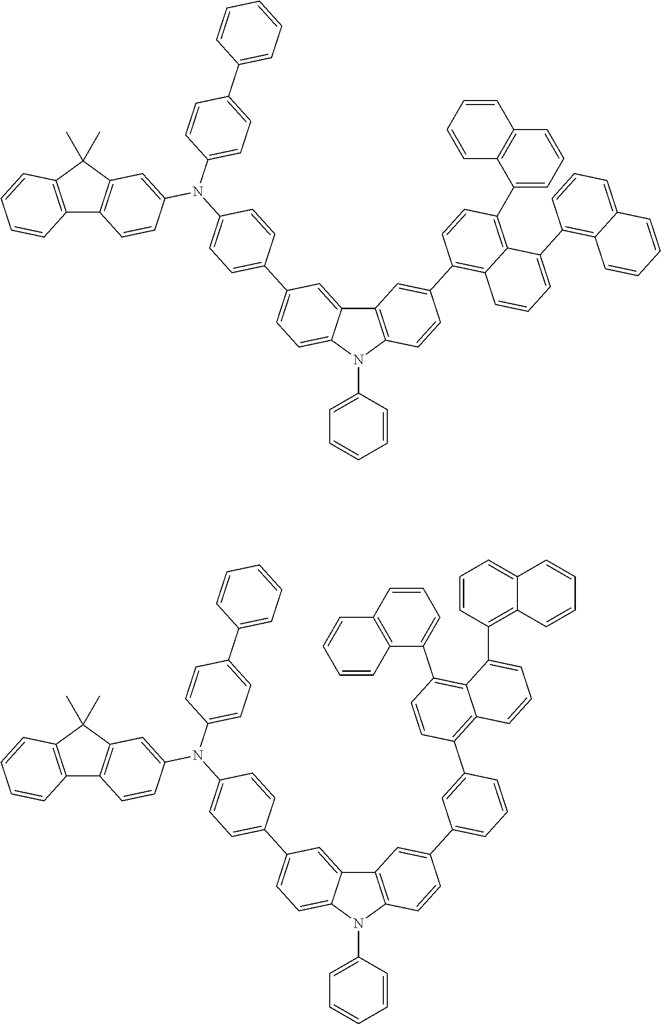 [SMILES: CC1(C)c2ccccc2-c2ccc(N(c3ccc(-c4ccccc4)cc3)c3ccc(-c4ccc5c(c4)c4cc(-c6ccc(-c7cccc8ccccc78)c7c(-c8cccc9ccccc89)cccc67)ccc4n5-c4ccccc4)cc3)cc21 ; CC1(C)c2ccccc2-c2ccc(N(c3ccc(-c4ccccc4)cc3)c3ccc(-c4ccc5c(c4)c4cc(-c6cccc(-c7ccc(-c8cccc9ccccc89)c8c(-c9cccc%10ccccc9%10)cccc78)c6)ccc4n5-c4ccccc4)cc3)cc21]

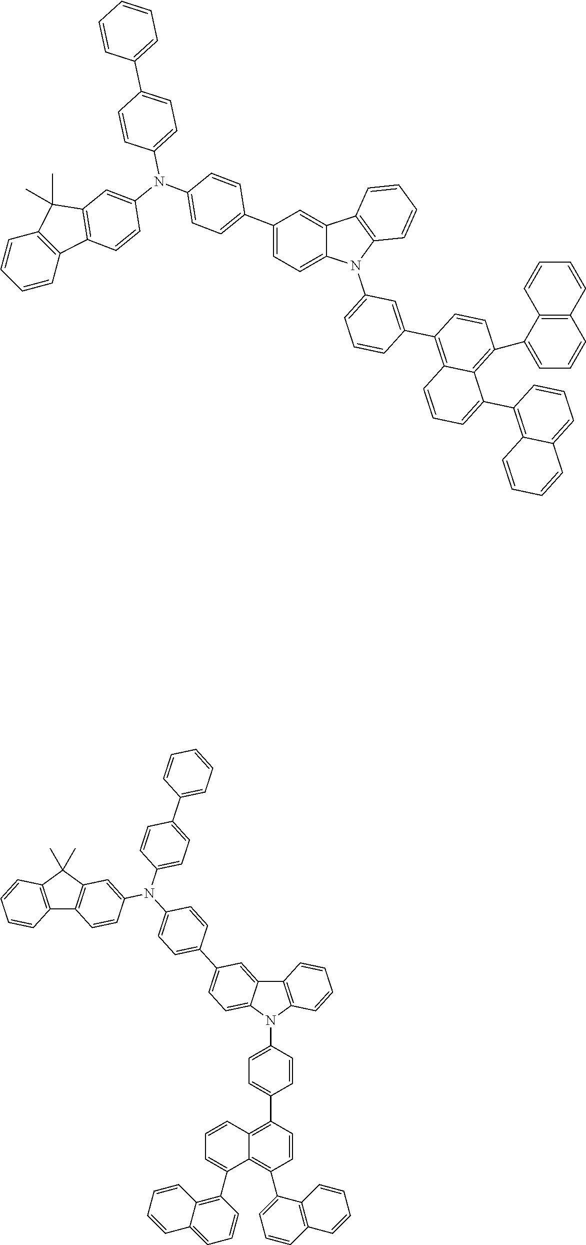 [SMILES: CC1(C)c2ccccc2-c2ccc(N(c3ccc(-c4ccccc4)cc3)c3ccc(-c4ccc5c(c4)c4ccccc4n5-c4cccc(-c5ccc(-c6cccc7ccccc67)c6c(-c7cccc8ccccc78)cccc56)c4)cc3)cc21 ; CC1(C)c2ccccc2-c2ccc(N(c3ccc(-c4ccccc4)cc3)c3ccc(-c4ccc5c(c4)c4ccccc4n5-c4ccc(-c5ccc(-c6cccc7ccccc67)c6c(-c7cccc8ccccc78)cccc56)cc4)cc3)cc21]

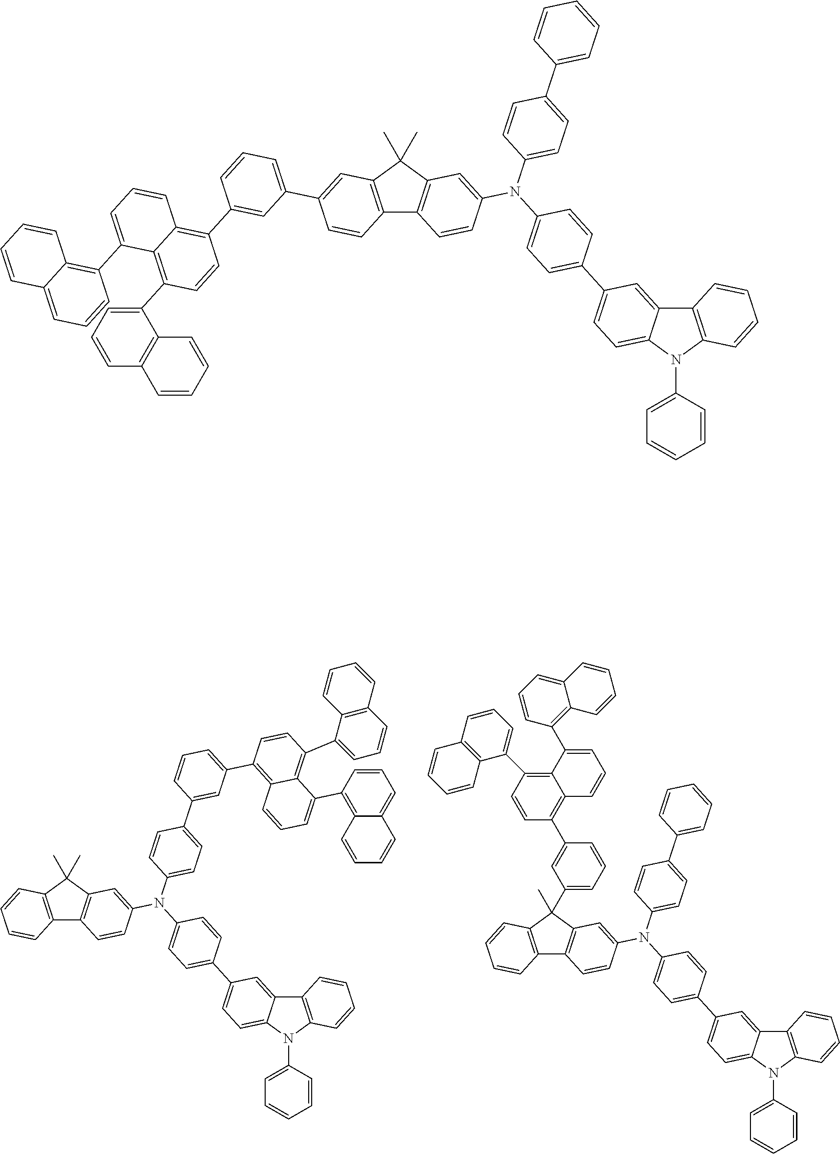

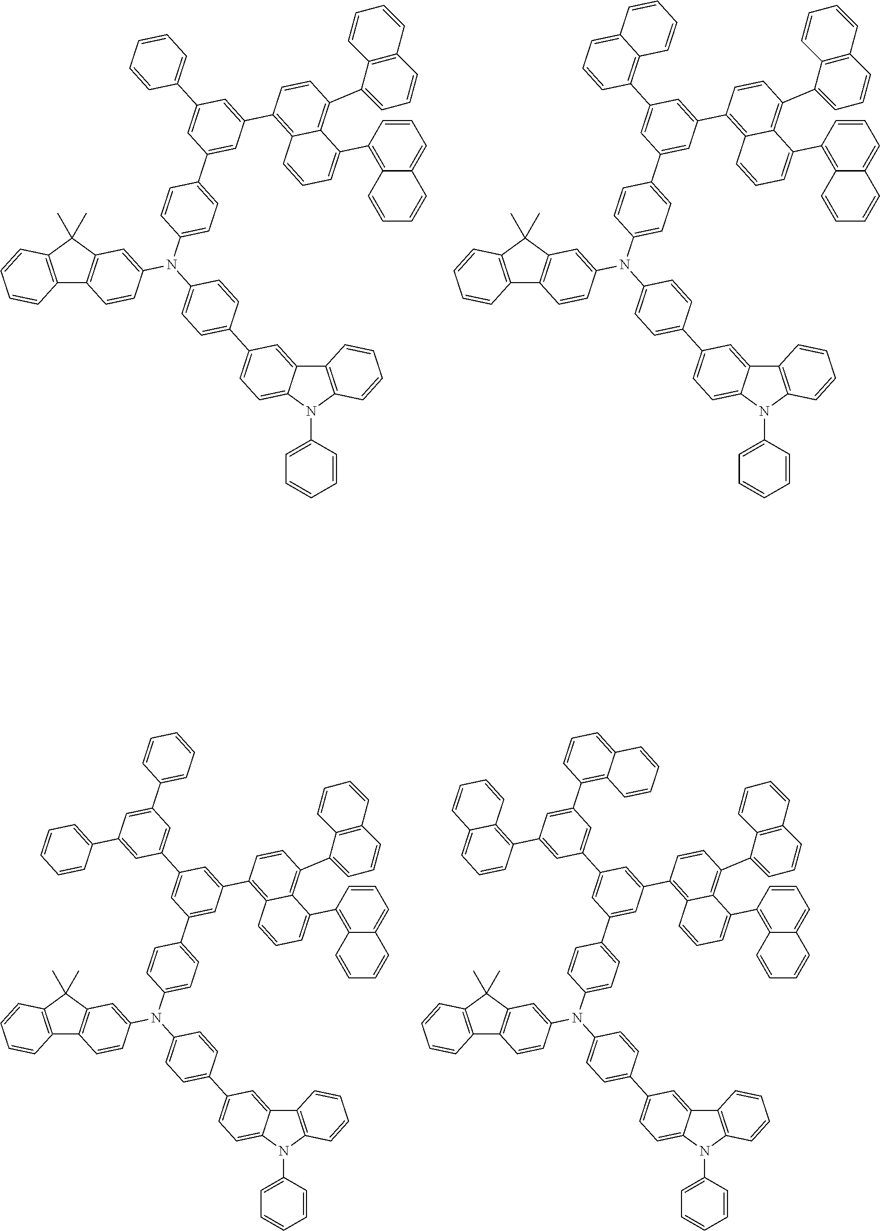

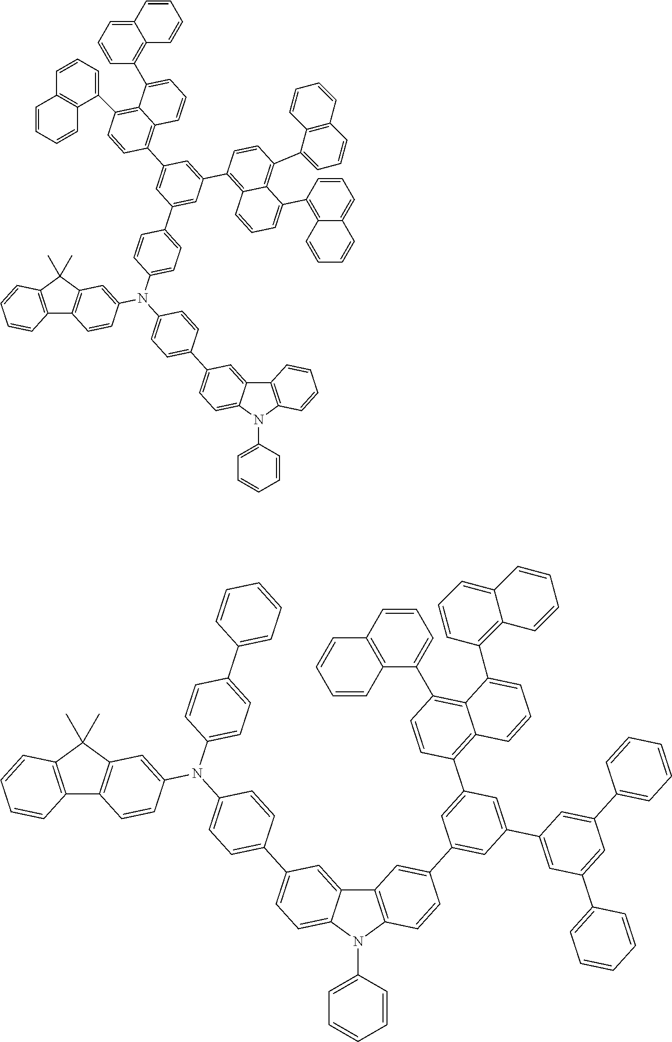 [SMILES: CC1(C)c2ccccc2-c2ccc(N(c3ccc(-c4cc(-c5ccc(-c6cccc7ccccc67)c6c(-c7cccc8ccccc78)cccc56)cc(-c5ccc(-c6cccc7ccccc67)c6c(-c7cccc8ccccc78)cccc56)c4)cc3)c3ccc(-c4ccc5c(c4)c4ccccc4n5-c4ccccc4)cc3)cc21 ; CC1(C)c2ccccc2-c2ccc(N(c3ccc(-c4ccccc4)cc3)c3ccc(-c4ccc5c(c4)c4cc(-c6cc(-c7cc(-c8ccccc8)cc(-c8ccccc8)c7)cc(-c7ccc(-c8cccc9ccccc89)c8c(-c9cccc%10ccccc9%10)cccc78)c6)ccc4n5-c4ccccc4)cc3)cc21]

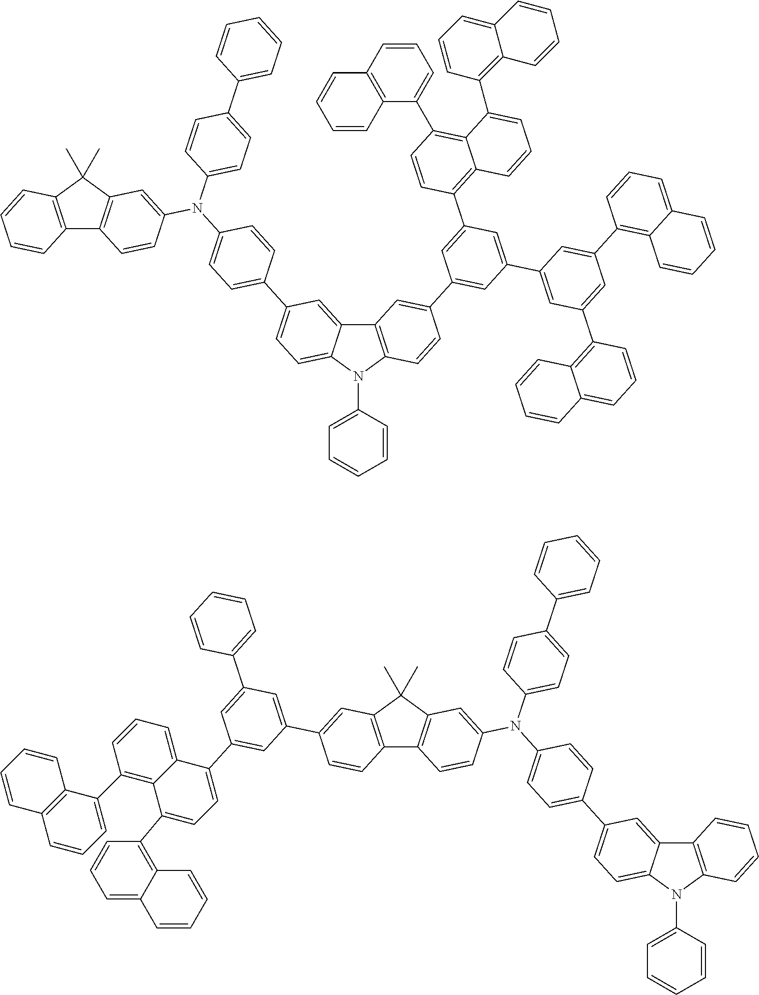 [SMILES: CC1(C)c2ccccc2-c2ccc(N(c3ccc(-c4ccccc4)cc3)c3ccc(-c4ccc5c(c4)c4cc(-c6cc(-c7cc(-c8cccc9ccccc89)cc(-c8cccc9ccccc89)c7)cc(-c7ccc(-c8cccc9ccccc89)c8c(-c9cccc%10ccccc9%10)cccc78)c6)ccc4n5-c4ccccc4)cc3)cc21 ; CC1(C)c2cc(-c3cc(-c4ccccc4)cc(-c4ccc(-c5cccc6ccccc56)c5c(-c6cccc7ccccc67)cccc45)c3)ccc2-c2ccc(N(c3ccc(-c4ccccc4)cc3)c3ccc(-c4ccc5c(c4)c4ccccc4n5-c4ccccc4)cc3)cc21]

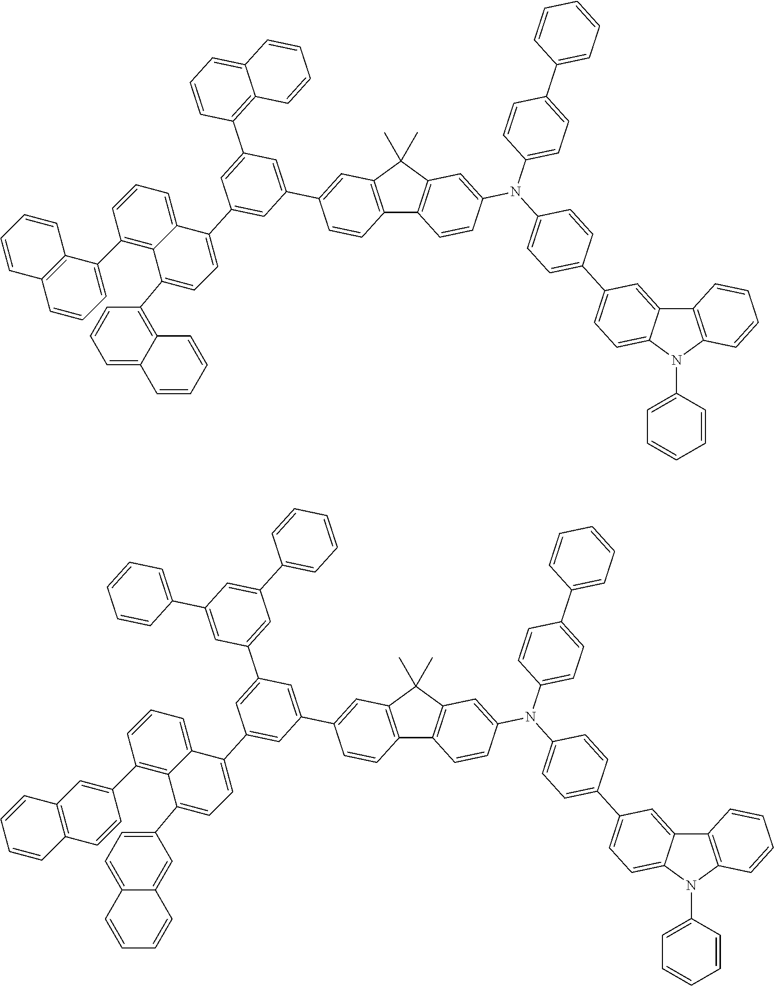 [SMILES: CC1(C)c2cc(-c3cc(-c4cccc5ccccc45)cc(-c4ccc(-c5cccc6ccccc56)c5c(-c6cccc7ccccc67)cccc45)c3)ccc2-c2ccc(N(c3ccc(-c4ccccc4)cc3)c3ccc(-c4ccc5c(c4)c4ccccc4n5-c4ccccc4)cc3)cc21 ; CC1(C)c2cc(-c3cc(-c4cc(-c5ccccc5)cc(-c5ccccc5)c4)cc(-c4ccc(-c5ccc6ccccc6c5)c5c(-c6ccc7ccccc7c6)cccc45)c3)ccc2-c2ccc(N(c3ccc(-c4ccccc4)cc3)c3ccc(-c4ccc5c(c4)c4ccccc4n5-c4ccccc4)cc3)cc21]

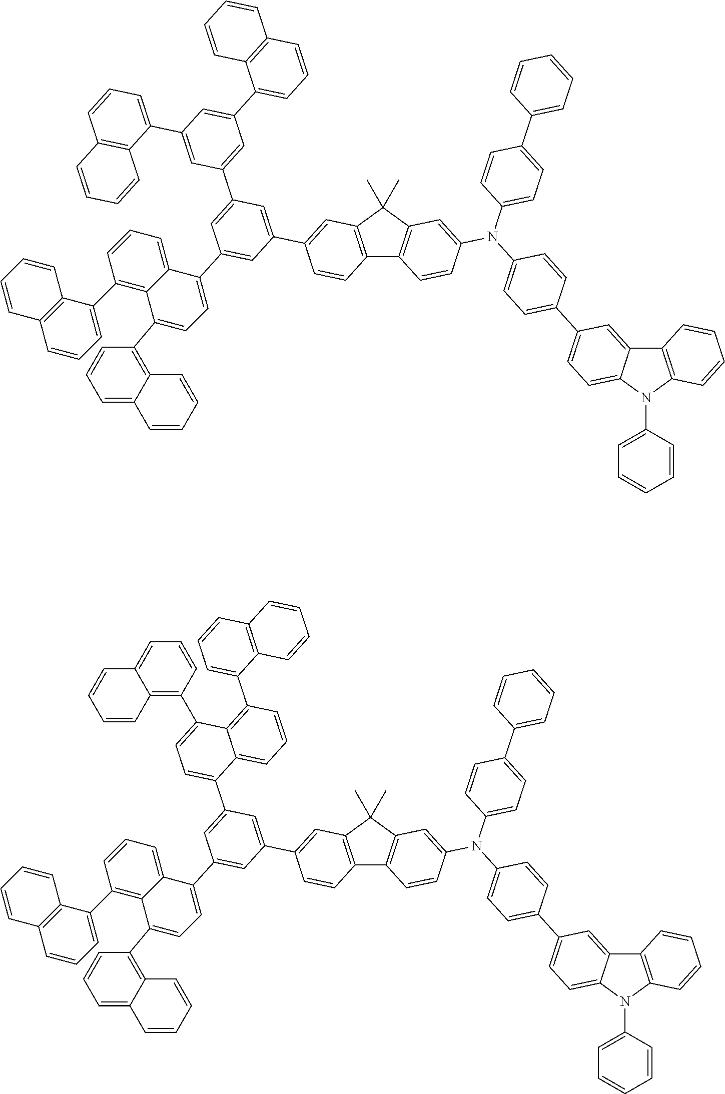 [SMILES: CC1(C)c2cc(-c3cc(-c4cc(-c5cccc6ccccc56)cc(-c5cccc6ccccc56)c4)cc(-c4ccc(-c5cccc6ccccc56)c5c(-c6cccc7ccccc67)cccc45)c3)ccc2-c2ccc(N(c3ccc(-c4ccccc4)cc3)c3ccc(-c4ccc5c(c4)c4ccccc4n5-c4ccccc4)cc3)cc21 ; CC1(C)c2cc(-c3cc(-c4ccc(-c5cccc6ccccc56)c5c(-c6cccc7ccccc67)cccc45)cc(-c4ccc(-c5cccc6ccccc56)c5c(-c6cccc7ccccc67)cccc45)c3)ccc2-c2ccc(N(c3ccc(-c4ccccc4)cc3)c3ccc(-c4ccc5c(c4)c4ccccc4n5-c4ccccc4)cc3)cc21]

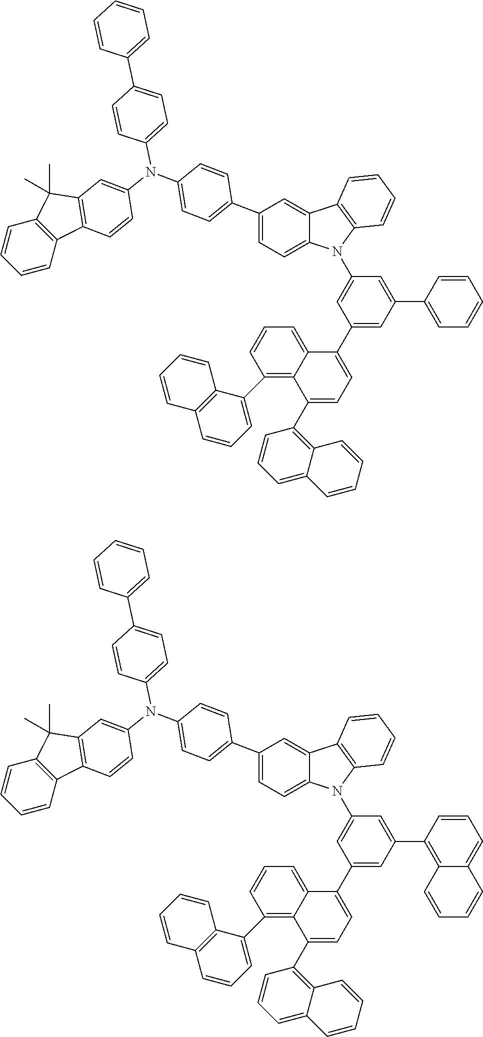 [SMILES: CC1(C)c2ccccc2-c2ccc(N(c3ccc(-c4ccccc4)cc3)c3ccc(-c4ccc5c(c4)c4ccccc4n5-c4cc(-c5ccccc5)cc(-c5ccc(-c6cccc7ccccc67)c6c(-c7cccc8ccccc78)cccc56)c4)cc3)cc21 ; CC1(C)c2ccccc2-c2ccc(N(c3ccc(-c4ccccc4)cc3)c3ccc(-c4ccc5c(c4)c4ccccc4n5-c4cc(-c5cccc6ccccc56)cc(-c5ccc(-c6cccc7ccccc67)c6c(-c7cccc8ccccc78)cccc56)c4)cc3)cc21]

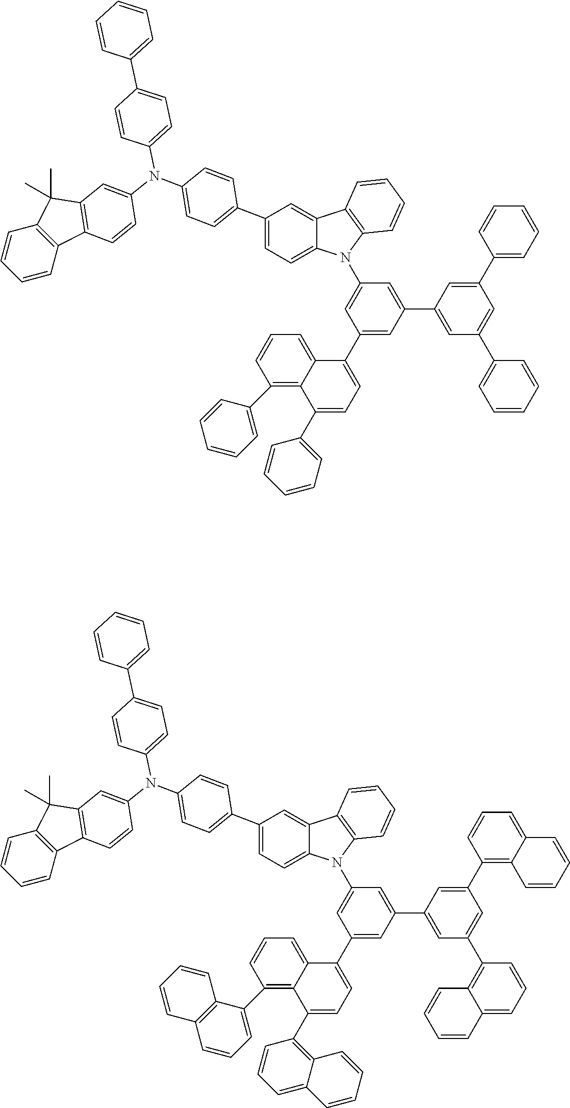 [SMILES: CC1(C)c2ccccc2-c2ccc(N(c3ccc(-c4ccccc4)cc3)c3ccc(-c4ccc5c(c4)c4ccccc4n5-c4cc(-c5cc(-c6ccccc6)cc(-c6ccccc6)c5)cc(-c5ccc(-c6ccccc6)c6c(-c7ccccc7)cccc56)c4)cc3)cc21 ; CC1(C)c2ccccc2-c2ccc(N(c3ccc(-c4ccccc4)cc3)c3ccc(-c4ccc5c(c4)c4ccccc4n5-c4cc(-c5cc(-c6cccc7ccccc67)cc(-c6cccc7ccccc67)c5)cc(-c5ccc(-c6cccc7ccccc67)c6c(-c7cccc8ccccc78)cccc56)c4)cc3)cc21]

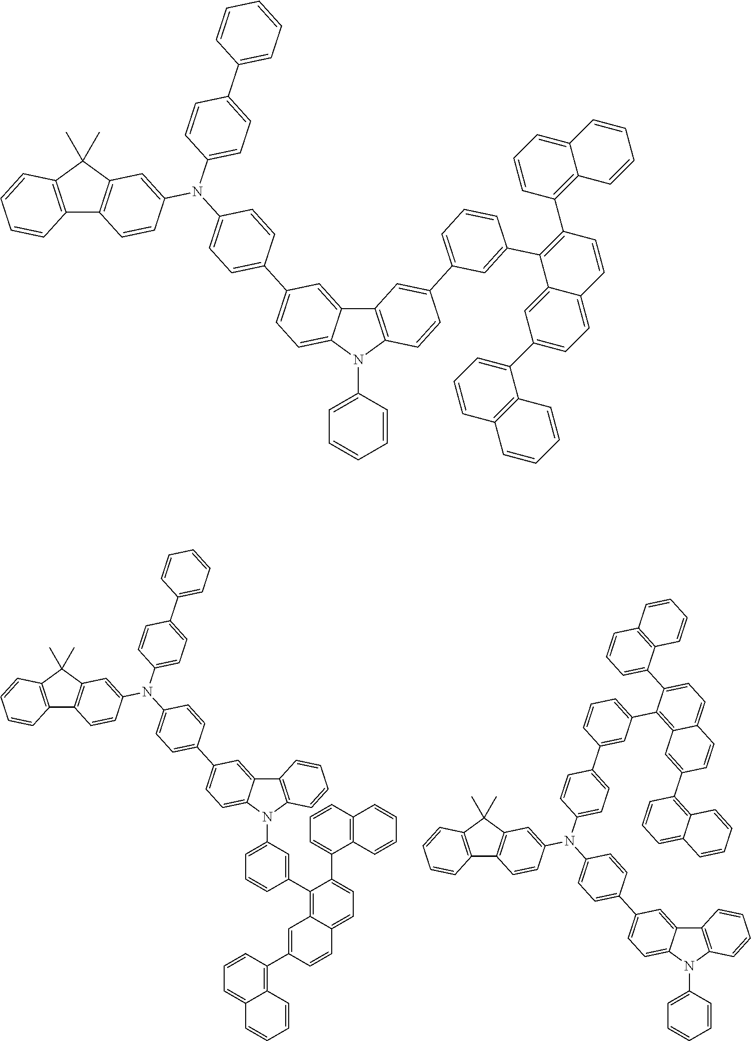

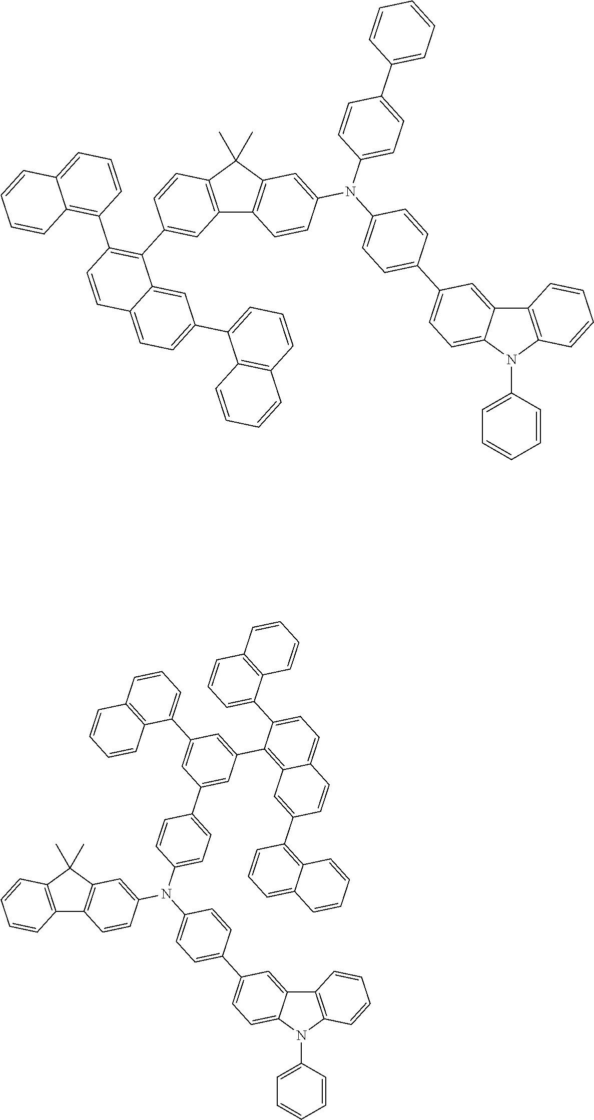 [SMILES: CC1(C)c2ccc(-c3c(-c4cccc5ccccc45)ccc4ccc(-c5cccc6ccccc56)cc34)cc2-c2ccc(N(c3ccc(-c4ccccc4)cc3)c3ccc(-c4ccc5c(c4)c4ccccc4n5-c4ccccc4)cc3)cc21 ; CC1(C)c2ccccc2-c2ccc(N(c3ccc(-c4cc(-c5cccc6ccccc56)cc(-c5c(-c6cccc7ccccc67)ccc6ccc(-c7cccc8ccccc78)cc56)c4)cc3)c3ccc(-c4ccc5c(c4)c4ccccc4n5-c4ccccc4)cc3)cc21]

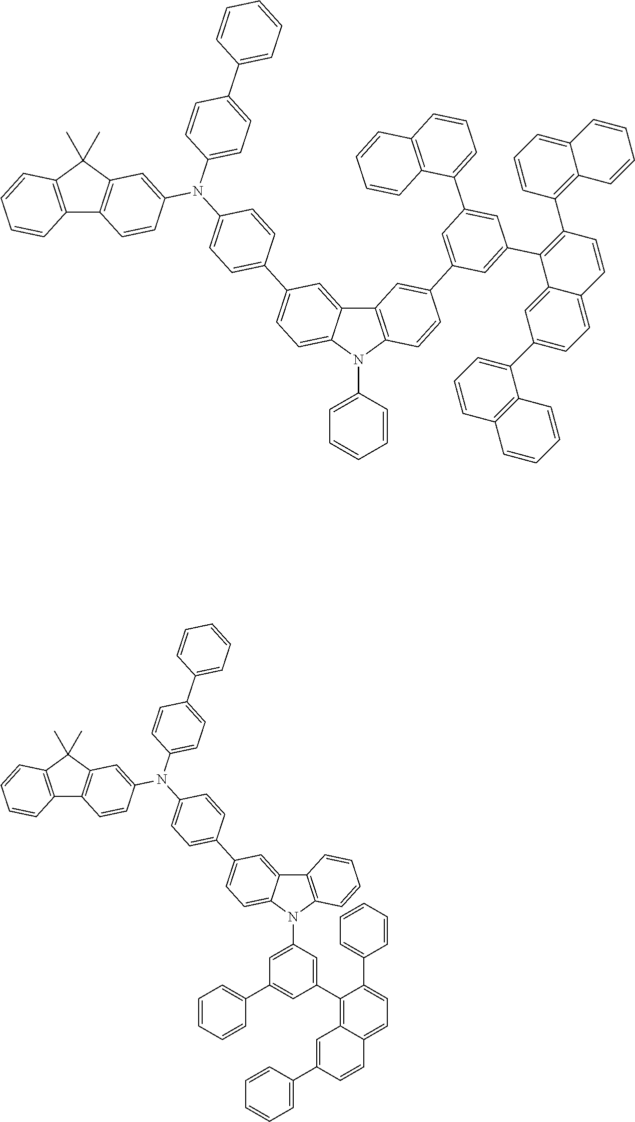 [SMILES: CC1(C)c2ccccc2-c2ccc(N(c3ccc(-c4ccccc4)cc3)c3ccc(-c4ccc5c(c4)c4cc(-c6cc(-c7cccc8ccccc78)cc(-c7c(-c8cccc9ccccc89)ccc8ccc(-c9cccc%10ccccc9%10)cc78)c6)ccc4n5-c4ccccc4)cc3)cc21 ; CC1(C)c2ccccc2-c2ccc(N(c3ccc(-c4ccccc4)cc3)c3ccc(-c4ccc5c(c4)c4ccccc4n5-c4cc(-c5ccccc5)cc(-c5c(-c6ccccc6)ccc6ccc(-c7ccccc7)cc56)c4)cc3)cc21]

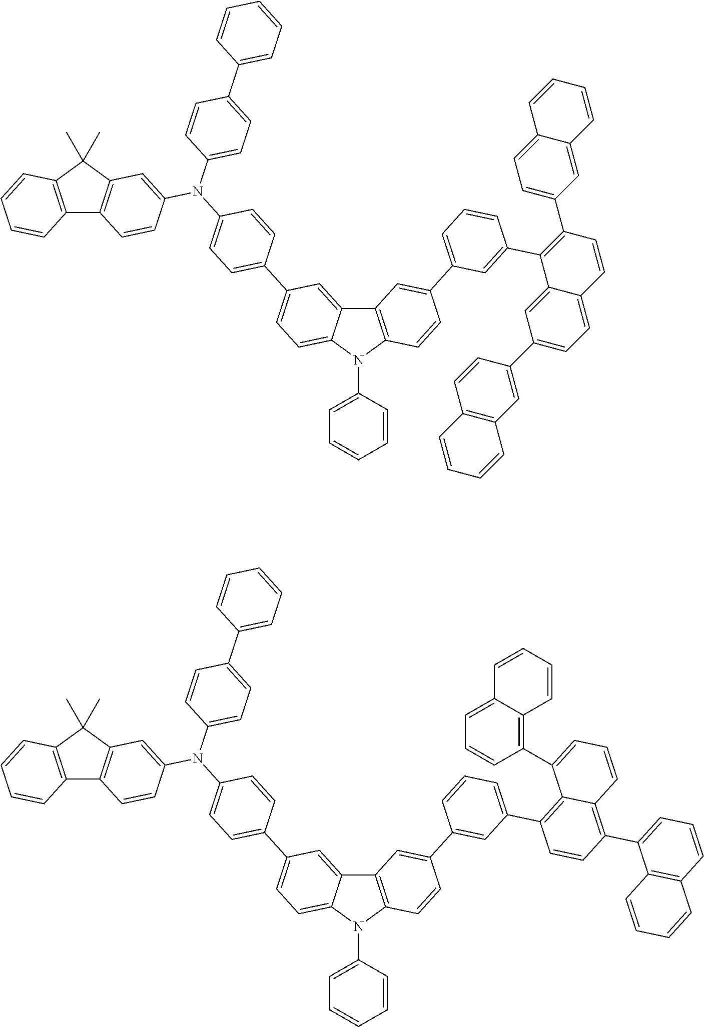 [SMILES: CC1(C)c2ccccc2-c2ccc(N(c3ccc(-c4ccccc4)cc3)c3ccc(-c4ccc5c(c4)c4cc(-c6cccc(-c7c(-c8ccc9ccccc9c8)ccc8ccc(-c9ccc%10ccccc%10c9)cc78)c6)ccc4n5-c4ccccc4)cc3)cc21 ; CC1(C)c2ccccc2-c2ccc(N(c3ccc(-c4ccccc4)cc3)c3ccc(-c4ccc5c(c4)c4cc(-c6cccc(-c7ccc(-c8cccc9ccccc89)c8cccc(-c9cccc%10ccccc9%10)c78)c6)ccc4n5-c4ccccc4)cc3)cc21]

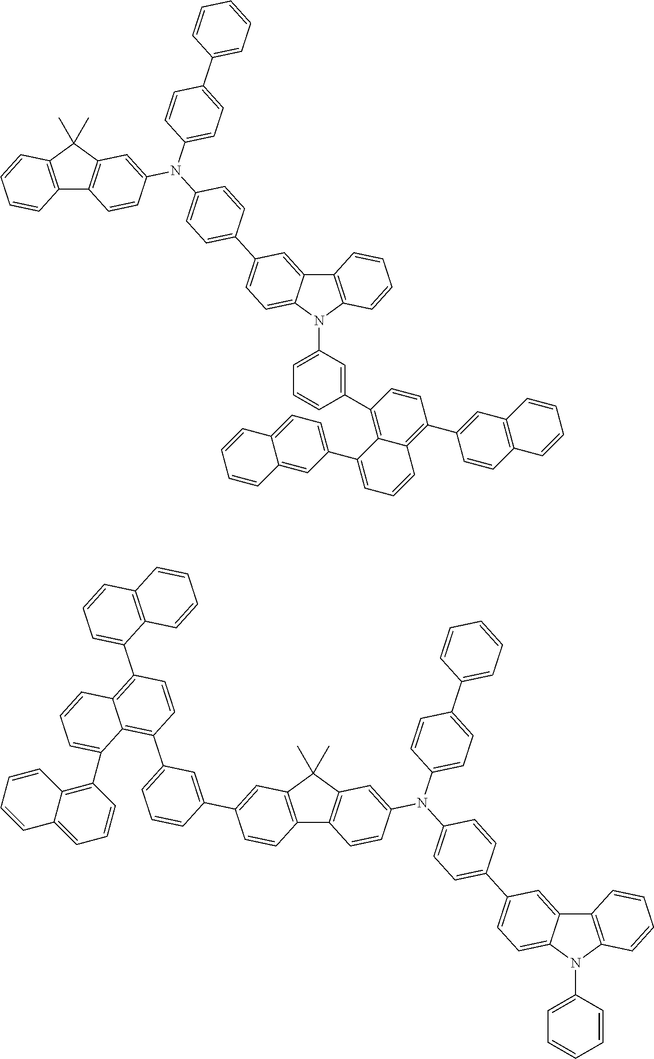 [SMILES: CC1(C)c2ccccc2-c2ccc(N(c3ccc(-c4ccccc4)cc3)c3ccc(-c4ccc5c(c4)c4ccccc4n5-c4cccc(-c5ccc(-c6ccc7ccccc7c6)c6cccc(-c7ccc8ccccc8c7)c56)c4)cc3)cc21 ; CC1(C)c2cc(-c3cccc(-c4ccc(-c5cccc6ccccc56)c5cccc(-c6cccc7ccccc67)c45)c3)ccc2-c2ccc(N(c3ccc(-c4ccccc4)cc3)c3ccc(-c4ccc5c(c4)c4ccccc4n5-c4ccccc4)cc3)cc21]

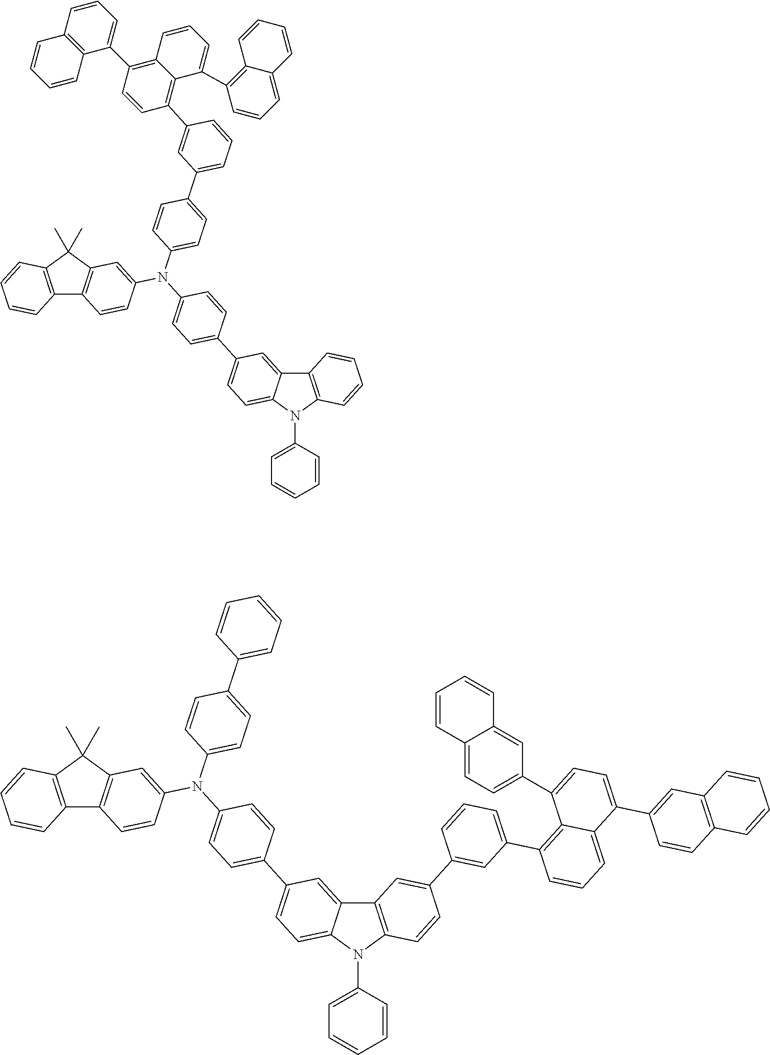 [SMILES: CC1(C)c2ccccc2-c2ccc(N(c3ccc(-c4cccc(-c5ccc(-c6cccc7ccccc67)c6cccc(-c7cccc8ccccc78)c56)c4)cc3)c3ccc(-c4ccc5c(c4)c4ccccc4n5-c4ccccc4)cc3)cc21 ; CC1(C)c2ccccc2-c2ccc(N(c3ccc(-c4ccccc4)cc3)c3ccc(-c4ccc5c(c4)c4cc(-c6cccc(-c7cccc8c(-c9ccc%10ccccc%10c9)ccc(-c9ccc%10ccccc%10c9)c78)c6)ccc4n5-c4ccccc4)cc3)cc21]

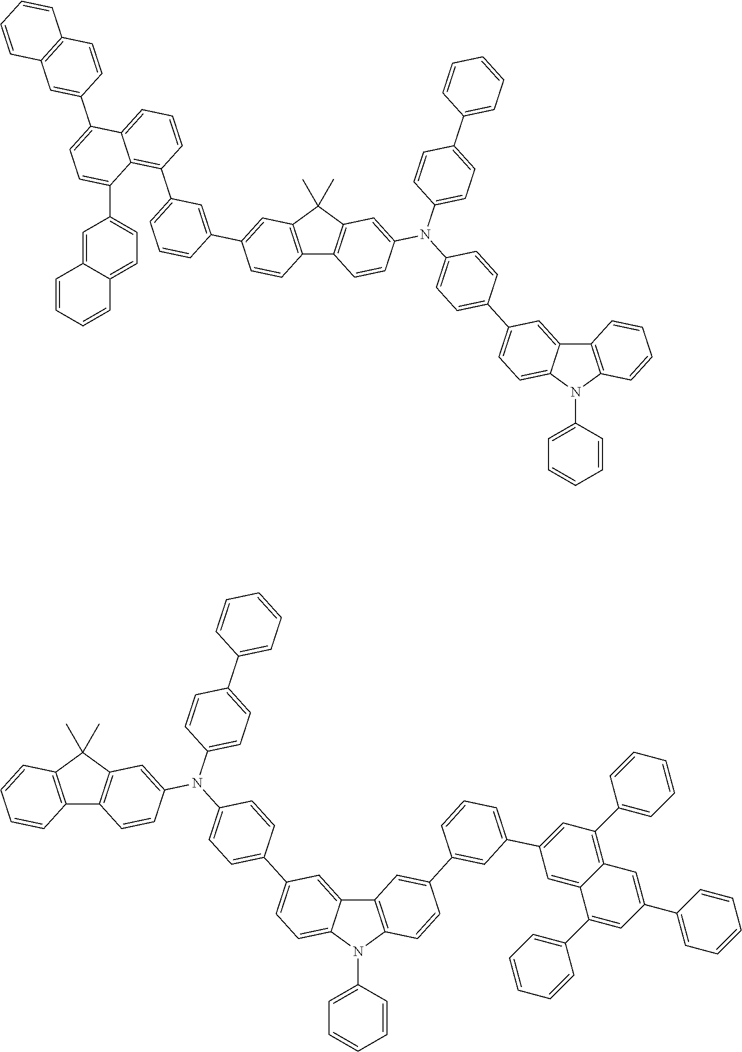 [SMILES: CC1(C)c2cc(-c3cccc(-c4cccc5c(-c6ccc7ccccc7c6)ccc(-c6ccc7ccccc7c6)c45)c3)ccc2-c2ccc(N(c3ccc(-c4ccccc4)cc3)c3ccc(-c4ccc5c(c4)c4ccccc4n5-c4ccccc4)cc3)cc21 ; CC1(C)c2ccccc2-c2ccc(N(c3ccc(-c4ccccc4)cc3)c3ccc(-c4ccc5c(c4)c4cc(-c6cccc(-c7cc(-c8ccccc8)c8cc(-c9ccccc9)cc(-c9ccccc9)c8c7)c6)ccc4n5-c4ccccc4)cc3)cc21]

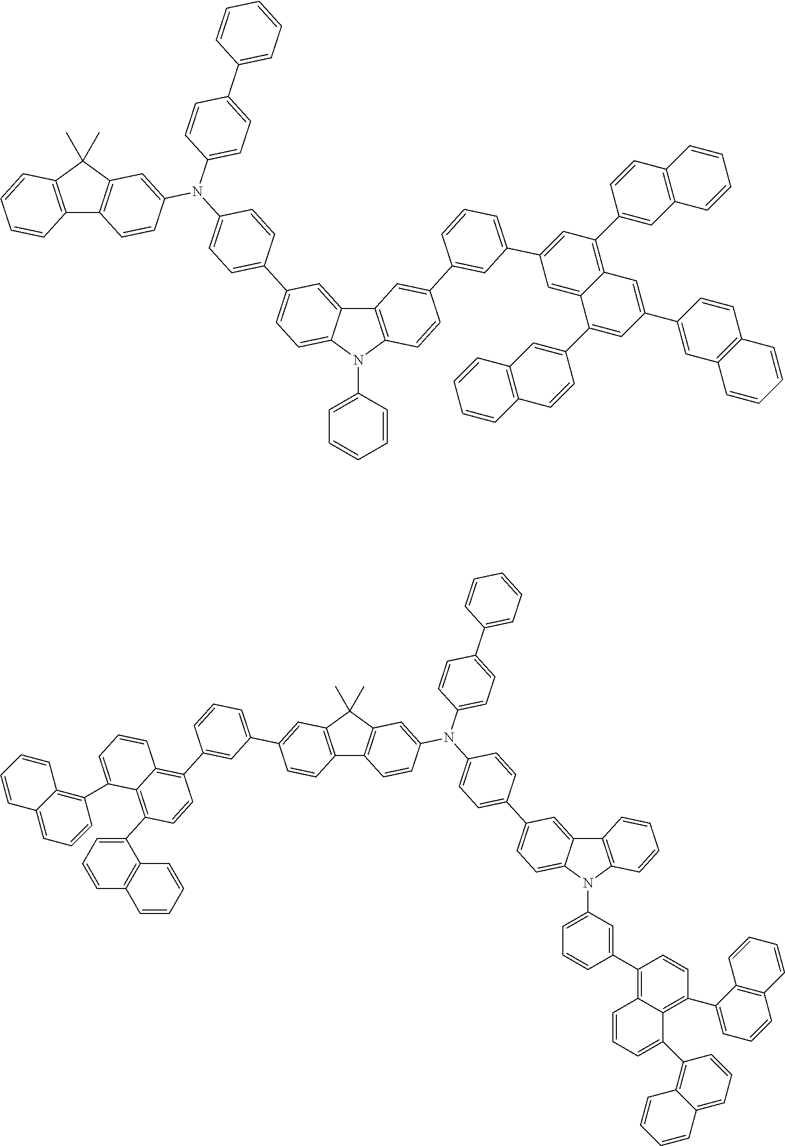 [SMILES: CC1(C)c2ccccc2-c2ccc(N(c3ccc(-c4ccccc4)cc3)c3ccc(-c4ccc5c(c4)c4cc(-c6cccc(-c7cc(-c8ccc9ccccc9c8)c8cc(-c9ccc%10ccccc%10c9)cc(-c9ccc%10ccccc%10c9)c8c7)c6)ccc4n5-c4ccccc4)cc3)cc21 ; CC1(C)c2cc(-c3cccc(-c4ccc(-c5cccc6ccccc56)c5c(-c6cccc7ccccc67)cccc45)c3)ccc2-c2ccc(N(c3ccc(-c4ccccc4)cc3)c3ccc(-c4ccc5c(c4)c4ccccc4n5-c4cccc(-c5ccc(-c6cccc7ccccc67)c6c(-c7cccc8ccccc78)cccc56)c4)cc3)cc21]

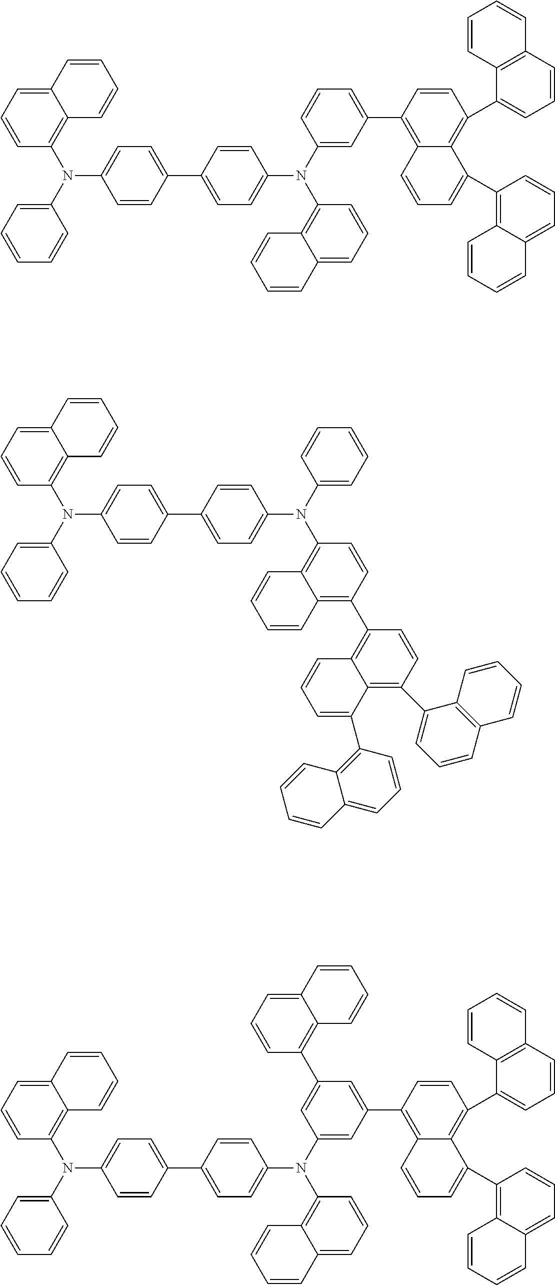 [SMILES: c1ccc(N(c2ccc(-c3ccc(N(c4cccc(-c5ccc(-c6cccc7ccccc67)c6c(-c7cccc8ccccc78)cccc56)c4)c4cccc5ccccc45)cc3)cc2)c2cccc3ccccc23)cc1 ; c1ccc(N(c2ccc(-c3ccc(N(c4ccccc4)c4ccc(-c5ccc(-c6cccc7ccccc67)c6c(-c7cccc8ccccc78)cccc56)c5ccccc45)cc3)cc2)c2cccc3ccccc23)cc1 ; c1ccc(N(c2ccc(-c3ccc(N(c4cc(-c5cccc6ccccc56)cc(-c5ccc(-c6cccc7ccccc67)c6c(-c7cccc8ccccc78)cccc56)c4)c4cccc5ccccc45)cc3)cc2)c2cccc3ccccc23)cc1]

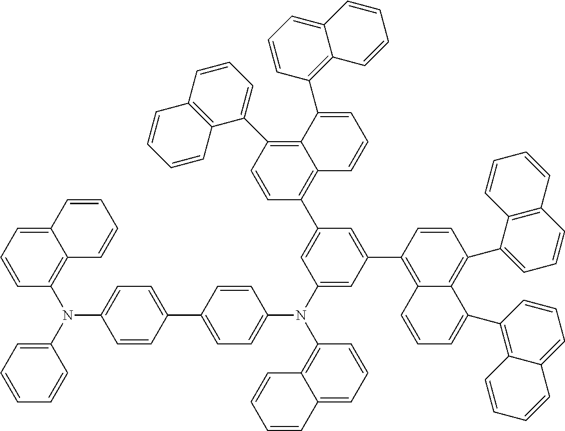 [SMILES: c1ccc(N(c2ccc(-c3ccc(N(c4cc(-c5ccc(-c6cccc7ccccc67)c6c(-c7cccc8ccccc78)cccc56)cc(-c5ccc(-c6cccc7ccccc67)c6c(-c7cccc8ccccc78)cccc56)c4)c4cccc5ccccc45)cc3)cc2)c2cccc3ccccc23)cc1]

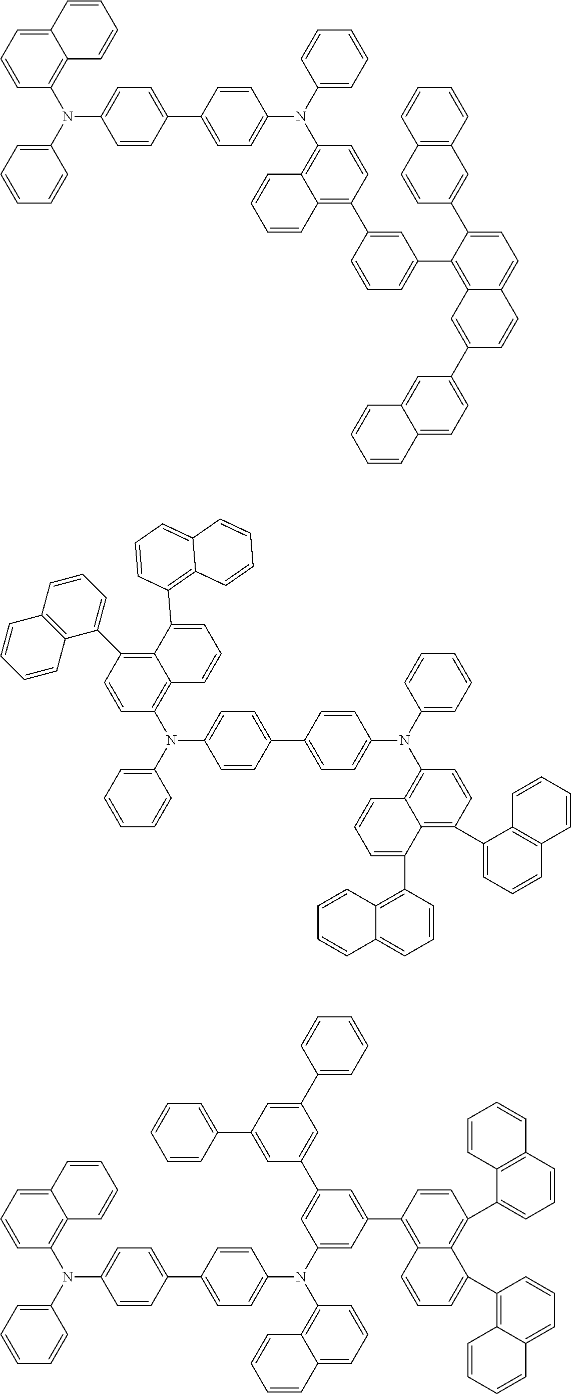 [SMILES: c1ccc(N(c2ccc(-c3ccc(N(c4ccccc4)c4ccc(-c5cccc(-c6c(-c7ccc8ccccc8c7)ccc7ccc(-c8ccc9ccccc9c8)cc67)c5)c5ccccc45)cc3)cc2)c2cccc3ccccc23)cc1 ; c1ccc(N(c2ccc(-c3ccc(N(c4ccccc4)c4ccc(-c5cccc6ccccc56)c5c(-c6cccc7ccccc67)cccc45)cc3)cc2)c2ccc(-c3cccc4ccccc34)c3c(-c4cccc5ccccc45)cccc23)cc1 ; c1ccc(-c2cc(-c3ccccc3)cc(-c3cc(-c4ccc(-c5cccc6ccccc56)c5c(-c6cccc7ccccc67)cccc45)cc(N(c4ccc(-c5ccc(N(c6ccccc6)c6cccc7ccccc67)cc5)cc4)c4cccc5ccccc45)c3)c2)cc1]

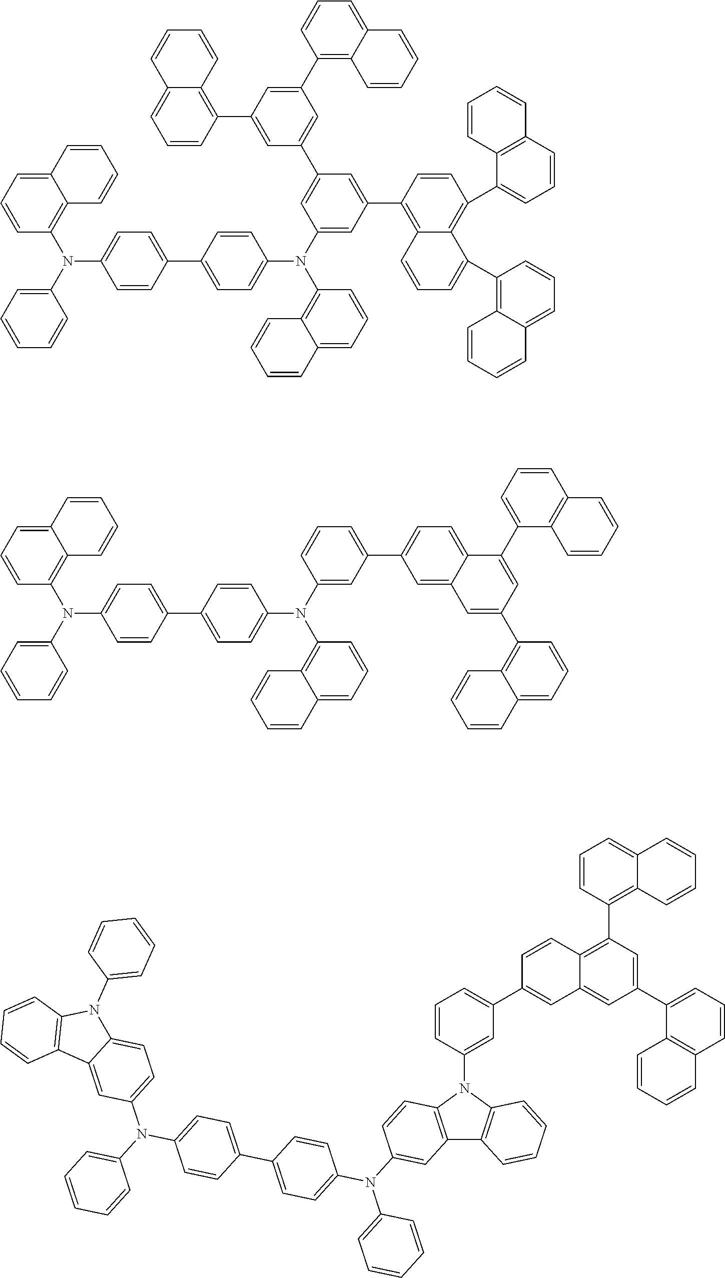 [SMILES: c1ccc(N(c2ccc(-c3ccc(N(c4cc(-c5cc(-c6cccc7ccccc67)cc(-c6cccc7ccccc67)c5)cc(-c5ccc(-c6cccc7ccccc67)c6c(-c7cccc8ccccc78)cccc56)c4)c4cccc5ccccc45)cc3)cc2)c2cccc3ccccc23)cc1 ; c1ccc(N(c2ccc(-c3ccc(N(c4cccc(-c5ccc6c(-c7cccc8ccccc78)cc(-c7cccc8ccccc78)cc6c5)c4)c4cccc5ccccc45)cc3)cc2)c2cccc3ccccc23)cc1 ; c1ccc(N(c2ccc(-c3ccc(N(c4ccccc4)c4ccc5c(c4)c4ccccc4n5-c4cccc(-c5ccc6c(-c7cccc8ccccc78)cc(-c7cccc8ccccc78)cc6c5)c4)cc3)cc2)c2ccc3c(c2)c2ccccc2n3-c2ccccc2)cc1]

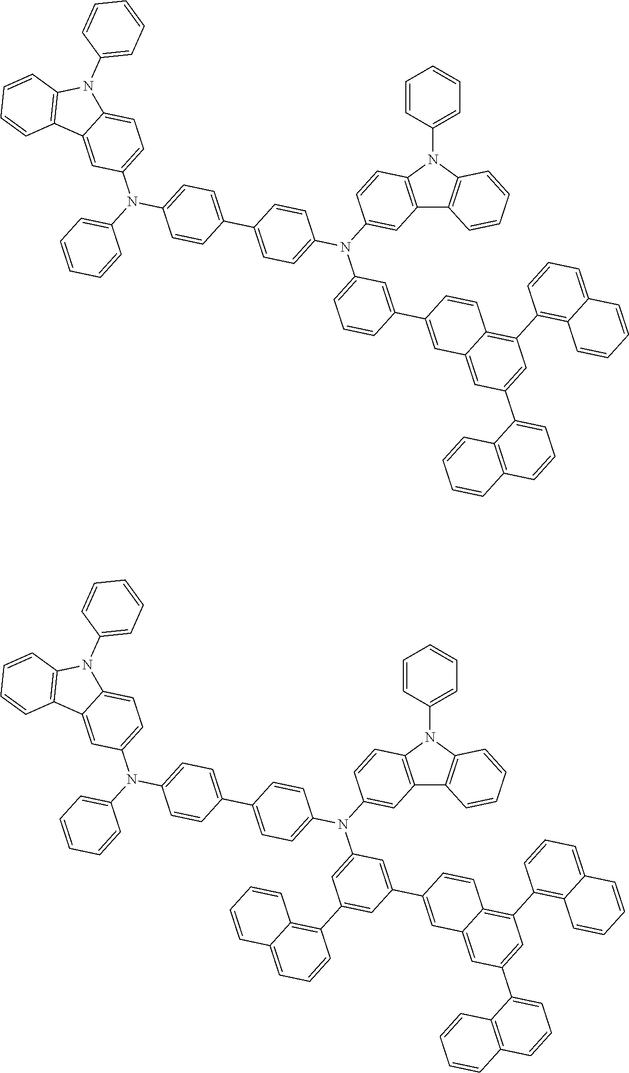 [SMILES: c1ccc(N(c2ccc(-c3ccc(N(c4cccc(-c5ccc6c(-c7cccc8ccccc78)cc(-c7cccc8ccccc78)cc6c5)c4)c4ccc5c(c4)c4ccccc4n5-c4ccccc4)cc3)cc2)c2ccc3c(c2)c2ccccc2n3-c2ccccc2)cc1 ; c1ccc(N(c2ccc(-c3ccc(N(c4cc(-c5ccc6c(-c7cccc8ccccc78)cc(-c7cccc8ccccc78)cc6c5)cc(-c5cccc6ccccc56)c4)c4ccc5c(c4)c4ccccc4n5-c4ccccc4)cc3)cc2)c2ccc3c(c2)c2ccccc2n3-c2ccccc2)cc1]

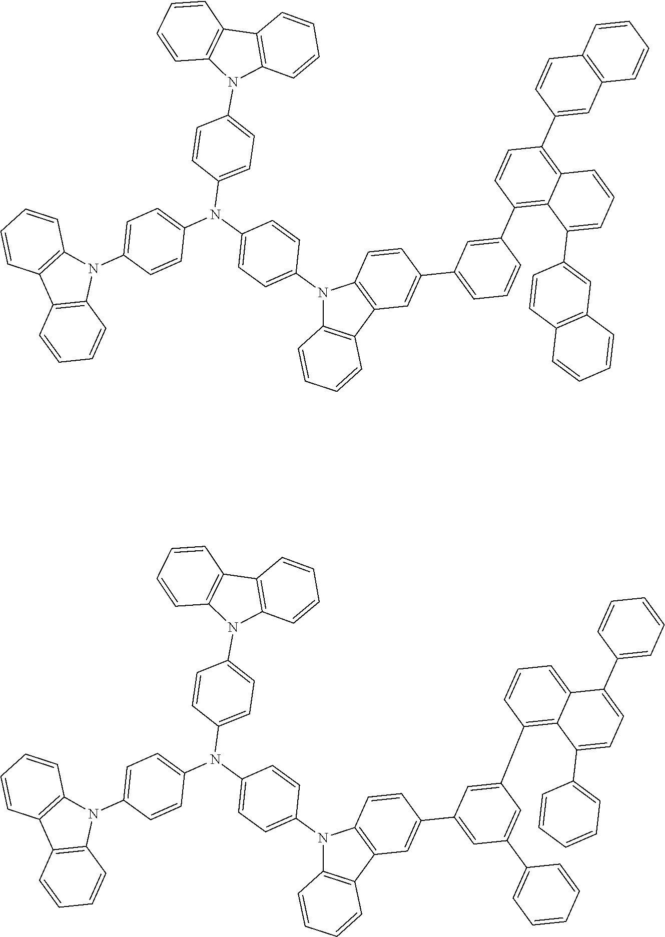 [SMILES: c1cc(-c2ccc3c(c2)c2ccccc2n3-c2ccc(N(c3ccc(-n4c5ccccc5c5ccccc54)cc3)c3ccc(-n4c5ccccc5c5ccccc54)cc3)cc2)cc(-c2ccc(-c3ccc4ccccc4c3)c3cccc(-c4ccc5ccccc5c4)c23)c1 ; c1ccc(-c2cc(-c3ccc4c(c3)c3ccccc3n4-c3ccc(N(c4ccc(-n5c6ccccc6c6ccccc65)cc4)c4ccc(-n5c6ccccc6c6ccccc65)cc4)cc3)cc(-c3cccc4c(-c5ccccc5)ccc(-c5ccccc5)c34)c2)cc1]

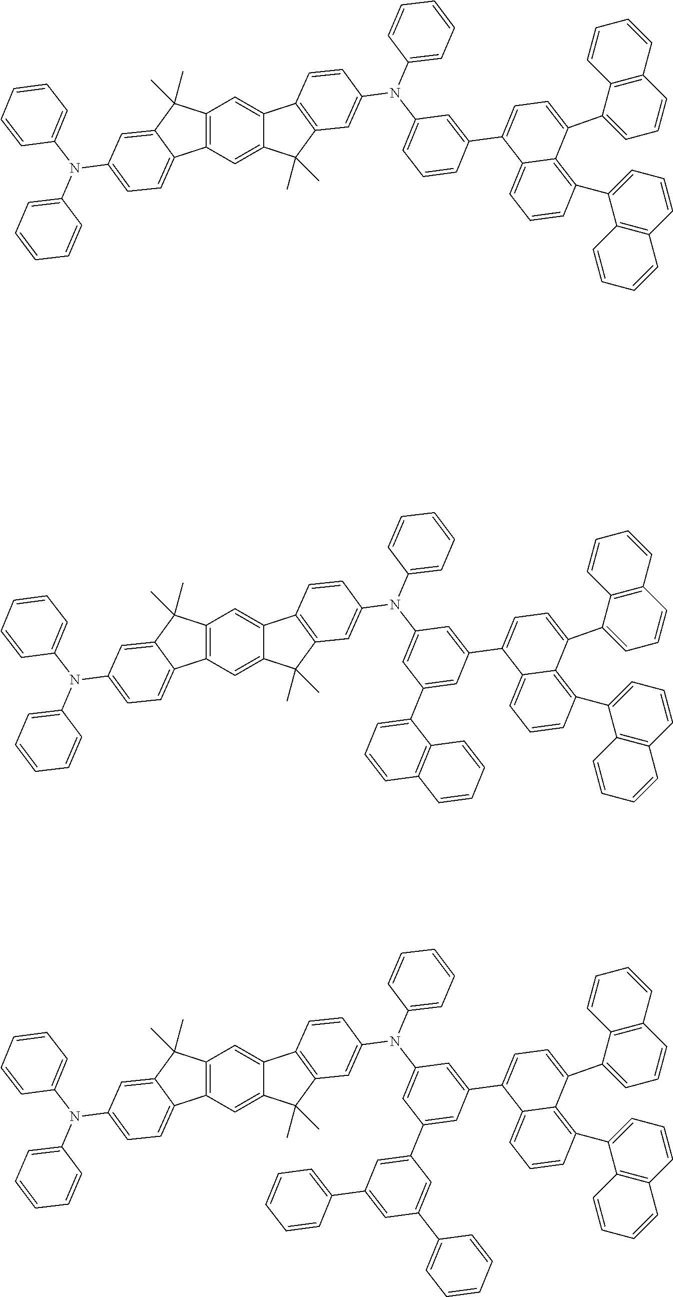 [SMILES: CC1(C)c2cc(N(c3ccccc3)c3ccccc3)ccc2-c2cc3c(cc21)-c1ccc(N(c2ccccc2)c2cccc(-c4ccc(-c5cccc6ccccc56)c5c(-c6cccc7ccccc67)cccc45)c2)cc1C3(C)C ; CC1(C)c2cc(N(c3ccccc3)c3ccccc3)ccc2-c2cc3c(cc21)-c1ccc(N(c2ccccc2)c2cc(-c4cccc5ccccc45)cc(-c4ccc(-c5cccc6ccccc56)c5c(-c6cccc7ccccc67)cccc45)c2)cc1C3(C)C ; CC1(C)c2cc(N(c3ccccc3)c3ccccc3)ccc2-c2cc3c(cc21)-c1ccc(N(c2ccccc2)c2cc(-c4cc(-c5ccccc5)cc(-c5ccccc5)c4)cc(-c4ccc(-c5cccc6ccccc56)c5c(-c6cccc7ccccc67)cccc45)c2)cc1C3(C)C]

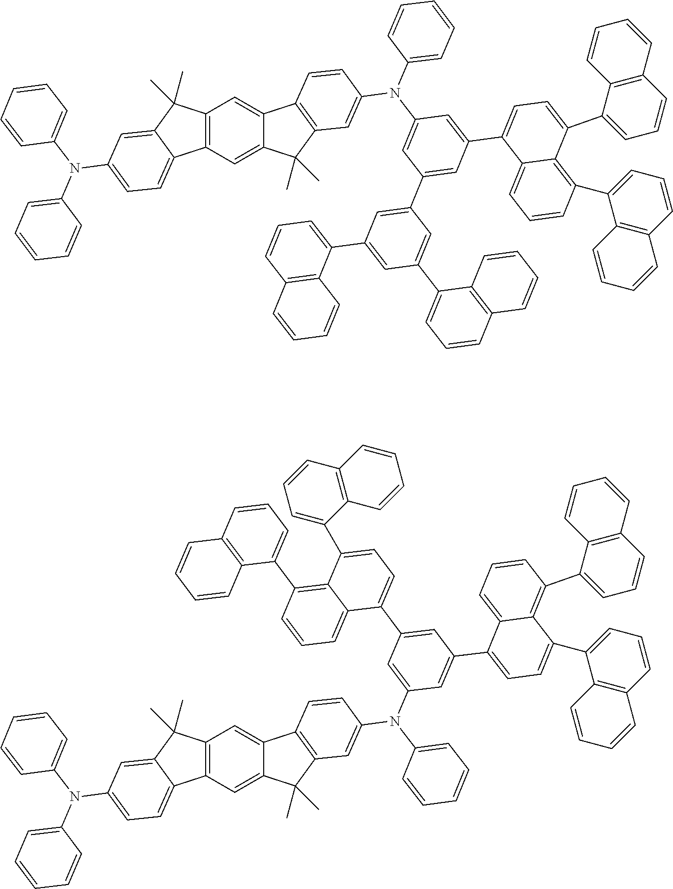 [SMILES: CC1(C)c2cc(N(c3ccccc3)c3ccccc3)ccc2-c2cc3c(cc21)-c1ccc(N(c2ccccc2)c2cc(-c4cc(-c5cccc6ccccc56)cc(-c5cccc6ccccc56)c4)cc(-c4ccc(-c5cccc6ccccc56)c5c(-c6cccc7ccccc67)cccc45)c2)cc1C3(C)C ; CC1(C)c2cc(N(c3ccccc3)c3ccccc3)ccc2-c2cc3c(cc21)-c1ccc(N(c2ccccc2)c2cc(-c4ccc(-c5cccc6ccccc56)c5c(-c6cccc7ccccc67)cccc45)cc(-c4ccc(-c5cccc6ccccc56)c5c(-c6cccc7ccccc67)cccc45)c2)cc1C3(C)C]

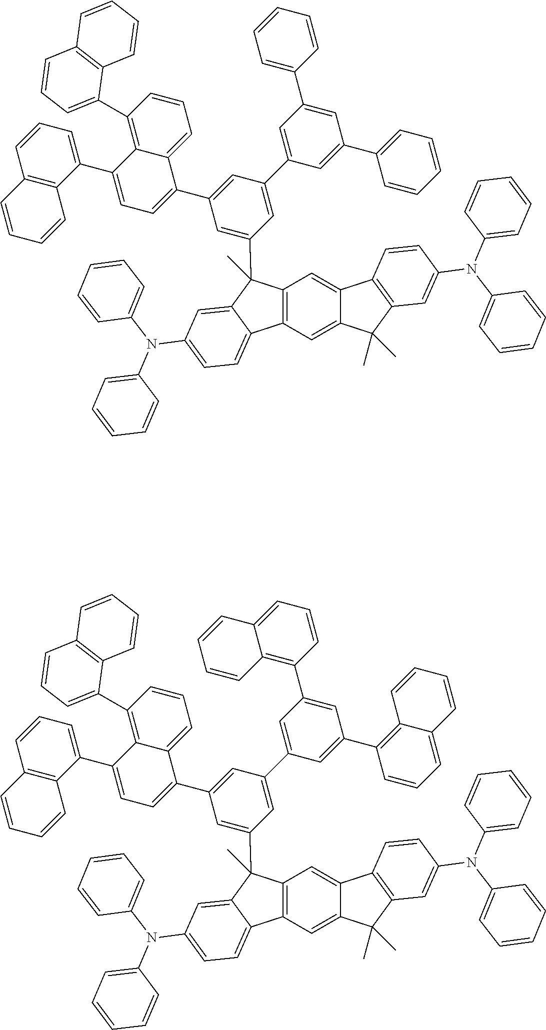 [SMILES: CC1(C)c2cc(N(c3ccccc3)c3ccccc3)ccc2-c2cc3c(cc21)-c1ccc(N(c2ccccc2)c2ccccc2)cc1C3(C)c1cc(-c2cc(-c3ccccc3)cc(-c3ccccc3)c2)cc(-c2ccc(-c3cccc4ccccc34)c3c(-c4cccc5ccccc45)cccc23)c1 ; CC1(C)c2cc(N(c3ccccc3)c3ccccc3)ccc2-c2cc3c(cc21)-c1ccc(N(c2ccccc2)c2ccccc2)cc1C3(C)c1cc(-c2cc(-c3cccc4ccccc34)cc(-c3cccc4ccccc34)c2)cc(-c2ccc(-c3cccc4ccccc34)c3c(-c4cccc5ccccc45)cccc23)c1]

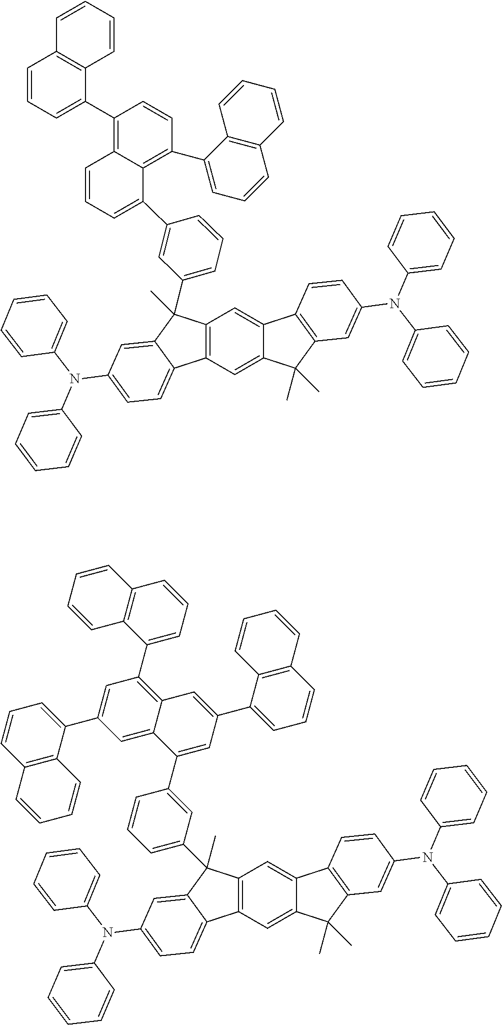 [SMILES: CC1(C)c2cc(N(c3ccccc3)c3ccccc3)ccc2-c2cc3c(cc21)-c1ccc(N(c2ccccc2)c2ccccc2)cc1C3(C)c1cccc(-c2cccc3c(-c4cccc5ccccc45)ccc(-c4cccc5ccccc45)c23)c1 ; CC1(C)c2cc(N(c3ccccc3)c3ccccc3)ccc2-c2cc3c(cc21)-c1ccc(N(c2ccccc2)c2ccccc2)cc1C3(C)c1cccc(-c2cc(-c3cccc4ccccc34)cc3c(-c4cccc5ccccc45)cc(-c4cccc5ccccc45)cc23)c1]

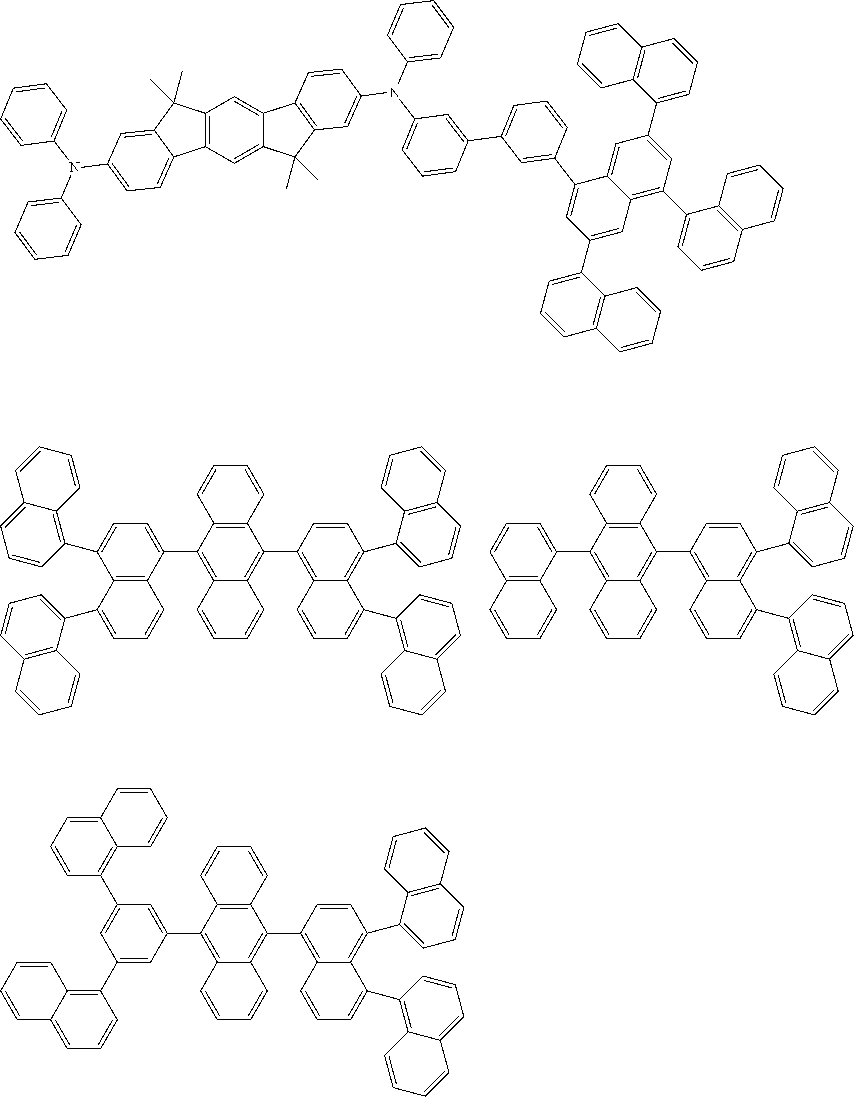

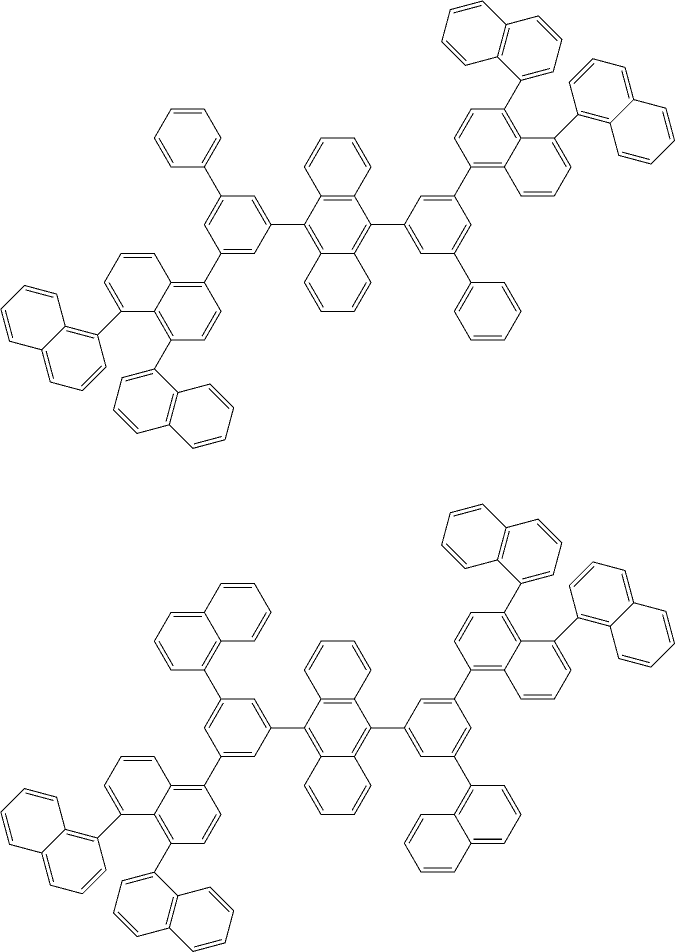

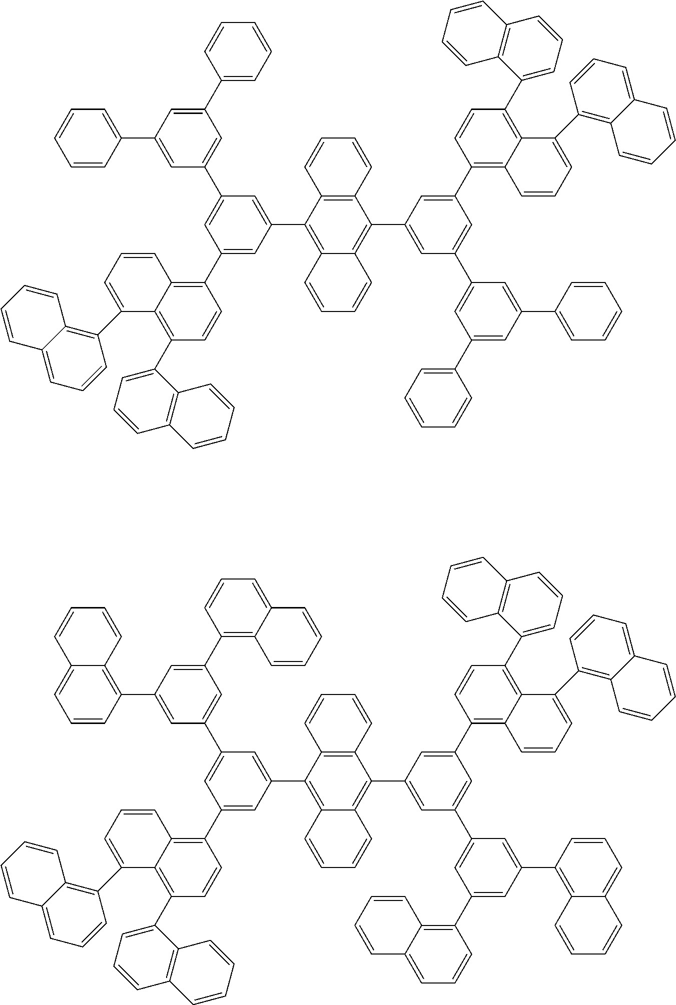

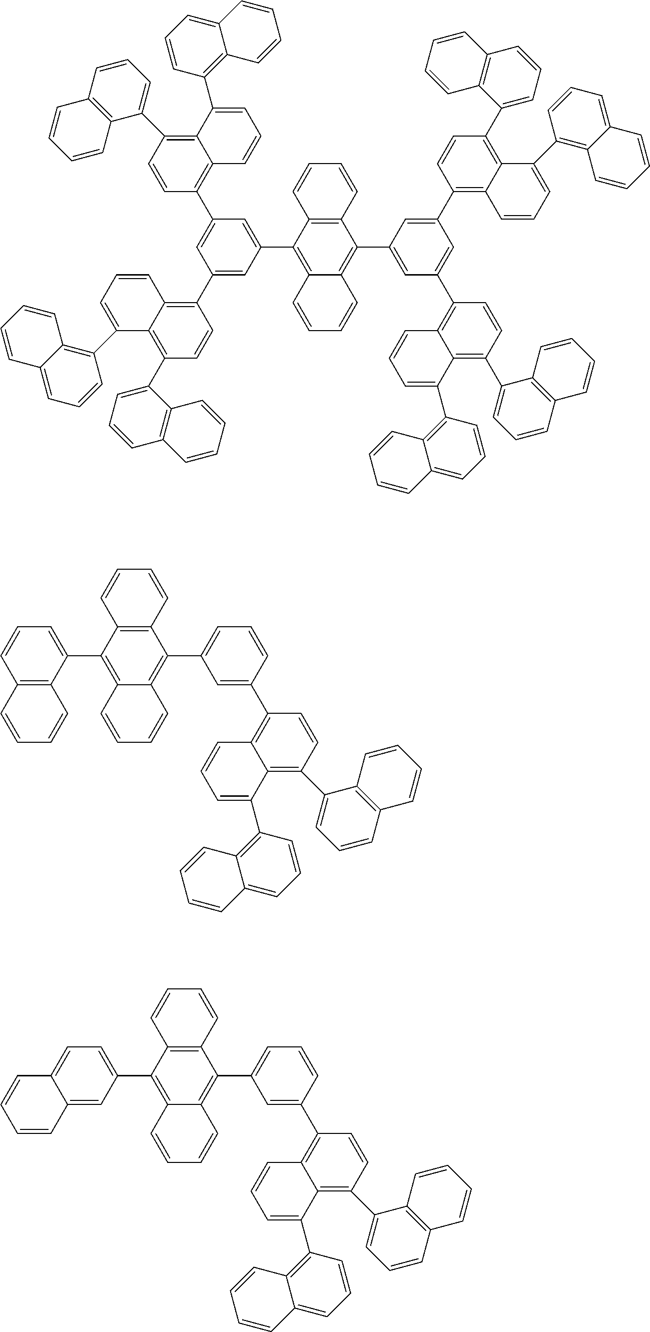

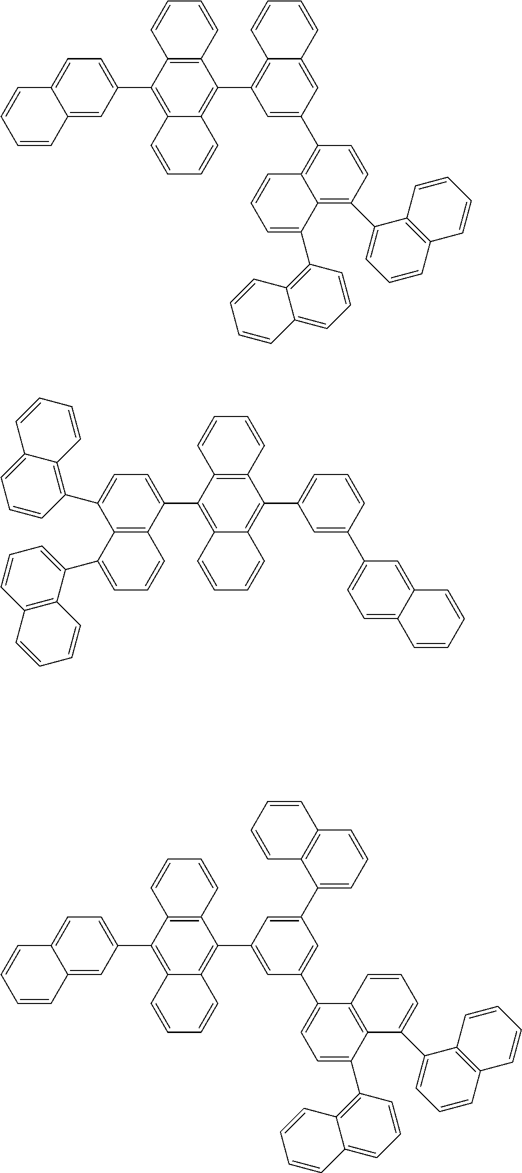

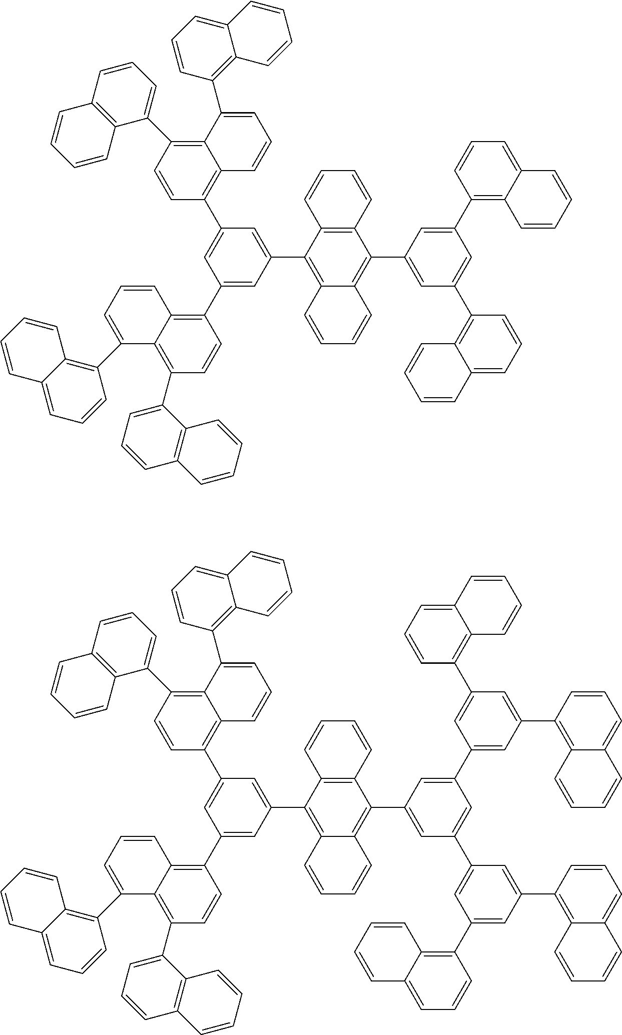

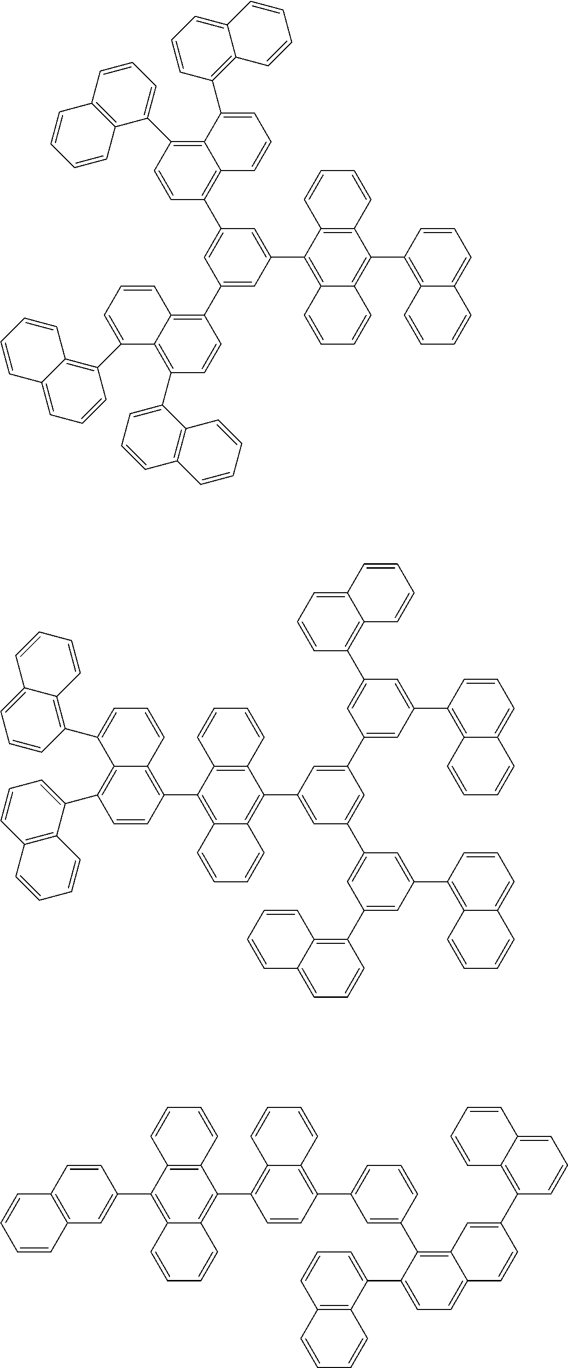

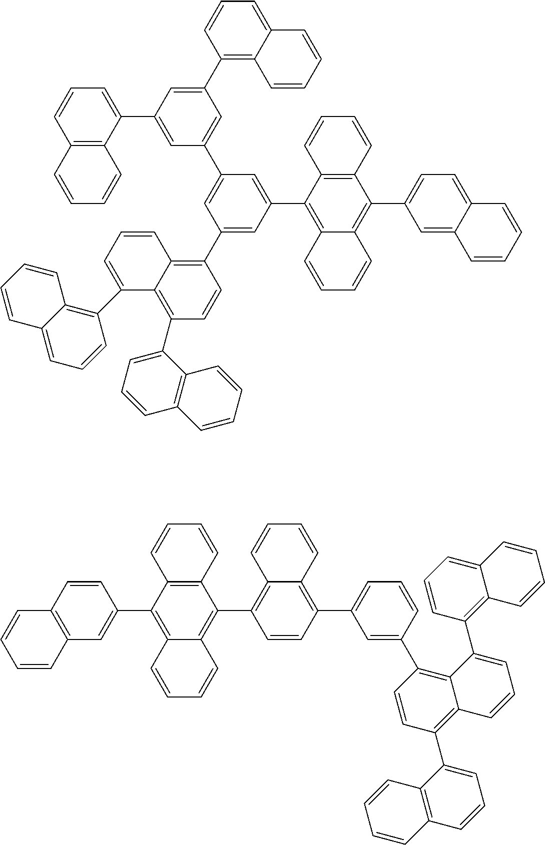

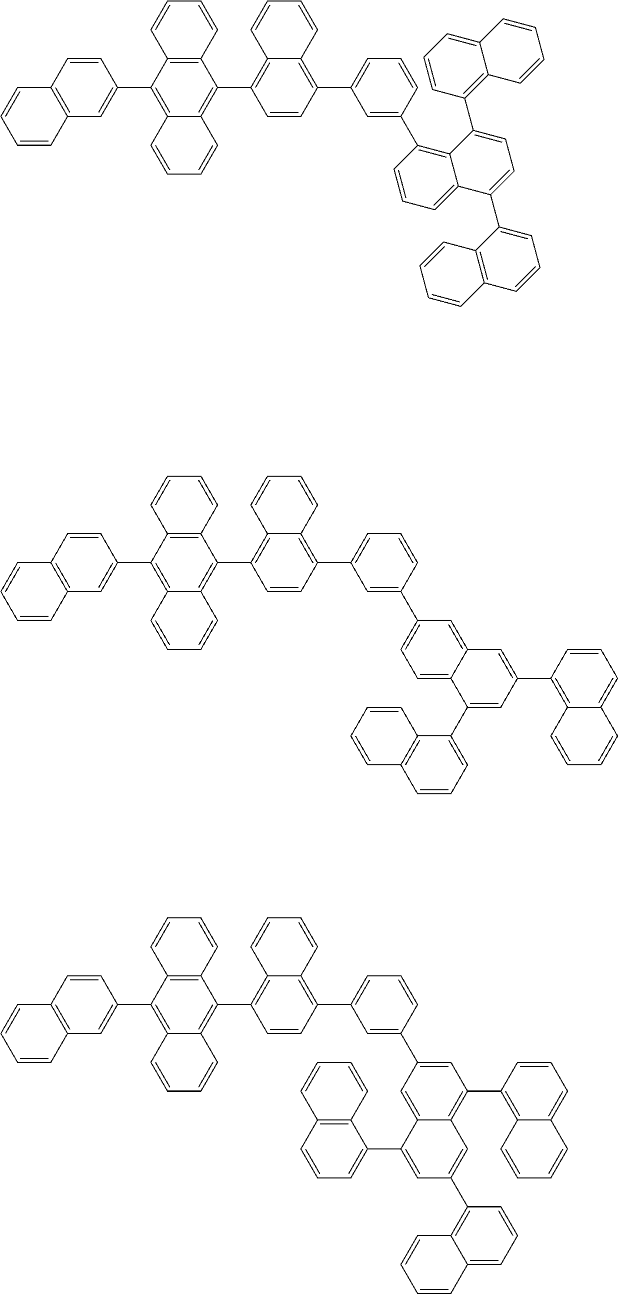

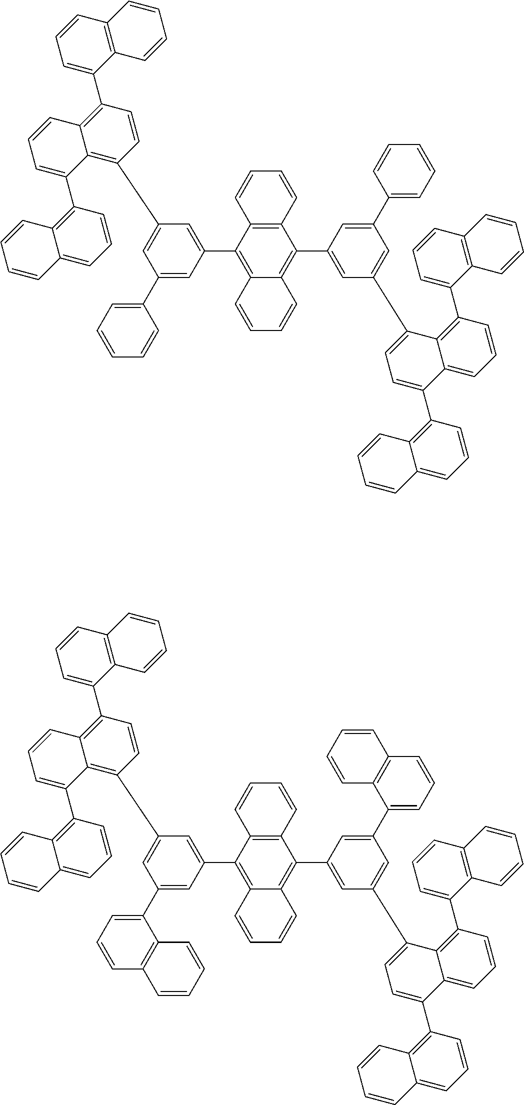

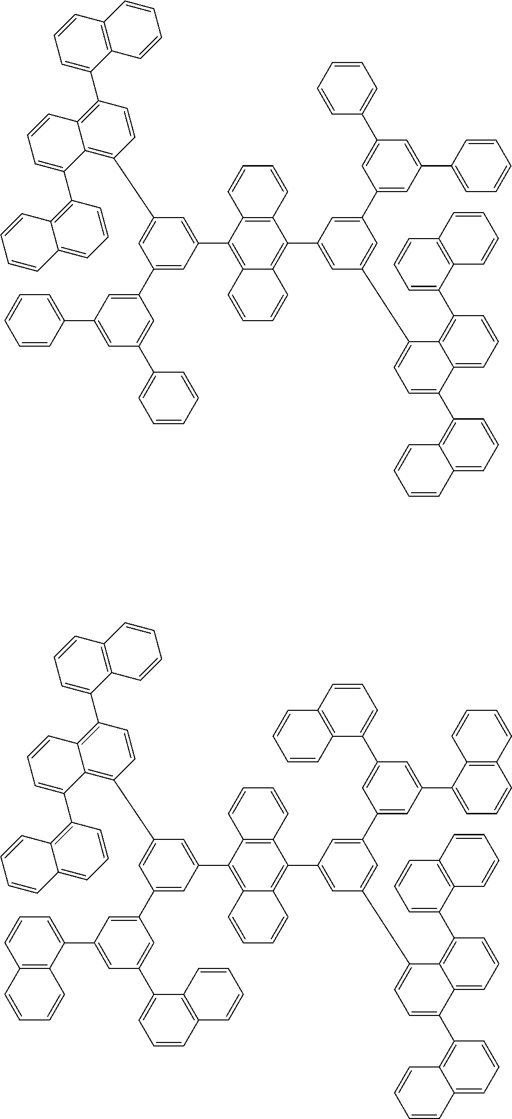

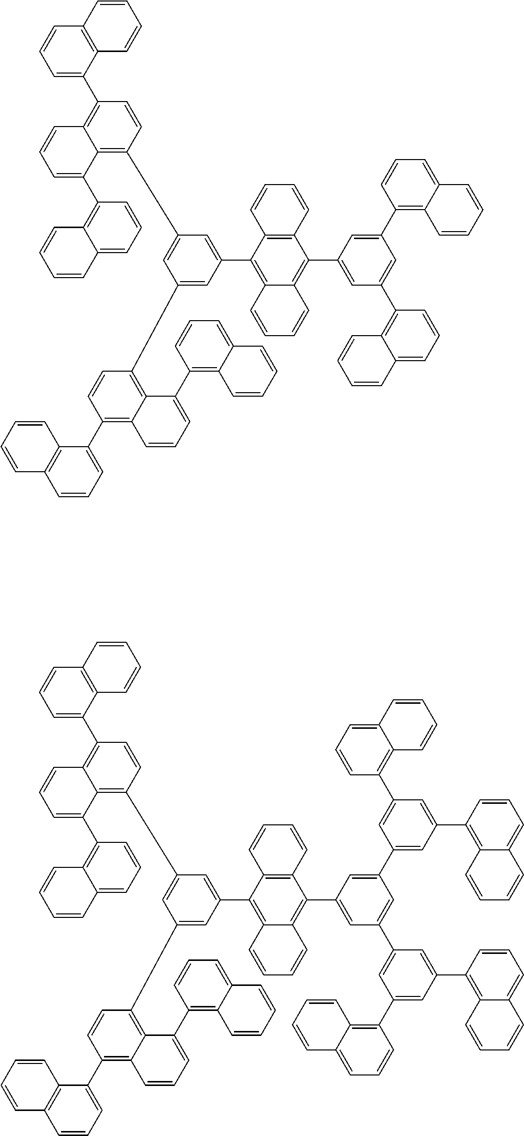

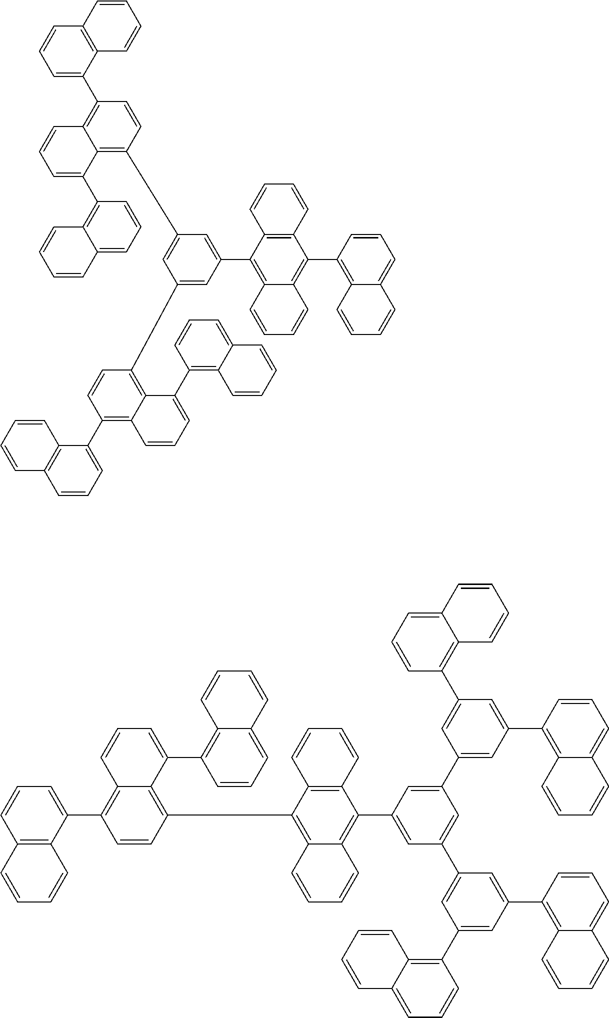

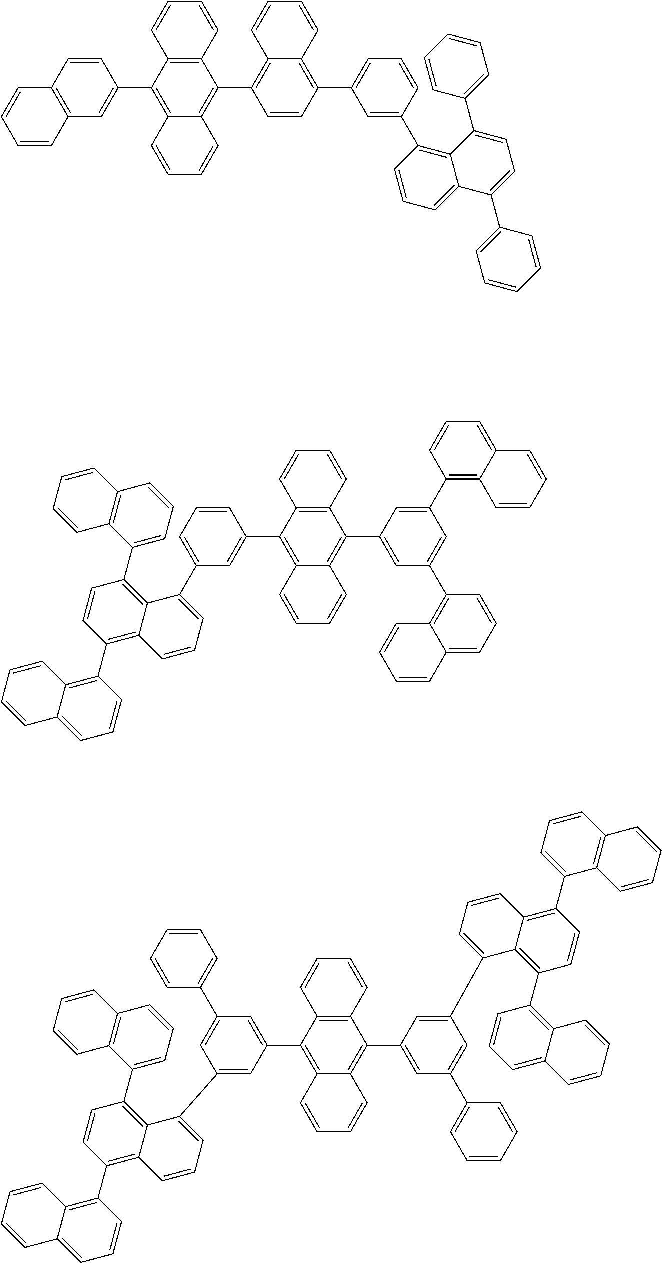

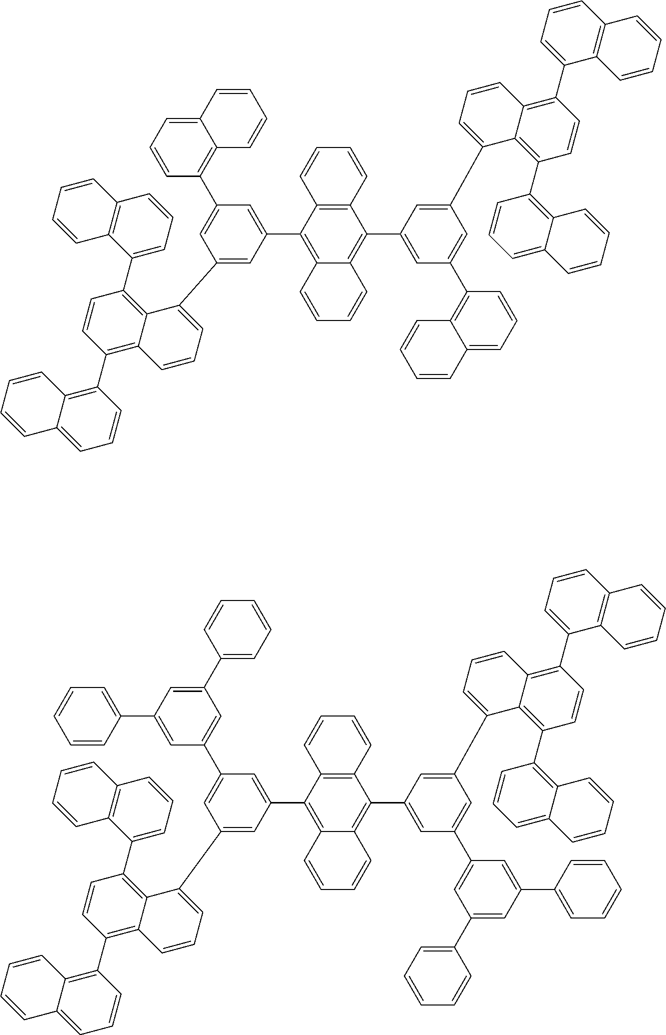

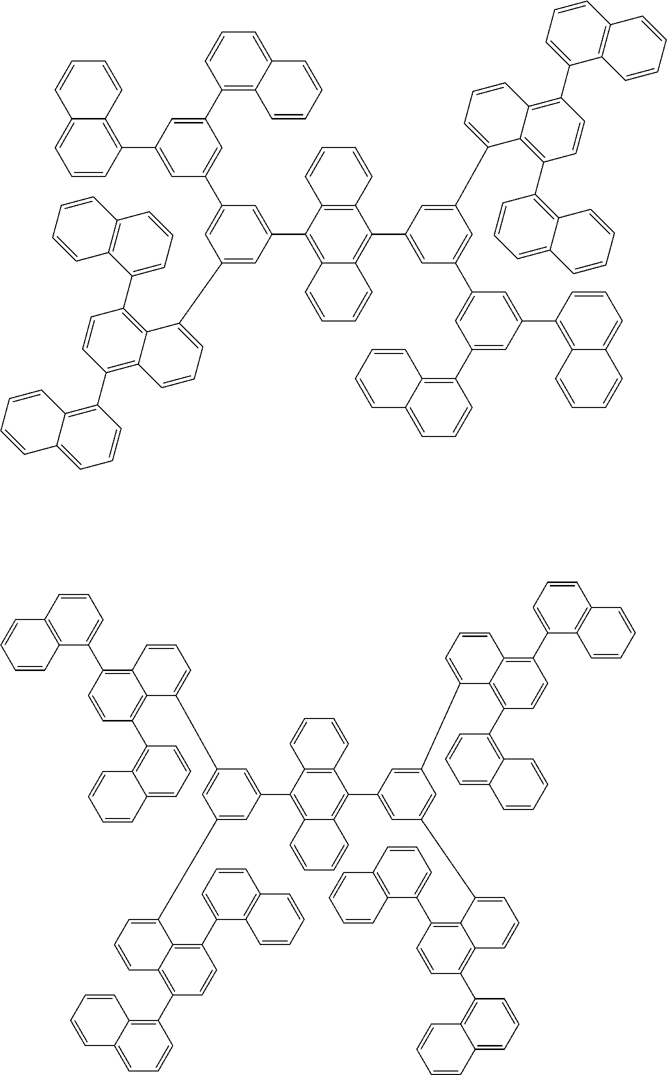

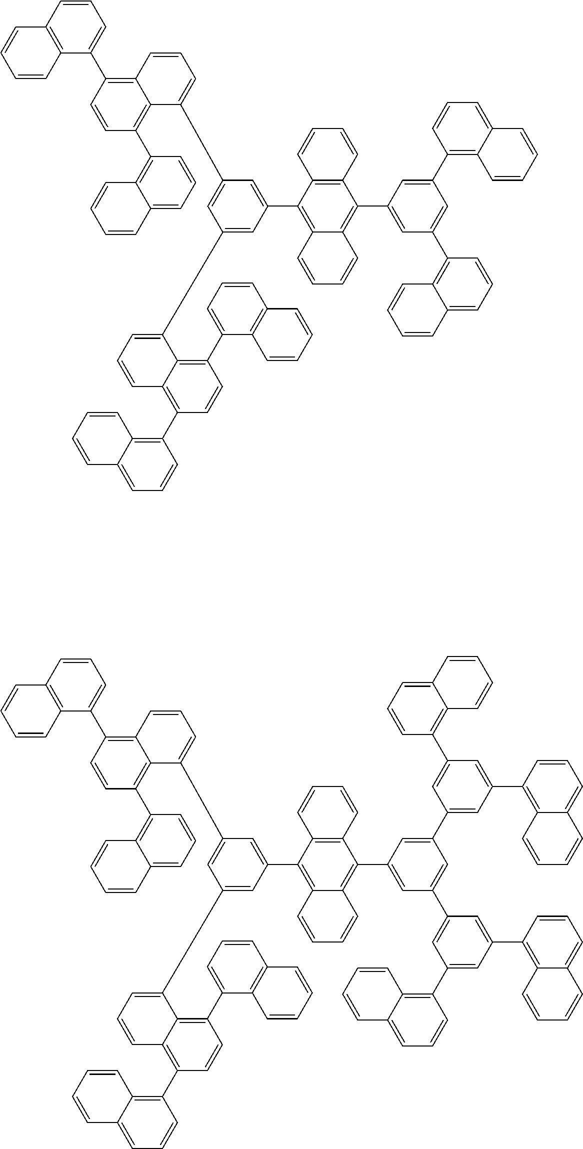

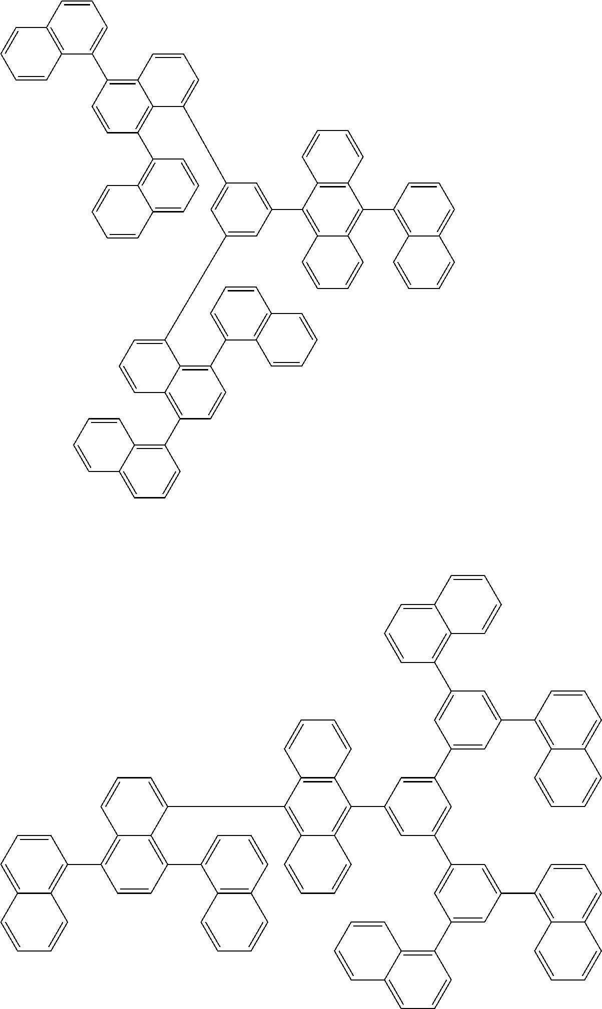

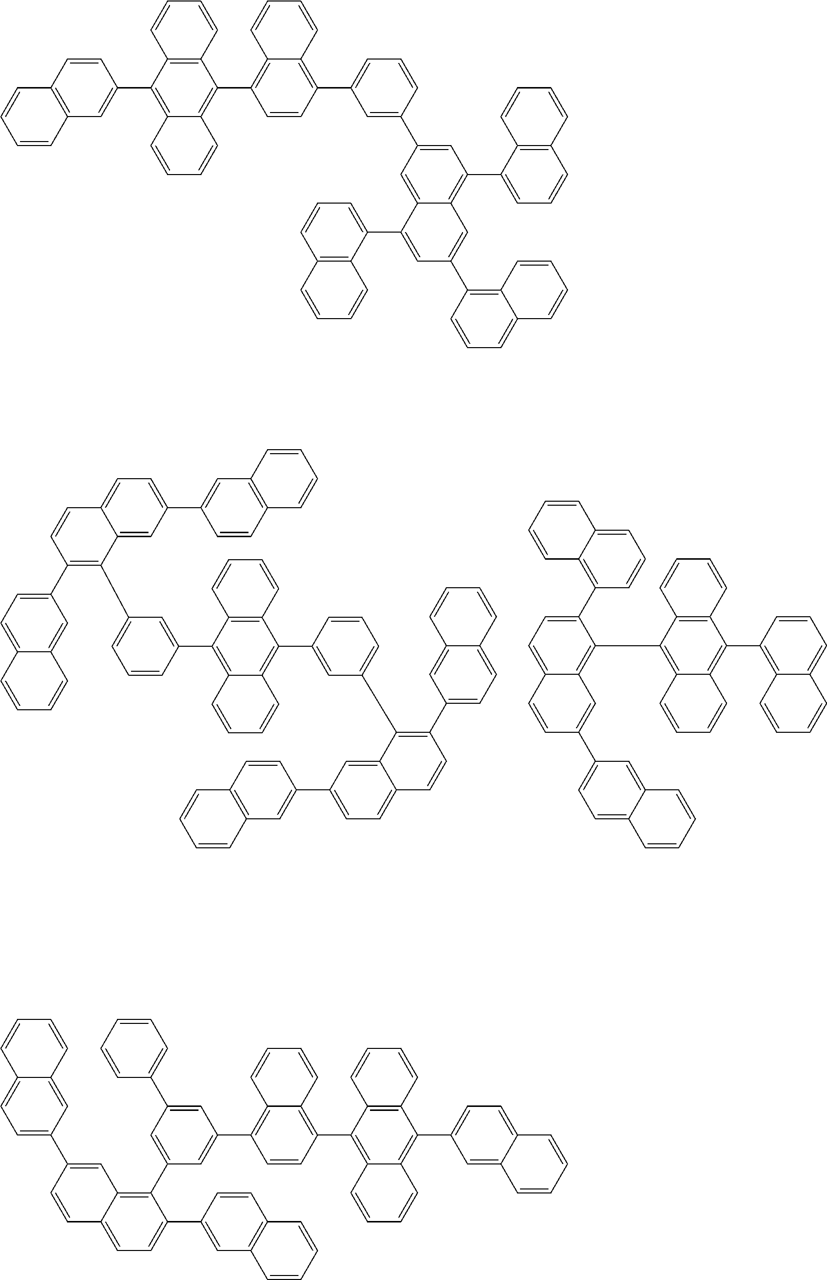

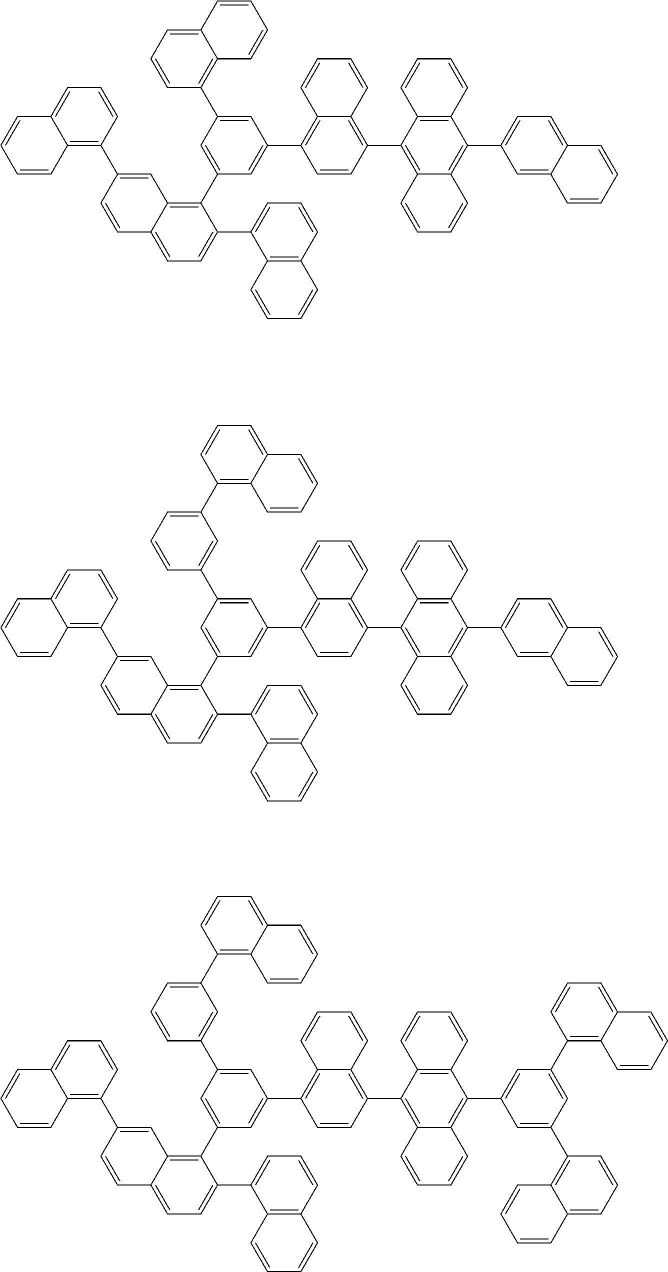

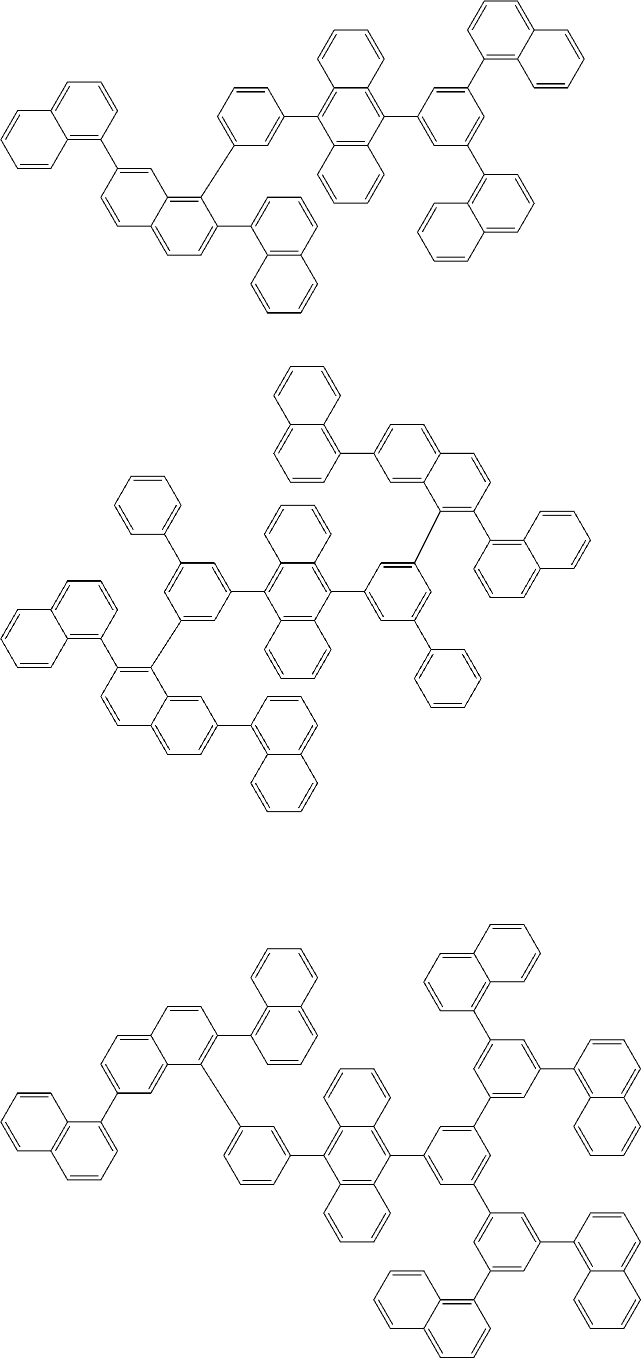

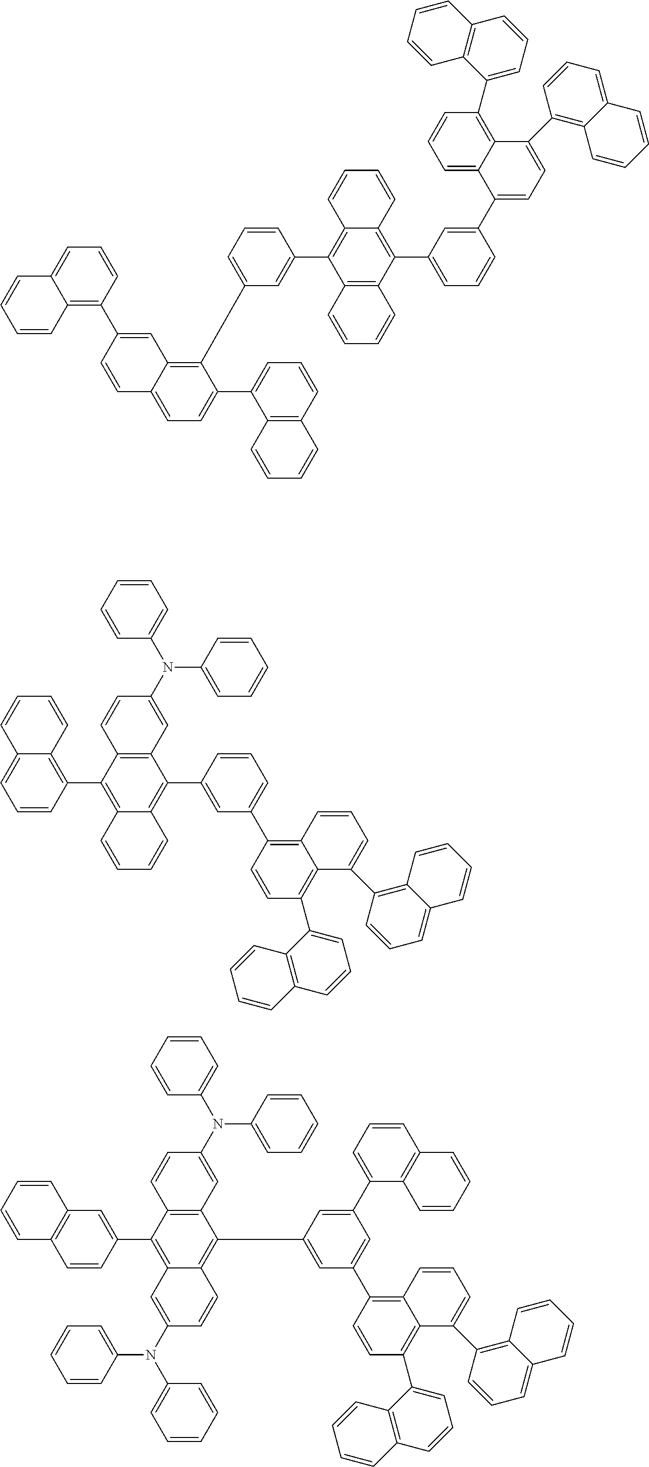

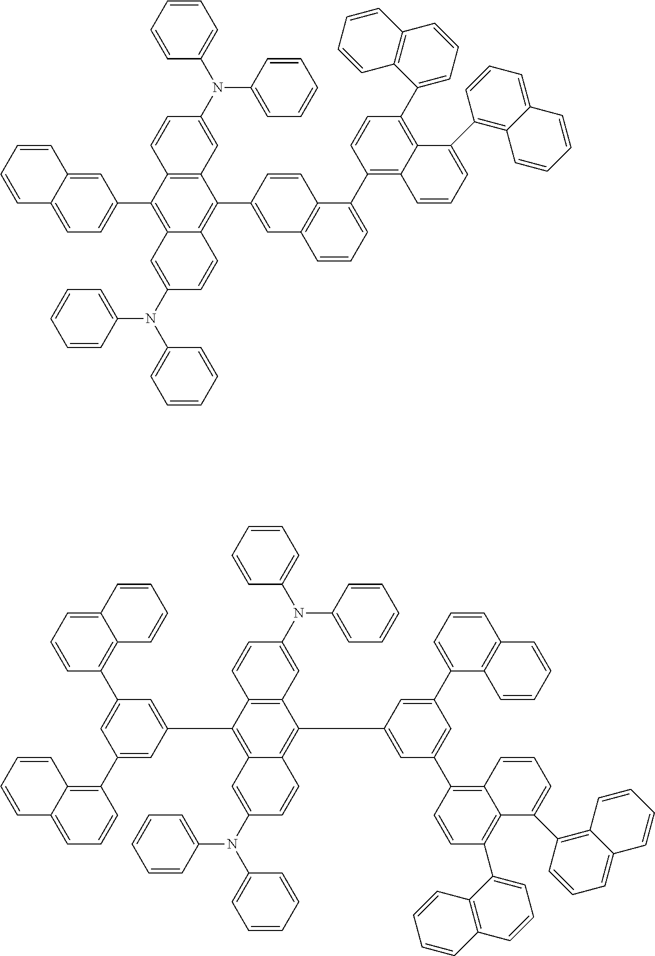 [SMILES: c1ccc(N(c2ccccc2)c2ccc3c(-c4ccc5c(-c6ccc(-c7cccc8ccccc78)c7c(-c8cccc9ccccc89)cccc67)cccc5c4)c4cc(N(c5ccccc5)c5ccccc5)ccc4c(-c4ccc5ccccc5c4)c3c2)cc1 ; c1ccc(N(c2ccccc2)c2ccc3c(-c4cc(-c5cccc6ccccc56)cc(-c5ccc(-c6cccc7ccccc67)c6c(-c7cccc8ccccc78)cccc56)c4)c4cc(N(c5ccccc5)c5ccccc5)ccc4c(-c4cc(-c5cccc6ccccc56)cc(-c5cccc6ccccc56)c4)c3c2)cc1]

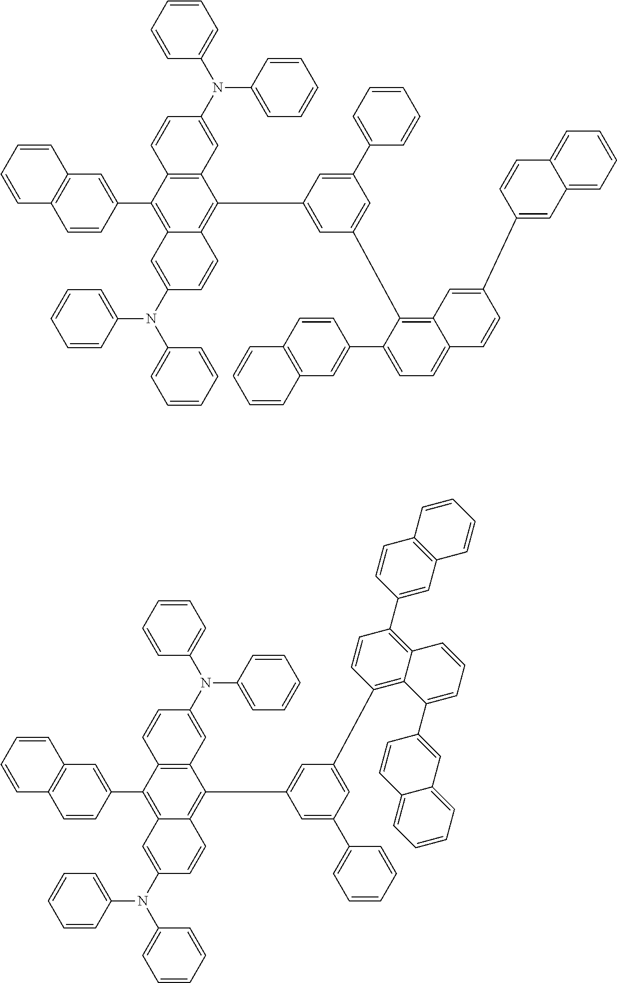 [SMILES: c1ccc(-c2cc(-c3c(-c4ccc5ccccc5c4)ccc4ccc(-c5ccc6ccccc6c5)cc34)cc(-c3c4ccc(N(c5ccccc5)c5ccccc5)cc4c(-c4ccc5ccccc5c4)c4ccc(N(c5ccccc5)c5ccccc5)cc34)c2)cc1 ; c1ccc(-c2cc(-c3c4ccc(N(c5ccccc5)c5ccccc5)cc4c(-c4ccc5ccccc5c4)c4ccc(N(c5ccccc5)c5ccccc5)cc34)cc(-c3ccc(-c4ccc5ccccc5c4)c4cccc(-c5ccc6ccccc6c5)c34)c2)cc1]

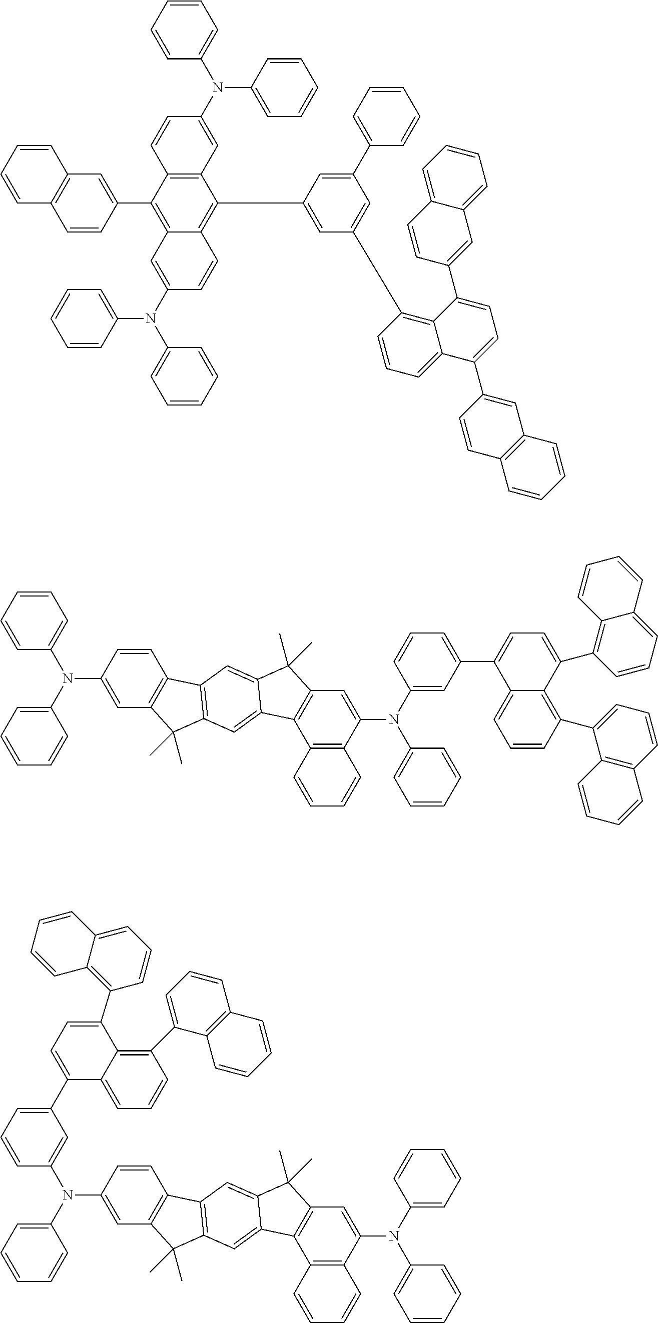 [SMILES: c1ccc(-c2cc(-c3c4ccc(N(c5ccccc5)c5ccccc5)cc4c(-c4ccc5ccccc5c4)c4ccc(N(c5ccccc5)c5ccccc5)cc34)cc(-c3cccc4c(-c5ccc6ccccc6c5)ccc(-c5ccc6ccccc6c5)c34)c2)cc1 ; CC1(C)c2cc(N(c3ccccc3)c3ccccc3)ccc2-c2cc3c(cc21)-c1c(cc(N(c2ccccc2)c2cccc(-c4ccc(-c5cccc6ccccc56)c5c(-c6cccc7ccccc67)cccc45)c2)c2ccccc12)C3(C)C ; CC1(C)c2cc(N(c3ccccc3)c3cccc(-c4ccc(-c5cccc6ccccc56)c5c(-c6cccc7ccccc67)cccc45)c3)ccc2-c2cc3c(cc21)-c1c(cc(N(c2ccccc2)c2ccccc2)c2ccccc12)C3(C)C]

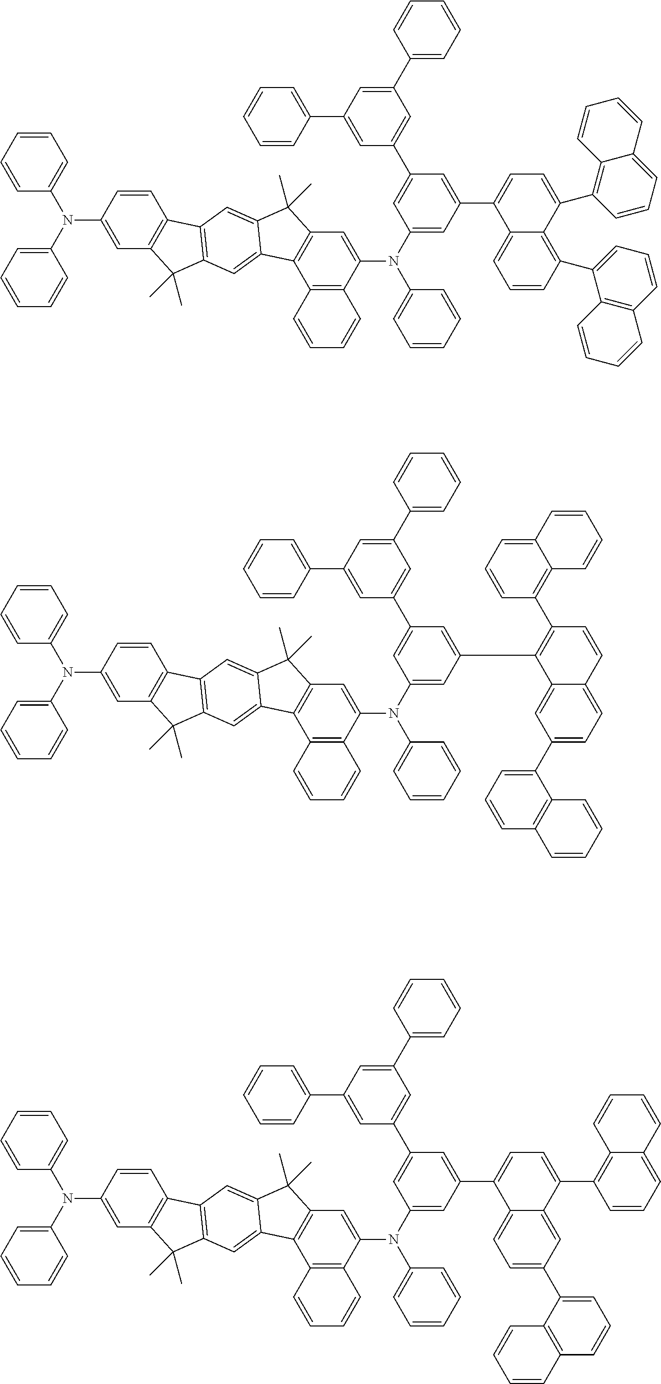

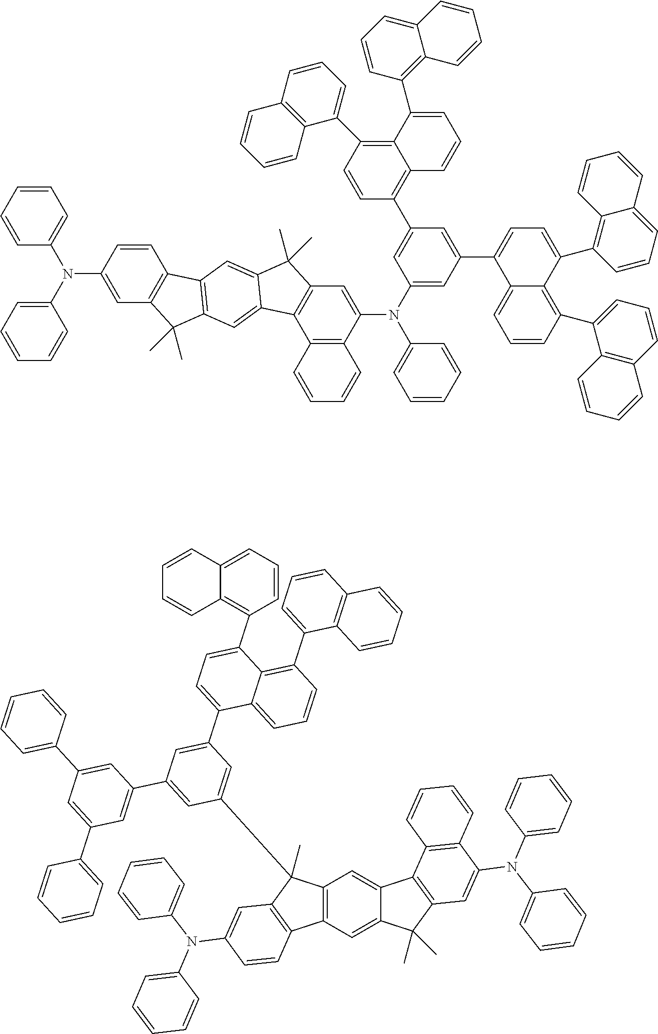 [SMILES: CC1(C)c2cc(N(c3ccccc3)c3ccccc3)ccc2-c2cc3c(cc21)-c1c(cc(N(c2ccccc2)c2cc(-c4ccc(-c5cccc6ccccc56)c5c(-c6cccc7ccccc67)cccc45)cc(-c4ccc(-c5cccc6ccccc56)c5c(-c6cccc7ccccc67)cccc45)c2)c2ccccc12)C3(C)C ; CC1(C)c2cc3c(cc2-c2c1cc(N(c1ccccc1)c1ccccc1)c1ccccc21)C(C)(c1cc(-c2cc(-c4ccccc4)cc(-c4ccccc4)c2)cc(-c2ccc(-c4cccc5ccccc45)c4c(-c5cccc6ccccc56)cccc24)c1)c1cc(N(c2ccccc2)c2ccccc2)ccc1-3]

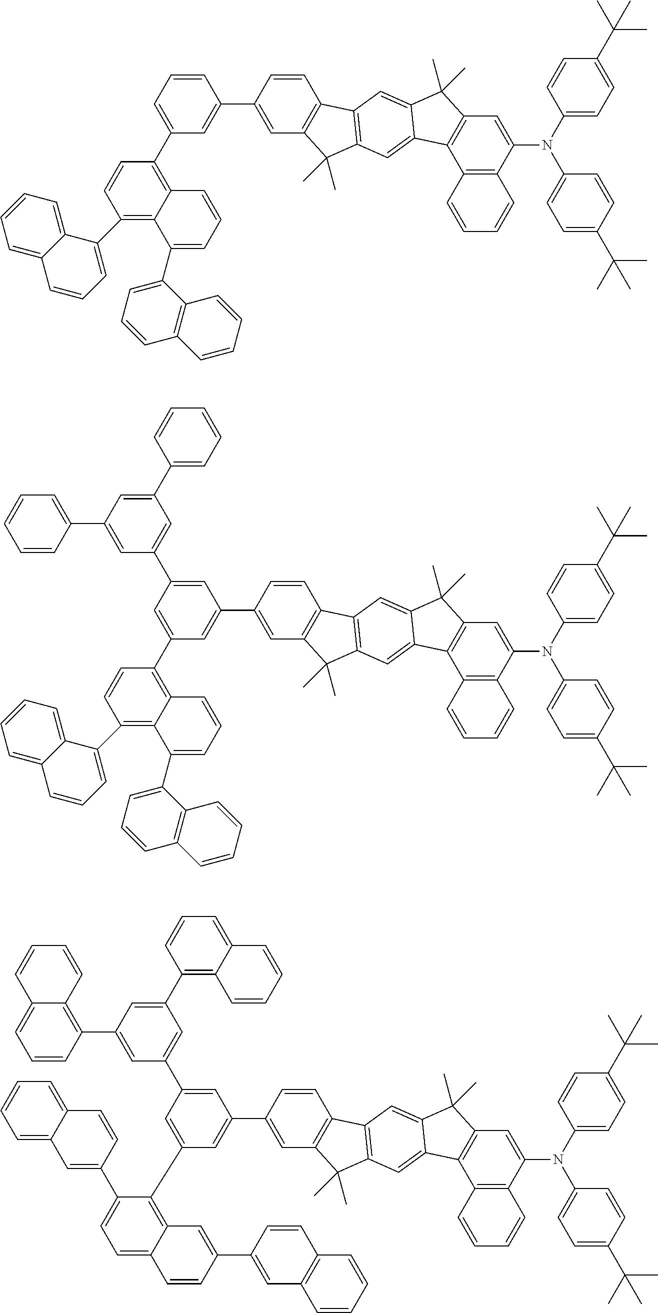

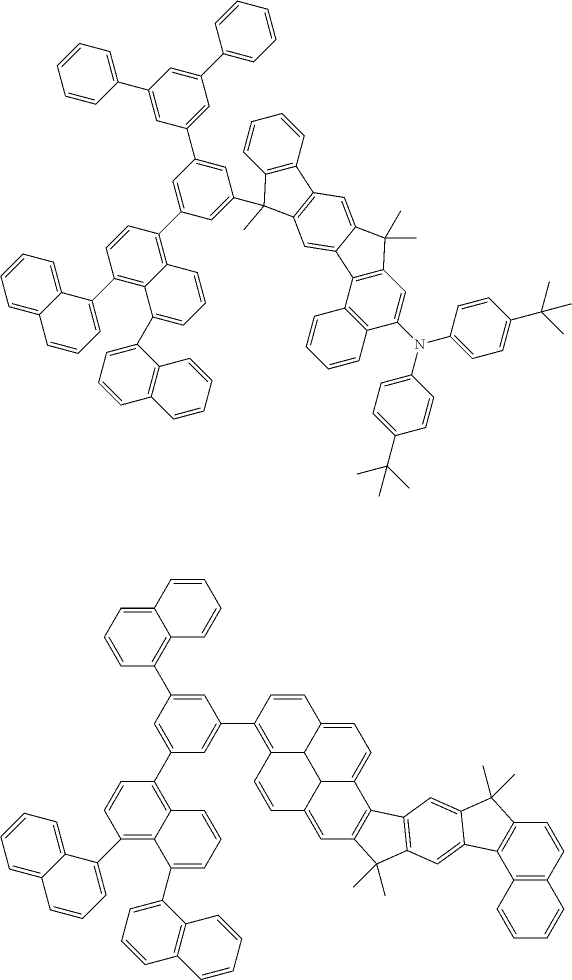

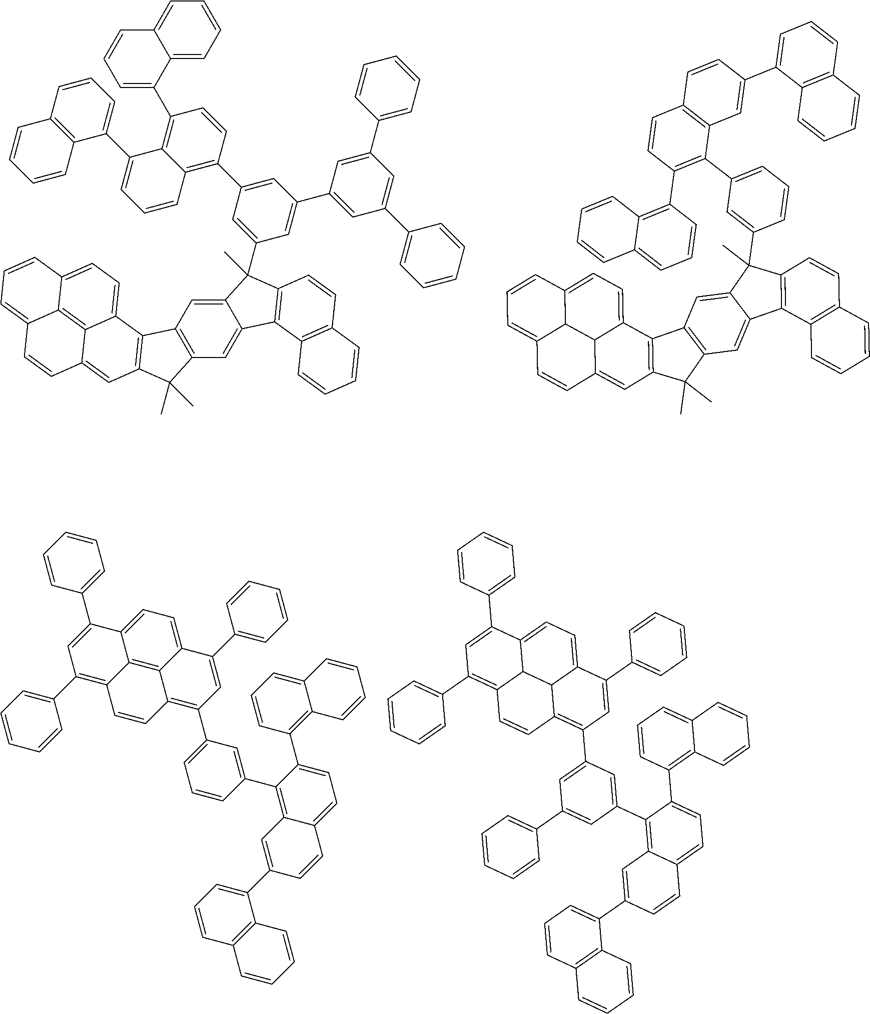

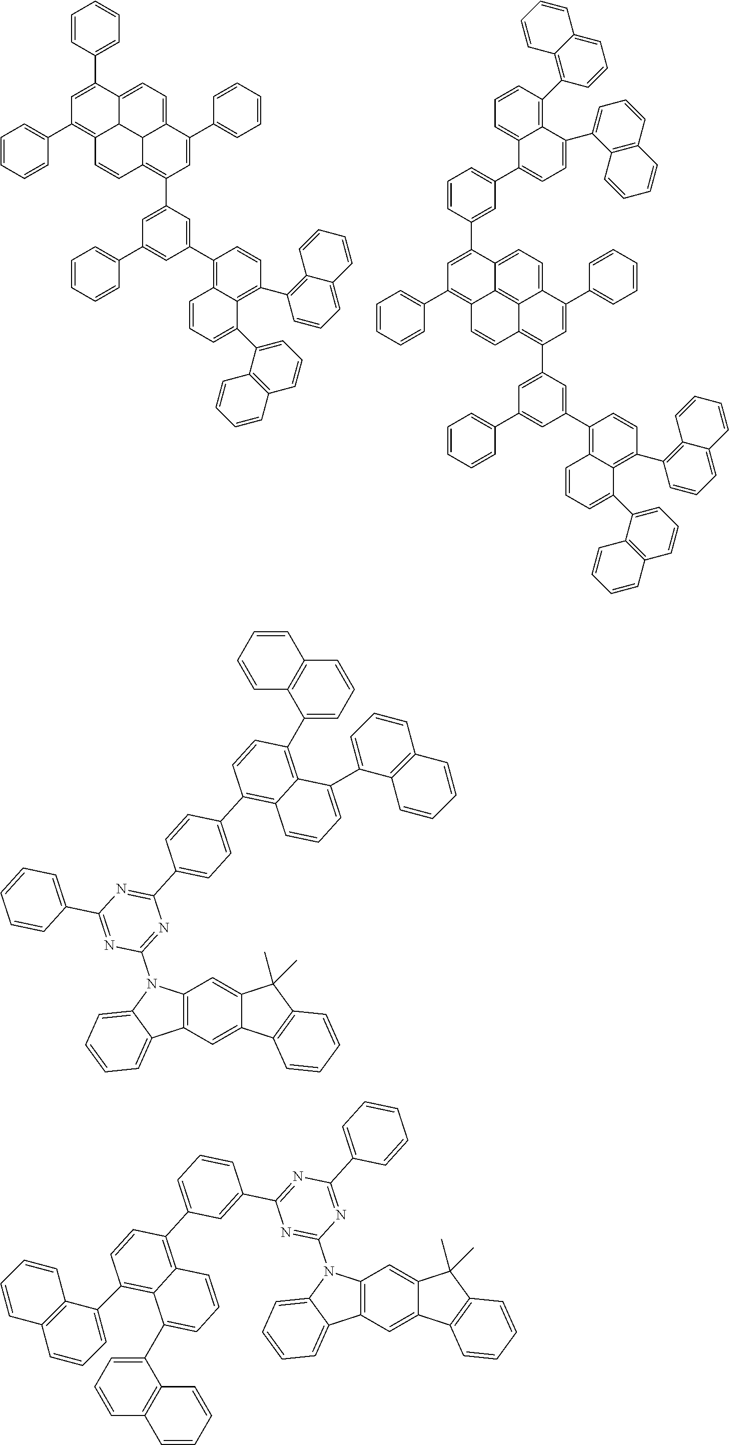

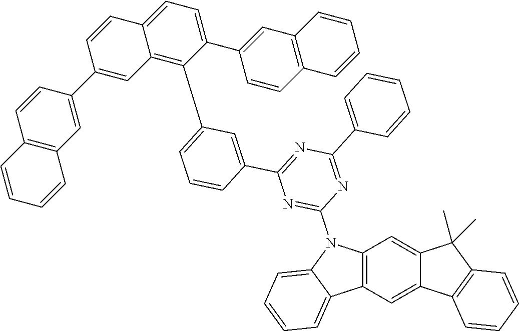 [SMILES: CC1(C)c2ccccc2-c2cc3c4ccccc4n(-c4nc(-c5ccccc5)nc(-c5cccc(-c6c(-c7ccc8ccccc8c7)ccc7ccc(-c8ccc9ccccc9c8)cc67)c5)n4)c3cc21]

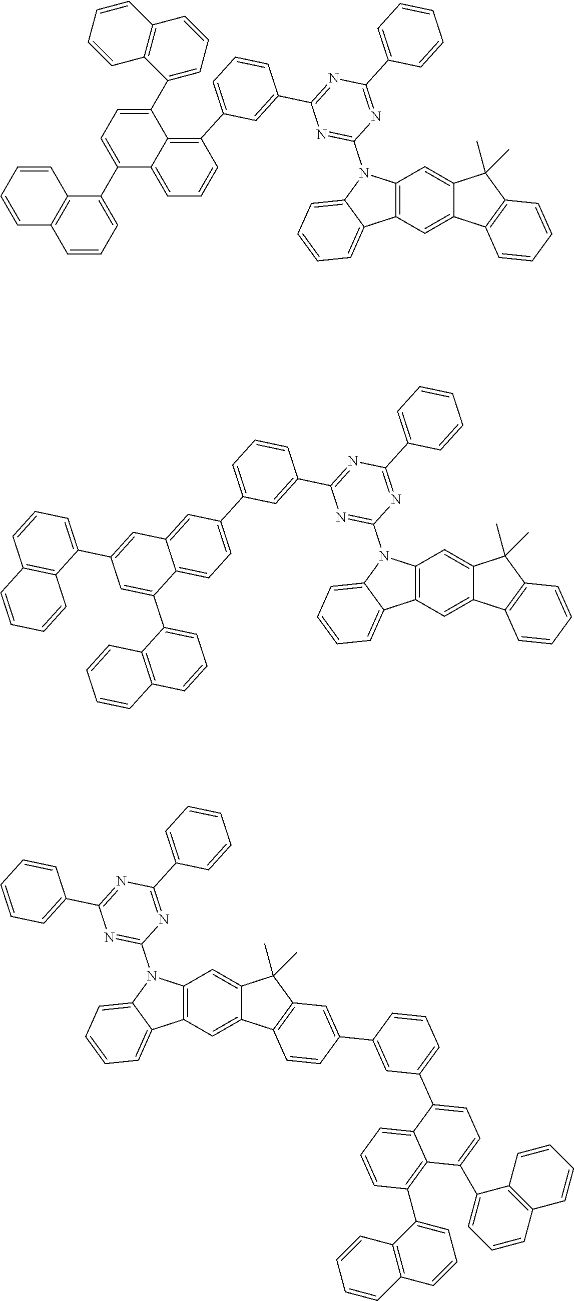 [SMILES: CC1(C)c2ccccc2-c2cc3c4ccccc4n(-c4nc(-c5ccccc5)nc(-c5cccc(-c6cccc7c(-c8cccc9ccccc89)ccc(-c8cccc9ccccc89)c67)c5)n4)c3cc21 ; CC1(C)c2ccccc2-c2cc3c4ccccc4n(-c4nc(-c5ccccc5)nc(-c5cccc(-c6ccc7c(-c8cccc9ccccc89)cc(-c8cccc9ccccc89)cc7c6)c5)n4)c3cc21 ; CC1(C)c2cc(-c3cccc(-c4ccc(-c5cccc6ccccc56)c5c(-c6cccc7ccccc67)cccc45)c3)ccc2-c2cc3c4ccccc4n(-c4nc(-c5ccccc5)nc(-c5ccccc5)n4)c3cc21]

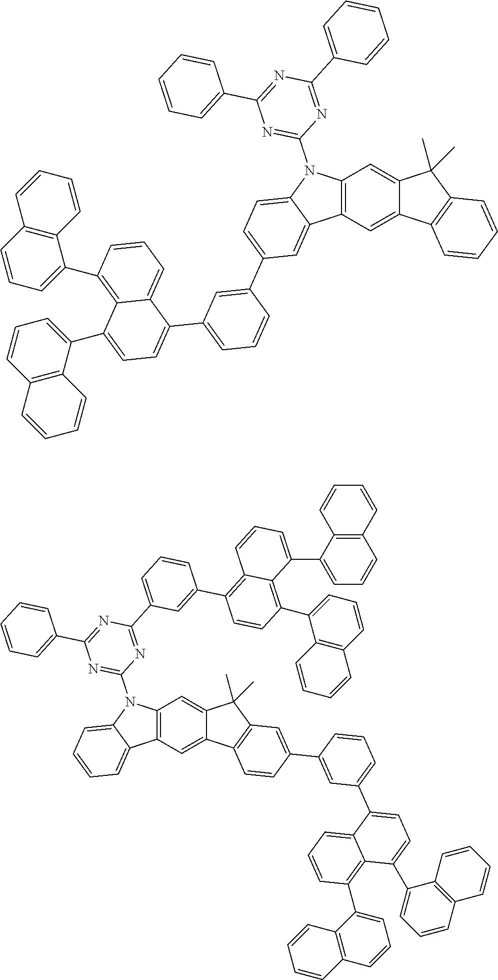 [SMILES: CC1(C)c2ccccc2-c2cc3c4cc(-c5cccc(-c6ccc(-c7cccc8ccccc78)c7c(-c8cccc9ccccc89)cccc67)c5)ccc4n(-c4nc(-c5ccccc5)nc(-c5ccccc5)n4)c3cc21 ; CC1(C)c2cc(-c3cccc(-c4ccc(-c5cccc6ccccc56)c5c(-c6cccc7ccccc67)cccc45)c3)ccc2-c2cc3c4ccccc4n(-c4nc(-c5ccccc5)nc(-c5cccc(-c6ccc(-c7cccc8ccccc78)c7c(-c8cccc9ccccc89)cccc67)c5)n4)c3cc21]

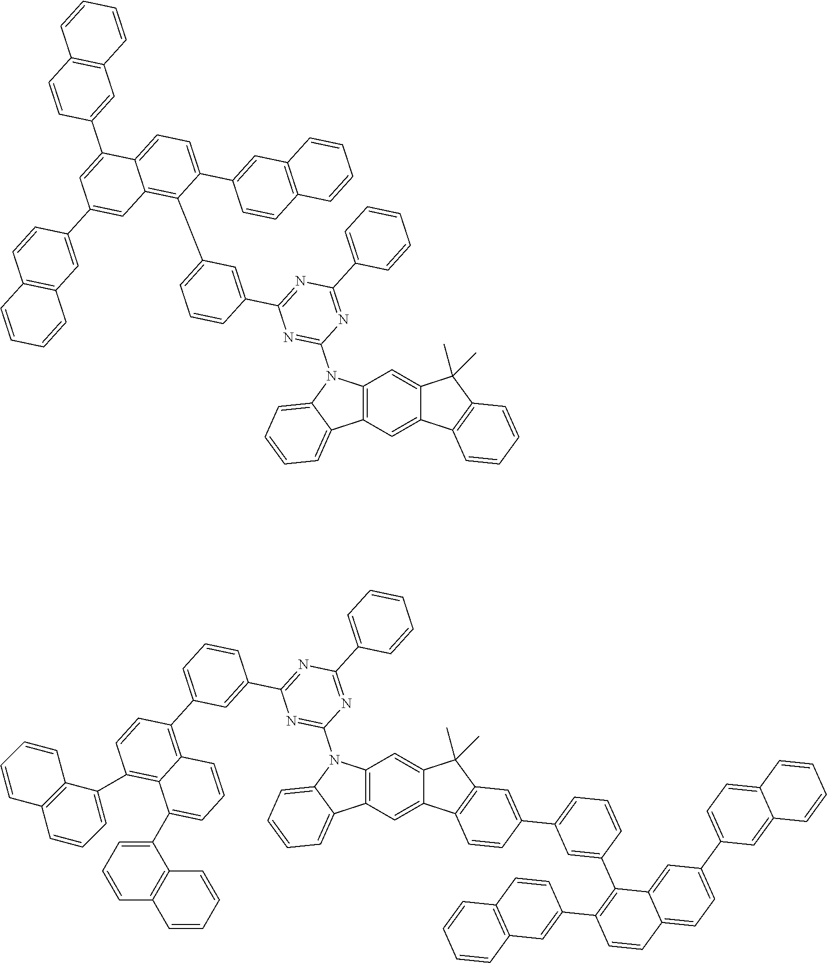 [SMILES: CC1(C)c2ccccc2-c2cc3c4ccccc4n(-c4nc(-c5ccccc5)nc(-c5cccc(-c6c(-c7ccc8ccccc8c7)ccc7c(-c8ccc9ccccc9c8)cc(-c8ccc9ccccc9c8)cc67)c5)n4)c3cc21 ; CC1(C)c2cc(-c3cccc(-c4c(-c5ccc6ccccc6c5)ccc5ccc(-c6ccc7ccccc7c6)cc45)c3)ccc2-c2cc3c4ccccc4n(-c4nc(-c5ccccc5)nc(-c5cccc(-c6ccc(-c7cccc8ccccc78)c7c(-c8cccc9ccccc89)cccc67)c5)n4)c3cc21]

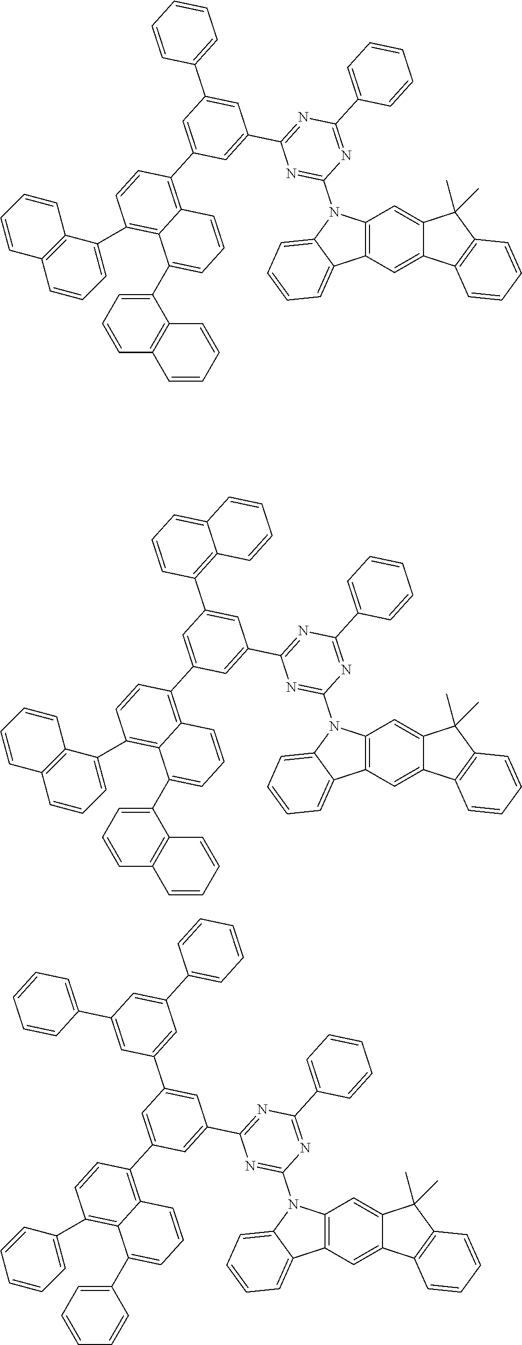

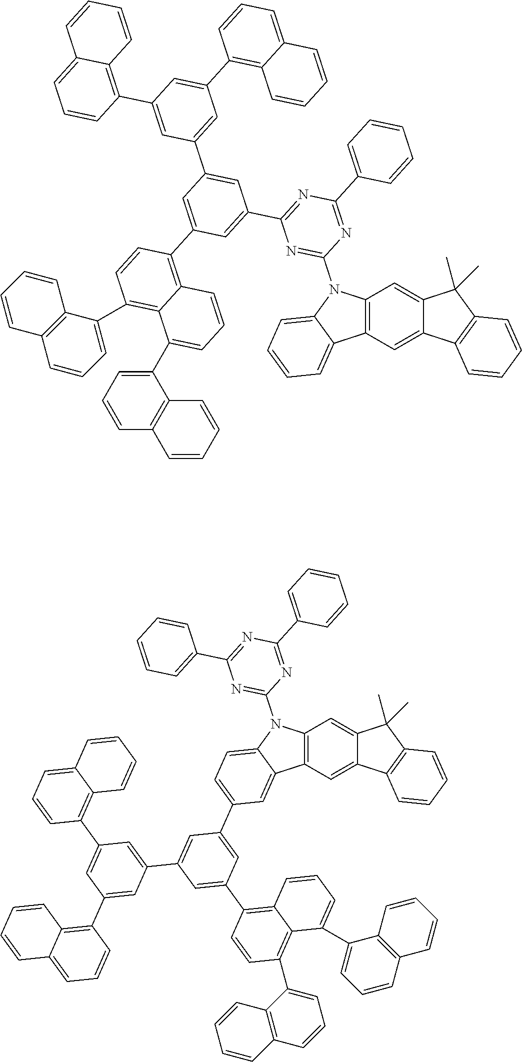 [SMILES: CC1(C)c2ccccc2-c2cc3c4ccccc4n(-c4nc(-c5ccccc5)nc(-c5cc(-c6cc(-c7cccc8ccccc78)cc(-c7cccc8ccccc78)c6)cc(-c6ccc(-c7cccc8ccccc78)c7c(-c8cccc9ccccc89)cccc67)c5)n4)c3cc21 ; CC1(C)c2ccccc2-c2cc3c4cc(-c5cc(-c6cc(-c7cccc8ccccc78)cc(-c7cccc8ccccc78)c6)cc(-c6ccc(-c7cccc8ccccc78)c7c(-c8cccc9ccccc89)cccc67)c5)ccc4n(-c4nc(-c5ccccc5)nc(-c5ccccc5)n4)c3cc21]

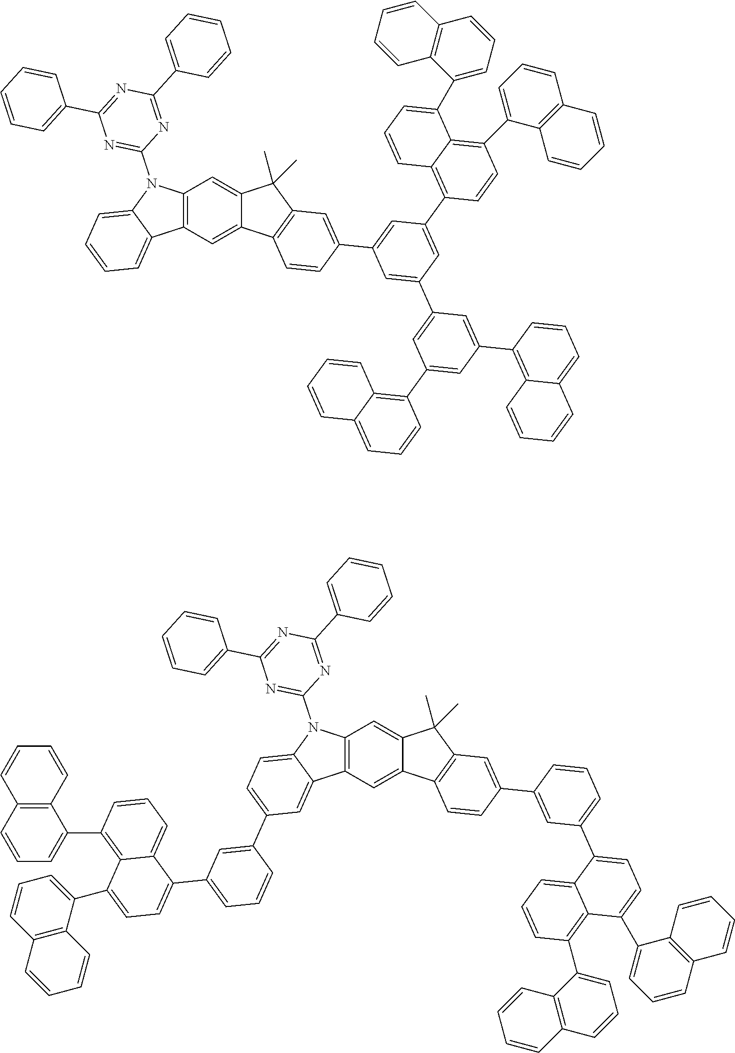 [SMILES: CC1(C)c2cc(-c3cc(-c4cc(-c5cccc6ccccc56)cc(-c5cccc6ccccc56)c4)cc(-c4ccc(-c5cccc6ccccc56)c5c(-c6cccc7ccccc67)cccc45)c3)ccc2-c2cc3c4ccccc4n(-c4nc(-c5ccccc5)nc(-c5ccccc5)n4)c3cc21 ; CC1(C)c2cc(-c3cccc(-c4ccc(-c5cccc6ccccc56)c5c(-c6cccc7ccccc67)cccc45)c3)ccc2-c2cc3c4cc(-c5cccc(-c6ccc(-c7cccc8ccccc78)c7c(-c8cccc9ccccc89)cccc67)c5)ccc4n(-c4nc(-c5ccccc5)nc(-c5ccccc5)n4)c3cc21]

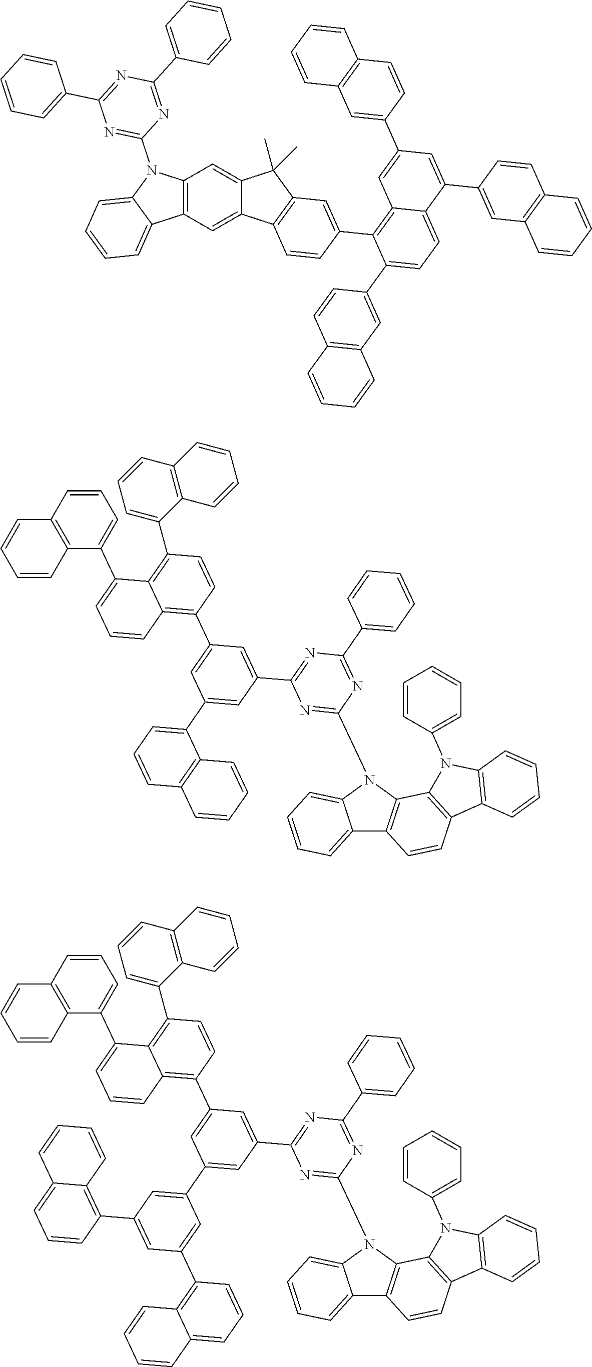 [SMILES: CC1(C)c2cc(-c3c(-c4ccc5ccccc5c4)ccc4c(-c5ccc6ccccc6c5)cc(-c5ccc6ccccc6c5)cc34)ccc2-c2cc3c4ccccc4n(-c4nc(-c5ccccc5)nc(-c5ccccc5)n4)c3cc21 ; c1ccc(-c2nc(-c3cc(-c4cccc5ccccc45)cc(-c4ccc(-c5cccc6ccccc56)c5c(-c6cccc7ccccc67)cccc45)c3)nc(-n3c4ccccc4c4ccc5c6ccccc6n(-c6ccccc6)c5c43)n2)cc1 ; c1ccc(-c2nc(-c3cc(-c4cc(-c5cccc6ccccc56)cc(-c5cccc6ccccc56)c4)cc(-c4ccc(-c5cccc6ccccc56)c5c(-c6cccc7ccccc67)cccc45)c3)nc(-n3c4ccccc4c4ccc5c6ccccc6n(-c6ccccc6)c5c43)n2)cc1]

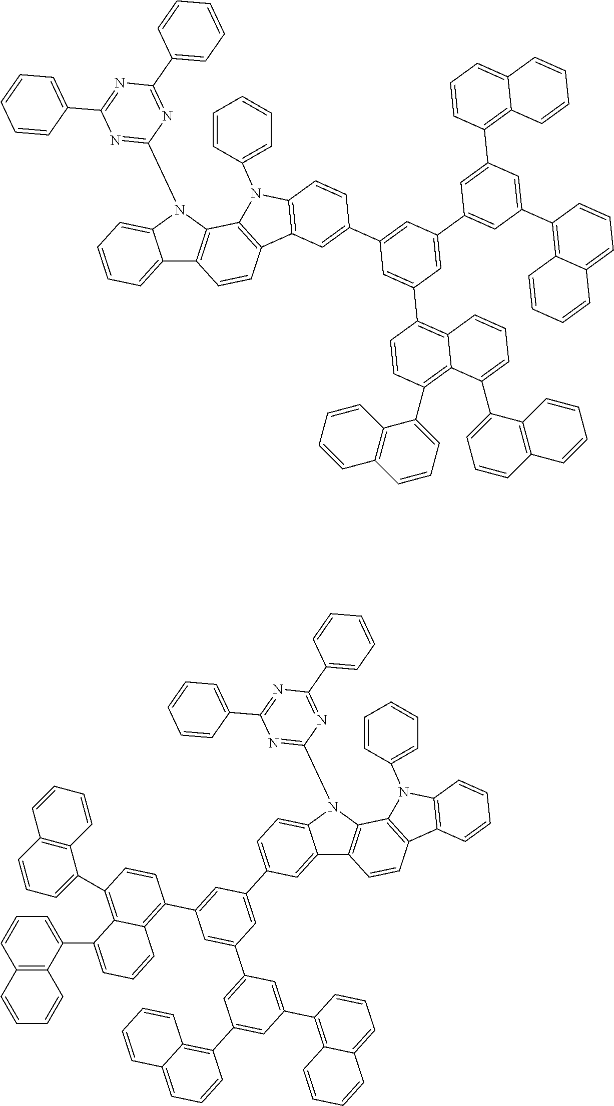 [SMILES: c1ccc(-c2nc(-c3ccccc3)nc(-n3c4ccccc4c4ccc5c6cc(-c7cc(-c8cc(-c9cccc%10ccccc9%10)cc(-c9cccc%10ccccc9%10)c8)cc(-c8ccc(-c9cccc%10ccccc9%10)c9c(-c%10cccc%11ccccc%10%11)cccc89)c7)ccc6n(-c6ccccc6)c5c43)n2)cc1 ; c1ccc(-c2nc(-c3ccccc3)nc(-n3c4ccc(-c5cc(-c6cc(-c7cccc8ccccc78)cc(-c7cccc8ccccc78)c6)cc(-c6ccc(-c7cccc8ccccc78)c7c(-c8cccc9ccccc89)cccc67)c5)cc4c4ccc5c6ccccc6n(-c6ccccc6)c5c43)n2)cc1]

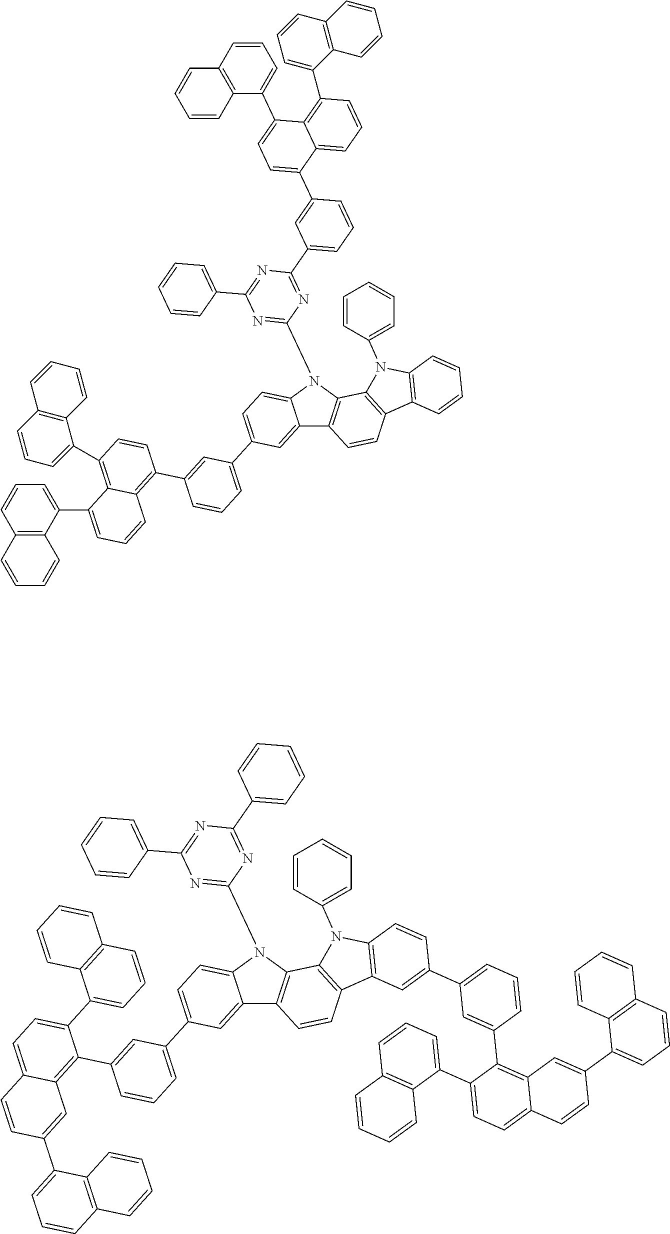 [SMILES: c1ccc(-c2nc(-c3cccc(-c4ccc(-c5cccc6ccccc56)c5c(-c6cccc7ccccc67)cccc45)c3)nc(-n3c4ccc(-c5cccc(-c6ccc(-c7cccc8ccccc78)c7c(-c8cccc9ccccc89)cccc67)c5)cc4c4ccc5c6ccccc6n(-c6ccccc6)c5c43)n2)cc1 ; c1ccc(-c2nc(-c3ccccc3)nc(-n3c4ccc(-c5cccc(-c6c(-c7cccc8ccccc78)ccc7ccc(-c8cccc9ccccc89)cc67)c5)cc4c4ccc5c6cc(-c7cccc(-c8c(-c9cccc%10ccccc9%10)ccc9ccc(-c%10cccc%11ccccc%10%11)cc89)c7)ccc6n(-c6ccccc6)c5c43)n2)cc1]

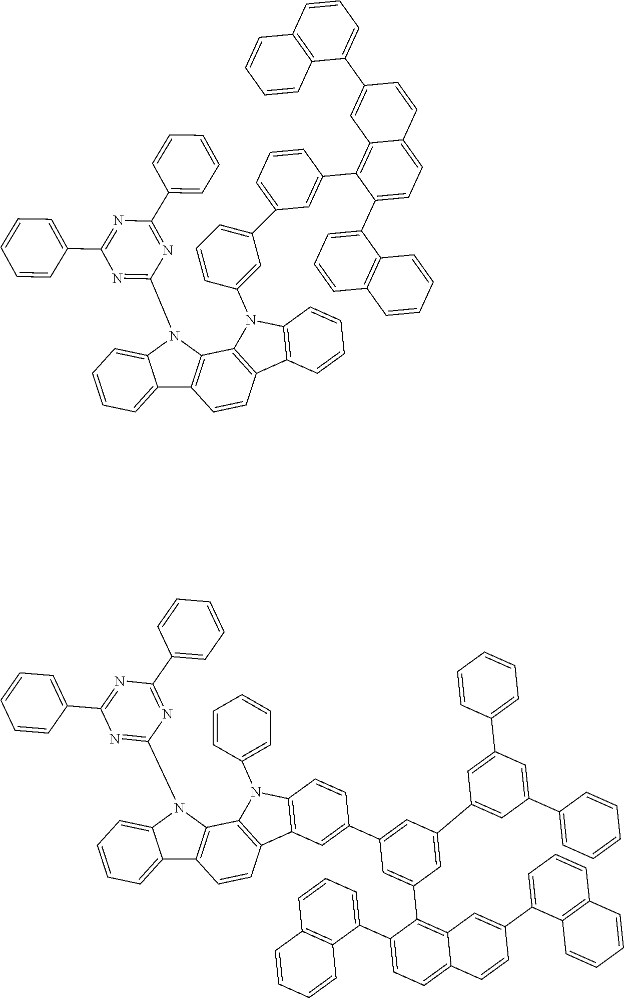 [SMILES: c1ccc(-c2nc(-c3ccccc3)nc(-n3c4ccccc4c4ccc5c6ccccc6n(-c6cccc(-c7cccc(-c8c(-c9cccc%10ccccc9%10)ccc9ccc(-c%10cccc%11ccccc%10%11)cc89)c7)c6)c5c43)n2)cc1 ; c1ccc(-c2cc(-c3ccccc3)cc(-c3cc(-c4ccc5c(c4)c4ccc6c7ccccc7n(-c7nc(-c8ccccc8)nc(-c8ccccc8)n7)c6c4n5-c4ccccc4)cc(-c4c(-c5cccc6ccccc56)ccc5ccc(-c6cccc7ccccc67)cc45)c3)c2)cc1]

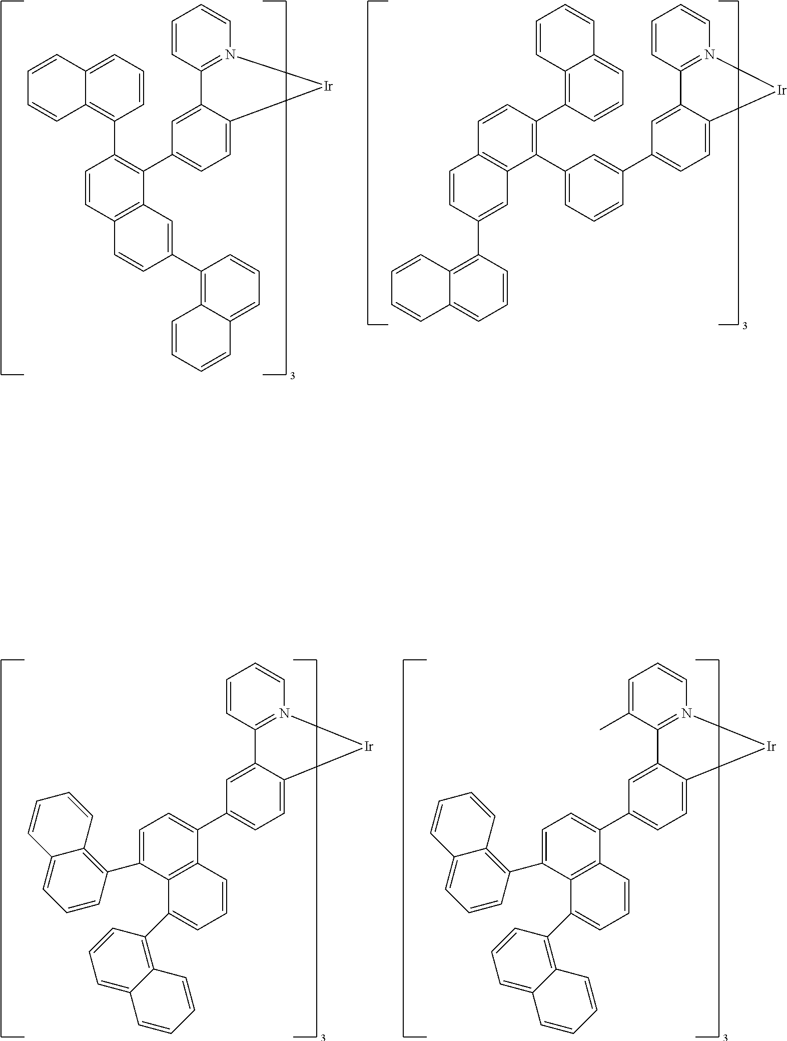

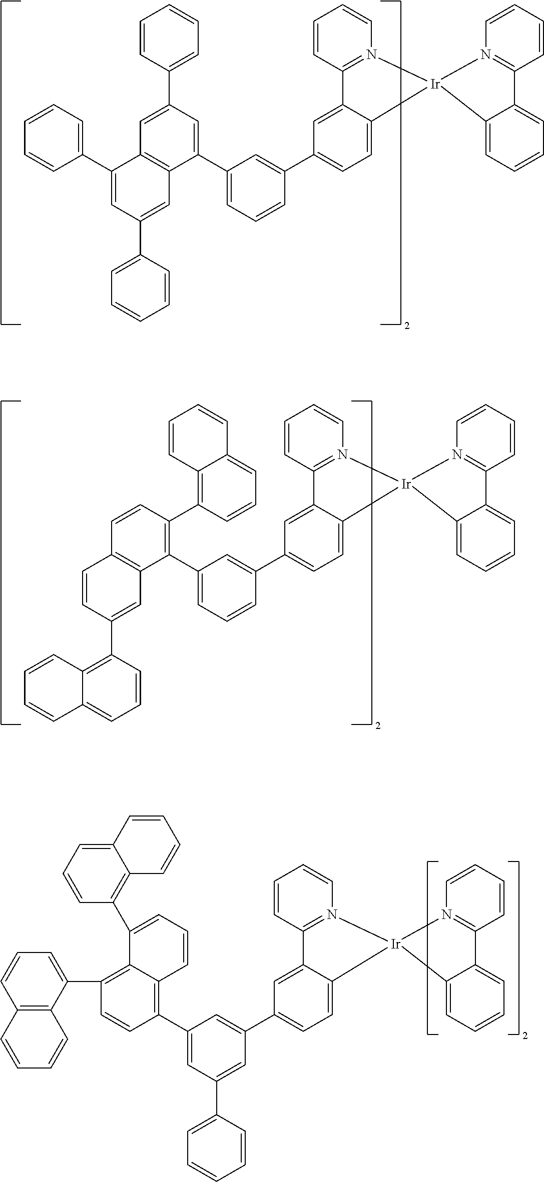

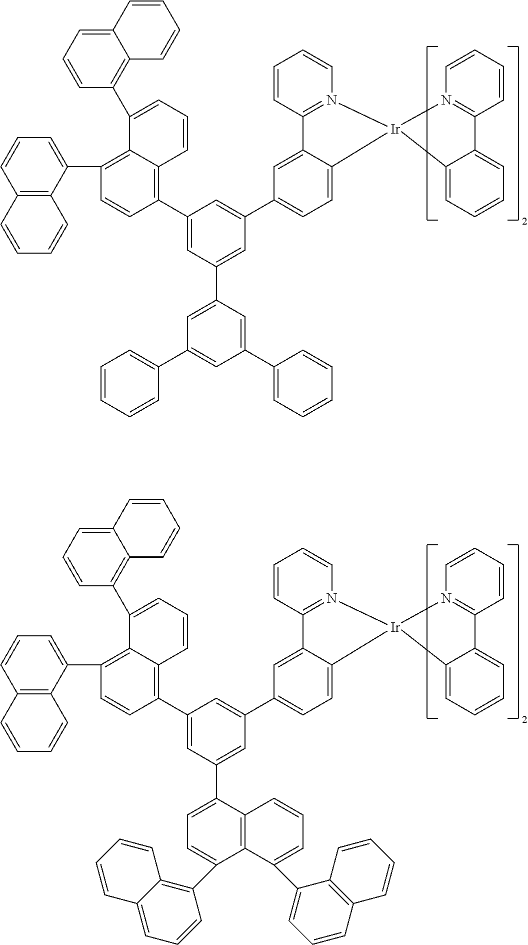

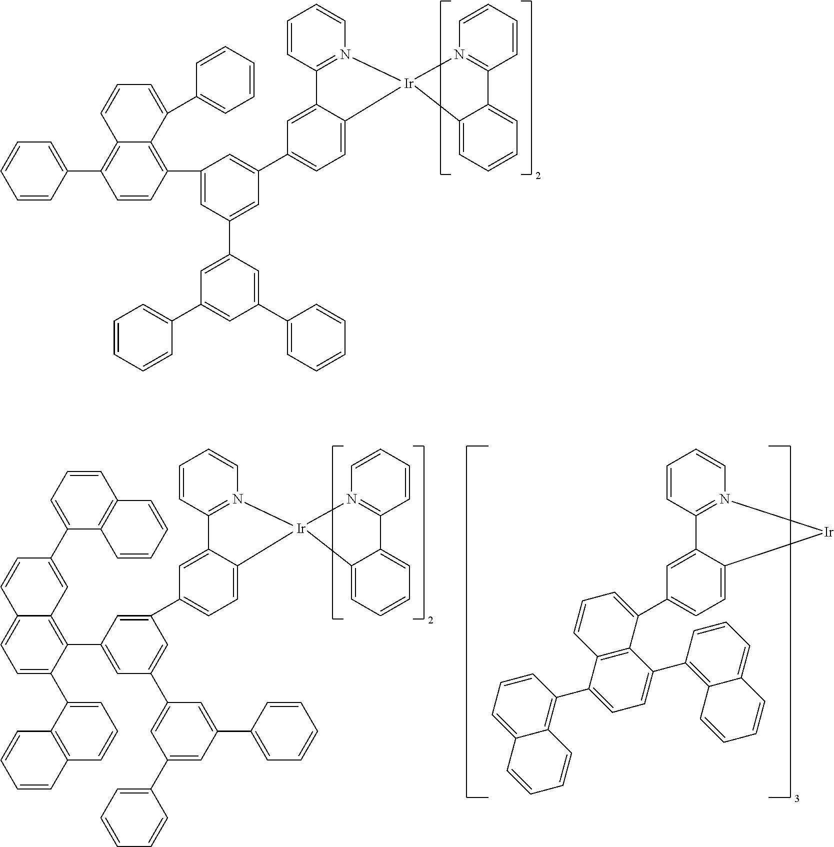

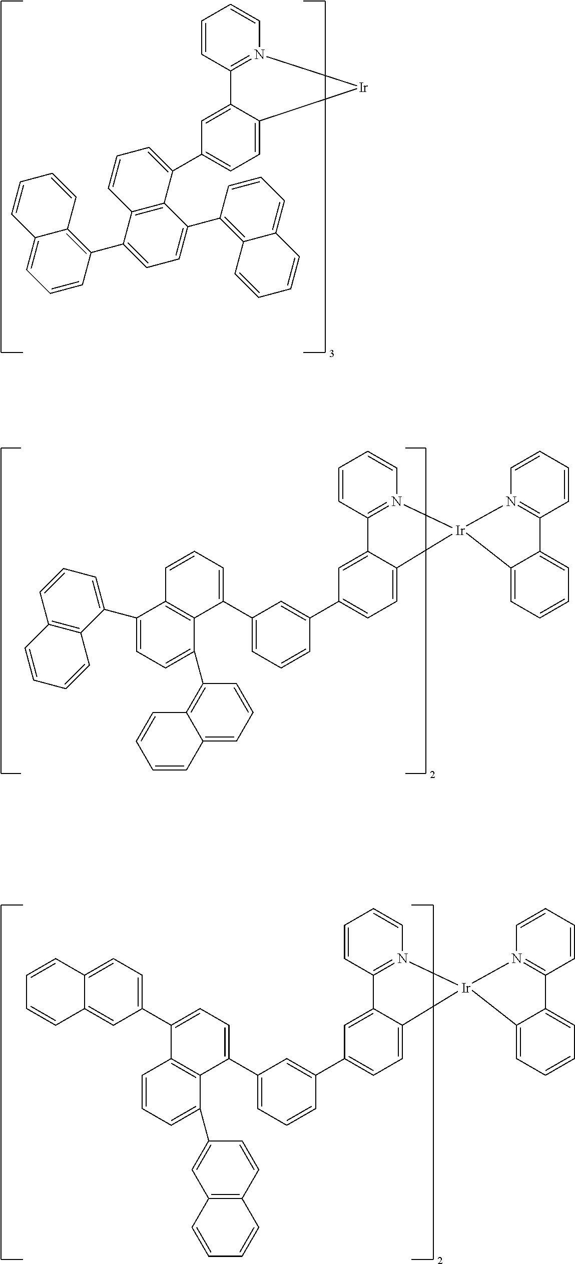

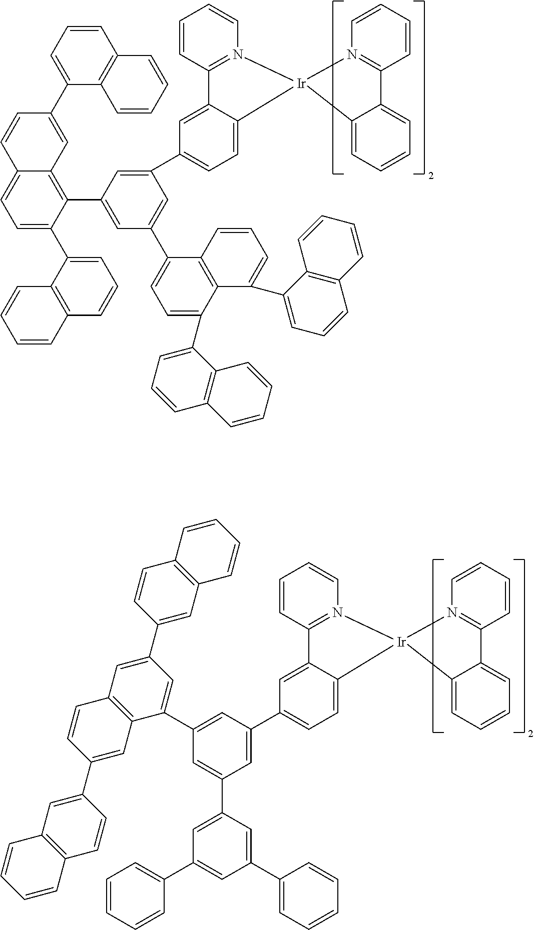

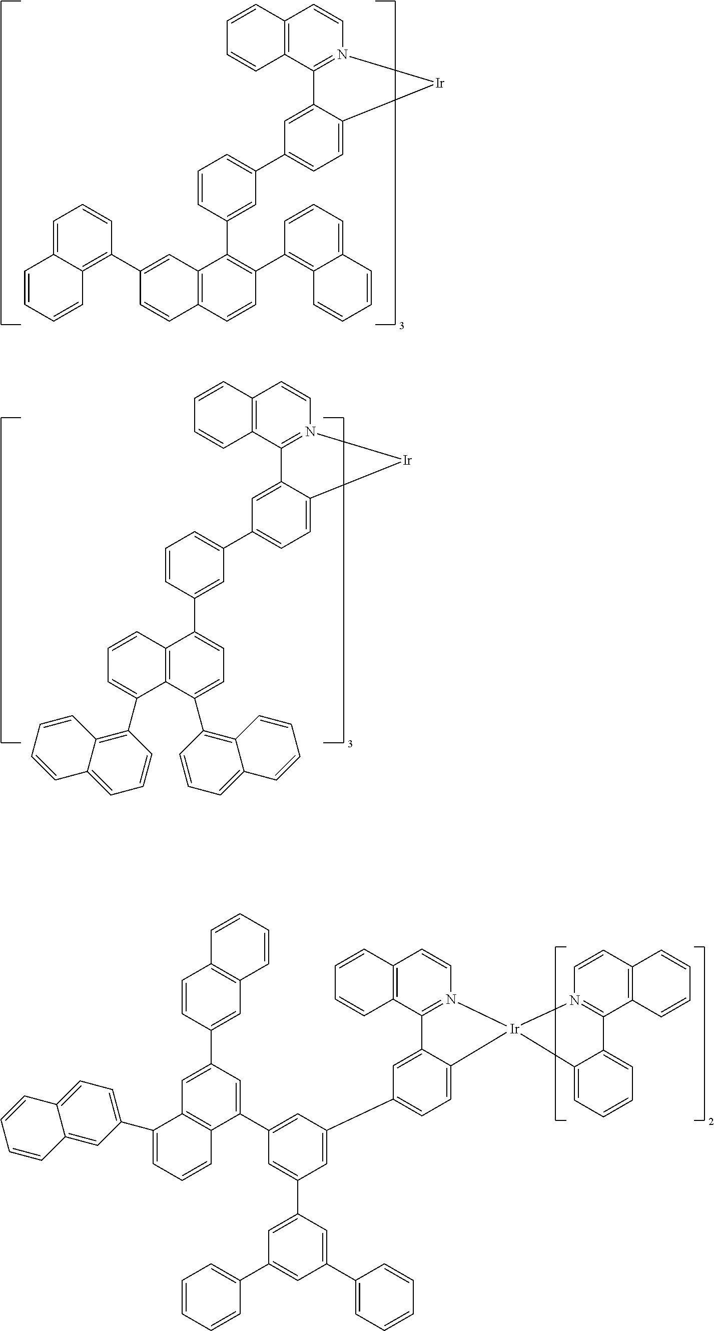

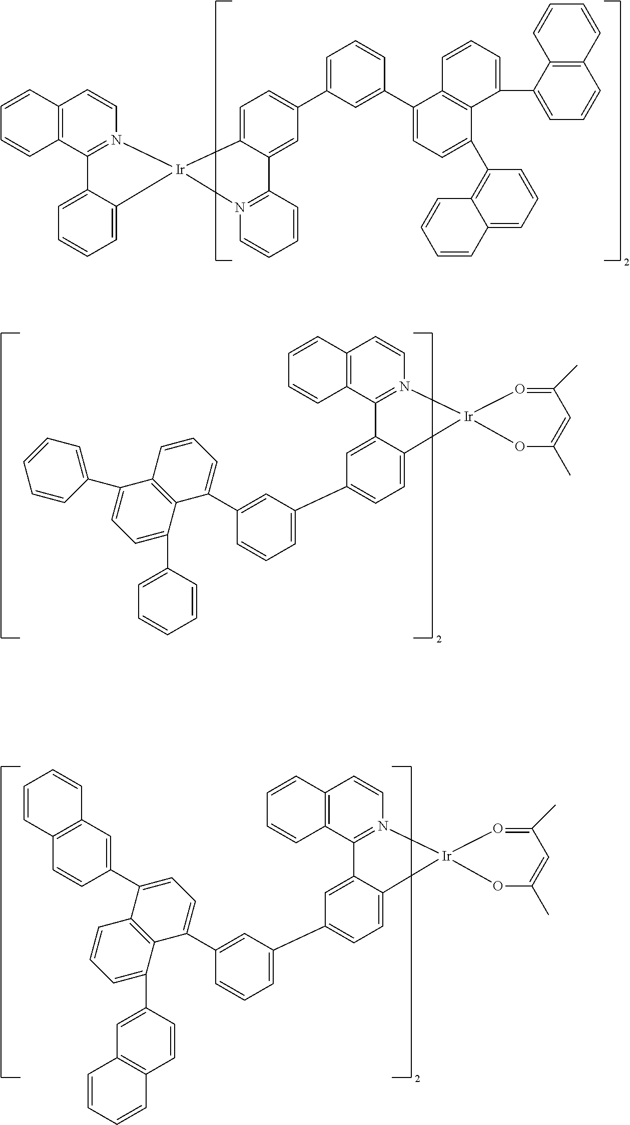

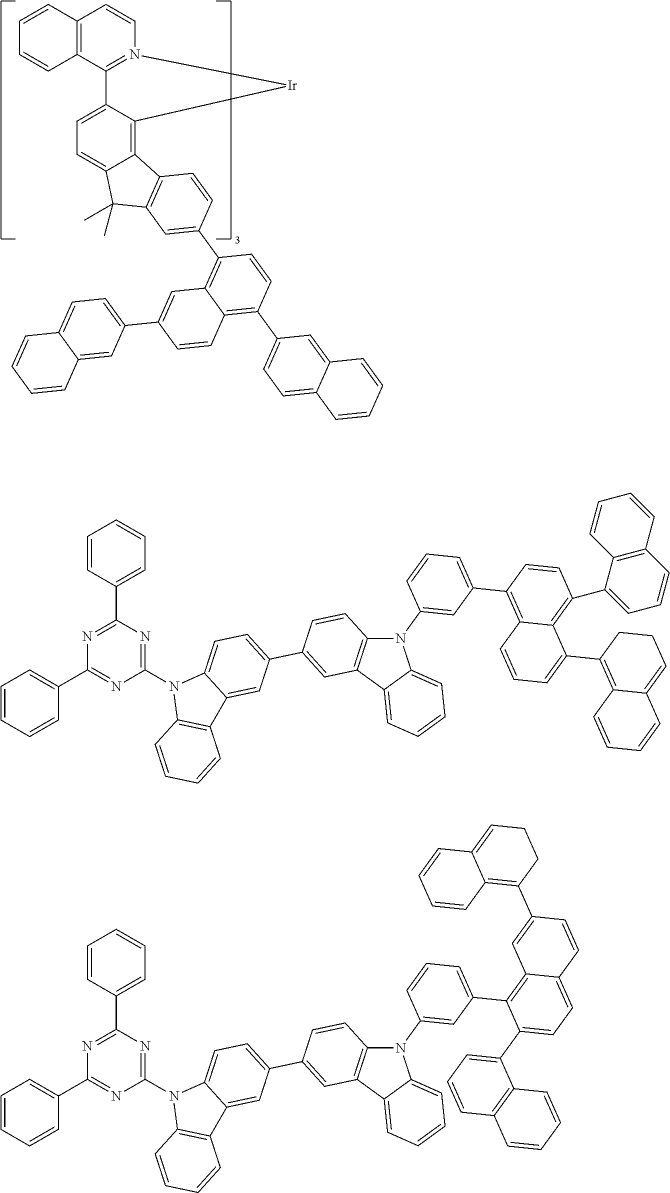

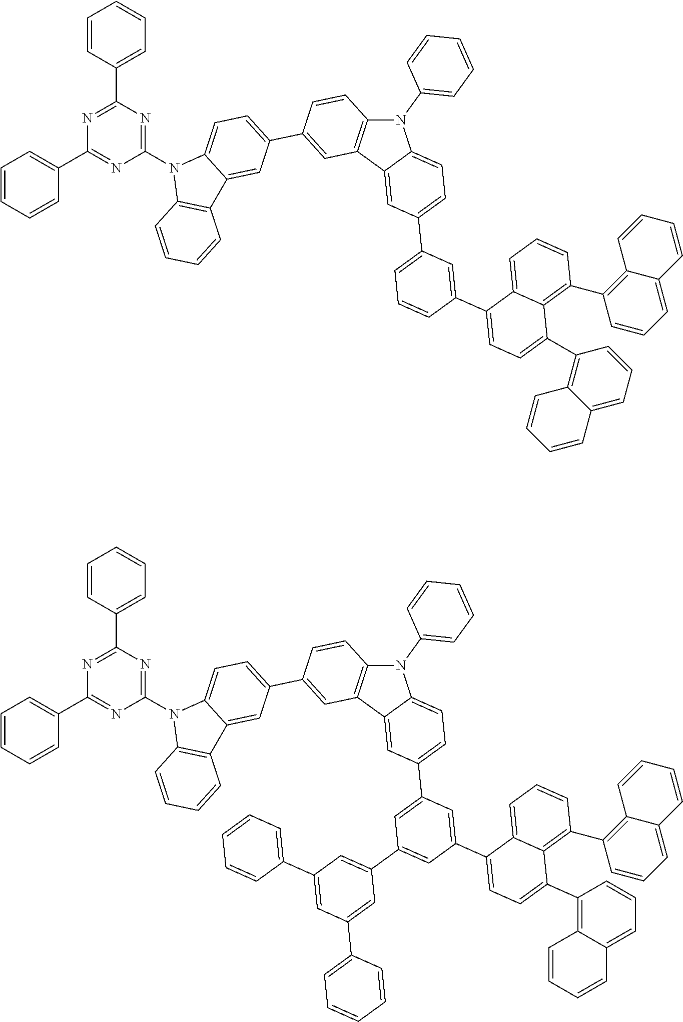 [SMILES: c1ccc(-c2nc(-c3ccccc3)nc(-n3c4ccccc4c4cc(-c5ccc6c(c5)c5cc(-c7cccc(-c8ccc(-c9cccc%10ccccc9%10)c9c(-c%10cccc%11ccccc%10%11)cccc89)c7)ccc5n6-c5ccccc5)ccc43)n2)cc1 ; c1ccc(-c2cc(-c3ccccc3)cc(-c3cc(-c4ccc5c(c4)c4cc(-c6ccc7c(c6)c6ccccc6n7-c6nc(-c7ccccc7)nc(-c7ccccc7)n6)ccc4n5-c4ccccc4)cc(-c4ccc(-c5cccc6ccccc56)c5c(-c6cccc7ccccc67)cccc45)c3)c2)cc1]

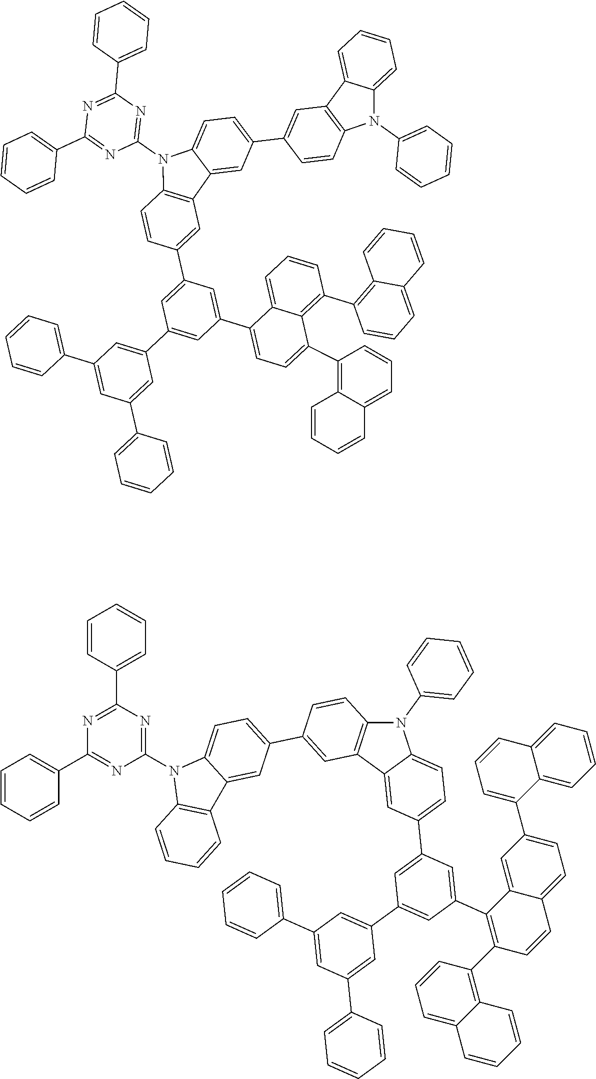 [SMILES: c1ccc(-c2cc(-c3ccccc3)cc(-c3cc(-c4ccc5c(c4)c4cc(-c6ccc7c(c6)c6ccccc6n7-c6ccccc6)ccc4n5-c4nc(-c5ccccc5)nc(-c5ccccc5)n4)cc(-c4ccc(-c5cccc6ccccc56)c5c(-c6cccc7ccccc67)cccc45)c3)c2)cc1 ; c1ccc(-c2cc(-c3ccccc3)cc(-c3cc(-c4ccc5c(c4)c4cc(-c6ccc7c(c6)c6ccccc6n7-c6nc(-c7ccccc7)nc(-c7ccccc7)n6)ccc4n5-c4ccccc4)cc(-c4c(-c5cccc6ccccc56)ccc5ccc(-c6cccc7ccccc67)cc45)c3)c2)cc1]

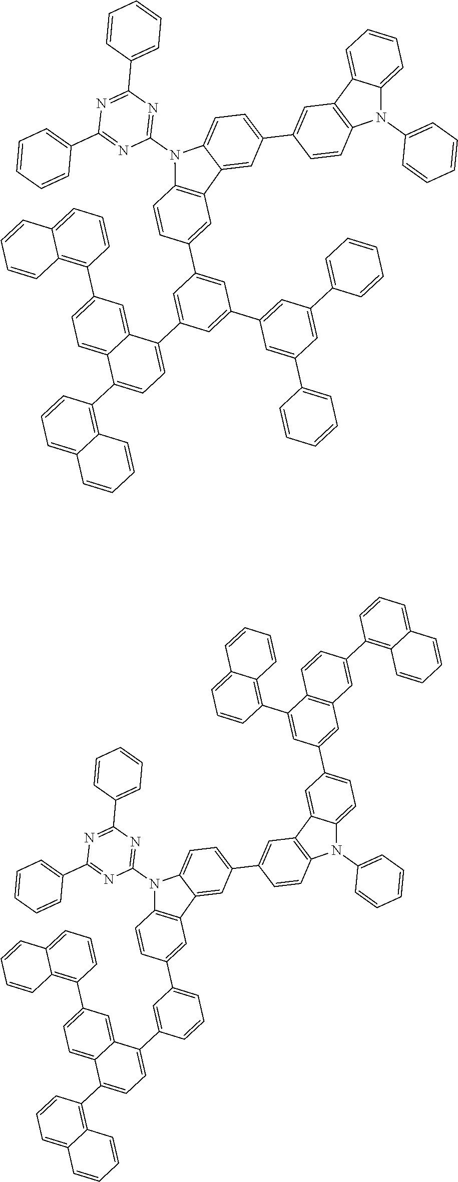 [SMILES: c1ccc(-c2cc(-c3ccccc3)cc(-c3cc(-c4ccc5c(c4)c4cc(-c6ccc7c(c6)c6ccccc6n7-c6ccccc6)ccc4n5-c4nc(-c5ccccc5)nc(-c5ccccc5)n4)cc(-c4ccc(-c5cccc6ccccc56)c5ccc(-c6cccc7ccccc67)cc45)c3)c2)cc1 ; c1ccc(-c2nc(-c3ccccc3)nc(-n3c4ccc(-c5cccc(-c6ccc(-c7cccc8ccccc78)c7ccc(-c8cccc9ccccc89)cc67)c5)cc4c4cc(-c5ccc6c(c5)c5cc(-c7cc(-c8cccc9ccccc89)c8ccc(-c9cccc%10ccccc9%10)cc8c7)ccc5n6-c5ccccc5)ccc43)n2)cc1]

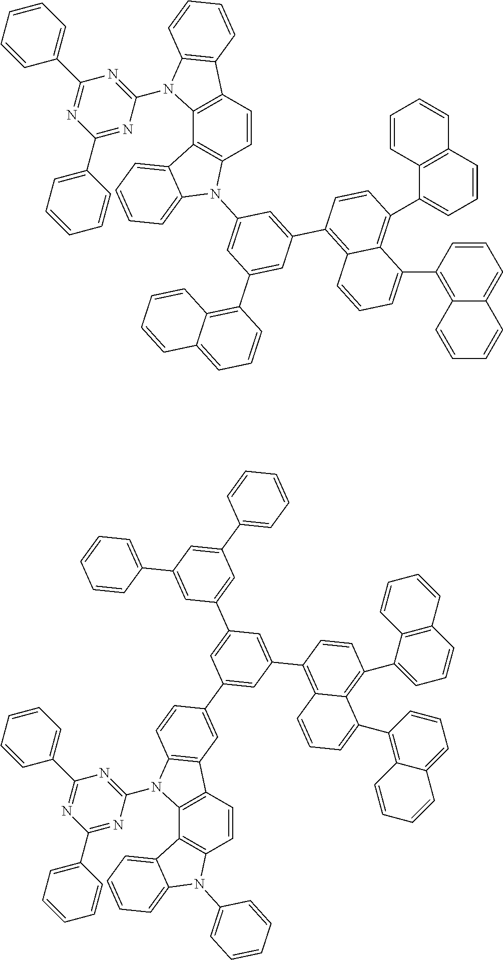 [SMILES: c1ccc(-c2nc(-c3ccccc3)nc(-n3c4ccccc4c4ccc5c(c6ccccc6n5-c5cc(-c6cccc7ccccc67)cc(-c6ccc(-c7cccc8ccccc78)c7c(-c8cccc9ccccc89)cccc67)c5)c43)n2)cc1 ; c1ccc(-c2cc(-c3ccccc3)cc(-c3cc(-c4ccc5c(c4)c4ccc6c(c7ccccc7n6-c6ccccc6)c4n5-c4nc(-c5ccccc5)nc(-c5ccccc5)n4)cc(-c4ccc(-c5cccc6ccccc56)c5c(-c6cccc7ccccc67)cccc45)c3)c2)cc1]

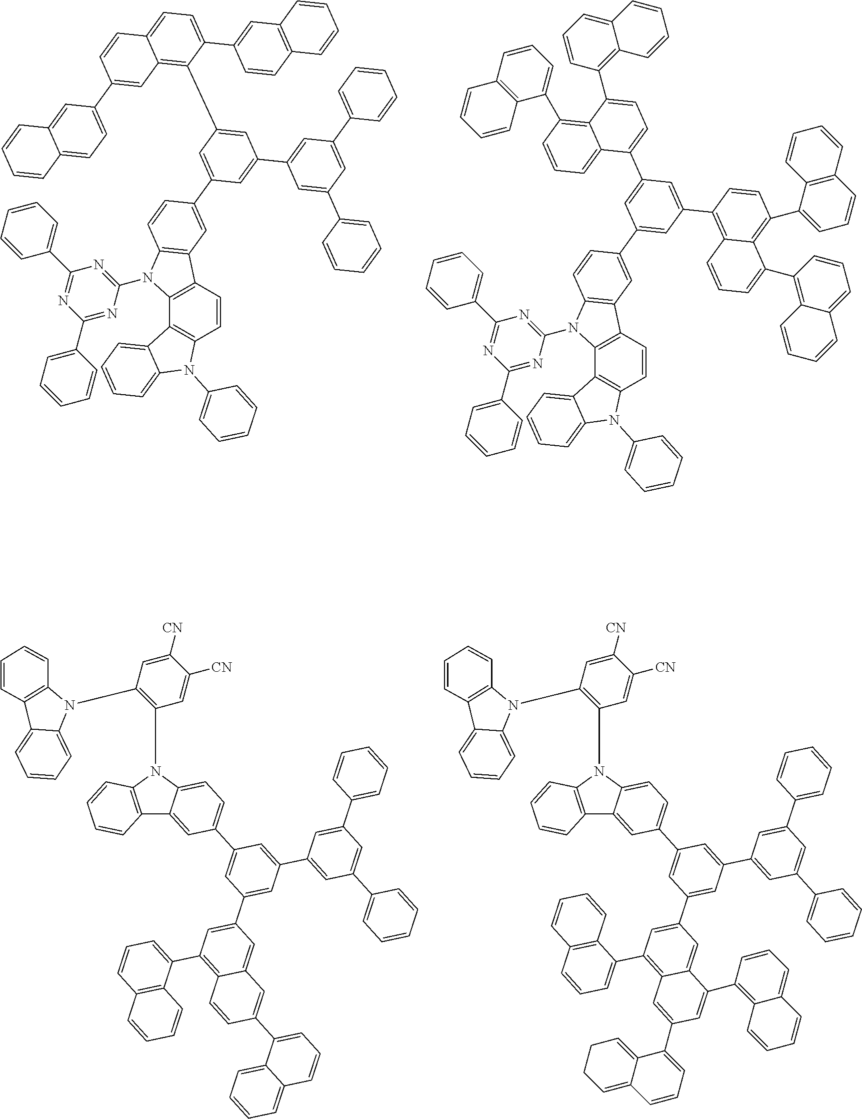

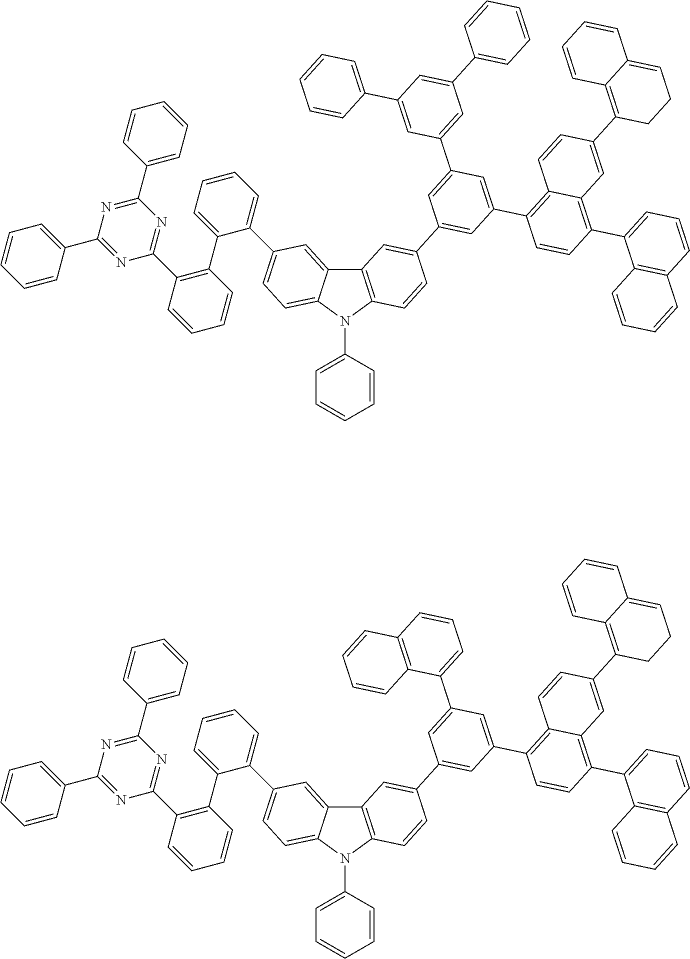 [SMILES: C1=c2ccccc2=C(c2ccc3c(-c4cc(-c5cc(-c6ccccc6)cc(-c6ccccc6)c5)cc(-c5ccc6c(c5)c5cc(-c7ccccc7-c7ccccc7-c7nc(-c8ccccc8)nc(-c8ccccc8)n7)ccc5n6-c5ccccc5)c4)ccc(-c4cccc5ccccc45)c3c2)CC1 ; C1=c2ccccc2=C(c2ccc3c(-c4cc(-c5ccc6c(c5)c5cc(-c7ccccc7-c7ccccc7-c7nc(-c8ccccc8)nc(-c8ccccc8)n7)ccc5n6-c5ccccc5)cc(-c5cccc6ccccc56)c4)ccc(-c4cccc5ccccc45)c3c2)CC1]

The method for synthesizing the organic functional compound of the present embodiment is using a raw material containing an active group to perform a reaction. These active raw materials include the structural units F and SG of the above general formula and at least one ionic group in each case, for example, bromine, iodine, boric acid or borate ester. Appropriate reactions for forming C—C linkage are well known to those skilled in the art and described in the literature, particularly appropriate and exemplary coupling reactions are the SUZUKI, STILLE and HECK coupling reactions.

The present embodiment also provides a formulation for preparing an organic electronic device, which comprises one organic solvent and the above organic functional compound.

In one embodiment, an organic functional compound can be used as a host material in the formulation.

In one embodiment, the formulation further comprises a light emitting material.

In a further embodiment, the formulation according to the present embodiment comprises a host material and a singlet light emitter.

In another further embodiment, the formulation according to this embodiment comprises a host material and a triplet light emitter.

In another further embodiment, the formulation according to the present embodiment comprises a host material and a thermally activated delayed fluorescent material.

In other further embodiments, the formulation according to the present embodiment includes a hole transport material (HTM), in a further embodiment, the HTM includes a crosslinkable group.

The formulation of the present embodiment is a solution or a suspension.

The formulation of the present embodiment may comprise 0.01-20 wt % of the organic functional compound. In another embodiment, the formulation may comprise 1.5-15 wt % of the organic functional compound. In still another embodiment, the formulation may comprise 0.2-10 wt % of the organic functional compound. In a further embodiment, the formulation may comprise 0.25-5 wt % of the organic functional compound.

The organic solvent in the formulation of the present embodiment is selected from: aromatic or heteroaromatic, ester, aromatic ketone or aromatic ether, aliphatic ketone or aliphatic ether, alicyclic or olefinic compound, or inorganic ester compound such as borate ester or phosphate ester, or a mixture of two or more organic solvents above. In one embodiment, the formulation comprises at least 50 wt % of aromatic or heteroaromatic solvent; in another embodiment, the formulation comprises at least 80 wt % of aromatic or heteroaromatic solvent; in still another embodiment, the formulation comprises at least 90 wt % of aromatic or heteroaromatic solvent.

Examples based on aromatic or heteroaromatic solvent according to the present embodiment include, but are not limited to, 1-tetralone, 3-phenoxytoluene, acetophenone, 1-methoxynaphthalene, p-diisopropylbenzene, amylbenzene, tetrahydronaphthalene, cyclohexylbenzene, chloronaphthalene, 1,4-dimethylnaphthalene, 3-isopropylbiphenyl, p-methylisopropylbenzene, dipentylbenzene, o-diethylbenzene, m-diethylbenzene, p-diethylbenzene, 1,2,3,4-tetramethylbenzene, 1,2,3,5-tetramethylbenzene, 1,2,4,5-tetramethylbenzene, butylbenzene, dodecylbenzene 1-methylnaphthalene, 1,2,4-trichlorobenzene, 1,3-dipropoxybenzene, 4,4-difluorodiphenylmethane, diphenyl ether, 1,2-dimethoxy-4-(1-propenyl)benzene, diphenylmethane, 2-phenylpyridine, 3-phenylpyridine, 2-phenoxymethyl ether, 2-phenoxytetrahydrofuran, ethyl-2-naphthyl ether, N-methyldiphenylamine, 4-isopropylbiphenyl, α,α-dichlorodiphenylmethane, 4-(3-phenylpropyl)pyridine, benzyl benzoate, 1,1-bis(3,4-dimethylphenyl)ethane, 2-isopropylnaphthalene, dibenzyl ether, and the like.

Exemplary organic solvents are aliphatic, alicyclic or aromatic hydrocarbon, amine, thiol, amide, nitrile, ester, ether, polyether, alcohol, glycol or polyol. Alcohol represents the appropriate category of solvents. Exemplary alcohol includes alkylcyclohexanol, especially methylated aliphatic alcohol, naphthol, and the like.

The organic solvent may also be a cycloalkane, such as decalin.

The organic solvent may be used alone or as a mixture of two or more organic solvents.

In some embodiments, the formulation according to the present embodiment comprises an organic functional compound as described above and at least one organic solvent, and further includes another organic solvent whose examples include, but are not limited to, methanol, ethanol, 2-methoxyethanol, dichloromethane, trichloromethane, chlorobenzene, o-dichlorobenzene, tetrahydrofuran, anisole, morpholine, toluene, o-xylene, m-xylene, p-xylene, 1,4-dioxane, acetone, methyl ethyl ketone, 1,2-dichloroethane, 3-phenoxy toluene, 1,1,1-trichloroethane, 1,1,2,2-tetrachloroethane, ethyl acetate, butyl acetate, dimethylformamide, dimethylacetamide, dimethyl sulfoxide, tetrahydronaphthalene, decalin, indene, and/or mixtures thereof.

The organic solvent that is particularly suitable for the present embodiment is a solvent whose Hansen solubility parameter is in the following range:

δ_{d} (dispersion force) is in the range of 17.0-23.2 MPa^{1/2}, especially in the range of 18.5-21.0 MPa^{1/2}.

δₚ (polarity force) is in the range of 0.2-12.5 MPa^{1/2}, especially in the range of 2.0-6.0 MPa^{1/2};

δₕ (hydrogen bonding force) is in the range of 0.9-14.2 MPa^{1/2}, especially in the range of 2.0-6.0 MPa^{1/2}.

In the formulation according to the present embodiment, the boiling point parameter of the organic solvent must be taken into account when selecting the organic solvent. In the present embodiment, the boiling point of the organic solvent is ≥150° C.; in another embodiment, the boiling point of the organic solvent is ≥180° C.; in still another embodiment, the boiling point of the organic solvent is ≥200° C.; in still another embodiment, the boiling point of the organic solvent is ≥250° C.; in still another embodiment, the boiling point of the organic solvent is ≥275° C. or ≥300° C. Boiling points in these ranges are beneficial for preventing clogging of the nozzle of the inkjet printing head. The organic solvent can be evaporated from the solvent system to form a film containing a functional material.

The formulation according to the present embodiment satisfies the following requirements in viscosity and surface tension:

1) Its viscosity is in the range of 1 cPs to 100 cPs at 25° C.;

2) Its surface tension is in the range of 19 dyne/cm to 50 dyne/cm at 25° C.

In the formulation according to the present embodiment, the surface tension parameter of the organic solvent must be taken into account when selecting the organic solvent. The suitable surface tension parameters of ink are suitable for a particular substrate and a particular printing method. For example, for inkjet printing, in a further embodiment, the surface tension of the organic solvent at 25° C. is in the range of about 19 dyne/cm to 50 dyne/cm; in another embodiment, the surface tension of the organic solvent at for example, 22 dyne/cm to 35 Dyne/cm; in one embodiment, the surface tension of the organic solvent at such as 25 dyne/cm to 33 dyne/cm.

In a exemplary embodiment, the surface tension of the ink according to the present embodiment at 25° C. is in the range of about 19 dyne/cm to 50 dyne/cm; In one embodiment, the surface tension of the ink according to the present embodiment at 25° C. is in the range of about for example, 22 dyne/cm to 35 dyne/cm; In still one embodiment, the surface tension of the ink according to the present embodiment at 25° C. is in the range of about, such as 25 dyne/cm to 33 dyne/cm.

In the formulation according to the present embodiment, the viscosity parameter of ink must be taken into account when selecting the organic solvent. The viscosity can be adjusted by different methods, such as by proper selection of organic solvent and the concentration of functional materials in the ink. In a exemplary embodiment, the viscosity of the organic solvent is less than 100 cps; In some exemplary embodiment, the viscosity of the organic solvent is for example, less than 50 cps; In some embodiment, the viscosity of the organic solvent is such as 1.5 to 20 cps. The viscosity herein refers to the viscosity during printing at the ambient temperature that is generally 15-30° C., in some exemplary embodiment, the viscosity herein refers to the viscosity during printing at the ambient temperature that is generally for example, 18-28° C., in some exemplary embodiment, the viscosity herein refers to the viscosity during printing at the ambient temperature that is generally such as 20-25° C., in one further embodiment, the viscosity herein refers to the viscosity during printing at the ambient temperature that is generally 23-25° C. The formulation so formulated will be particularly suitable for inkjet printing.

In a exemplary embodiment, the formulation according to the present embodiment has a viscosity at 25° C. in the range of about 1 cps to 100 cps; in some embodiment, the formulation according to the present embodiment has a viscosity at 25° C. in the range of about for example, 1 cps to 50 cps; in some embodiment, the formulation according to the present embodiment has a viscosity at 25° C. in the range of about 1.5 cps to 20 cps.

The ink obtained from the organic solvent satisfying the above-mentioned boiling point parameter, surface tension parameter and viscosity parameter can form a functional material film with a uniform thickness and composition property.

The disclosure also relates to the application of the formulation as printing ink in the preparation of an organic electronic device, for example, by a preparation method via printing or coating.

Wherein, suitable printing or coating techniques include, but are not limited to, inkjet printing, letterpress printing, screen printing, dip coating, spin coating, doctor blade coating, roller printing, twisting roller printing, lithography, flexography, rotary printing, spraying, brushing or pad printing, slot die coating, etc. Intaglio printing, screen printing and inkjet printing are preferred. The solution or suspension may additionally contain one or more components such as surface-active compound, lubricant, wetting agent, dispersant, hydrophobic agent, binder, etc., for adjusting the viscosity and the film forming property, enhancing the adhesion, and the like.

In the preparation method as described above, a functional layer is formed on the substrate and its thickness is controlled to be 5 nm to 1000 nm.

The present embodiment also provides a mixture comprises an organic functional compound or formulation according to the present embodiment and at least another organic functional material. Another organic functional material may be selected from hole (also called electron hole) injection materials (HIM), hole transport materials (HTM), hole blocking materials (HBM), electron injection materials (EIM), electron transport materials (ETM), electron blocking materials (EBM), organic matrix materials (Host), singlet emitters (fluorescent light emitter), triplet emitters (phosphorescent light emitter), thermally excited delayed fluorescent materials (TADF material) or organic dyes.

The present embodiment further relates to an organic electronic device including at least one organic functional compound according to the present embodiment, or at least one functional layer that is prepared using the formulation according to the present embodiment. The organic electronic device includes at least one cathode, one anode, and one functional layer located between the cathode and the anode, wherein the functional layer includes at least one organic functional compound as described above.

In one embodiment, the organic electronic device is organic light-emitting diode (OLED), organic photovoltaic cell (OPV), organic light-emitting electrochemical cell (OLEEC), organic field effect transistor (OFET), organic light-emitting field effect transistor, organic laser, organic spintronic device, organic sensor, or organic plasmon emitting diode.

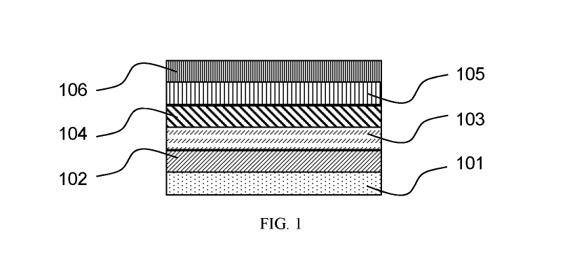

In a further embodiment, the above-mentioned organic electronic device is an electroluminescent device, particularly an OLED, whose structure is shown in FIG. 1 and comprises a substrate 101, an anode 102, and at least one light-emitting layer 104 and a cathode 106.

The substrate 101 may be opaque or transparent. A transparent substrate can be used to manufacture a transparent light-emitting device. The substrate 101 may be rigid or elastic. The substrate 101 may be plastic, metal, semiconductor wafer or glass. In one embodiment, the substrate 101 has a smooth surface. Surface defect-free substrate is a particularly ideal choice. In a further embodiment, the substrate 101 is flexible and may be selected from polymeric film or plastic, with glass transition temperature Tg of greater than 150° C., for example, greater than 200° C., such as greater than 250° C., or greater than 300° C. Suitable examples of flexible substrates are poly(ethylene terephthalate) (i.e. PET) and polyethylene glycol (2,6-naphthalene) (i.e. PEN).

The anode 102 may include a conductive metal, a metal oxide, or a conductive polymer. The anode 102 can easily inject holes into the hole injection layer (HIL) or the hole transport layer (HTL) or the light-emitting layer. In one embodiment, the absolute value of the difference between the work function of the anode 102 and the HOMO energy level or valence band energy level of the light emitter in the light-emitting layer or the p-type semiconductor material used as HIL or HTL or electron blocking layer (EBL) is less than 0.5, for example, less than 0.3 eV, such as less than 0.2 eV. Examples of anode 102 material include, but are not limited to, Al, Cu, Au, Ag, Mg, Fe, Co, Ni, Mn, Pd, Pt, ITO, aluminum doped zinc oxide (AZO), and the like. Other suitable anodes 102 are known and can be readily selected by skilled person in the art. The anode 102 may be deposited using any suitable technique, such as a suitable physical vapor deposition method, including radio frequency magnetron sputtering, vacuum thermal evaporation, e-beam, and the like. In some embodiments, the anode 102 is patterned. Patterned ITO conductive substrate is commercially available and can be used to prepare the device according to the present embodiment.

Cathode 106 may include a conductive metal or metal oxide. The cathode 106 can easily inject electrons into the EIL or ETL or directly into the light-emitting layer. In one embodiment, the absolute value of the difference between the work function of the cathode 106 and the LOMO energy level or the conduction band energy level of the light emitter in the light-emitting layer or the n-type semiconductor material used as electron injection layer (EIL) or electron transport layer (ETL) or an hole blocking layer (HBL) is less than 0.5, for example, less than 0.3 eV, such as less than 0.2 eV. In principle, all materials that can be used as a cathode 106 for OLED are possibly used as a cathode material for the device of the present embodiment. Examples of cathode materials comprise, but are not limited to: Al, Au, Ag, Ca, Ba, Mg, LiF/Al, MgAg alloy, BaF2/Al, Cu, Fe, Co, Ni, Mn, Pd, Pt, ITO, etc. The cathode 106 material may be deposited using any suitable technique, such as a suitable physical vapor deposition method, including radio frequency magnetron sputtering, vacuum thermal evaporation, e-beam, and the like.

OLED can also include other functional layers such as hole injection layer (HIL) or hole transport layer (HTL) 103, electron blocking layer (EBL), electron injection layer (EIL) or electron transport layer (ETL) 105, and hole blocking layer (HBL).

In one embodiment, in the organic light-emitting device according to the present embodiment, the hole injection layer (HIL) or the hole transport layer (HTL) 103 is prepared by printing the formulation of the present embodiment.

In some embodiments, in the light-emitting device according to the present embodiment, the electron injection layer (EIL) or the electron transport layer (ETL) 105 is prepared by printing the formulation of the present embodiment.

In a further embodiment, in the light-emitting device according to the present embodiment, the light-emitting layer (104) is prepared by printing the formulation of the present embodiment.

The light-emitting wavelength of the electroluminescent device according to the present embodiment is between 300 and 1000 nm, in one embodiment, the light-emitting wavelength of the electroluminescent device according to the present embodiment is between 350 and 900 nm, and in one embodiment, the light-emitting wavelength of the electroluminescent device according to the present embodiment is between 400 and 800 nm.

The present embodiment also relates to the application of the organic electronic device according to the present embodiment in various electronic equipment, including but are not limited to display equipment, lighting equipment, light source, sensor, and the like.

The present embodiment will be described in detail below with reference to the exemplary embodiments.

### EXAMPLE 1

### Synthesis of Compound 1

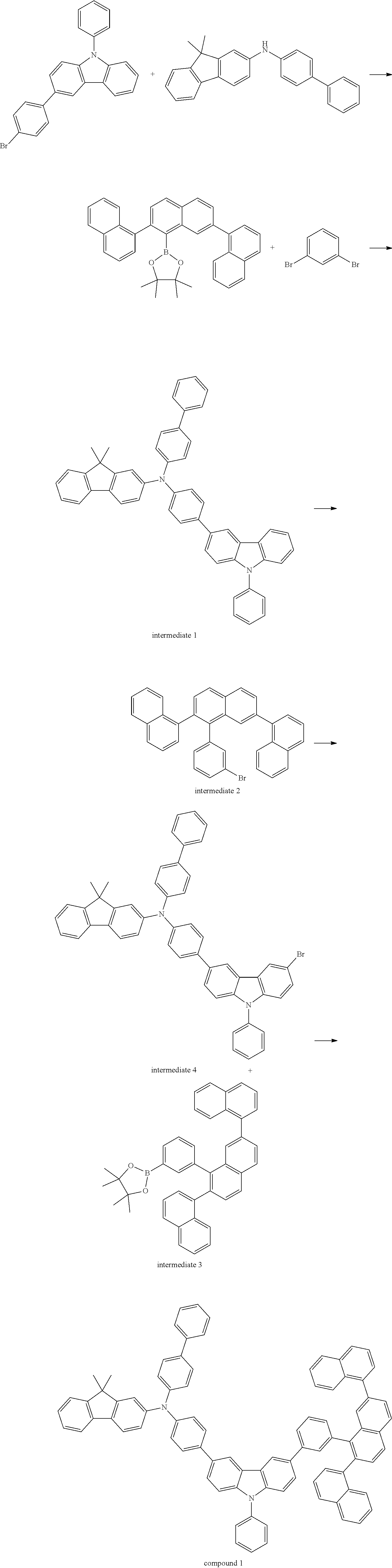

11.5 g (0.029 mol) 3-(4-bromophenyl)-9-phenyl-9H-carbazole, 10.5 g (0.029 mol) (N-([1,1′-biphenyl]-4-yl) -9,9-dimethyl-9H-fluoren-2-amine), 1.5 g Pd(dba)₂ and 8.6 g (0.087 mol) sodium tert-butoxide were successively added to 200 ml toluene and reacted overnight at 90° C. After mass spectrometry showed that the reaction was completed, the reaction liquid was poured into water, and then extracted twice with dichloromethane and spin dried. 12.9 g of white solid intermediate 1 was obtained by column chromatography with a yield of 64%.

32 g (0.063 mol) 2-([1,2′:7′,1″-ternaphthalen]-1′-yl)-4,4,5,5-tetramethyl-1,3,2-dioxaborolane was dissolved into 500 ml toluene, then 15 g dibromobenzene (0.063 mol), 1 g tetrakis(triphenylphosphine)palladium, 20 g potassium carbonate (0.147 mol), 60 ml water, and 60 ml of ethanol were successively added, and heated to 110° C. to react for 15 hours. TLC plate showed that the reaction was completed. The reaction liquid was added to water and extracted three times with dichloromethane. The organic phase was then dried and concentrated to give a crude product. 13.5 g of solid intermediate 2 was obtained by column chromatography with a yield of 40%.

13.5 g (0.025 mol) intermediate 2 was dissolved into 200 ml dioxane, and 6 g pinacol ester (0.035 mol), 0.7 g tetrakis(triphenylphosphine)palladium, 26.8 g potassium carbonate (0.19 mol), 100 ml water, 200 ml ethanol were added and then warmed to 105° C. TLC plate showed that the reaction was completed after reacting for 6 hours. The reaction liquid was added to 500 ml water and extracted three times with dichloromethane. The organic phases were combined, dried, spin dried to give a crude product. 10.3 g intermediate 3 as a white solid was obtained by column chromatography with a yield of 70.8%.

12.9 g (0.0186 mol) of the intermediate 1 was dissolved into 50 ml DMF, and then 4.1 g NBS (0.023 mol) was added and stirred at room temperature for 1.5 hours. Then 200 ml water was added, after suction filtration, 13.7 g (95% yield) product, i.e. intermediate 4, was obtained.

10.3 g (0.018 mol) intermediate 3 and 13.7 g (0.018 mol) intermediate 4 were successively dissolved into 300 ml toluene at room temperature, then 1.2 g of tetrakis(triphenylphosphine)palladium, 10 g potassium carbonate (0.74 mol), 60 ml water, 60 ml ethanol were successively added, and heated to 110° C. to react for 15 hours. TLC plate showed that the reaction was completed. The reaction liquid was added into water and extracted three times with dichloromethane. The organic phase was then dried and concentrated to give a crude product. 13 g solid compound 1 was obtained by column chromatography with a yield of 65%.

### EXAMPLE 2

### Synthesis of Compound 2

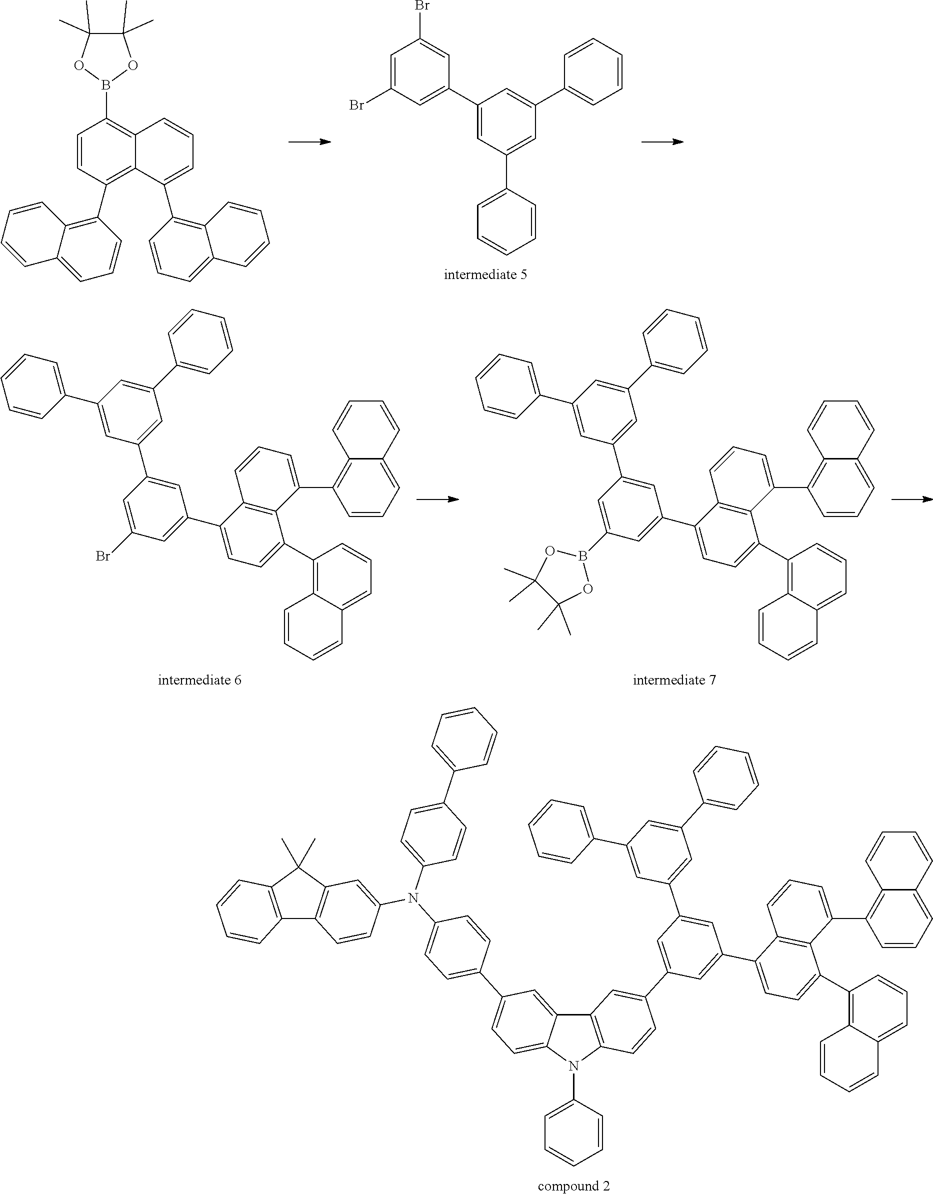

35.6 g (0.1 mol) 2-([1,1′:3′,1″-terphenyl]-5′-yl)-4,4,5,5-tetramethyl-1,3,2-dioxaborolane and 22 g (0.07 mol) tribromobenzene were dissolved into 300 ml toluene at room temperature, and then 3.2 g tetrakis(triphenylphosphine)palladium, 58 g potassium carbonate (0.44 mol), 100 ml water, and 100 ml ethanol were successively added and heated to 110° C. to react for 15 hours. TLC plate showed that the reaction was completed. The reaction liquid was added into water and extracted three times with dichloromethane. The organic phase was then dried and concentrated to give a crude product. 13 g solid intermediate 5 was obtained by column chromatography with a yield of 40%.

4.61 g (0.01 mol) intermediate 5 and 3.5 g (0.007 mol) 2-([1,1′:8′,1″-ternaphthalen]-4′-yl)-4,4,5,5-tetramethyl-1,3,2-dioxaborolane were dissolved into 100 ml toluene at room temperature, and then 0.4 g tetraki(striphenylphosphine)palladium, 5.8 g potassium carbonate (0.044 mol), 60 ml water, and 60 ml ethanol were successively added and heated to 110° C. to react for 15 hours. TLC plate showed that the reaction was completed. The reaction liquid was added to water and extracted three times with dichloromethane. The organic phase was then dried and concentrated to give a crude product. 2.5 g solid intermediate 6 was obtained by column chromatography with a yield of 50%.

2.5 g (0.0035 mol) intermediate 6 was dissolved into 100 ml dioxane, and 1 g pinacol ester (0.006 mol), 0.7 g tetrakis(triphenylphosphine)palladium, 1.8 g potassium carbonate (0.014 mol), 50 ml water, and 50 ml ethanol were added and then warmed to 105° C. TLC plate showed that the reaction was completed after reacting for 6 hours. The reaction liquid was added into 100 ml water and extracted three times with dichloromethane. The organic phases were combined, dried, spin dried to give a crude product. 1.9 g white solid intermediate 7 was obtained by column chromatography with a yield of 70%.

13.8 g (0.018 mol) intermediate 7 and 13.7 g (0.018 mol) intermediate 4 were dissolved into 300 ml toluene at room temperature, and then 1.2 g tetrakis(triphenylphosphine)palladium, 9.7 g potassium carbonate (0.074 mol), 60 ml water, and 60 ml ethanol were added and heated to 110° C. to react for 15 hours. TLC plate showed that the reaction was completed. The reaction liquid was added into water and extracted three times with dichloromethane. The organic phase was then dried and concentrated to give a crude product. 12 g solid compound 2 was obtained by column chromatography with a yield of 65%.

### EXAMPLE 3

### Synthesis of Compound 3

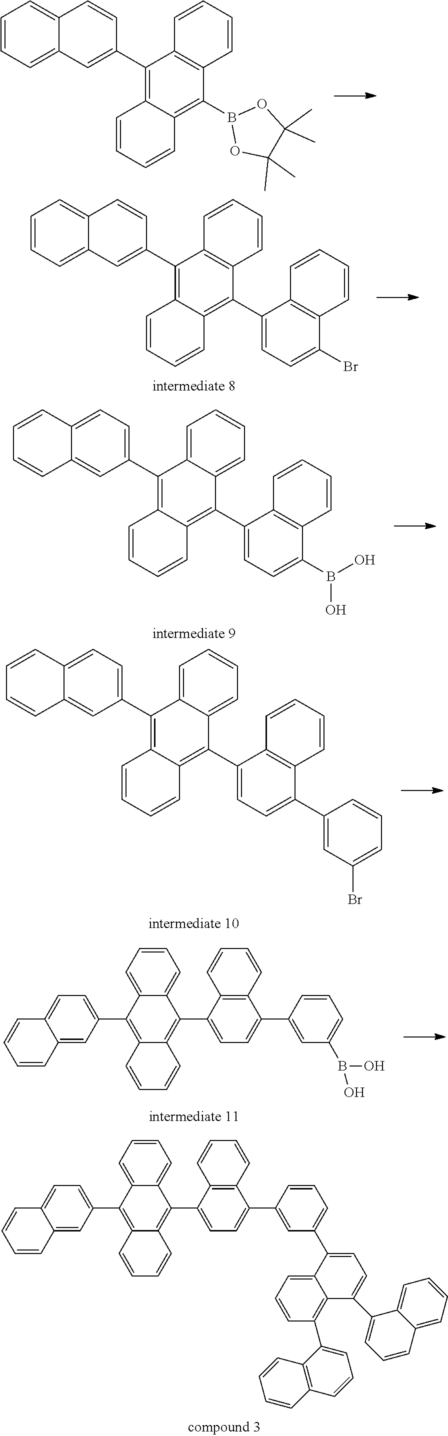

43 g (0.1 mol) 2-([1,1′:3′,1″-terphenyl]-5′-yl)-4,4,5,5-tetramethyl-1,3,2-dioxaborolane and 19.8 g (0.07 mol) 1,4-dibromonaphthalene were successively dissolved into 300 ml toluene at room temperature, and then 3.2 g tetrakis(triphenylphosphine)palladium, 58 g (0.44 mol) potassium carbonate, 100 ml water, and 100 ml ethanol were successively added and heated to 110° C. to react for 15 hours. TLC plate showed that the reaction was completed. The reaction liquid was added into water and extracted three times with dichloromethane. The organic phase was then dried and concentrated to give a crude product. 21.3 g solid intermediate 8 was obtained by column chromatography with a yield of 42%.

5 g (0.01 mol) intermediate 8 was dissolved into 100 ml anhydrous tetrahydrofuran at room temperature. After cooling to −78° C., 4.4 ml butyllithium was slowly added dropwise and kept at this temperature for 1 hour. Then 2.5 g (0.017 mol) triethyl borate was added and naturally warmed to room temperature to react for 10 hours. TLC plate showed that the reaction was completed. 2 N HCl was added and stirred for 2 hour. The reaction liquid was added into water and extracted three times with dichloromethane. The organic phase was then dried and concentrated to give a crude product. The crude product was recrystallized with ethyl ether to give 3.8 g intermediate 9, with a yield of 80%.

47.7 g (0.1 mol) intermediate 9 and 28 g (0.1 mol) o-bromoiodobenzene were successively dissolved into 300 ml toluene at room temperature. And then 5.2 g tetrakis(triphenylphosphine)palladium, 58 g (0.44 mol) potassium carbonate, 100 ml water, and 100 ml ethanol were added successively and heated to 110° C. to react for 15 hours. TLC plate showed that the reaction was completed. The reaction liquid was added into water and extracted three times with dichloromethane. The organic phase was then dried and concentrated to give a crude product. 41 g solid intermediate 10 was obtained by column chromatography with a yield of 70%.

6 g (0.01 mol) intermediate 10 was dissolved into 100 ml anhydrous tetrahydrofuran at room temperature. After cooling to −78° C., 4.4 ml butyllithium was slowly added dropwise and kept at this temperature for 1 hour. Then 2.5.g (0.017 mol) triethyl borate was added and naturally warmed to room temperature to react for 10 hours. TLC plate showed that the reaction was completed. 2 N HCl was added and stirred for 2 hour. The reaction liquid was added to water and extracted three times with dichloromethane. The organic phase was then dried and concentrated to give a crude product. The crude product was recrystallized with ethyl ether to give 3.3 g intermediate 11, with a yield of 60%.

27.5 g (0.05 mol) intermediate 11 and 25 g (0.05 mol) intermediate 2 were successively dissolved into 300 ml toluene at room temperature. And then 6.2 g tetrakis(triphenylphosphine)palladium, 29 g (0.22 mol) potassium carbonate, 100 ml water, and 100 ml ethanol were added successively and heated to 110° C. to react for 15 hours. TLC plate showed that the reaction was completed. The reaction liquid was added to water and extracted three times with dichloromethane. The organic phase was then dried and concentrated to give a crude product. 26 g solid compound 3 was obtained by column chromatography with a yield of 60%.

### EXAMPLE 4

### Synthesis of Compound 4

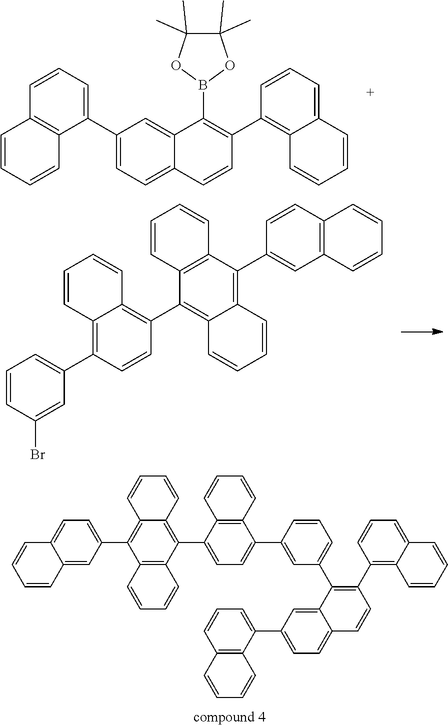

10.3 g (0.018 mol) 2-([1,2′:7′,1″-ternaphthalen]-1′-yl)-4,4,5,5-tetramethyl-1,3,2-dioxaborolane and 13.7 g (0.018 mol) intermediate 10 were successively dissolved into 300 ml toluene at room temperature, and then 1.2 g tetrakis(triphenylphosphine)palladium, 10 g potassium carbonate (0.74 mol), 60 ml water, and 60 ml ethanol were successively added and heated to 110° C. to react for 15 hours. TLC plate showed that the reaction was completed. The reaction liquid was added to water and extracted three times with dichloromethane. The organic phase was then dried and concentrated to give a crude product. 13 g solid compound 4 was obtained by column chromatography with a yield of 65%.

### EXAMPLE 5

### Synthesis of Compound 5

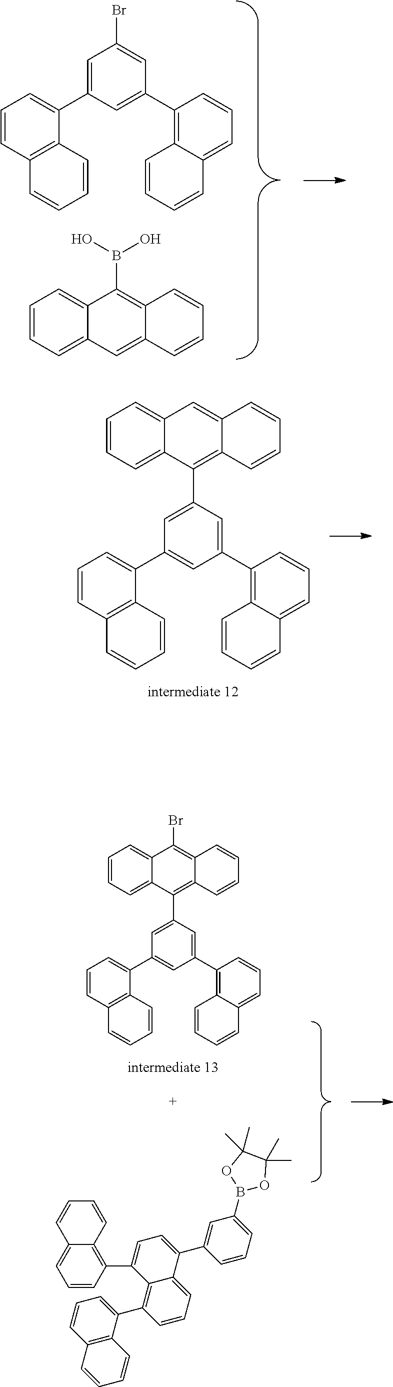

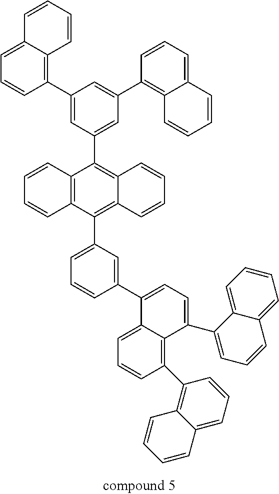 [SMILES: c1cc(-c2c3ccccc3c(-c3cc(-c4cccc5ccccc45)cc(-c4cccc5ccccc45)c3)c3ccccc23)cc(-c2ccc(-c3cccc4ccccc34)c3c(-c4cccc5ccccc45)cccc23)c1]

8.2 g (0.037 mol) anthracen-9-ylboronic acid, 12.6 g (0.031 mol) 1,1′-(5-bromo-1,3-phenylene)dinaphthalene, 1.2 g tetrakis(triphenylphosphine)palladium, 17 g (0.124 mol) potassium carbonate were dissolved into 200 ml dioxane and 50 ml water to react at 95° C. for 4 hours. The reaction liquid was poured into water and extracted twice with dichloromethane. The organic phases were combined, dried, spin dried to obtain 10.5 g solid intermediate 12 by column chromatography with a yield of 67%.

10.5 g (0.021 mol) intermediate 12 was dissolved into 50 ml DMF, 4.1 g NBS (0.023 mol) was added and stirred at room temperature for 1.5 hours. Then 200 ml water was added and suction filtration was conducted to obtain 11.5 g intermediate 13 with a yield of 95%.

11.5 g (0.02 mol) intermediate 13, 11.6 g (0.02 mol) 2-(3-([1,1′:8′,1″-ternaphthalen]-4′-yl)phenyl)-4,4,5,5-tetramethyl-1,3,2-dioxaborolane, 1 g tetrakis(triphenylphosphine)palladium, 13 g potassium carbonate (0.095 mol) were dissolved into 200 ml dioxane and 50 ml water, and then heated to 95° C. to react 3 hours, the spot plate showed that the reaction was completed. The reaction liquid was added into water and extracted twice with dichloromethane. The organic phases were combined, dried, spin dried. 12.6 g white product that is compound 5 was obtained by column chromatography with a yield of 66%.

### EXAMPLE 6

### Synthesis of Compound 6

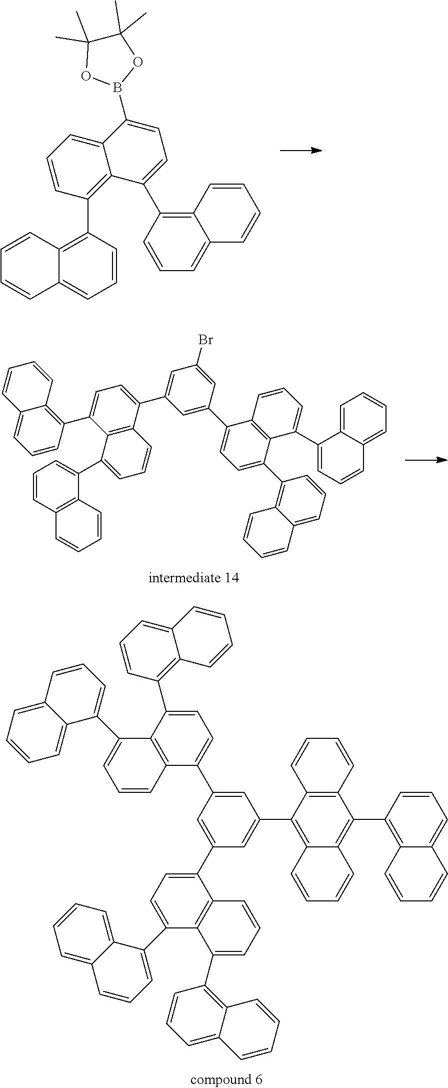

46 g (0.092 mol) 2-([1,1′:8′,1″-ternaphthalen]-4′-yl)-4,4,5,5-tetramethyl-1,3,2-dioxaborolane was dissolved into 500 ml toluene, then 11.4 g tribromobenzene (0.037 mol), 1.8 g tetrakis(triphenylphosphine)palladium, 20 g potassium carbonate (0.147 mol), 60 ml water, and 60 ml ethanol were successively added, and heated to 110° C. to react for 15 hours. TLC plate showed that the reaction was completed. The reaction liquid was added into water and extracted three times with dichloromethane. The organic phase was then dried and concentrated to give a crude product. 13.4 g solid intermediate 14 was obtained by column chromatography with a yield of 40%.

13.4 g (0.0147 mol) intermediate 14 and 5.1 g (0.0147 mol) (10-(naphthalen-1-yl)anthracen-9-yl)boronic acid, 1 g tetrakis(triphenylphosphine)palladium, 8.1 g potassium carbonate (0.06 mol) were dissolved in 200 ml dioxane and 50 ml water, and then heated to 95° C. to react for 3 hours. The spot plate showed that the reaction was completed. The reaction liquid was poured into water and extracted twice with dichloromethane. The organic phases were combined, dried, spin dried. After column chromatography, 12 g white product, i.e. compound 6, was obtained, with a yield of 72%.

### EXAMPLE 7

### Synthesis of Compound 7

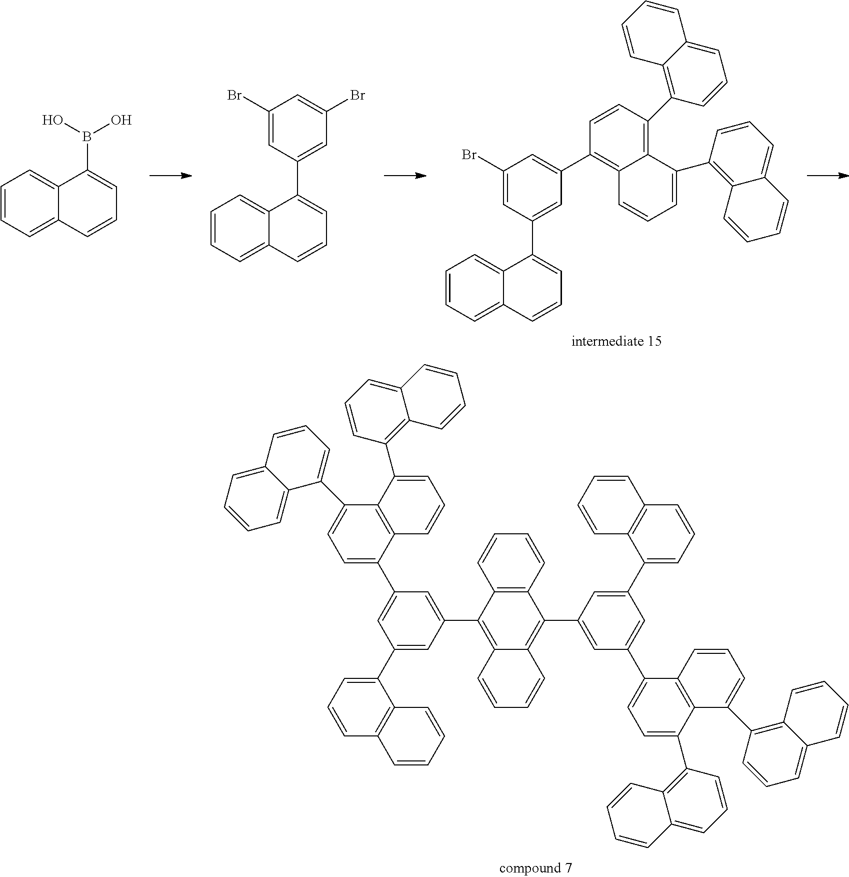

At room temperature, 53.7 g (0.171 mol) tribromobenzene and 29.5 g (0.171 mol) naphthalen-1-ylboronic acid were successively added into a two-mouth flask which contains 500 ml dioxane. 94.5 g K₂CO₃ (0.684 mol) was dissolved to 300 ml water and added to the above system. 2 g Pd(pph₃)₄ was then added. After three times of nitrogen replacement, the temperature was increased to 80° C. to react for 2.5 hours. TLC plate showed that the reaction was completed. After cooling to room temperature, dichloromethane was added. After washing with water, spin drying was performed. 34.8 g solid was obtained by column chromatography with a field of 57%.

34.8 g (0.096 mol) of the solid obtained above, 48 g (0.096 mol) 2-([1,1′:8′,1″-ternaphthalen]-4′-yl)-4,4,5,5-tetramethyl-1,3,2-dioxaborolane were successively dissolved into 500 ml toluene at room temperature, then 1.8 g tetraki(striphenylphosphine)palladium, 20 g potassium carbonate (0.147 mol), 60 ml water, and 60 ml ethanol were added successively, and heated to 110° C. to react for 15 hours. TLC plate showed that the reaction was completed. The reaction liquid was added into water and extracted three times with dichloromethane. The organic phase was then dried and concentrated to give a crude product. 21 g solid intermediate 15 was obtained by column chromatography with a yield of 33%.

21 g (0.032 mol) intermediate 15 and 6.2 g (0.014 mol) 9,10-bis(4,4,5,5-tetramethyl-1,3,2-dioxaborolan-2-yl)anthracene were dissolved successively into 300 ml toluene at room temperature, then 1.8 g tetraki(striphenylphosphine)palladium, 20 g potassium carbonate (0.147 mol), 60 ml water, and 60 ml ethanol were added successively, and heated to 110° C. to react for 15 hours. TLC plate showed that the reaction was completed. The reaction liquid was added into water and extracted three times with dichloromethane. The organic phase was then dried and concentrated to give a crude product. 10.4 g solid compound 7 was obtained by column chromatography with a yield of 54%.

### EXAMPLE 8

### Synthesis of Compound 8

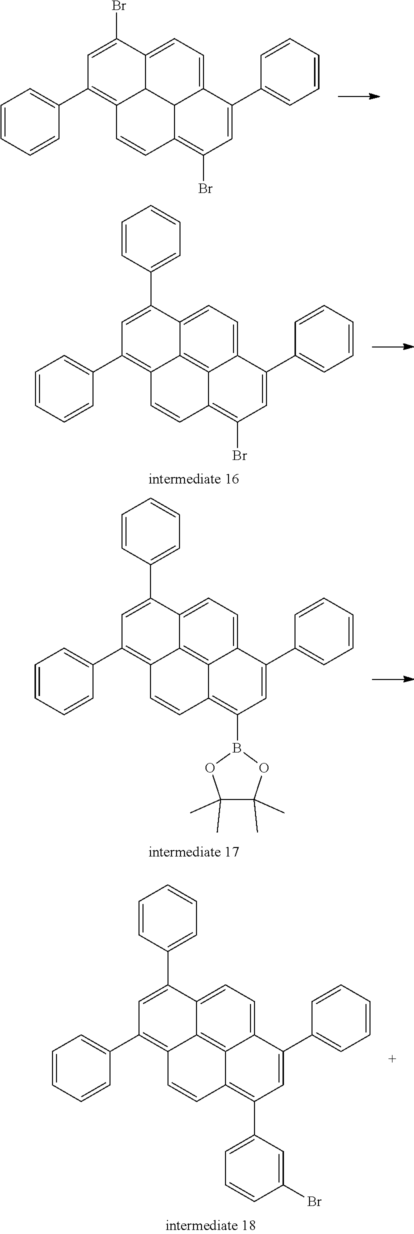

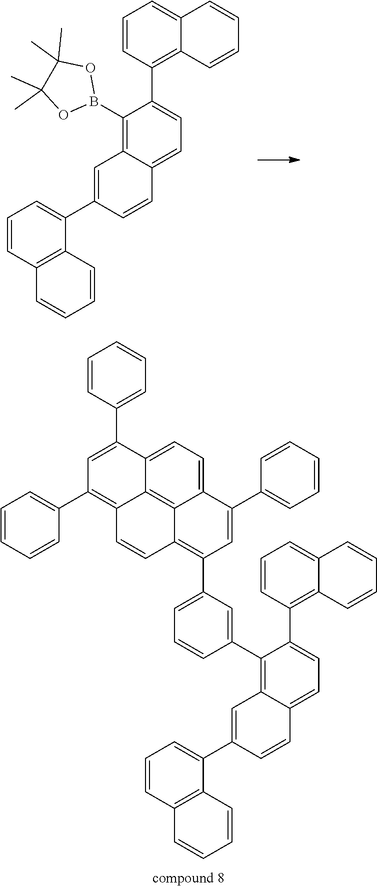

At room temperature, 51 g (0.1 mol) 1,6-dibromo-3,8-diphenylpyrene and 14.3 g (0.07 mol) phenylboronic acid were successively added into a two-mouth flask which contains 500 ml dioxane. 90 g K₂CO₃ (0.684 mol) was dissolved in 300 ml water and added to the above system. 7 g Pd(pph₃)₄ was then added. After three times of nitrogen replacement, the temperature was increased to 80° C. to react for 2.5 hours. TLC plate showed that the reaction was completed. After cooling to room temperature, dichloromethane was added. After washing with water, spin drying was performed. 20 g a solid intermediate 16 was obtained by column chromatography with a yield of 56%.

25.4 g (0.05 mol) intermediate 16 was dissolved into 500 ml dioxane, and 21 g pinacol ester (0.085 mol), 3.7 g tetrakis(triphenylphosphine)palladium, 26 g potassium carbonate (0.2 mol), 100 ml water, and 100 ml ethanol were added and then warmed to 105° C. TLC plate showed that the reaction was completed after reacting for 6 hours. The reaction liquid was added to 100 ml water and extracted three times with dichloromethane. The organic phases were combined, dried, spin dried to give a crude product. After column chromatography, 16.7 g white solid intermediate 17 was obtained with a yield of 70%.

At room temperature, 5.6 g intermediate 17 (0.01 mol) and 2.8 g o-bromoiodobenzene (0.01 mol) were successively added to a two-mouth flask which contains 100 ml dioxanetherein. 9 g (0.0684 mol) K₂CO₃ was dissolved in 50 ml water and added to the above system. 1 g Pd(pph3)4 was then added. After three times of nitrogen replacement, the temperature was raised to 80° C. to react for 2.5 hours. TLC plate showed that the reaction was completed. After cooling to room temperature, dichloromethane was added. After washing with water, spin drying was performed. 4.1 g solid intermediate 18 was obtained by column chromatography with a yield of 70%.

At room temperature, 11.7 g (0.02 mol) intermediate 18 and 10 g (0.02 mol) 2-([1,2′:7′,1″-ternaphthalen]-1′-yl)-4,4,5,5-tetramethyl-1,3,2-dioxaborolane were successively added into a two-mouth flask which contains 500 ml dioxane therein. 90 g K₂CO₃ (0.684 mol) was dissolved in 300 ml water and added to the above system. 2 g Pd (pph₃)₄ was then added. After three times of nitrogen replacement, the temperature was raised to 80° C. to react for 2.5 hours. The TLC plate showed that the reaction was completed. After cooling to room temperature, dichloromethane was added. After washing with water, spin-drying was performed. 11.3 g solid compound 8 was obtained by column chromatography with a yield of 64%.

### EXAMPLE 9

### Synthesis of Compound 9

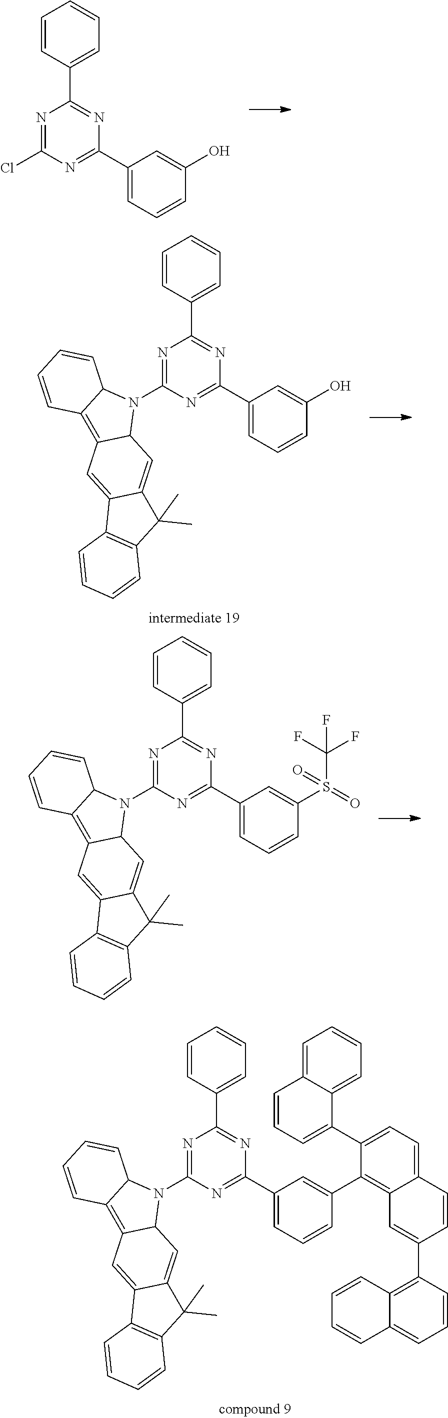

10.8 g (0.038 mol) 11,11-dimethyl-4a,5,11,12b-tetrahydroindeno[1,2-b]carbazole was dissolved into 40 ml DMF at 0° C., 1.-2 g NaH (0.050 mol) was added in batches. After keeping at this temperature for 30 minutes, 10.74 g (0.038 mol) 3-(4-chloro-6-phenyl-1,3,5-triazin-2-yl)phenol was added in batches and reacted at room temperature for 1 hour. Then this system was poured into water and extracted twice with dichloromethane, and then dried, spin-dried. 12.3 g intermediate 19 was obtained by column chromatography with a yield of 61%.

12.3 g intermediate 19 was dissolved into 100 ml dichloromethane and cooled to 0° C. Trifluoromethanesulfonic anhydride was slowly added dropwise and kept at this temperature for 30 minutes. Then the reaction liquid was poured into water, and conducted liquid separation. The organic phase was dried and spin dried to give 17 g solid.

9.6 g (0.0189 mol) 1′-bromo-1,2′:7′,1″-ternaphthalene was dissolved into THF, PdCl₂(dppf) and 3 g LiBr was added. After cooling to 0° C., 17 g solid obtained in the previous step solved in THF was slowly added dropwise to this system. After the addition was completed, the temperature was raised to room temperature to react overnight The reaction liquid was added into water, extracted with dichloromethane, and then dried and spin dried. 12 g of the final product, i.e. compound 9, was obtained by column chromatography with a yield of 57.9%.

### EXAMPLE 10

### Synthesis of Compound 10

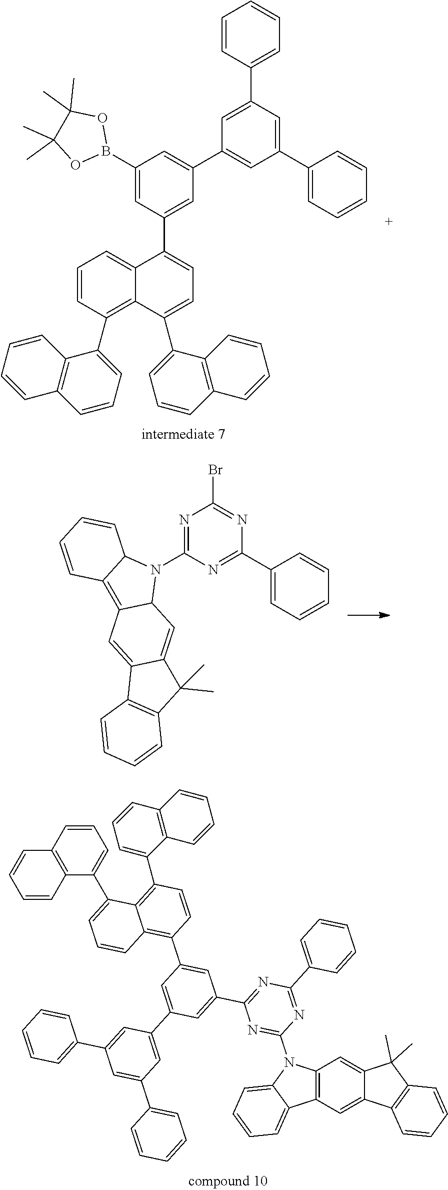

20.3 g (0.024 mol) intermediate 7 was dissolved into 100 ml dioxane, then 13 g (0.024 mol) 5-(4-bromo-6-phenyl-1,3,5-triazin-2-yl)-7,7-dimethyl-4a,5,5a,7-tetrahydroindeno[2,1-b]carbazole, 1.3 g tetrakis(triphenylphosphine)palladium (0.0017 mol), 8.9 g potassium carbonate (0.6 mol), 50 ml water, and 50 ml ethanol were added, and then warmed to 105° C. TLC plate showed that the reaction was completed after reacting for 12 hours. The reaction liquid was added into 200 ml water and extracted three times with dichloromethane. The organic phases were combined, dried, spin dried to give a crude product. 14 g white solid compound 10 was obtained by column chromatography with a yield of 52%.

### EXAMPLE 11

### Synthesis of Compound 11

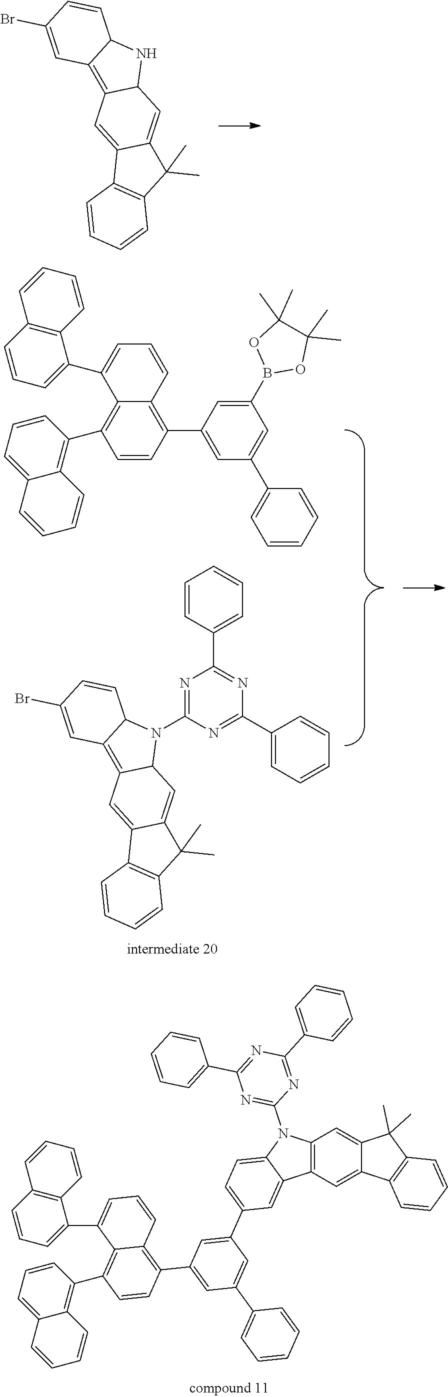

19 g (0.05 mol) 2-bromo-7,7-dimethyl-4a,5,5a,7-tetrahydroindeno[2,1-b]carbazole was dissolved into 40 ml DMF at 0° C., 1.9 g NaH (0.050 mol) was added in batches. After keeping at this temperature for 30 minutes, 14 g (0.05 mol) 2-chloro-4,6-diphenyl-1,3,5-triazine was added in batches and reacted at room temperature for 1 hour. Then this system was poured into water, extracted twice with dichloromethane, and then dried, spin-dried. 18.6 g intermediate 20 was obtained by column chromatography with a yield of 61%.

18.6 g (0.031 mol) intermediate 20 was dissolved into 500 ml dioxane, and then 20 g (0.031 mol) 2-(5-([1,1′:8′,1″-ternaphthalen]-4′-yl)-[1,1′-biphenyl]-3-yl)-4,4,5,5-tetramethyl-1,3,2-dioxaborolane, 2.3 g tetrakis(triphenylphosphine)palladium, 20 g potassium carbonate (0.155 mol), 100 ml water, and 100 ml ethanol were added and then warmed to 105° C. TLC plate showed that the reaction was completed after reacting for 12 hours. The reaction liquid was added to 200 ml water and extracted three times with dichloromethane. The organic phases were combined, dried, spin dried to give a crude product. 18 g white solid compound 11 was obtained by column chromatography with a yield of 56%.

### EXAMPLE 12

### Synthesis of Compound 12

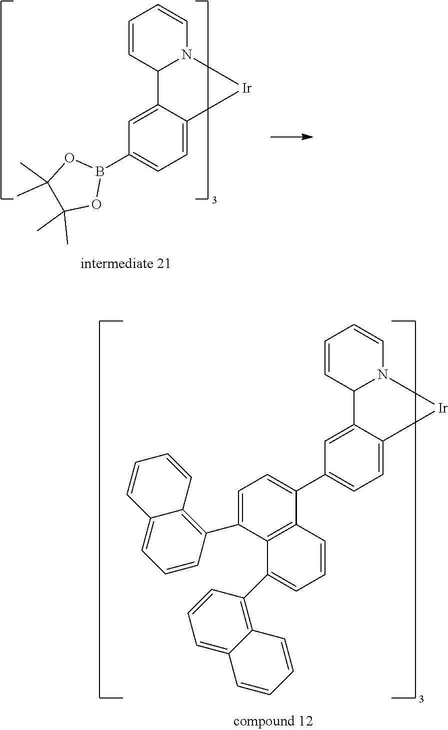

20 g (0.0156 mol) intermediate 21 was dissolved into 500 ml dioxane, 28.7 g 4′-bromo-1,1′:8′,1″-ternaphthalene (0.0624 mol), 2.6 g palladium acetate, 28 g potassium phosphate (0.136 mol), 8 g tris(2-methylphenyl)phosphine, 100 ml water, and 200 ml toluene were added and then warmed to 115° C. The TLC plate showed that the reaction was completed after reacting for 24 hour. The reaction liquid was added to 500 ml water and extracted three times with dichloromethane. The organic phases were combined, dried, spin dried to give a crude product. 16 g white solid compound 12 was obtained by column chromatography with a yield of 80%.

### EXAMPLE 13

### Synthesis of Compound 13

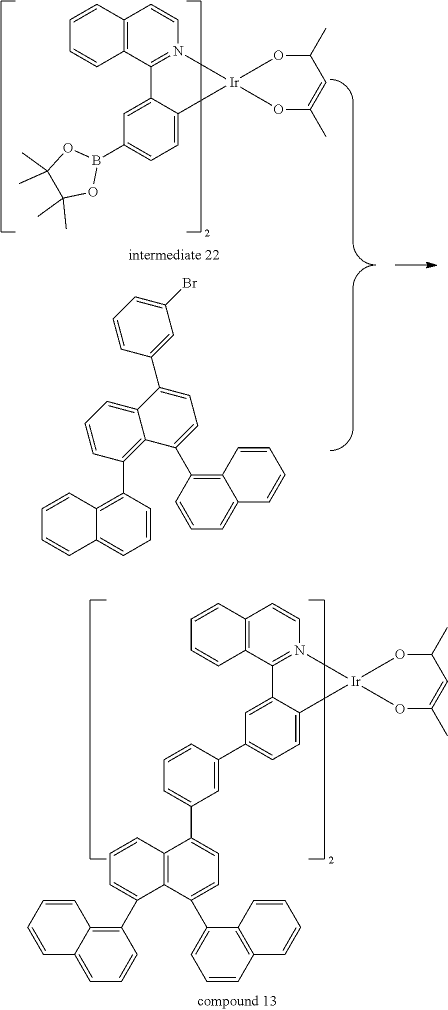

23.1 g (0.0195 mol) intermediate 22 was dissolved into 300 ml dioxane, 31.2 g (0.06 mol) 4′-(3-bromophenyl)-1,1′:8′,1″-ternaphthalene, 2 g palladium acetate, 34 g potassium phosphate (0.16 mol), 8 g tris(2-methylphenyl)phosphine, 450 ml water, and 300 ml toluene were added and then warmed to 115° C. The TLC plate showed that the reaction was completed after reacting for 24 hour. The reaction liquid was extracted three times with dichloromethane. The organic phases were combined, dried, spin dried to give a crude product. 20 g white solid compound 13 was obtained by column chromatography with a yield of 70%.

### EXAMPLE 14

### Synthesis of Compound 14

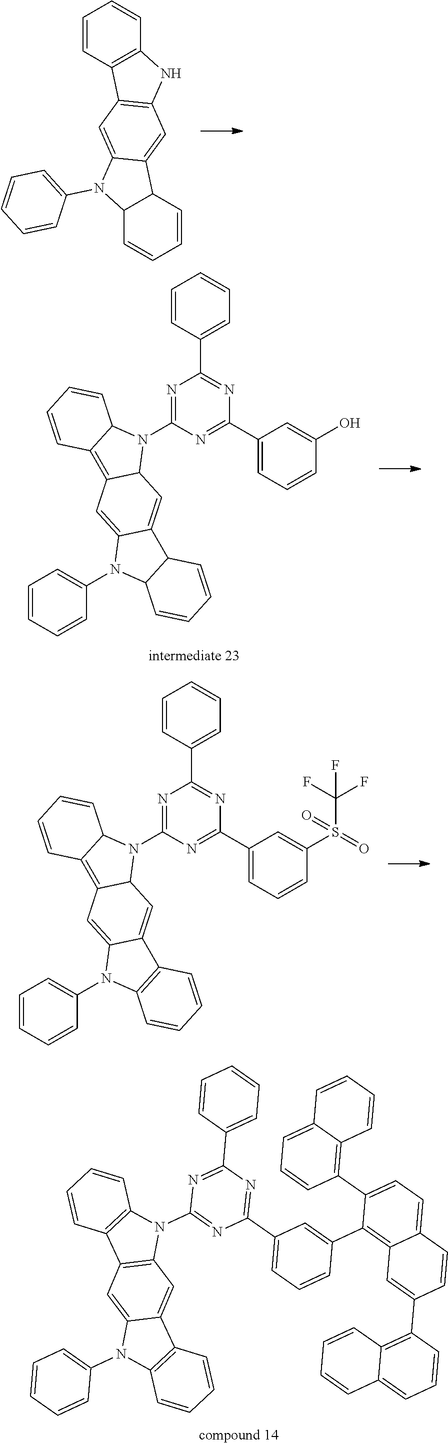

28 g (0.085 mol) 5-phenyl-4a,5,11,12b-tetrahydroindolo[3,2-b]carbazole was dissolved into 150 ml DMF at 0° C., 3.4 g (0.085 mol) NaH was added in batches. After keeping at this temperature for 30 minutes, 24 g 3-(4-chloro-6-phenyl-1,3,5-triazin-2-yl)phenol (0.085 mol) was added in batches and reacted at room temperature for 1 hour. Then this system was poured into water and extracted twice with dichloromethane, then dried, and spin dried. 27.6 g intermediate 23 was obtained by column chromatography with a yield of 56%.

27.6 g intermediate 23 was dissolved into 100 ml dichloromethane and cooled to 0° C. Trifluoromethanesulfonic anhydride was slowly added dropwise and kept at this temperature for 30 minutes. Then the reaction liquid was poured into water, and conducted liquid separation. The organic phase was dried and then spin dried to give a solid.

21.7 g (0.0473 mol) 1′-bromo-1,2′:7,1″-ternaphthalene was dissolved into THF, PdCl₂(dppf) and 8 g LiBr was added. After cooling to 0° C., the solid obtained in the previous step solved in THF was slowly added dropwise to this system. After the addition was completed, the temperature was raised to room temperature to react overnight. The reaction liquid was poured into water, extracted with dichloromethane, and then dried and spin dried. 15.6 g compound 14 was obtained by column chromatography with a yield of 35%.

### EXAMPLE 15

### Preparation of an Organic Formulation

A vial in which the stirrer was placed was cleaned and transferred to the glove box. 9.8 g 3-phenoxytoluene solvent was prepared in the vial. 0.19 g compound 6 and 0.01 g compound 8 were weighed in the glove box and added to the solvent system in the vial, and then stirred to mix. Stirring at 60° C. until the organic mixture was completely dissolved, and then cooling to room temperature. The resulted organic mixture solution was filtered through a 0.2 μm PTFE filter film. Sealing and Saving

The viscosity of the organic formulation was tested by a DV-I Prime Brookfield rheometer; the surface tension of the organic formulation was tested by a SITA bubble pressure tensiometer.

Though the above tests, the resulted organic formulation had a viscosity of 6.4±0.5 cPs and a surface tension of 34.1±0.5 dyne/cm.

### EXAMPLE 16

### Preparation of OLED Device

The preparation steps of an OLED device which contains ITO/HIL (50 nm)/HTL (35 nm)/EML (95 wt % compound 6:5 wt % compound 8) (25 nm)/ETL (28 nm)/LiF (1 nm)/Al (150 nm)/cathode were as follows:

a. Cleaning conductive glass substrate: when the conductive glass substrate is used for the first time, various solvents such as chloroform, ketone, and isopropyl alcohol can be used for cleaning, followed by UV ozone plasma treatment;

b. Preparing HIL (50 nm), HTL (35 nm), ETL (28 nm) by thermal evaporation in high vacuum (1×10⁻⁶ mbar); preparing EML (25 nm) by spin coating with solution;

c. Preparing a cathode by thermal evaporation LiF/Al (1 nm/150 nm) in high vacuum (1×10⁻⁶ mbar);

d. Packaging the device with ultraviolet curable resin in a nitrogen glove box.

The current-voltage (J-V) characteristics of each OLED device were characterized by characterization equipment while the important parameters such as efficiency, lifetime, and external quantum efficiency were recorded. Though the tests, the prepared blue light device had a color coordinate of (0.149, 0.083), a luminous efficiency of 6.7 cd/A, and a half life of 10,000 hours at 500 nits.

The above-mentioned organic functional compound used for preparing an organic electronic device includes an organic functional structural unit and a solubilizing structural unit, and has good solubility and film forming property, meanwhile, the organic functional compound well maintains performance of the functional structural unit thereof in the device. The organic functional compound, and the formulation, mixture and the like containing the organic functional compound, have good printability and film forming property, and facilitate the realization of high-performance small-molecule organic electronic device, especially organic electroluminescent device, by solution processing, especially printing processes, thereby providing a low-cost, high-efficiency technical solution for preparation.

Various technical features of the above embodiments can be combined in any manner. In order to make the description be concise, the present disclosure does not describe all the possible combinations of respective technical features of the above-mentioned embodiments. However, as long as combinations of these technical features do not contradict with one another, they should be regarded as being within the scope recorded in the present specification.

The foregoing examples merely show some embodiments of the present disclosure, which are described specifically and in detail, but they are not intended to limit the protection scope of the present disclosure. It should be noted that variations and improvements will become apparent to those skilled in the art without departing from the concept of the present disclosure, and these are all within the protection scope of the present disclosure. Therefore, the scope of the present disclosure is defined by the appended claims.

1. An organic functional compound for preparing an electronic device, wherein the compound has a general structural formula of
FSG]ₖ
wherein, F is an organic functional structural unit, SG is a solubilizing structural unit, k is an integer of 1-10; multiple SG are the same or different when k is greater than 1;
the solubilizing structural unit SG has a general structural unit of 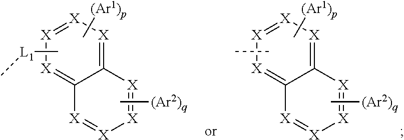
wherein, L₁, Ar¹ and Ar² are each independently selected from aryl or heteroaryl groups, p is an integer of 0-3, q is an integer of 0-4, and p+q≥2;
X is selected from N or CR¹, adjacent Xs are not simultaneously N, and X is C at the position where Ar¹ and Ar² are connected;
R¹ is selected from at least one of the following groups: H; D; linear alkyl containing 1-20 carbon atoms, linear alkoxy containing 1-20 carbon atoms or linear thioalkoxy containing 1-20 carbon atoms; branched or cyclic alkyl containing 3-20 carbon atoms, branched or cyclic alkoxy containing 3-20 carbon atoms or branched or cyclic thioalkoxy containing 3-20 carbon atoms; silyl; substituted ketone group containing 1-20 carbon atoms; alkoxycarbonyl containing 2-20 carbon atoms; aryloxycarbonyl containing 7-20 carbon atoms; cyano; carbamoyl; haloformyl; formyl; isocyano; isocyanate group; thiocyanate group; isothiocyanate group; hydroxy; nitro; CF₃; Cl; Br; F; crosslinkable group; substituted or unsubstituted aromatic or heteroaromatic ring system containing 5-40 ring atoms; aryloxy group containing 5-40 ring atoms or heteroaryloxy group containing 5-40 ring atoms; and any combination thereof; wherein one or more of the groups each is able to combine with the ring bonded thereto to form a monocyclic or polycyclic aliphatic or aromatic ring system.

2. The organic functional compound of claim 1, wherein the organic functional compound has a molecular weight of at least 600 g/mol.

3. The organic functional compound of claim 1, wherein the organic functional structural unit F is selected from groups consisting of: hole injection materials, hole transport materials, hole blocking materials, electron injection materials, electron transport materials, electron blocking materials, organic matrix materials, singlet emitters, triplet emitters, thermally activated delayed fluorescent materials and organic dyes.

4. The organic functional compound of claim 1, wherein the solubilizing structural unit SG is selected from groups shown by the following structural formulas: 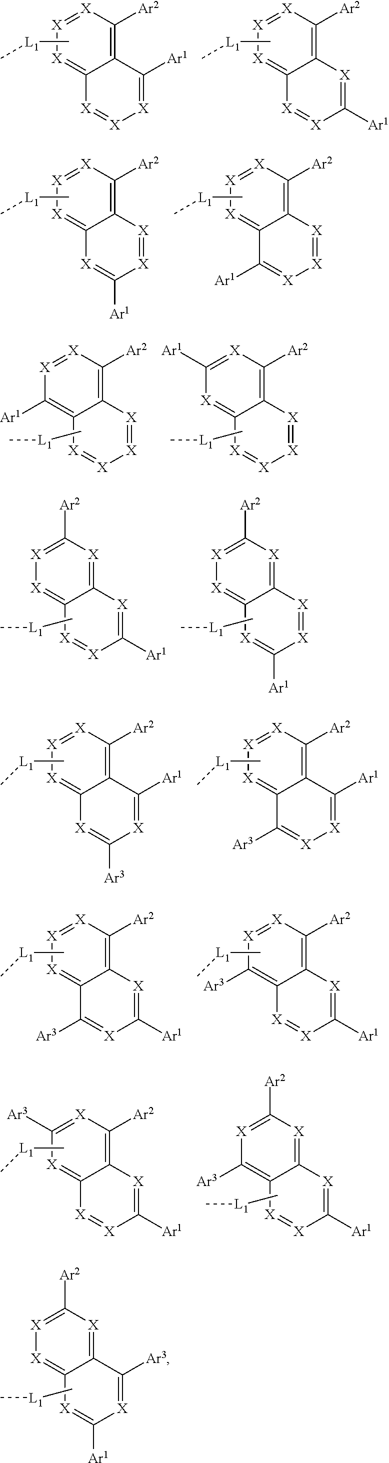
Ar³ is selected from aryl or heteroaryl groups.

5. The organic functional compound of claim 1, wherein the solubilizing structural unit SG is selected from groups shown by the following structural formulas: 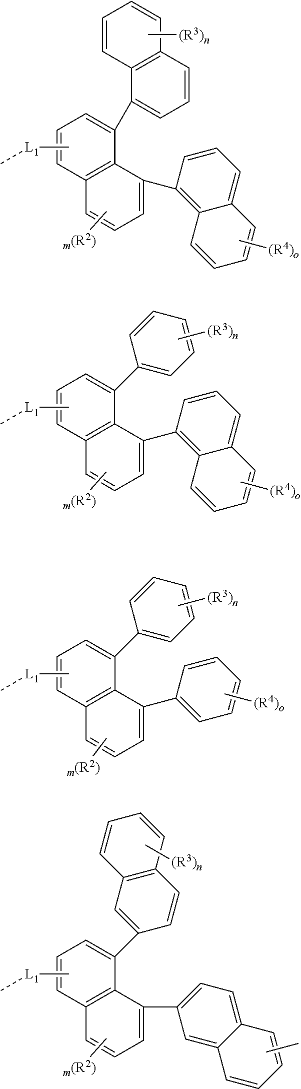 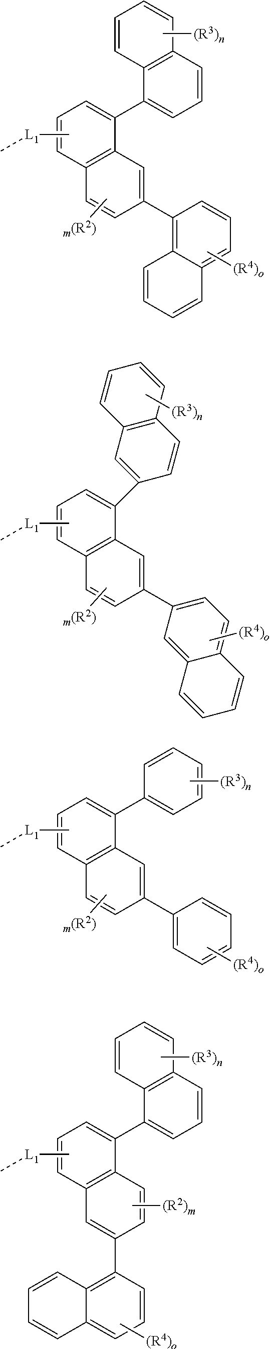 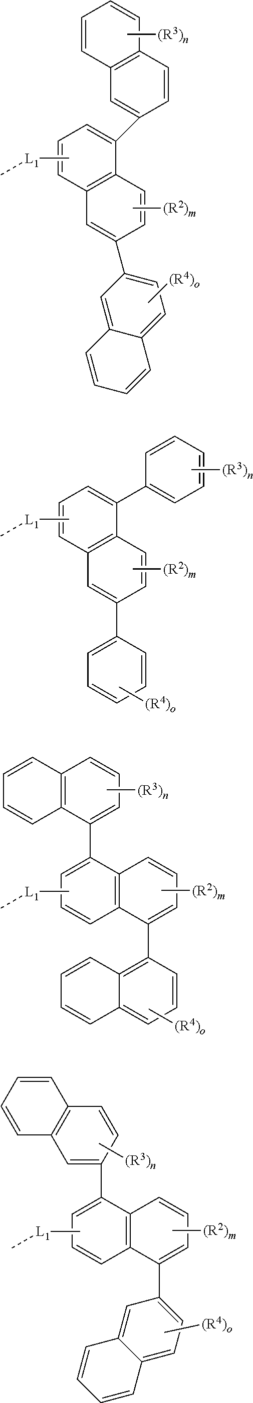 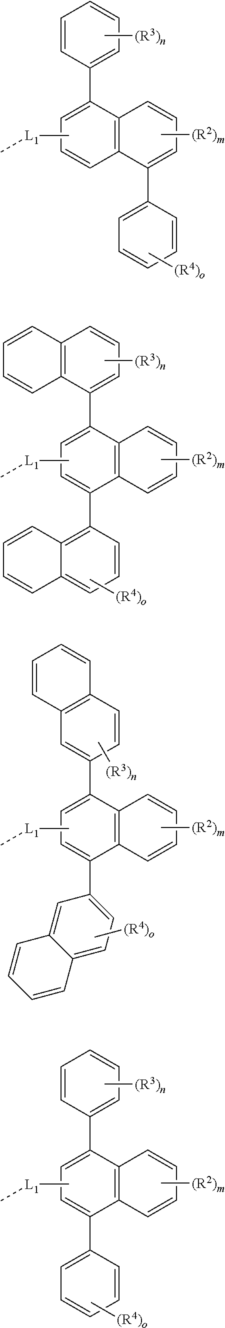 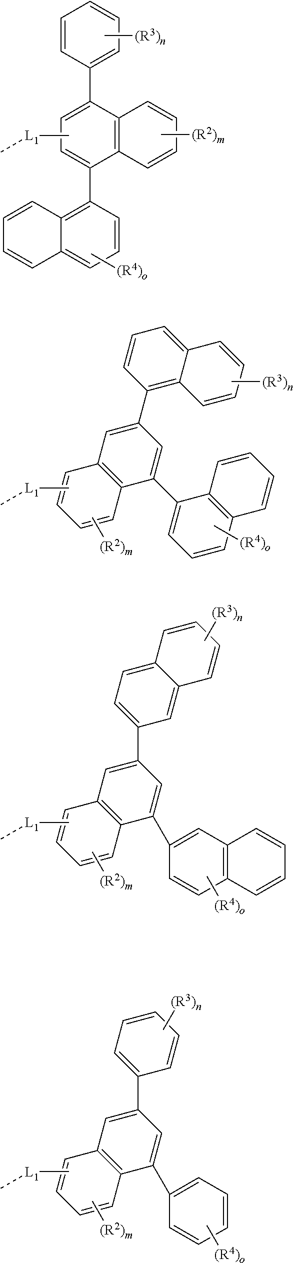 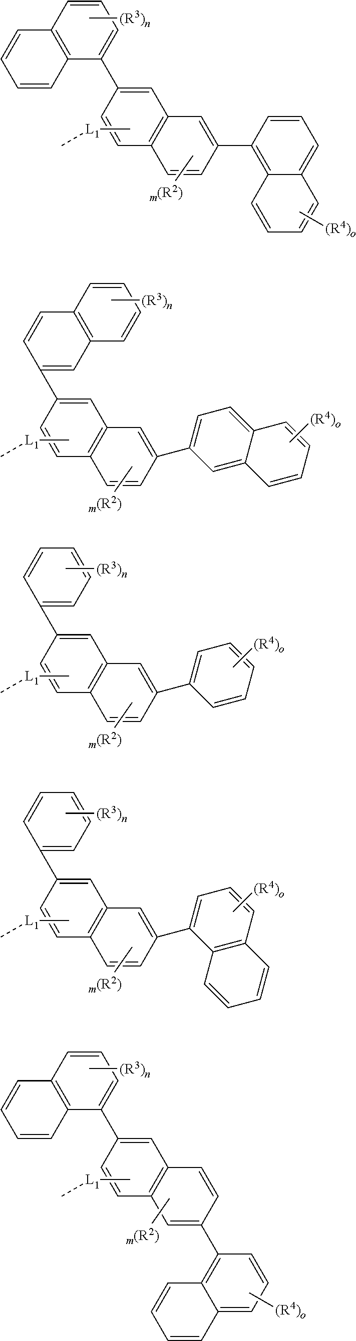 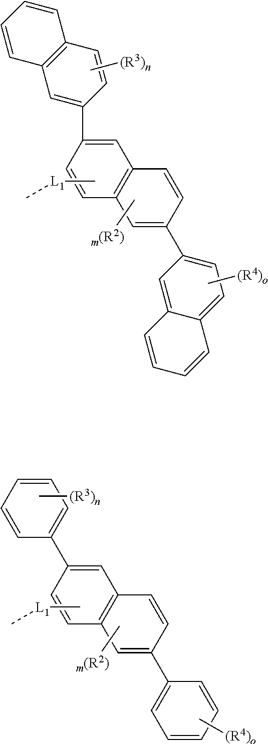
wherein, R², R³ and R⁴ are each independently selected from at least one of the following groups: H; D; linear alkyl containing 1-20 carbon atoms, linear alkoxy containing 1-20 carbon atoms or linear thioalkoxy containing 1-20 carbon atoms; branched or cyclic alkyl containing 3-20 carbon atoms, branched or cyclic alkoxy containing 3-20 carbon atoms or branched or cyclic thioalkoxy containing 3-20 carbon atoms; silyl; substituted ketone group containing 1-20 carbon atoms; alkoxycarbonyl group containing 2-20 carbon atoms; aryloxycarbonyl group containing 7-20 carbon atoms; cyano; carbamoyl; haloformyl; formyl; isocyano; isocyanate; thiocyanate; isothiocyanate; hydroxy; nitro; CF₃; Cl; Br; F; crosslinkable group; substituted or unsubstituted aromatic or heteroaromatic ring system containing 5-40 ring atoms; aryloxy group containing 5-40 ring atoms or heteroaryloxy group containing 5-40 ring atoms; and any combination thereof; wherein one or more of the groups each is able to combine with the ring bonded thereto to form a monocyclic or polycyclic aliphatic or aromatic ring system,
m is selected from 0, 1, 2, 3, 4 or 5; n and o are each independently selected from 0, 1, 2, 3, 4, 5, 6 or 7.

6. The organic functional compound of claim 1, wherein the total amount of SP³ hybridized groups in the organic functional compound is not more than 30% of the total molecular weight thereof.

7. The organic functional compound of claim 1, wherein the organic functional compound has a glass transition temperature of not less than 100° C.

8. The organic functional compound of claim 1, wherein weight ratio of the organic functional structural unit F to the solubilizing structural unit SG is (2:1)-(1:20).

9. A formulation for preparing an organic electronic device, comprising one organic functional compound according to claim 1 and one organic solvent.

10. The formulation of claim 9, wherein the organic functional compound is a host material.

11. The formulation of claim 9, wherein the formulation further comprises a light emitting material.

12. The formulation of claim 9, wherein the organic solvent is selected from at least one of the following group: aromatic compound, heteroaromatic compound, ester compound, aromatic ketone compound, aromatic ether compound, aliphatic ketone compound, aliphatic ether compound, alicyclic compound, olefin compound, and inorganic ester compound.

13. The formulation of claims 9, wherein the formulation has a viscosity in the range of 1 cPs to 100 cPs at 25° C.; and/or
the formulation has a surface tension in the range of 19 dyne/cm to 50 dyne/cm at 25° C.

14-15. (canceled)

16. An organic electronic device, comprising one organic functional compound of claim 1.

17. The organic electronic device of claim 16, wherein the organic electronic device is selected from groups consisting of an organic light-emitting diode, an organic photovoltaic cell, an organic light-emitting cell, an organic field effect transistor, an organic light-emitting field effect transistor, an organic laser, and an organic spintronic device, an organic sensor or an organic plasmon emitting diode.

18. (canceled)

19-20. (canceled)

21. The organic functional compound of claim 4, L1 is selected from the following structures:

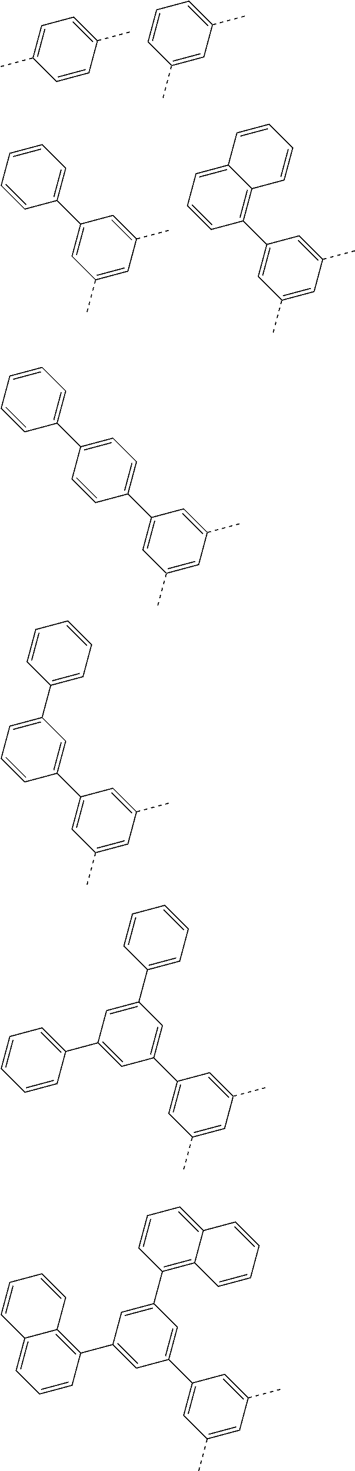

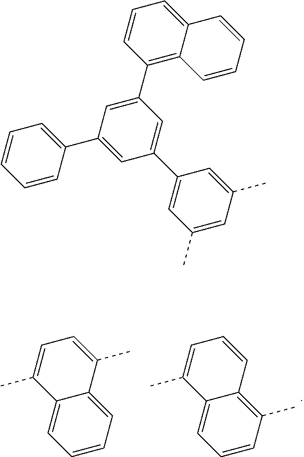

22. The formulation of claim 9, wherein the formulation comprises 0.01-20 wt % of the organic functional compound.

23. The formulation of claim 9, wherein the organic solvent is selected from group consisting of: 1-tetralone, 3-phenoxytoluene, acetophenone, 1-methoxynaphthalene, p-diisopropylbenzene, amylbenzene, tetrahydronaphthalene, cyclohexylbenzene, chloronaphthalene, 1,4-dimethylnaphthalene, 3-isopropylbiphenyl, p-methylisopropylbenzene, dipentylbenzene, o-diethylbenzene, m-diethylbenzene, p-diethylbenzene, 1,2,3,4-tetramethylbenzene, 1,2,3,5-tetramethylbenzene, 1,2,4,5-tetramethylbenzene, butylbenzene, dodecylbenzene 1-methylnaphthalene, 1,2,4-trichlorobenzene, 1,3-dipropoxybenzene, 4,4-difluorodiphenylmethane, diphenyl ether, 1,2-dimethoxy-4-(1-propenyl)benzene, diphenylmethane, 2-phenylpyridine, 3-phenylpyridine, 2-phenoxymethyl ether, 2-phenoxytetrahydrofuran, ethyl-2-naphthyl ether, N-methyldiphenylamine, 4-isopropylbiphenyl, α,α-dichlorodiphenylmethane, 4-(3-phenylpropyl)pyridine, benzyl benzoate, 1,1-bis(3,4-dimethylphenyl)ethane, 2-isopropylnaphthalene, and dibenzyl ether.

24. The organic electronic device of claim 16, wherein the organic electronic device comprises a substrate, an anode, and at least one light-emitting layer and a cathode.

25. The organic electronic device of claim 24, wherein the organic electronic device further includes at least one functional layer, and the function layer is selected from hole injection layer (HIL) or hole transport layer (HTL), electron blocking layer (EBL), electron injection layer (EIL) or electron transport layer (ETL), and hole blocking layer (HBL)
